(19) United States
(12) Patent Application Publication
(10) Pub. No.: US 2019/0310250 A1
(43) Pub. Date: Oct. 10, 2019

(54) METHODS FOR THE USE OF GALECTIN 3 BINDING PROTEIN DETECTED IN THE URINE FOR MONITORING THE SEVERITY AND PROGRESSION OF LUPUS NEPHRITIS

(71) Applicant: Merck Patent GmbH, Darmstadt (DE)

(72) Inventors: Lukas Shinji OKITSU, Arlington, MA (US); Jaromir VLACH, Redding, CT (US); Nuruddeen Lewis, Andover, MA (US); Julie DEMARTINO, Westfield, NJ (US); Roberto BASSI, San Rafael, CA (US); Wen-Rong LIE, St. Louis, MO (US)

(21) Appl. No.: 16/469,345

(22) PCT Filed: Dec. 18, 2017
(86) PCT No.: PCT/US17/67114

§ 371 Date: Jun. 13, 2019

Related U.S. Application Data
(60) Provisional application No. 62/435,235, filed on Dec. 16, 2016.

(51) Int. Cl. G01N 33/564; G01N 33/70

(52) U.S. Cl. CPC G01N 33/564; G01N 2800/347; G01N 2800/104; G01N 33/70

(57) ABSTRACT

Embodiments of the present invention describe compositions and methods incorporating the measurement of LGALS3BP in the urine of patients diagnosed with lupus nephritis (LN) in order to monitor the severity and progression of said LN.

### PRIORITY CLAIM

This application claims the benefit of U.S. Provisional Application Ser. No. 62/435,235, filed on Dec. 16, 2016, which is, hereby, incorporated by reference.

The instant application contains a Sequence Listing which has been submitted electronically in ASCII format and is, hereby, incorporated by reference in its entirety. Said ASCII copy, created on Dec. 15, 2017, is named P16-214WO_SL.txt and is 433,834 bytes in size.

### FIELD OF THE INVENTION

The invention relates generally to the detection of LGALS3BP in urine within methodologies for detecting and monitoring the progression of lupus nephritis (LN).

### BACKGROUND OF THE INVENTION

Systemic lupus erythematosus (SLE) is an autoimmune disorder characterized by the formation of autoantibody-containing immune complexes (ICs) that trigger inflammation, tissue damage, and premature mortality (Tsokos G C, N Engl J Med (2011); 365:2110-2121). SLE ICs often contain nucleic acids that are recognized by numerous innate immune receptors that can initiate pathological mechanisms leading to production of cytokines, and ultimately to immune responses leading to organ damage. Due to the great clinical diversity and idiopathic nature of SLE, management of SLE depends on its specific manifestations and severity. Therefore, medications suggested to treat SLE are not necessarily effective for the treatment of all manifestations and complications such as lupus nephritis (LN). The pathogenesis of LN is believed to derive from deposition of immune complexes in the kidney glomeruli that initiates an inflammatory response causing kidney damage (Davidson A2016, Nature Reviews Rheumatology 12:143-153). An estimated 30-60% of patients with SLE develop nephritis over the course of their disease that requires medical evaluation and treatment. LN is a progressive disease, running a course of clinical exacerbations and remissions. Late stage LN is characterized by irreversible scarring in the kidney, which cannot be treated with current SLE drugs, necessitating a kidney transplant (Lionaki S et al., World Journal of Transplantation, 2014, 4(3): 176-182).

General indications of lupus nephritis are foamy or bloody urine due to compromised kidney filtering function leading to high urinary protein concentration. Lupus nephritis is diagnosed by kidney biopsy (Schwartz N et al., Curr Opin Rheumatol. 2014). Renal function can be measured by blood urea nitrogen (BUN) testing, serum creatinine assessment, urinalysis (total protein, red blood cells and cellular casts), spot urine test for creatinine and protein concentration, or 24-hour urine test for creatinine clearance and protein excretion. Proper monitoring of kidney disease in LN is currently not possible as biopsies are invasive and usually only performed for initial diagnosis. Although kidney function can be approximated using current tests, they all fail to estimate the level of causal inflammation (Zickert A, et al., Lupus Sci Med 2014, 1:e000018; Alvarado et al. Lupus 2014, 23: 840). Without the ability to assess the inflammatory state of the kidney, physicians cannot accurately assess the effectiveness of their treatments, as these treatments are directed to resolve the ongoing inflammation. Accurate monitoring of the causal inflammation in the kidney could help physicians with aggressive treatment decisions and a treat-to-target approach, thereby slowing disease progression, improving patient's lives, and lowering health care costs by preventing the need for expensive kidney transplants.

SLE is treated with antimalarials, corticosteroids, non-steroidal anti-inflammatory drugs (NSAIDs), immunosuppressants and biologics such as Belimumab (BAFF neutralization) and Rituximab (B cell depletion). While many patients fail to respond or respond only partially to the standard of care medications listed above, the long-term use of high doses of corticosteroids and cytotoxic therapies may have profound side effects such as bone marrow suppression, increased infections with opportunistic organisms, irreversible ovarian failure, alopecia, and increased risk of malignancy. Infectious complications coincident with active SLE and its treatment with immunosuppressive medications are the most common cause of death in patients with SLE. Therefore, there is a need for alternative diagnostics, which can better provide a definitive diagnosis of SLE/LN and monitor disease activity to allow more targeted aggressive treatment with fewer side effects.

Galectin-3 binding protein [other aliases include: LGALS3BP (and all related polymorphisms), uG3BP, G3BP, Mac2-BP, p90, Lectin Galactoside-Binding Soluble 3 Binding Protein, BTBD17B, CyCAP, gp90, L3 antigen, M2BP, Mac-2-binding protein, MAC-2-BP and TANGO10B] is the gene product of a ubiquitously expressed gene that belongs to the scavenger receptor family (Koths, K. et al. 1993 J. Biol. Chem. 268:14245). The 585 amino acid (aa) human protein contains an 18 aa signal sequence and four domains (Hohenester, E. et al. 1999 Nat. Struct. Biol. 6:228; Muller, S. A. et al. 1999 J. Mol. Biol. 291:801; Hellstern, S. et al. 2002 J. Biol. Chem. 277:15690). Domain 1 is a group A scavenger receptor domain, domain 2 is a BTB/POZ domain that strongly mediates dimerization, and domain 3 is an IVR domain, that is also found following the POZ domain in Drosophila Kelch protein. Although little is known about domain 4, recombinant domains 3 and 4 reproduce the solid-phase adhesion profile of full-length Galectin-3BP. Glycosylation at seven N-linked sites, generates a molecular size of 85-97 kDa (Ullrich, A. et al. (1994) J. Biol. Chem. 269:18401). Galectin-3BP dimers form linear and ring-shaped oligomers, most commonly decamers and dodecamers. LGALS3BP is a protein secreted by certain types of tumor cells wherein expression levels correlate with tumor progression (Grassadonia, A. et al. 2004 Glycoconj. J. 19:551). Apart from its direct effect on tumor cell proliferation/survival, LGALS3BP can also upregulate expression of vascular endothelial growth factor and promote angiogenesis. Its levels are augmented during HIV-1 infection and its activity is believed to reduce infectivity of HIV-1 through interference with the maturation and incorporation of envelope proteins into virions (Lodermeyer V et al. Retrovirology. 2013 24; 10:111). Serum levels of LGALS3BP are increased in patients with Behcet's disease and correlate with disease activity (Lee Y J et al. Clin Exp Rheumatol. 2007 25(4 Suppl 45):541-5). Increased levels of plasma LGALS3BP are also observed in certain cohorts of SLE patients (Nielsen C T et al. Lupus Sci Med. 2014 19; 1(1)). LGALS3BP has an IRF7 regulatory element in its promoter (Heinig M et al. Nature. 2010 23; 467(7314):460-4) indicating regulation by type I IFN and explaining its link to viral infections and inflammation.

There is an urgent, yet still unmet, need for use in clinical medicine and biomedical research for improved non-invasive tools to: i) identify if SLE is about to manifest as LN, ii) evaluating changes in renal pathophysiology in LN in subjects already diagnosed with LN and iii) evaluating disease progression/regression in subject already diagnosed with LN.

### SUMMARY OF THE INVENTION

The present invention provides compositions and methods of assessing the present and ongoing renal inflammation status in a mammalian subject with or at a risk of developing LN, by detecting the quantity (e.g., determining the level) of Galectin-3 binding protein (LGALS3BP) in a body fluid sample. The present invention also provides a method of monitoring the effectiveness of a treatment for renal pathophysiology in LN by determining the level of LGALS3BP in the body fluid before and in particular after treatments designed to treat flares associated with LN. The properties and characteristics of LGALS3BP as a predictive marker allow for its use in this manner for the early detection of renal pathophysiology in LN or changes in renal pathophysiology in LN status in the context of LN.

In one embodiment, the present invention provides a method for the early detection of a renal pathophysiology in LN in a mammal, comprising the steps of: i) obtaining or providing a sample of a body fluid from a mammal that is not experiencing an acute renal disease in LN, the body fluid selected from the group consisting of urine, plasma, and serum; ii) detecting (e.g., determining) the level of LGALS3BP in the sample (e.g., using an antibody against LGALS3BP); and iii) evaluating the renal pathophysiology in LN status of the subject, based on the level of LGALS3BP in the sample. The evaluation of the renal pathophysiology in LN status can be used to determine whether the renal pathophysiology in LN is sub-clinical, stable, or progressing (i.e., progressive renal disease). The method also provides an evaluation of the renal status as a progressive or worsening renal pathophysiology in LN with only a single sampling and assay.

In one embodiment the present invention provides a method for the detection of any change in a renal pathophysiology in LN status of a mammal, comprising the steps of: i) obtaining a first sample of a body fluid from a mammal exhibiting at least one symptom of SLE, the body fluid selected from the group consisting of urine, plasma, and serum (in a preferred embodiment said body fluid is urine); ii) detecting (e.g., determining) the level of LGALS3BP in the first sample (e.g., using an antibody against LGALS3BP); iii) obtaining at least one subsequent sample of the body fluid from the mammal after a period of time after obtaining the first sample; iv) detecting (e.g., determining) the level of LGALS3BP in at least one subsequent sample (e.g., using an antibody against LGALS3BP); and v) evaluating the renal pathophysiology in LN status of the mammal, based on comparing the level of LGALS3BP in the at least one subsequent sample to the level of LGALS3BP in the first sample. Generally, a higher level of LGALS3BP in the subsequent sample is an indication of the worsening renal pathophysiology in LN status in the subject demonstrating at least one symptom of SLE which indicates the imminent progression of SLE into LN, while a similar or reduced level of LGALS3BP in the subsequent sample is an indication of an improvement in the renal pathophysiology in LN status and an indicator SLE of said subject is not about to progress into LN.

In one embodiment the present invention provides a method of monitoring the effectiveness of a treatment for renal pathophysiology in LN in a mammal, comprising the steps of: i) providing or obtaining a baseline sample of a body fluid from a mammal experiencing at least one symptom of LN, the body fluid selected from the group consisting of urine, plasma, and serum (in a preferred embodiment said body fluid is urine); ii) detecting (e.g., determining) the level of LGALS3BP in the baseline sample (e.g., using an antibody against LGALS3BP); iii) providing at least one treatment for the renal pathophysiology in LN to the mammal; iv) providing or obtaining at least one post-treatment sample of the body fluid from the mammal; v) detecting (e.g., determining) the level of LGALS3BP in the post-treatment sample (e.g., using an antibody against LGALS3BP); and vi) evaluating the effectiveness of the treatment, based on comparing the level of LGALS3BP in the post-treatment sample to the level of LGALS3BP in the baseline sample.

One embodiment of the present invention provides a method of identifying the extent of renal pathophysiology in LN in a mammal over time, comprising the steps of: i) obtaining at least one first sample of a body fluid at a first time from a mammal that is experiencing at least one symptom of LN, the body fluid selected from the group consisting of urine, plasma, and serum (in a preferred embodiment said body fluid is urine); ii) detecting (e.g., determining) the level of LGALS3BP in the first sample (e.g., using an antibody against LGALS3BP); iii) obtaining at least one subsequent sample of the body fluid at a time subsequent to the first time, from the mammal; iv) detecting (e.g., determining) the level of LGALS3BP in at least one subsequent sample (e.g., using an antibody against LGALS3BP); and v) determining the extent of the renal pathophysiology in LN in the mammal over time, based on comparing the level of LGALS3BP in at least one subsequent sample to the level of LGALS3BP in the first sample. Typically, the mammalian subject is a human. Where more than one subsequent sample is drawn, they are typically obtained and provided intermittently from the subject, and at predetermined times, ranging from one or more days, to one or more weeks, to one or more months, to one or more years. Other sampling regimens also may be employed. In one embodiment, the mammalian subject is also evaluated to determine if the subject is experiencing another condition that may contribute to the level of LGALS3BP in the sample, such condition including, but limited to, an acute bacterial or viral infection, acute inflammation, an acute or chronic injury to another organ or cancer. Such another condition may not effect or cause an injury to the kidney. However, such condition on its own can contribute the amount of LGALS3BP detected in the urine, making it difficult to distinguish such LGALS3BP from LGALS3BP that is expressed as a direct result of a renal pathophysiology in LN. Some types of other conditions can effect high levels of LGALS3BP that can overwhelm the concentration of LGALS3BP resulting from the renal injury.

A variety of protein detection formats are contemplated, including, but not limited to, ELISA (enzyme linked immunosorbent assay), SMC immunoassay technology (Single Molecule Counting) and Western Blot.

In some embodiments assay devices, in particular ELISA devices, comprise coated microtiter plates. In some embodiments, a capture reagent (i.e., LGALS3BP antibody) is applied in the wells of a microtiter plate. In this assay, a test sample (e.g., blood or urine) potentially containing an analyte of interest (e.g., LGALS3BP) is placed in the wells of a microtiter plate that contain the immobilized capture reagent. The analyte specifically binds the immobilized antibody; then, unbound materials are washed away leaving primarily the analyte-antibody complex bound to the plate. This complex can be detected in a variety of manners, such as by use of a labelled detector reagent, e.g., labeled LGALS3BP antibody. One advantage of the microtiter plate format is that multiple samples can be tested simultaneously (together with controls) each in one or more different wells of the same plate; thus, permitting high-throughput analysis of numerous samples.

In some embodiments, a competitive ELISA assay is utilized (see e.g., U.S. Pat. Nos. 5,958,715, and 5,484,707, each of which is herein incorporated by reference). The competitive ELISA may be quantitative or non-quantitative. In a competitive ELISA, the wells of a microtiter plate are first coated with a fusion protein comprising all or a fragment of LGALS3BP. The sample to be tested is added to the plate along with an antibody that is specific for LGALS3BP. The LGALS3BP in the sample competes for binding to the antibody with the immobilized peptide. The plate is washed and the antibody bound to the immobilized LGALS3BP polypeptide is then detected using any suitable method (e.g., a secondary antibody comprising a label or a group reactive with an enzymatic detection system). The amount of signal is inversely proportional to the amount of LGALS3BP present in the sample (e.g., a high signal is indicative of low amounts of LGALS3BP being present in the sample).

In some embodiments, the immunoassay devices of the present invention permit the performance of relatively inexpensive, disposable, membrane-based assays for the visual identification of the presence (or absence) of an analyte in a liquid sample. Such devices are usually formatted as freestanding dipsticks (e.g., test strips) or as devices having some sort of housing. Typically, an immunoassay device of the present invention can be used with as little as about 200 microliters of liquid sample, and detection of an analyte in the sample can (but need not) be complete within 2-5 minutes. In preferred embodiments, no ancillary instrumentation is required to perform such tests, and such devices easily can be used in clinics, laboratories and field locations.

In some embodiments, the ELISA is an immunochromatographic “sandwich” assay. In general, sandwich immunochromatographic procedures call for mixing the sample that may contain the analyte to be assayed for example, LGALS3BP, with an antibody specific for LGALS3BP. The antibody, i.e., detector reagent, is mobile and typically is linked to a label or another signaling reagent, such as dyed latex, a colloidal metal sol, or a radioisotope. This mixture is then applied to a chromatographic medium containing a band or zone of immobilized antibodies that recognize LGALS3BP (i.e., the capture antibody or reagent). The chromatographic medium often is in the form of a strip that resembles a dipstick. When the complex of LGALS3BP and the detector reagent reaches the zone of the immobilized capture antibody on the chromatographic medium, binding occurs and the detector reagent complex is localized at the zone. This indicates the presence of the molecule to be assayed. This technique can be used to obtain quantitative or semi-quantitative results. Examples of sandwich immunoassays performed on test strips are described in U.S. Pat. Nos. 4,168,146 and 4,366,241, each of which is incorporated herein by reference.

In some embodiments a “Western blot” format is used to detect proteins of interest. Western Blot refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

In another embodiment of the present invention, there is provided a method for generating a result useful in diagnosing and non-invasively monitoring renal pathology using samples obtained from a mammalian subject. The method includes: obtaining a dataset associated with the samples, wherein the dataset comprises protein expression levels for markers selected from the group consisting of: urinary creatinine and proteinuria expressed as a ratio of urine protein: creatinine (uPCR); and inputting the dataset into an analytical process that uses the data to generate a result useful in diagnosing and monitoring the renal pathology.

In some embodiments, the definition of lupus nephritis comprises one or more of: lupus nephritis, idiopathic immune-complex glomerulonephritis, glomerular nephritis, tubulo-interstitial nephritis.

In some embodiments, the diagnostic aspects of the present invention can better inform when invasive kidney biopsies and/or changes in therapeutic regimes should be considered. A diagnostic kidney biopsy should be done to guide therapy when a lupus patient presents with clinical evidence of new kidney inflammation such as the detection of increased levels of LGALS3BP as provided by the diagnostic embodiments of the present invention.

In some embodiments renal classification of lupus nephritis comprises one or more of:

Class I disease (minimal mesangial glomerulonephritis) in its histology has a normal appearance under a light microscope, but mesangsial deposits are visible under an electron microscope. At this stage urinalysis is normal.

Class II disease (mesangial proliferative glomerulonephritis) is noted by mesangial hypercellularity and matrix expansion. Microscopic hematuria with or without proteinuria may be seen. Hypertension, nephrotic syndrome, and acute kidney insufficiency are very rare at this stage.

Class III disease (focal glomerulonephritis) is indicated by sclerotic lesions involving less than 50% of the glomeruli, which can be segmental or global, and active or chronic, with endocapillary or extracapillary proliferative lesions. Under the electron microscopy, subendothelial deposits are noted, and some mesangial changes may be present. Immunofluorescence reveals positively for IgG, IgA, IgM, C3, and C1q (indicative of immune complex deposits). Clinically, hematuria and proteinuria are present, with or without nephrotic syndrome, hypertension, and elevated serum creatinine. Diffuse proliferative lupus nephritis as seen in a pathology specimen.

Class IV disease (diffuse proliferative nephritis) is both the most severe, and the most common subtype. More than 50% of glomeruli are involved. Lesions can be segmental or global, and active or chronic, with endocapillary or extracapillary proliferative lesions. Under electron microscopy, subendothelial deposits are noted, and some mesangial changes may be present. Clinically, hematuria and proteinuria are present, frequently with nephrotic syndrome, hypertension, hypocomplementemia, elevated anti-dsDNA titers and elevated serum creatinine.

Class V disease (membranous glomerulonephritis) is characterized by diffuse thickening of the glomerular capillary wall (segmentally or globally), with diffuse membrane thickening, and subepithelial deposits seen under the electron microscope. Clinically, stage V presents with signs of nephrotic syndrome. Microscopic hematuria and hypertension may also been seen. Stage V also can also lead to thrombotic complications such as renal vein thromboses or pulmonary emboli.

Class VI, or advanced sclerosing lupus nephritis. It is represented by global sclerosis involving more than 90% of glomeruli, and represents healing of prior inflammatory injury. Active glomerulonephritis is not usually present. This stage is characterized by slowly progressive kidney dysfunction, with relatively bland urine sediment. Response to immunotherapy is usually poor. A tubuloreticular inclusion within capillary endothelial cells is also characteristic of lupus nephritis, and can be seen under an electron microscope in all stages. It is not diagnostic however, as it exists in other conditions such as HIV infection. It is thought to be due to the chronic interferon exposure.

As reported in the data presented in the instant application, unless otherwise stated, LGALS3BP is measured in ng/ml. LGALS3BP/creatinine ratios are ng LGALS3BP/mg creatinine per ml of urine.

In some embodiments, the renal pathophysiology in LN of lupus nephritis comprises one or more of: presence of mesangial immune deposits, presence of sub-endothelial immune deposits, presence of sub-epithelial immune deposits, tubulo-interstitial inflammation, tubulo-interstitial fibrosis, tubulo-interstitial sclerosis, sclerosis, crescentic glomerulonephritis (presence of crescentic lesions or extracapillary proliferation), extracapillary proliferation, endocapillary proliferation, proliferative glomerulonephritis, focal glomerulopathy (or focal glomerulonephritis), focal segmental glomerulopathy (or focal segmental glomerulonephritis), segmental glomerulopathy (or segmental glomerulonephritis), membranous glomerulopathy, glomerular basement membrane abnormalities (such as thickening), glomerulosclerosis (or glomerular sclerosis), mesangial hypercellularity (or mesangial proliferation), mesangial matrix expansion, mesangial fibrosis.

In some embodiments, the analytical process is a Linear Discriminant Analysis model. Further, in some embodiments, the analytical process can include use of a predictive model. In some embodiments, the analytical process comprises comparing the obtained dataset with a reference dataset.

In some embodiments, the reference dataset comprises protein expression levels obtained from one or more healthy control subjects. In other embodiments, the method further comprises obtaining a statistical measure of a similarity of the obtained dataset to the reference dataset.

In some embodiments, the method further comprises using the classification for diagnosis, staging, prognosis, kidney inflammation levels, assessing extent of progression, monitoring a therapeutic response, predicting a renal-interstitial inflammation (INF) episode, or distinguishing stable from unstable manifestations of renal-interstitial inflammation (INF) in subjects presenting at least one symptom of LN.

### BRIEF DESCRIPTION OF THE DRAWINGS

FIG. 1 shows LGALS3BP mRNA expression levels in PBMCs isolated from HC and LN patients with low or high IFN-a signature.

FIG. 2A presents data showing that LGALS3BP is induced by inflammatory stimuli including but not limited to IFN-a with LGALS3BP expression by QPCR using RNA extracted from in vitro differentiated primary human macrophages activated with indicated stimuli for 6 h. Expression between samples was normalized using HPRT1 as a housekeeping gene.

FIG. 2B presents additional data showing that LGALS3BP is induced by inflammatory stimuli including but not limited to IFN-a with LGALS3BP measured by ELISA in supernatants of in vitro differentiated primary human macrophages activated with indicated stimuli for 20 h.

FIG. 3 shows LGALS3BP protein levels in serum, urine and plasma. LGALS3BP plasma and urine levels were measured in healthy control donors, SLE and LN patients by ELISA. Urinary LGALS3BP protein levels were significantly higher (P<0.0001, 1-way Anova with Tukey post test) in LN patients vs SLE patients or healthy controls. This difference is not noted in serum obtained from the same subjects. No linear correlation exist between plasma and urine levels.

FIG. 4A shows gene expression levels of LGALS3BP in the glomeruli and tubulointerstitium of kidney tissue sections from HC and LN patients. A total of 46 samples (n=14 HC and 32 LN) from the European Renal cDNA Bank were processed and used for microarray analysis as described (Berthier et al., JI 2012). Biopsy sections were manually micro dissected into glomerular and tubulointerstitial compartments and gene expression profiling was performed using the Human Genome U133A Affymetrix GeneChip arrays, wherein, gene expression levels for LGALS3BP were significantly higher in both the glomeruli (p=9.221e-12) and the tubulointerstitium (p=1.511e-4) as compared to HC.

FIG. 4B shows gene expression levels of CCL2 (MCP-1) in the glomeruli and tubulointerstitium of kidney biopsies from HC and LN patients. A total of 46 samples (n=14 HC and 32 LN) from the European Renal cDNA Bank were processed and used for microarray analysis as described (Berthier et al., JI 2012). Biopsy sections were manually microdissected into glomerulus and tubulointerstitial compartments and gene expression profiling was performed using the Human Genome U133A Affymetrix GeneChip arrays, wherein, gene expression levels for CCL2 (MCP-1) were not equivalent between HC and LN samples in both the glomeruli and tubulointerstitium.

FIG. 4C shows gene expression levels of TNFSF12 in the glomeruli and tubulointerstitium of kidney biopsies from HC and LN patients. A total of 46 samples (n=14 HC and 32 LN) from the European Renal cDNA Bank were processed and used for microarray analysis as described (Berthier et al., JI 2012). Biopsy sections were manually microdissected into glomerular and tubulointerstitial compartments and gene expression profiling was performed using the Human Genome U133A Affymetrix GeneChip arrays, wherein, TNFSF12 gene expression levels were significantly higher in LN glomeruli (p=0.017) but significantly lower in tubuolointerstitium (p=9.08e-5).

FIG. 4D shows galectin 3 binding protein expression in kidney biopsies from healthy volunteers (HC), LN patients with and without tubulointerstitial nephritis (TIN), diabetes mellitus (DM) and IgA nephropathy (IgAN) patients. Galectin 3 binding protein (light areas), was stained with antibodies analyzed by fluorescence microscopy.

FIG. 5 shows LGALS3BP mRNA expression in the BXSB-Yaa LN mouse model. Diseased mice were euthanized at 20 weeks of age and kidney LGALS3BP expression analyzed by NanoString and normalized to hprt1 expression. Control mice are young (9 weeks) BXSX-Yaa mice before onset of disease. Kidney damage was assessed by histology.

FIG. 6A shows total LGALS3BP normalized to urinary creatinine ratios in the urine of healthy controls (HC), lupus nephritis (LN), and systemic lupus erythematosus (SLE) donors.

FIG. 6B shows total protein to creatinine ratios in the urine of healthy controls (HC), lupus nephritis (LN), and systemic lupus erythematosus (SLE) donors.

FIG. 6C shows urinary albumin to creatinine ratios in the urine of healthy controls (HC), lupus nephritis (LN), and systemic lupus erythematosus (SLE) donors.

FIG. 7A shows correlations of urinalysis measurements, wherein, albumin to creatinine ratios and total protein to creatinine ratios correlated well to one another with a correlation coefficient of 0.95.

FIG. 7B shows correlations of urinalysis measurements, wherein, LGALS3BP to creatinine ratios positively correlate with total protein to creatinine ratios (R=0.494).

FIG. 7C shows correlations of urinalysis measurements, wherein, LGALS3BP to creatinine ratios positively correlate with albumin to creatinine ratios (R=0.484).

FIG. 8A shows changes in urinary protein measurements in patients across multiple visits. All values are presented as normalized to creatinine levels. Each dot represents a sample and each line represents a donor. The color of the line represents the disease group with LN samples colored purple, SLE samples colored cyan, and HC samples colored dark gray.

FIG. 8B shows changes in albumin measurements in patients across multiple visits. All values are presented as normalized to creatinine levels. Each dot represents a sample and each line represents a donor. The color of the line represents the disease group with LN samples colored purple, SLE samples colored cyan, and HC samples colored dark gray.

FIG. 8C shows changes in LGALS3BP measurements in patients across multiple visits. All values are presented as normalized to creatinine levels. Each dot represents a sample and each line represents a donor. The color of the line represents the disease group with LN samples colored purple, SLE samples colored cyan, and HC samples colored dark gray.

FIG. 9 shows binding curves of selected anti-LGALS3BP monoclonal antibodies. Serial dilutions of monoclonal antibodies identified in antibody phage library screens were tested for binding in an ELISA using microtiter plates coated with full length recombinant human LGALS3BP. Monoclonal antibody binding to plate-bound LGALS3BP was detected with a secondary anti-Ig antibody conjugated to horseradish peroxidase (HRP). Binding was revealed using HRP substrate and optical density was measured at 450 nm.

FIG. 10A and FIG. 10B show anti-LGALS3BP monoclonal antibody pairing for sandwich ELISA. 100 ng/mL recombinant LGALS3BP (FIG. 10B) was used as analyte and compared to buffer only control (FIG. 10A). Antibodies were conjugated to beads and tested in a multiplex Luminex assay to determine best pairs. Each antibody was detected in a different channel allowing the evaluation of the pairs in the same environment. Values are arbitrary units from the Luminex reader. Columns are capture antibodies, rows are detection antibodies.

FIG. 11A shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb1-mAb9). LGALS3BP concentrations are in ng/ml for urine samples from healthy controls (healthy), lupus nephritis patients (LN) and extrarenal systemic lupus erythematosus (SLE) patients.

FIG. 11B shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb3-mAb11). LGALS3BP concentrations are in ng/ml for urine samples from healthy controls (healthy), lupus nephritis patients (LN) and extrarenal systemic lupus erythematosus (SLE) patients.

FIG. 11C shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb3-mAb22). LGALS3BP concentrations are in ng/ml for urine samples from healthy controls (healthy), lupus nephritis patients (LN) and extrarenal systemic lupus erythematosus (SLE) patients.

FIG. 11D shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb114-mAb116). LGALS3BP concentrations are in ng/ml for urine samples from healthy controls (healthy), lupus nephritis patients (LN) and extrarenal systemic lupus erythematosus (SLE) patients.

FIG. 12A shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb103-mAb116). LGALS3BP concentrations are in ng/ml for urine samples from healthy controls (healthy), lupus nephritis patients (LN) and extrarenal systemic lupus erythematosus (SLE) patients.

FIG. 12B shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb109-mAb116). LGALS3BP concentrations are in ng/ml for urine samples from healthy controls (healthy), lupus nephritis patients (LN) and extrarenal systemic lupus erythematosus (SLE) patients.

FIG. 12C shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb110-mAb116). LGALS3BP concentrations are in ng/ml for urine samples from healthy controls (healthy), lupus nephritis patients (LN) and extrarenal systemic lupus erythematosus (SLE) patients.

FIG. 12D shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb112-mAb116). LGALS3BP concentrations are in ng/ml for urine samples from healthy controls (healthy), lupus nephritis patients (LN) and extrarenal systemic lupus erythematosus (SLE) patients.

FIG. 13A shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb105-mAb116). LGALS3BP concentrations are in ng/ml for urine samples from healthy controls (healthy), lupus nephritis patients (LN) and extrarenal systemic lupus erythematosus (SLE) patients.

FIG. 13B shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb29-mAb116). LGALS3BP concentrations are in ng/ml for urine samples from healthy controls (healthy), lupus nephritis patients (LN) and extrarenal systemic lupus erythematosus (SLE) patients.

FIG. 13C shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb113-mAb116). LGALS3BP concentrations are in ng/ml for urine samples from healthy controls (healthy), lupus nephritis patients (LN) and extrarenal systemic lupus erythematosus (SLE) patients.

FIG. 13D shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb102-mAb103). LGALS3BP concentrations are in ng/ml for urine samples from healthy controls (healthy), lupus nephritis patients (LN) and extrarenal systemic lupus erythematosus (SLE) patients.

FIG. 14A shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb103-mAb103). LGALS3BP concentrations are in ng/ml for urine samples from healthy controls (healthy), lupus nephritis patients (LN) and extrarenal systemic lupus erythematosus (SLE) patients.

FIG. 14B shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb109-mAb103). LGALS3BP concentrations are in ng/ml for urine samples from healthy controls (healthy), lupus nephritis patients (LN) and extrarenal systemic lupus erythematosus (SLE) patients.

FIG. 14C shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb114-mAb103). LGALS3BP concentrations are in ng/ml for urine samples from healthy controls (healthy), lupus nephritis patients (LN) and extrarenal systemic lupus erythematosus (SLE) patients.

FIG. 14D shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb110-mAb103). LGALS3BP concentrations are in ng/ml for urine samples from healthy controls (healthy), lupus nephritis patients (LN) and extrarenal systemic lupus erythematosus (SLE) patients. (SLE) patients.

FIG. 15A shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb116-mAb103). LGALS3BP concentrations are in ng/ml for urine samples from healthy controls (healthy), lupus nephritis patients (LN) and extrarenal systemic lupus erythematosus (SLE) patients.

FIG. 15B shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb112-mAb103). LGALS3BP concentrations are in ng/ml for urine samples from healthy controls (healthy), lupus nephritis patients (LN) and extrarenal systemic lupus erythematosus (SLE) patients.

FIG. 15C shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb105-mAb103). LGALS3BP concentrations are in ng/ml for urine samples from healthy controls (healthy), lupus nephritis patients (LN) and extrarenal systemic lupus erythematosus (SLE) patients.

FIG. 15D shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb25-mAb103). LGALS3BP concentrations are in ng/ml for urine samples from healthy controls (healthy), lupus nephritis patients (LN) and extrarenal systemic lupus erythematosus (SLE) patients.

FIG. 16A shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb26-mAb103). LGALS3BP concentrations are in ng/ml for urine samples from healthy controls (healthy), lupus nephritis patients (LN) and extrarenal systemic lupus erythematosus (SLE) patients.

FIG. 16B shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb29-mAb103). LGALS3BP concentrations are in ng/ml for urine samples from healthy controls (healthy), lupus nephritis patients (LN) and extrarenal systemic lupus erythematosus (SLE) patients.

FIG. 16C shows a monoclonal antibody pair evaluated for use in a sandwich ELISA to capture and detect LGALS3BP in human urine samples. Graphs are derived from Luminex pairing experiments. Shown is ‘capture mAb-detection mAb’ (i.e., mAb113-mAb103). LGALS3BP concentrations are in ng/ml for urine samples from healthy controls (healthy), lupus nephritis patients (LN) and extrarenal systemic lupus erythematosus (SLE) patients.

FIG. 17 presents data which shows LGALS3BP is stable in urine under various storage conditions. Urine samples from 3 LN patients (stored at −80 C) were thawed and stored under different conditions: repeated freeze-thaws, room temperature for 1 h or 18 h, 37 C or 4 C or −20 C overnight. LGALS3BP levels in urine samples were measured by sandwich ELISA. Shown are mean+SEM of technical duplicates from 3 LN patients.

FIG. 18 shows urinary LGALS3BP concentrations (ng/ml) are significantly elevated in LN patients from different patient cohorts. LGALS3BP was measured with our prototype kit in urine samples from indicated controls and patients. LN patients were obtained from two different cohorts, from two different locations in the US. LGALS3BP levels are significantly higher in both LN cohorts compared to all other groups (P<0.0001, one-way ANOVA with Tukey's multiple comparisons test). Grey area depicts range of healthy control samples.

FIG. 19 presents LGALS3BP to creatinine ratios in urine samples from HC, SLE, LN and IgAN.

FIG. 20 presents the same data of FIG. 19 reformatted so that urinary protein to creatinine ratio (UPCR) is the metric presented in the y-axis.

FIG. 21A LGALS3BP shows better separation of LN patients from extrarenal SLE patients and healthy controls than CCL2 (MCP-1). Urinary LGALS3BP was measured in samples from indicated groups and normalized to urine creatinine levels. **P<0.01, ****P<0.00001, one-way ANOVA with Tukey's multiple comparisons test.

FIG. 21B LGALS3BP shows better separation of LN patients from extrarenal SLE patients and healthy controls than CCL2 (MCP-1). Urinary CCL2 (MCP-1) was measured in samples from indicated groups and normalized to urine creatinine levels. **P<0.01, ****P<0.00001, one-way ANOVA with Tukey's multiple comparisons test.

FIG. 22A and FIG. 22B described data confirming that detection of urinary LGALS3BP gives better sensitivity and specificity for detecting LN than CCL2 (MCP-1). Receiver operating characteristics (ROC) curves of urinary LGALS3BP/creatinine (Cr) and CCL2 (MCP-1)/creatinine ratios for distinguishing LN from healthy controls (HC) or extrarenal SLE (SLE).

FIG. 23A shows correlations of urinalysis measurements, wherein, albumin to creatinine ratios and total protein to creatinine ratios closely correlated to one another with a correlation coefficient of 0.965.

FIG. 23B shows correlations of urinalysis measurements (using the reagents associated the diagnostic kit presented in the Experimental section of the instant application), wherein, LGALS3BP to creatinine ratios show weak positive correlation with total protein to creatinine ratios

(r=0.494).

FIG. 24 shows correlations of urinalysis measurements (using the reagents associated the diagnostic kit presented in the Experimental section of the instant application), wherein, LGALS3BP to creatinine ratios show weak positive correlation with albumin to creatinine ratios (r=0.484).

FIG. 25 describes data showing urinary LGALS3BP/creatinine ratios in different kidney disease groups. The graph shows increased levels of LGALS3BP preferentially in LN when active (flaring). This shows a disease-specific pattern in uG3BP expression and a trend that is driven by active inflammation in the context of LN.

FIG. 26A shows means for urinary LGALS3BP/creatinine ratios in different kidney disease groups. Urinary LGALS3BP concentrations (ng/ml) were normalized to creatinine concentration (mg/ml), natural log transformed and outliers were excluded for data analysis. JMP pro v12 is used including ANOVA and Wilcoxon non parametric multiple comparison.

FIG. 26B shows significant p values between comparison groups. Urinary LGALS3BP data were normalized to creatinine concentration, natural log transformed and outliers were excluded for data analysis. JMP pro v12 is used including ANOVA and Wilcoxon non parametric multiple comparison.

FIG. 27A, FIG. 27B and FIG. 27C show weak positive correlation between urinary LGALS3BP/creatinine and urinary protein/creatinine ratios in LN irrespective of disease status (all, active or in remission)

FIG. 28A shows urinary protein to creatinine ratios (UPCR) in International Society of Nephrology (ISN)/Renal Pathology Society (RPS) classification of LN in active disease versus patients in remission. UPCR is associated with kidney damage and always higher in active disease regardless of ISN/RPS class.

FIG. 28B shows urinary LGALS3BP/creatinine ratios International Society of Nephrology (ISN)/Renal Pathology Society (RPS) classification of LN in active disease versus patients in remission. Urinary LGALS3BP/creatinine levels are elevated in active disease compared to remission in class II to IV but not V. Class II to IV are inflammatory forms of LN while class V is less inflammatory, further support for urinary LGALS3BP being a readout of active inflammation in the kidney.

FIG. 29 shows the fluctuation, over time, of urinary LGALS3BP/creatinine levels in LN patients. LN patient urine was monitored monthly.

FIG. 30 shows how the initiation of LN-specific treatments reduces urinary LGALS3BP levels over time. Specifically, newly diagnosed LN patients were put on Eurolupus treatment (specific) and urinary LGALS3BP levels tracked over time.

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For embodiment, reference to “a” includes a single as well as two or more; reference to “an” includes a single as well as two or more; reference to “the” includes a single as well as two or more and so forth.

Each embodiment of the present disclosure described herein is to be applied mutatis mutandis to each and every other embodiment unless specifically stated otherwise.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for embodiment, in Sambrook, Fritsch & Maniatis, Molecular Cloning: A Laboratory Manual, Cold Spring Harbor Laboratories, New York, Second Edition (1989), whole of Vols I, II, and III; Benny K. C. Lo, Antibody Engineering: Methods and Protocols, (2004) Humana Press, Vol. 248; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al., pp 35-81; Sproat et al., pp 83-115; and Wu et al., pp 135-151; Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara Biochem. Biophys. Res. Commun 73: 336-342, 1976; Merrifield J. Am. Chem. Soc. 85: 2149-2154, 1963; Barany and Merrifield (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Muller, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky Int. J. Peptide Protein Res. 25: 449-474, 1985; Handbook of Experimental Immunology, Vols. I-IV (D. M. Weir and C. C. Blackwell, eds., 1986, Blackwell Scientific Publications); and Animal Cell Culture: Practical Approach, 3rd edn (John R. W. Masters, ed., 2000), ISBN 0199637970, whole of text.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Preferred embodiments of the present invention are based on the role that LGALS3BP plays as a predictive marker in quantitating levels of kidney inflammation in LN.

An exemplary full length human LGALS3BP polypeptide sequence (SEQ ID NO: 1) is as follows:

MTPPRLFWVWLLVAGTQGVNDGDMRLADGGATNQGRVEIFYRGQWGTVCD
NLWDLTDASVVCRALGFENATQALGRAAFGQGSGPIMLDEVQCTGTEASL
ADCKSLGWLKSNCRHERDAGVVCTNETRSTHTLDLSRELSEALGQIFDSQ
RGCDLSISVNVQGEDALGFCGHTVILTANLEAQALWKEPGSNVTMSVDAE
CVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVP
TDLLQLLLPRSDLAVPSELALLKAVDTWSWGERASHEEVEGLVEKIRFPM
MLPEELFELQFNLSLYWSHEALFQKKTLQALEFHTVPFQLLARYKGLNLT
EDTYKPRIYTSPTWSAFVTDSSWSARKSQLVYQSRRGPLVKYSSDYFQAP
SDYRYYPYQSFQTPQHPSFLFQDKRVSWSLVYLPTIQSCWNYGFSCSSDE
LPVLGLTKSGGSDRTIAYENKALMLCEGLFVADVTDFEGWKAAIPSALDT
NSSKSTSSFPCPAGHFNGFRTVIRPFYLTNSSGVD

### DEFINITIONS

“Inflammation” is used herein in the general medical sense of the word and may be an acute or chronic; simple or suppurative; localized or disseminated; cellular and tissue response initiated or sustained by any number of chemical, physical or biological agents or combination of agents.

“Inflammatory state” is used to indicate the relative biological condition of a subject resulting from inflammation, or characterizing the degree of inflammation.

The terms “patient” and “subject” are used in this disclosure to refer to a mammal being treated or in need of treatment for a condition such as LN. The terms include human patients and volunteers, non-human mammals such as a non-human primates, large animal models and rodents.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art. The sample is blood, urine, spinal fluid, lymph, mucosal secretions, prostatic fluid, semen, haemolymph or any other body fluid known in the art for a subject. The sample is also a tissue sample.

“Therapy” includes all interventions whether biological, chemical, physical, or combination of the foregoing, intended to sustain or alter the monitored biological condition of a subject.

The term “isolated protein” is intended to mean a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, for embodiment, at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antigen binding domain, this term does not encompass an antibody naturally-occurring within a subject's body that is the product of natural recombination that occurs during B cell maturation. However, if such an antibody is isolated, it is to be considered an isolated protein comprising an antigen binding domain. Similarly, if nucleic acid encoding the protein is isolated and expressed using recombinant means, the resulting protein is a recombinant protein comprising an antigen binding domain A recombinant protein also encompasses a protein expressed by artificial recombinant means when it is within a cell, tissue or subject, for embodiment, in which it is expressed.

The term “Ig fusion protein which specifically binds to LGALS3BP” shall be taken to include an Ig fusion protein (including, but not limited to, an anti-LGALS3BP antibody) capable of binding to LGALS3BP in the manner described and/or claimed herein.

The term “polypeptide” or “polypeptide chain” will be understood to mean a series of contiguous amino acids linked by peptide bonds.

As used herein, the term “antigen binding domain” shall be taken to mean a region of an antibody that is capable of specifically binding to an antigen, that is, a V_{H} or a V_{L} or an Fv comprising both a V_{H} and a V_{L}. The antigen binding domain need not be in the context of an entire antibody, for embodiment, it can be in isolation (e.g., a domain antibody) or in another form (e.g., scFv).

For the purposes for the present disclosure, the term “antibody” includes a protein capable of specifically binding to one or a few closely related antigens (e.g., LGALS3BP) by virtue of an antigen binding domain contained within a Fv. This term includes four chain antibodies (e.g., two light (L) chains and two heavy (H) chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (^{˜}50 to 70 kDa each) covalently linked and two light chains (¹⁸ 23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s) Heavy chains of mammals are of one of the following types α, δ, ε, γ, or μ. Each light chain is also covalently linked to one of the heavy chains For embodiment, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are approximately 110 amino acids in length) and one or more constant domains at the C-terminus. The constant domain of the light chain (CL which is approximately 110 amino acids in length) is aligned with and disulfide bonded to the first constant domain of the heavy chain (C_{H}1 which is 330 to 440 amino acids in length). The light chain variable region is aligned with the variable region of the heavy chain The antibody heavy chain can comprise 2 or more additional C_{H} domains (such as, C_{H}2, C_{H}3 and the like) and can comprise a hinge region between the C_{H}1 and C_{H}2 constant domains Antibodies can be of any type (e.g., IgG, IgE, IgM, IgD, IgA, and IgY), class (e.g., IgG1, IgG2, IgG3, IgG4, IgA1 and IgA2) or subclass.

As used herein, “variable region” refers to the portions of the light and/or heavy chains of an antibody as defined herein that is capable of specifically binding to an antigen and, includes amino acid sequences of complementarity determining regions (CDRs), that is, CDR1, CDR2, and CDR3, and framework regions (FRs). For embodiment, the variable region comprises three or four FRs (e.g., FR1, FR2, FR3 and optionally FR4) together with three CDRs. V_{H} refers to the variable region of the heavy chain V_{L} refers to the variable region of the light chain.

As used herein, the term “complementarity determining regions” (syn. CDRs, i.e., CDR1, CDR2, and CDR3) refers to the amino acid residues of an antibody variable region the presence of which are major contributors to specific antigen binding. Each variable region domain (V_{H} or V_{L}) typically has three CDR regions identified as CDR1, CDR2 and CDR3. In one embodiment, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another embodiment, the amino acid positions assigned to CDRs and FRs are defined according to the Enhanced Chothia Numbering Scheme. According to the numbering system of Kabat, V_{H}FRs and CDRs are positioned as follows: residues 1 to 30 (FR1), 31 to 35 (CDR1), 36 to 49 (FR2), 50 to 65 (CDR2), 66 to 94 (FR3), 95 to 102 (CDR3) and 103 to 113 (FR4). According to the numbering system of Kabat, V_{L}FRs and CDRs are positioned as follows: residues 1 to 23 (FR1), 24 to 34 (CDR1), 35 to 49 (FR2), 50 to 56 (CDR2), 57 to 88 (FR3), 89 to 97 (CDR3) and 98 to 107 (FR4). The present disclosure is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Pliikthun J. Mol. Biol. 309: 657-670, 2001; or the IMGT system discussed in Giudicelli et al., Nucleic Acids Res. 25: 206-211 1997. In one embodiment, the CDRs are defined according to the Kabat numbering system.

As used herein, the term “Fv” shall be taken to mean any protein, whether comprised of multiple polypeptides or a single polypeptide, in which a V_{L} and a V_{H} associate and form a complex having an antigen binding domain that is capable of specifically binding to an antigen. The V_{H} and the V_{L} which form the antigen binding domain can be in a single polypeptide chain or in different polypeptide chains. Furthermore, a Fv of the disclosure (as well as any protein of the disclosure) may have multiple antigen binding domains which may or may not bind the same antigen. This term shall be understood to encompass fragments directly derived from an antibody as well as proteins corresponding to such a fragment produced using recombinant means. In some embodiments, the V_{H} is not linked to a heavy chain constant domain (C_{H}) 1 and/or the V_{L} is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, for embodiment, C_{H}2 or C_{H}3 domain, for embodiment, a minibody.

A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means.

A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner A Fab′ fragment can also be produced by recombinant means.

A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a Ig fusion protein which specifically binds to LGALS3BP or an antigen binding domain thereof with an antigen means that the interaction is dependent upon the presence of a particular structure (e.g., an antigenic determinant or epitope) on the antigen. For embodiment, an antibody recognizes and binds to a specific protein structure rather than to proteins generally. If an antibody binds to epitope “A”, the presence of a molecule containing epitope “A” (or free, unlabeled “A”), in a reaction containing labeled “A” and the antibody, will reduce the amount of labeled “A” bound to the antibody.

As used herein, the term “specifically binds” shall be taken to mean that a protein of the disclosure (e.g., an anti-LGALS3BP antibody) reacts or associates more frequently, more rapidly, with greater duration and/or with greater affinity with a particular antigen or cell expressing same than it does with alternative antigens or cells. For embodiment, a protein that specifically binds to an antigen binds that antigen with greater affinity, avidity, more readily, and/or with greater duration than it binds to other antigens. For embodiment, a protein binds to LGALS3BP with materially greater affinity than it does to other immunoglobulin superfamily ligands or to antigens commonly recognized by polyreactive natural antibodies (i.e., by naturally occurring antibodies known to bind a variety of antigens naturally found in humans) It is also understood by reading this definition that, for embodiment, a protein that specifically binds to a first antigen may or may not specifically bind to a second antigen. As such, “specific binding” does not necessarily require exclusive binding or non-detectable binding of another antigen, this is meant by the term “selective binding”.

As used herein, the term “epitope” (syn. “antigenic determinant”) shall be understood to mean a region of LGALS3BP to which a protein comprising an antigen binding domain of an antibody binds. This term is not necessarily limited to the specific residues or structure to which the protein makes contact. For embodiment, this term includes the region spanning amino acids contacted by the protein and/or at least 5 to 10 or 2 to 5 or 1 to 3 amino acids outside of this region. In some embodiments, the epitope is a linear series amino acids. An epitope may also comprise a series of discontinuous amino acids that are positioned close to one another when LGALS3BP is folded, that is, a “conformational epitope”. The skilled artisan will also be aware that the term “epitope” is not limited to peptides or polypeptides. For embodiment, the term “epitope” includes chemically active surface groupings of molecules such as sugar side chains, phosphoryl side chains, or sulfonyl side chains, and, in certain embodiments, may have specific three dimensional structural characteristics, and/or specific charge characteristics. An epitope or peptide or polypeptide comprising same can be administered to an animal to generate antibodies against the epitope.

As used herein, the term “diagnosis”, and variants thereof such as, but not limited to, “diagnose”, “diagnosed” or “diagnosing” includes any primary diagnosis of a clinical state or diagnosis of recurrent disease.

### METHODS

The following methods were used to source and prepare materials (including, but not limited to, human and non-human tissues, cells and proteins) used in the following Experimental Examples section in the instant patent application.

### In Vitro Stimulation of Human Macrophages

Human PBMCs were isolated from buffy coat preparations of healthy donors (New York Blood Center) using Ficoll Paque Plus (GE Health Sciences) according to the manufacturer's instructions. Monocytes were purified by adherence to plastic for 90 minutes and subsequently differentiated to macrophages by culture with 100 ng/ml GM-CSF (Sargramostim, Sanofi) in RPMI 1640 (Gibco) containing Pen/Strep and 10% heat inactivated fetal bovine serum (Corning). On day 7 inflammatory stimuli (recombinant IFNα, CpG for TLR9, LPS for TLR4, small molecule agonist for TLR7/8 and IFNα) were added and LGALS3BP mRNA measured by qCPR after 6 h and LGALS3BP protein by ELISA after 20 h. mRNA was measured with Taqman technology (Applied Biosystems) and HPRT1 used as a housekeeping gene for normalization. Samples were run on an Applied Biosystems QuantStudio instrument. LGALS3BP protein was measured with a commercially available ELISA kit (Abnova).

### LGALS3BP Expression in Blood

Patient whole blood was collected and PBMCs were isolated by Ficoll density centrifugation. PBMCs were frozen at −80° C. in 90% fetal calf serum containing 10% DMSO. When ready for further analysis, cells were rapidly thawed, lysed with Buffer RLT (Qiagen) containing 1% (3-mercaptoethanol, and RNA was extracted using the RNeasy mini kit (Qiagen). This was followed by DNAse1 treatment and additional cleanup using SPRI beads (Life Technologies). RNA-seq was subsequently performed using the Smartseq2 protocol. Data are presented as FPKM values.

### LGALS3BP Expression in Kidneys from LN Patients and Healthy Controls

Human renal biopsies were collected after obtaining informed consent, processed, and used for microarray analysis. Detailed method information can be found in the original reference (Berthier C C et al., JI 2012). This data was accessed from the GEO database under GSE32591. The linear expression data are shown.

### LGALS3BP Expression in BXSB-Yaa Model

All procedures using animals were performed in accordance with all local and national laws and regulations regarding animal care. Male BXSB-Yaa mice were purchased from Jackson. At 20 weeks of age mice were euthanized via CO2 asphyxiation and blood was collected via the vena cava. At the conclusion of studies kidneys were collected, fixed in formalin and shipped to HistoTox Labs where they were processed for hematoxylin and eosin staining and scored for histological evidence of damage by a trained pathologist. The scoring system used was modified from a previously published system (Chan, O., Madaio, M. P., and Shlomchik, M. J. 1997. The roles of B cells in MRL/lpr murine lupus. Ann N Y Acad Sci 815:75-87) and evaluates kidney sections based on glomerular crescents, protein casts, interstitial inflammation, and vasculitis and a total histology score is obtained based on a composite score of these parameters.

### Plasma and Urine Collection

Whole blood and freshly voided urine was obtained from healthy patients or SLE and LN patients. Whole blood was collected in heparin tubes and shipped at ambient temperature. Plasma was collected by spinning whole blood at 720×g for 10 minutes. Plasma was collected and centrifuged again for 15 mins at 2000×g to remove platelets. All samples were stored at −80 C.

### Antibodies/Library Based Methods

The present disclosure also encompasses screening of libraries of antibodies or proteins comprising antigen binding domains thereof (e.g., comprising variable regions thereof) to identify a Ig fusion protein which specifically binds to LGALS3BP of the disclosure. For embodiment, a library comprising a V_{H} of the disclosure and a plurality of V_{L} regions can be screened to identify a Ig fusion protein which specifically binds to LGALS3BP of the disclosure.

Embodiments of libraries contemplated by this disclosure include naïve libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3rd Ed, vols. 1-3, Cold Spring Harbor Laboratory Press, 2001) and used to encode and display proteins using a method known in the art. Other techniques for producing libraries of proteins are described in, for embodiment in U.S. Pat. No. 6,300,064 (e.g., a HuCAL library of Morphosys AG), U.S. Pat. Nos. 5,885,793, 6,204,023, 6,291,158, or 6,248,516.

The Ig fusion protein which specifically binds to LGALS3BPs according to the disclosure may be soluble secreted proteins or may be presented as a fusion protein on the surface of a cell, or particle (e.g., a phage or other virus, a ribosome or a spore). Various display library formats are known in the art. For embodiment, the library is an in vitro display library (e.g., a ribosome display library, a covalent display library or a mRNA display library, e.g., as described in U.S. Pat. No. 7,270,969). In yet another embodiment, the display library is a phage display library wherein proteins comprising antigen binding domains of antibodies are expressed on phage, for embodiment, as described in U.S. Pat. Nos. 6,300,064, 5,885,793, 6,204,023, 6,291,158, or 6,248,516. Other phage display methods are known in the art and are contemplated by the present disclosure. Similarly, methods of cell display are contemplated by the disclosure, for embodiment, bacterial display libraries, for embodiment, as described in U.S. Pat. No. 5,516,637; yeast display libraries, for embodiment, as described in U.S. Pat. No. 6,423,538; or a mammalian display library.

Methods for screening display libraries are known in the art. In one embodiment, a display library of the present disclosure is screened using affinity purification, for embodiment, as described in Scopes (In: Protein purification: principles and practice, Third Edition, Springer Verlag, 1994). Methods of affinity purification typically involve contacting proteins comprising antigen binding domains displayed by the library with a target antigen (e.g., LGALS3BP) and, following washing, eluting those domains that remain bound to the antigen.

Any variable regions or scFvs identified by screening are readily modified into a complete antibody, if desired. Exemplary methods for modifying or reformatting variable regions or scFvs into a complete antibody are described, for embodiment, in Jones et al., J. Immunol. Methods 354: 85-90, 2010; or Jostock et al., J. Immunol. Methods, 289: 65-80, 2004. Alternatively, or additionally, standard cloning methods are used, e.g., as described in Ausubel et al., (In: Current Protocols in Molecular Biology. Wiley Interscience, ISBN 047 150338, 1987), and/or (Sambrook et al., (In: Molecular Cloning: Molecular Cloning: A Laboratory Manual, Cold Spring Harbor Laboratories, New York, Third Edition 2001).

In one embodiment, the present disclosure provides a method of producing or isolating a Ig fusion protein which specifically binds to LGALS3BP of the disclosure by screening a display library, for embodiment, a phage display library, for embodiment, as described in U.S. Pat. Nos. 6,300,064 and/or 5,885,793. For embodiment, the present inventors have isolated scFvs by biopanning a human scFv immunoglobulin gene library by rounds of selection against full length recombinant human LGALS3BP. Once isolated, a Ig fusion protein which specifically binds to LGALS3BP of the invention can be cloned and expressed and optionally reformatted as, for embodiment, an IgG1 antibody using known methods in the art.

In one embodiment, the present disclosure provides a method of producing a Ig fusion protein which specifically binds to LGALS3BP, the method comprising:
(i) screening a Ig fusion protein which specifically binds to LGALS3BP preparation or library for a binding protein that binds to the extracellular domain of LGALS3BP, for embodiment, the extracellular domain of recombinant human LGALS3BP; and
(ii) isolating a Ig fusion protein which specifically binds to LGALS3BP having a desired binding affinity for the extracellular domain of LGALS3BP.

In one embodiment, a Ig fusion protein which specifically binds to LGALS3BP preparation is screened. A LGALS3BP preparation may be made by, for embodiment, immunizing an animal with a LGALS3BP antigen so as to produce antibodies that react with the extracellular domain of LGALS3BP.

In another embodiment, a Ig fusion protein which specifically binds to LGALS3BP library is screened. The library may be a phage library, for embodiment, a scFv phage library or a Fab phage library.

In one embodiment, the method comprises producing a population of phage particles displaying at their surface a population of binding molecules having a range of binding specificities for a target LGALS3BP epitope or antigen. Such phage particles comprise a phagemid genome comprising a nucleic acid encoding the binding protein. This nucleic acid can be isolated, cloned and expressed in a recombinant system to produce the Ig fusion protein which specifically binds to LGALS3BP of the invention.

Exemplary cells used for expressing a Ig fusion protein which specifically binds to LGALS3BP of the disclosure are CHO cells, myeloma cells or HEK cells. The cell may further comprise one or more genetic mutations and/or deletions that facilitate expression of a modified antibody. One non-limiting embodiment is a deletion of a gene encoding an enzyme required for fucosylation of an expressed immunoglobulin or antibody.

### Protein Purification

Following production/expression, a Ig fusion protein which specifically binds to LGALS3BP of the disclosure is purified using a method known in the art. Such purification provides the protein of the disclosure substantially free of nonspecific protein, acids, lipids, carbohydrates, and the like. In one embodiment, the protein will be in a preparation wherein more than about 90% (e.g., 95%, 98% or 99%) of the protein in the preparation is a Ig fusion protein which specifically binds to LGALS3BP of the disclosure.

Standard methods of peptide purification are employed to obtain an isolated Ig fusion protein which specifically binds to LGALS3BP of the disclosure, including but not limited to various high-pressure (or performance) liquid chromatography (HPLC) and non-HPLC polypeptide isolation protocols, such as size exclusion chromatography, ion exchange chromatography, hydrophobic interaction chromatography, mixed mode chromatography, phase separation methods, electrophoretic separations, precipitation methods, salting in/out methods, immunochromatography, and/or other methods.

### Ig Fusion Protein which Specifically Binds to LGALS3BPs/Anti-LGALS3BP Antibodies

Selected embodiments of the present invention are based on the inventors' production of human antibodies that bind specifically to LGALS3BP. These human anti-LGALS3BP antibodies derived from a phage display library of human scFv sequences; the obtained scFv phage clone reformatted as an IgG1 mAb.

The present disclosure is broadly directed to a Ig fusion protein which specifically binds to LGALS3BP comprising an antigen binding domain which specifically binds to LGALS3BP.

### Qqq

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 32, the V_{H}CDR2 comprises the amino acid sequence shown in SEQ ID NO: 33 and the V_{H}CDR3 the amino acid sequence shown in amino acids of SEQ ID NO: 34 and a light chain variable region (V_{L}) which comprises three complementarity determining regions (CDRs), wherein, V_{L}CDR1 comprises the amino acid sequence shown in SEQ ID NO: 35, the V_{L}CDR2 comprises the amino acid sequence shown in SEQ ID NO: 36 and the V_{L}CDR3 comprises the amino acid sequence shown in SEQ ID NO: 37. A condensation of the three V_{H}CDRs and the three V_{L}CDRs of the LGALS3BP Ig fusion protein recited in the aforementioned paragraph is shown in the amino acids of SEQ ID NO: 2.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 38, the V_{H}CDR2 comprises the amino acid sequence shown in SEQ ID NO: 39 and the V_{H}CDR3 the amino acid sequence shown in amino acids of SEQ ID NO: 40 and a light chain variable region (V_{L}) which comprises three complementarity determining regions (CDRs), wherein, V_{L}CDR1 comprises the amino acid sequence shown in SEQ ID NO: 41, the V_{L}CDR2 comprises the amino acid sequence shown in SEQ ID NO: 42 and the V_{L}CDR3 comprises the amino acid sequence shown in SEQ ID NO: 43. A condensation of the three V_{H}CDRs and the three V_{L}CDRs of the LGALS3BP Ig fusion protein recited in the aforementioned paragraph is shown in the amino acids of SEQ ID NO: 3.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 44, the V_{H}CDR2 comprises the amino acid sequence shown in SEQ ID NO: 45 and the V_{H}CDR3 the amino acid sequence shown in amino acids of SEQ ID NO: 46 and a light chain variable region (V_{L}) which comprises three complementarity determining regions (CDRs), wherein, V_{L}CDR1 comprises the amino acid sequence shown in SEQ ID NO: 47, the V_{L}CDR2 comprises the amino acid sequence shown in SEQ ID NO: 48 and the V_{L}CDR3 comprises the amino acid sequence shown in SEQ ID NO: 49. A condensation of the three V_{H}CDRs and the three V_{L}CDRs of the LGALS3BP Ig fusion protein recited in the aforementioned paragraph is shown in the amino acids of SEQ ID NO: 4.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 50, the V_{H}CDR2 comprises the amino acid sequence shown in SEQ ID NO: 51 and the V_{H}CDR3 the amino acid sequence shown in amino acids of SEQ ID NO: 52 and a light chain variable region (V_{L}) which comprises three complementarity determining regions (CDRs), wherein, V_{L}CDR1 comprises the amino acid sequence shown in SEQ ID NO: 53, the V_{L}CDR2 comprises the amino acid sequence shown in SEQ ID NO: 54 and the V_{L}CDR3 comprises the amino acid sequence shown in SEQ ID NO: 55. A condensation of the three V_{H}CDRs and the three V_{L}CDRs of the LGALS3BP Ig fusion protein recited in the aforementioned paragraph is shown in the amino acids of SEQ ID NO: 5

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 56, the V_{H}CDR2 comprises the amino acid sequence shown in SEQ ID NO: 57 and the V_{H}CDR3 the amino acid sequence shown in amino acids of SEQ ID NO: 58 and a light chain variable region (V_{L}) which comprises three complementarity determining regions (CDRs), wherein, V_{L}CDR1 comprises the amino acid sequence shown in SEQ ID NO: 59, the V_{L}CDR2 comprises the amino acid sequence shown in SEQ ID NO: 60 and the V_{L}CDR3 comprises the amino acid sequence shown in SEQ ID NO: 61. A condensation of the three V_{H}CDRs and the three V_{L}CDRs of the LGALS3BP Ig fusion protein recited in the aforementioned paragraph is shown in the amino acids of SEQ ID NO: 6.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 62, the V_{H}CDR2 comprises the amino acid sequence shown in SEQ ID NO: 63 and the V_{H}CDR3 the amino acid sequence shown in amino acids of SEQ ID NO: 64 and a light chain variable region (V_{L}) which comprises three complementarity determining regions (CDRs), wherein, V_{L}CDR1 comprises the amino acid sequence shown in SEQ ID NO: 65, the V_{L}CDR2 comprises the amino acid sequence shown in SEQ ID NO: 66 and the V_{L}CDR3 comprises the amino acid sequence shown in SEQ ID NO: 67. A condensation of the three V_{H}CDRs and the three V_{L}CDRs of the LGALS3BP Ig fusion protein recited in the aforementioned paragraph is shown in the amino acids of SEQ ID NO: 7.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 68, the V_{H}CDR2 comprises the amino acid sequence shown in SEQ ID NO: 69 and the V_{H}CDR3 the amino acid sequence shown in amino acids of SEQ ID NO: 70 and a light chain variable region (V_{L}) which comprises three complementarity determining regions (CDRs), wherein, V_{L}CDR1 comprises the amino acid sequence shown in SEQ ID NO: 71, the V_{L}CDR2 comprises the amino acid sequence shown in SEQ ID NO: 72 and the V_{L}CDR3 comprises the amino acid sequence shown in SEQ ID NO: 73. A condensation of the three V_{H}CDRs and the three V_{L}CDRs of the LGALS3BP Ig fusion protein recited in the aforementioned paragraph is shown in the amino acids of SEQ ID NO: 8.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 74, the V_{H}CDR2 comprises the amino acid sequence shown in SEQ ID NO: 75 and the V_{H}CDR3 the amino acid sequence shown in amino acids of SEQ ID NO: 76 and a light chain variable region (V_{L}) which comprises three complementarity determining regions (CDRs), wherein, V_{L}CDR1 comprises the amino acid sequence shown in SEQ ID NO: 77, the V_{L}CDR2 comprises the amino acid sequence shown in SEQ ID NO: 78 and the V_{L}CDR3 comprises the amino acid sequence shown in SEQ ID NO: 79. A condensation of the three V_{H}CDRs and the three V_{L}CDRs of the LGALS3BP Ig fusion protein recited in the aforementioned paragraph is shown in the amino acids of SEQ ID NO: 9.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 80, the V_{H}CDR2 comprises the amino acid sequence shown in SEQ ID NO: 81 and the V_{H}CDR3 the amino acid sequence shown in amino acids of SEQ ID NO: 82 and a light chain variable region (V_{L}) which comprises three complementarity determining regions (CDRs), wherein, V_{L}CDR1 comprises the amino acid sequence shown in SEQ ID NO: 83, the V_{L}CDR2 comprises the amino acid sequence shown in SEQ ID NO: 84 and the V_{L}CDR3 comprises the amino acid sequence shown in SEQ ID NO: 85. A condensation of the three V_{H}CDRs and the three V_{L}CDRs of the LGALS3BP Ig fusion protein recited in the aforementioned paragraph is shown in the amino acids of SEQ ID NO: 10.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 86, the V_{H}CDR2 comprises the amino acid sequence shown in SEQ ID NO: 87 and the V_{H}CDR3 the amino acid sequence shown in amino acids of SEQ ID NO: 88 and a light chain variable region (V_{L}) which comprises three complementarity determining regions (CDRs), wherein, V_{L}CDR1 comprises the amino acid sequence shown in SEQ ID NO: 89, the V_{L}CDR2 comprises the amino acid sequence shown in SEQ ID NO: 90 and the V_{L}CDR3 comprises the amino acid sequence shown in SEQ ID NO: 91. A condensation of the three V_{H}CDRs and the three V_{L}CDRs of the LGALS3BP Ig fusion protein recited in the aforementioned paragraph is shown in the amino acids of SEQ ID NO: 11.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 92, the V_{H}CDR2 comprises the amino acid sequence shown in SEQ ID NO: 93 and the V_{H}CDR3 the amino acid sequence shown in amino acids of SEQ ID NO: 94 and a light chain variable region (V_{L}) which comprises three complementarity determining regions (CDRs), wherein, V_{L}CDR1 comprises the amino acid sequence shown in SEQ ID NO: 95, the V_{L}CDR2 comprises the amino acid sequence shown in SEQ ID NO: 96 and the V_{L}CDR3 comprises the amino acid sequence shown in SEQ ID NO: 97. A condensation of the three V_{H}CDRs and the three V_{L}CDRs of the LGALS3BP Ig fusion protein recited in the aforementioned paragraph is shown in the amino acids of SEQ ID NO: 12.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 98, the V_{H}CDR2 comprises the amino acid sequence shown in SEQ ID NO: 99 and the V_{H}CDR3 the amino acid sequence shown in amino acids of SEQ ID NO: 100 and a light chain variable region (V_{L}) which comprises three complementarity determining regions (CDRs), wherein, V_{L}CDR1 comprises the amino acid sequence shown in SEQ ID NO: 101, the V_{L}CDR2 comprises the amino acid sequence shown in SEQ ID NO: 102 and the V_{L}CDR3 comprises the amino acid sequence shown in SEQ ID NO: 103. A condensation of the three V_{H}CDRs and the three V_{L}CDRs of the LGALS3BP Ig fusion protein recited in the aforementioned paragraph is shown in the amino acids of SEQ ID NO: 13.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 104, the V_{H} CDR2 comprises the amino acid sequence shown in SEQ ID NO: 105 and the V_{H}CDR3 the amino acid sequence shown in amino acids of SEQ ID NO: 106 and a light chain variable region (V_{L}) which comprises three complementarity determining regions (CDRs), wherein, V_{L}CDR1 comprises the amino acid sequence shown in SEQ ID NO: 107, the V_{L}CDR2 comprises the amino acid sequence shown in SEQ ID NO: 108 and the V_{L}CDR3 comprises the amino acid sequence shown in SEQ ID NO: 109. A condensation of the three V_{H}CDRs and the three V_{L} CDRs of the LGALS3BP Ig fusion protein recited in the aforementioned paragraph is shown in the amino acids of SEQ ID NO: 14.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 110, the V_{H} CDR2 comprises the amino acid sequence shown in SEQ ID NO: 111 and the V_{H}CDR3 the amino acid sequence shown in amino acids of SEQ ID NO: 112 and a light chain variable region (V_{L}) which comprises three complementarity determining regions (CDRs), wherein, V_{L}CDR1 comprises the amino acid sequence shown in SEQ ID NO: 113, the V_{L}CDR2 comprises the amino acid sequence shown in SEQ ID NO: 114 and the V_{L}CDR3 comprises the amino acid sequence shown in SEQ ID NO: 115. A condensation of the three V_{H}CDRs and the three V_{L} CDRs of the LGALS3BP Ig fusion protein recited in the aforementioned paragraph is shown in the amino acids of SEQ ID NO: 15.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 116, the V_{H} CDR2 comprises the amino acid sequence shown in SEQ ID NO: 117 and the V_{H}CDR3 the amino acid sequence shown in amino acids of SEQ ID NO: 118 and a light chain variable region (V_{L}) which comprises three complementarity determining regions (CDRs), wherein, V_{L}CDR1 comprises the amino acid sequence shown in SEQ ID NO: 119, the V_{L}CDR2 comprises the amino acid sequence shown in SEQ ID NO: 120 and the V_{L}CDR3 comprises the amino acid sequence shown in SEQ ID NO: 121. A condensation of the three V_{H}CDRs and the three V_{L} CDRs of the LGALS3BP Ig fusion protein recited in the aforementioned paragraph is shown in the amino acids of SEQ ID NO: 16.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 122, the V_{H} CDR2 comprises the amino acid sequence shown in SEQ ID NO: 123 and the V_{H}CDR3 the amino acid sequence shown in amino acids of SEQ ID NO: 124 and a light chain variable region (V_{L}) which comprises three complementarity determining regions (CDRs), wherein, V_{L}CDR1 comprises the amino acid sequence shown in SEQ ID NO: 125, the V_{L}CDR2 comprises the amino acid sequence shown in SEQ ID NO: 126 and the V_{L}CDR3 comprises the amino acid sequence shown in SEQ ID NO: 127. A condensation of the three V_{H}CDRs and the three V_{L} CDRs of the LGALS3BP Ig fusion protein recited in the aforementioned paragraph is shown in the amino acids of SEQ ID NO: 17.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 128, the V_{H} CDR2 comprises the amino acid sequence shown in SEQ ID NO: 129 and the V_{H}CDR3 the amino acid sequence shown in amino acids of SEQ ID NO: 130 and a light chain variable region (V_{L}) which comprises three complementarity determining regions (CDRs), wherein, V_{L}CDR1 comprises the amino acid sequence shown in SEQ ID NO: 131, the V_{L}CDR2 comprises the amino acid sequence shown in SEQ ID NO: 132 and the V_{L}CDR3 comprises the amino acid sequence shown in SEQ ID NO: 133. A condensation of the three V_{H}CDRs and the three V_{L} CDRs of the LGALS3BP Ig fusion protein recited in the aforementioned paragraph is shown in the amino acids of SEQ ID NO: 18.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 134, the V_{H} CDR2 comprises the amino acid sequence shown in SEQ ID NO: 135 and the V_{H}CDR3 the amino acid sequence shown in amino acids of SEQ ID NO: 136 and a light chain variable region (V_{L}) which comprises three complementarity determining regions (CDRs), wherein, V_{L}CDR1 comprises the amino acid sequence shown in SEQ ID NO: 137, the V_{L}CDR2 comprises the amino acid sequence shown in SEQ ID NO: 138 and the V_{L}CDR3 comprises the amino acid sequence shown in SEQ ID NO: 139. A condensation of the three V_{H}CDRs and the three V_{L} CDRs of the LGALS3BP Ig fusion protein recited in the aforementioned paragraph is shown in the amino acids of SEQ ID NO: 19.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 140, the V_{H} CDR2 comprises the amino acid sequence shown in SEQ ID NO: 141 and the V_{H}CDR3 the amino acid sequence shown in amino acids of SEQ ID NO: 142 and a light chain variable region (V_{L}) which comprises three complementarity determining regions (CDRs), wherein, V_{L}CDR1 comprises the amino acid sequence shown in SEQ ID NO: 143, the V_{L}CDR2 comprises the amino acid sequence shown in SEQ ID NO: 144 and the V_{L}CDR3 comprises the amino acid sequence shown in SEQ ID NO: 145. A condensation of the three V_{H}CDRs and the three V_{L} CDRs of the LGALS3BP Ig fusion protein recited in the aforementioned paragraph is shown in the amino acids of SEQ ID NO: 20.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 146, the V_{H} CDR2 comprises the amino acid sequence shown in SEQ ID NO: 147 and the V_{H}CDR3 the amino acid sequence shown in amino acids of SEQ ID NO: 148 and a light chain variable region (V_{L}) which comprises three complementarity determining regions (CDRs), wherein, V_{L}CDR1 comprises the amino acid sequence shown in SEQ ID NO: 149, the V_{L}CDR2 comprises the amino acid sequence shown in SEQ ID NO: 150 and the V_{L}CDR3 comprises the amino acid sequence shown in SEQ ID NO: 151. A condensation of the three V_{H}CDRs and the three V_{L} CDRs of the LGALS3BP Ig fusion protein recited in the aforementioned paragraph is shown in the amino acids of SEQ ID NO: 21.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 152, the V_{H} CDR2 comprises the amino acid sequence shown in SEQ ID NO: 153 and the V_{H}CDR3 the amino acid sequence shown in amino acids of SEQ ID NO: 154 and a light chain variable region (V_{L}) which comprises three complementarity determining regions (CDRs), wherein, V_{L}CDR1 comprises the amino acid sequence shown in SEQ ID NO: 155, the V_{L}CDR2 comprises the amino acid sequence shown in SEQ ID NO: 156 and the V_{L}CDR3 comprises the amino acid sequence shown in SEQ ID NO: 157. A condensation of the three V_{H}CDRs and the three V_{L} CDRs of the LGALS3BP Ig fusion protein recited in the aforementioned paragraph is shown in the amino acids of SEQ ID NO: 22.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 158, the V_{H} CDR2 comprises the amino acid sequence shown in SEQ ID NO: 159 and the V_{H}CDR3 the amino acid sequence shown in amino acids of SEQ ID NO: 160 and a light chain variable region (V_{L}) which comprises three complementarity determining regions (CDRs), wherein, V_{L}CDR1 comprises the amino acid sequence shown in SEQ ID NO: 161, the V_{L}CDR2 comprises the amino acid sequence shown in SEQ ID NO: 162 and the V_{L}CDR3 comprises the amino acid sequence shown in SEQ ID NO: 163. A condensation of the three V_{H}CDRs and the three V_{L} CDRs of the LGALS3BP Ig fusion protein recited in the aforementioned paragraph is shown in the amino acids of SEQ ID NO: 23.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 164, the V_{H} CDR2 comprises the amino acid sequence shown in SEQ ID NO: 165 and the V_{H}CDR3 the amino acid sequence shown in amino acids of SEQ ID NO: 166 and a light chain variable region (V_{L}) which comprises three complementarity determining regions (CDRs), wherein, V_{L}CDR1 comprises the amino acid sequence shown in SEQ ID NO: 167, the V_{L}CDR2 comprises the amino acid sequence shown in SEQ ID NO: 168 and the V_{L}CDR3 comprises the amino acid sequence shown in SEQ ID NO: 169. A condensation of the three V_{H}CDRs and the three V_{L} CDRs of the LGALS3BP Ig fusion protein recited in the aforementioned paragraph is shown in the amino acids of SEQ ID NO: 24.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 170, the V_{H} CDR2 comprises the amino acid sequence shown in SEQ ID NO: 171 and the V_{H}CDR3 the amino acid sequence shown in amino acids of SEQ ID NO: 172 and a light chain variable region (V_{L}) which comprises three complementarity determining regions (CDRs), wherein, V_{L}CDR1 comprises the amino acid sequence shown in SEQ ID NO: 173, the V_{L}CDR2 comprises the amino acid sequence shown in SEQ ID NO: 174 and the V_{L}CDR3 comprises the amino acid sequence shown in SEQ ID NO: 175. A condensation of the three V_{H}CDRs and the three V_{L} CDRs of the LGALS3BP Ig fusion protein recited in the aforementioned paragraph is shown in the amino acids of SEQ ID NO: 25.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 176, the V_{H} CDR2 comprises the amino acid sequence shown in SEQ ID NO: 177 and the V_{H}CDR3 the amino acid sequence shown in amino acids of SEQ ID NO: 178 and a light chain variable region (V_{L}) which comprises three complementarity determining regions (CDRs), wherein, V_{L}CDR1 comprises the amino acid sequence shown in SEQ ID NO: 179, the V_{L}CDR2 comprises the amino acid sequence shown in SEQ ID NO: 180 and the V_{L}CDR3 comprises the amino acid sequence shown in SEQ ID NO: 181. A condensation of the three V_{H}CDRs and the three V_{L} CDRs of the LGALS3BP Ig fusion protein recited in the aforementioned paragraph is shown in the amino acids of SEQ ID NO: 26.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 182, the V_{H} CDR2 comprises the amino acid sequence shown in SEQ ID NO: 183 and the V_{H}CDR3 the amino acid sequence shown in amino acids of SEQ ID NO: 184 and a light chain variable region (V_{L}) which comprises three complementarity determining regions (CDRs), wherein, V_{L}CDR1 comprises the amino acid sequence shown in SEQ ID NO: 185, the V_{L}CDR2 comprises the amino acid sequence shown in SEQ ID NO: 186 and the V_{L}CDR3 comprises the amino acid sequence shown in SEQ ID NO: 187. A condensation of the three V_{H}CDRs and the three V_{L} CDRs of the LGALS3BP Ig fusion protein recited in the aforementioned paragraph is shown in the amino acids of SEQ ID NO: 27.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 188, the V_{H} CDR2 comprises the amino acid sequence shown in SEQ ID NO: 189 and the V_{H}CDR3 the amino acid sequence shown in amino acids of SEQ ID NO: 190 and a light chain variable region (V_{L}) which comprises three complementarity determining regions (CDRs), wherein, V_{L}CDR1 comprises the amino acid sequence shown in SEQ ID NO: 191, the V_{L}CDR2 comprises the amino acid sequence shown in SEQ ID NO: 192 and the V_{L}CDR3 comprises the amino acid sequence shown in SEQ ID NO: 193. A condensation of the three V_{H}CDRs and the three V_{L} CDRs of the LGALS3BP Ig fusion protein recited in the aforementioned paragraph is shown in the amino acids of SEQ ID NO: 28.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 194, the V_{H} CDR2 comprises the amino acid sequence shown in SEQ ID NO: 195 and the V_{H}CDR3 the amino acid sequence shown in amino acids of SEQ ID NO: 196 and a light chain variable region (V_{L}) which comprises three complementarity determining regions (CDRs), wherein, V_{L}CDR1 comprises the amino acid sequence shown in SEQ ID NO: 197, the V_{L}CDR2 comprises the amino acid sequence shown in SEQ ID NO: 198 and the V_{L}CDR3 comprises the amino acid sequence shown in SEQ ID NO: 199. A condensation of the three V_{H}CDRs and the three V_{L} CDRs of the LGALS3BP Ig fusion protein recited in the aforementioned paragraph is shown in the amino acids of SEQ ID NO: 29.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 200, the V_{H} CDR2 comprises the amino acid sequence shown in SEQ ID NO: 201 and the V_{H}CDR3 the amino acid sequence shown in amino acids of SEQ ID NO: 202 and a light chain variable region (V_{L}) which comprises three complementarity determining regions (CDRs), wherein, V_{L}CDR1 comprises the amino acid sequence shown in SEQ ID NO: 203, the V_{L}CDR2 comprises the amino acid sequence shown in SEQ ID NO: 204 and the V_{L}CDR3 comprises the amino acid sequence shown in SEQ ID NO: 205. A condensation of the three V_{H}CDRs and the three V_{L} CDRs of the LGALS3BP Ig fusion protein recited in the aforementioned paragraph is shown in the amino acids of SEQ ID NO: 30.

In one embodiment, the present invention discloses a LGALS3BP Ig fusion protein which specifically binds to LGALS3BP, wherein, the Ig fusion protein comprises a heavy chain variable region (V_{H}) which comprises three complementarity determining regions (CDRs), wherein, V_{H}CDR1 comprises the amino acid sequence shown in SEQ ID NO: 206, the V_{H} CDR2 comprises the amino acid sequence shown in SEQ ID NO: 207 and the V_{H}CDR3 the amino acid sequence shown in amino acids of SEQ ID NO: 208 and a light chain variable region (V_{L}) which comprises three complementarity determining regions (CDRs), wherein, V_{L}CDR1 comprises the amino acid sequence shown in SEQ ID NO: 209, the V_{L}CDR2 comprises the amino acid sequence shown in SEQ ID NO: 210 and the V_{L}CDR3 comprises the amino acid sequence shown in SEQ ID NO: 211. A condensation of the three V_{H}CDRs and the three V_{L} CDRs of the LGALS3BP Ig fusion protein recited in the aforementioned paragraph is shown in the amino acids of SEQ ID NO: 31.

In one embodiment, the V_{H} and the V_{L} are in a single polypeptide chain. For embodiment, the Ig fusion protein which specifically binds to LGALS3BP is:
(i) a single chain Fv fragment (scFv); or
(ii) a dimeric scFv (di-scFv); or
(iii) (i) or (ii) linked to a Fc or a heavy chain constant domain (C_{H}) 2 and/or C_{H}3; or
(iv) (i) or (ii) linked to a protein that binds to an immune effector cell.

In selected embodiments of the present invention, it is contemplated that the V_{L} and V_{H} are in separate polypeptide chains For example, the Ig fusion protein which specifically binds to LGALS3BP is:
(i) a diabody; or
(ii) a triabody; or
(iii) a tetrabody; or
(iv) a Fab; or
(v) a F(ab′)2; or
(vi) a Fv; or
(vii) one of (i) to (vi) linked to a Fc or a C_{H}2 and/or C_{H}3

In preferred embodiments of the present invention the Ig fusion protein which specifically binds to LGALS3BPs of the present invention are full length antibodies.

Tables 1-7 present different amino acid sequences descriptive of the Ig fusion proteins which specifically binds to LGALS3BPs described by various embodiment of the present invention.

TABLE 1
VH & VL CDR SEQUENCES COMBINED

| mAb ID | HCDR1/HCDR2/HCDR3/LCDR1/LCDR2/LCDR3 | Seq ID No: |
|---|---|---|
| mAb1 | GlyPheThrPheSerSerTyrGlyIleSerTyrAspGlySerAsnLysAlaLysGlySerSerProTyrTyrTyrT | 2 |
| | yrGlyMetAspValGlnSerValSerThrAsnGlyAlaSerGlnGlnTyrAsnThrTrpProProValArg | |
| mAb2 | GlyPheThrValSerSerAsnTyrIleTyrSerGlyGlySerThrAlaArgAspThrAlaSerGlyGlyMetAsp | 3 |
| | ValGlnSerValSerSerAsnGlyAlaSerGlnGlnTyrGlyTyrSerGlnIleThr | |
| mAb3 | GlyPheThrPheSerSerTyrGlyIleSerGlySerGlyGlySerThrAlaLysAlaThrGlyTyrSerSerGlyTr | 4 |
| | pTyrGlyAlaTyrPheAspTyrGlnSerValSerSerSerTyrGlyAlaSerGlnGlnTyrGlySerSerProLeu | |
| | Thr | |
| mAb4 | GlyAspSerValSerSerAsnSerAlaAlaThrTyrTyrArgSerLysTrpTyrAsnAlaArgGluPheGlnAsp | 5 |
| | SerSerSerTrpTyrGluGlyArgAlaPheAspIleSerSerAspValGlyGlyTyrAsnTyrAspValSerSerS | |
| | erTyrAlaGlySerSerValVal | |
| mAb5 | GlyAspSerValSerSerAsnSerAlaAlaThrTyrTyrArgSerLysTrpTyrAsnAlaArgGlyGlyValGlyA | 6 |
| | laThrTrpTyrTyrGlyMetAspValLysLeuGlyAspLysTyrGlnAspSerGlnThrTrpAspSerSerThr | |
| | ValVal | |
| mAb6 | GlyPheThrPheSerSerTyrSerIleTrpTyrAspGlySerAsnLysAlaArgLeuGlySerGlyTrpSerLeu | 7 |
| | AspTyrSerSerAspValGlyGlyTyrAsnTyrAspValAsnSerSerTyrThrSerSerAsnThrLeuValVal | |
| mAb7 | GlyPheThrPheSerSerTyrProIleSerTyrAspGlySerAsnLysAlaArgValGlySerGlyGlyTrpThrP | 8 |
| | roAspTyrSerSerAspValGlyGlyTyrAsnTyrAspValSerSerSerTyrThrSerSerSerThrLeuValV | |
| | al | |
| mAb8 | GlyPheThrPheSerAsnAlaTrpIleLysSerLysAsnAspGlyGlyThrThrThrThrAlaProSerLeuMe | 9 |
| | tAspValSerSerTyrIleAlaThrAsnSerSerAspSerAlaAlaTrpAspAspSerLeuAsnAlaTyrVal | |
| mAb9 | GlyPheThrPheSerSerTyrProIleSerTyrAspGlySerAsnLysAlaArgValGlySerGlyGlyTrpThrP | 10 |
| | roAspTyrSerSerAspIleGlyGlyTyrAsnTyrGluValSerSerSerTyrThrSerSerSerThrLeuValVal | |
| mAb10 | GlyPheThrPheSerSerTyrProIleSerTyrAspGlySerAsnLysAlaArgValGlySerGlyGlyTrpThrP | 11 |
| | roAspTyrSerSerAspValGlyGlyTyrAsnTyrGluValSerSerSerTyrThrSerSerSerThrLeuValV | |
| | al | |
| mAb11 | GlyPheThrPheSerSerTyrProIleSerTyrAspGlySerAsnLysAlaArgValGlySerGlyGlyTrpThrP | 12 |
| | roAspTyrSerSerAspValGlyGlyTyrAsnTyrAspValSerSerSerTyrThrSerSerSerThrLeuValV | |
| | al | |
| mAb12 | GlyPheThrValSerSerAsnTyrIleTyrSerGlyGlySerThrAlaArgAspLeuHisSerAlaAlaGlyPheA | 13 |
| | spTyrGlyAsnAsnTyrGluAsnAsnGlyThrTrpAspSerSerLeuAsnValGlyVal | |
| mAb13 | GlyPheThrValSerSerAsnTyrIleTyrSerGlyGlySerThrAlaArgAspPheGluGlySerGlyAlaLeu | 14 |
| | AspValAsnIleGlyAspLysArgTyrAspThrGlnValTrpAspThrAspThrAsnHisAlaVal | |
| mAb14 | GlyPheThrPheSerAsnAlaTrplIeLysSerLysAsnAspGlyGlyThrThrThrThrAlaProSerLeuMe | 15 |
| | tAspValIleLeuGlyHisTyrHisGlyLysAspAsnAsnSerArgAspArgSerGlyThrGlnValLeu | |
| mAb15 | GlyPheThrValSerSerAsnTyrIleTyrSerGlyGlySerThrAlaArgAspLeuSerTyrSerAspAlaPhe | 16 |
| | AspIleSerSerAsnIleGlyAsnAsnTyrAspAsnAspGlyThrTrpAspAsnSerLeuSerAlaValVal | |
| mAb16 | GlyPheThrPheSerSerTyrGlyIleTrpTyrAspGlyAsnAsnLysAlaArgAspAsnSerGlySerTyrAs | 17 |
| | nTrpPheAsnProSerSerAspValGlyGlyTyrAsnTyrGluValSerSerSerTyrSerGlySerAsnAsnL | |
| | euValVal | |
| mAb22 | GlyPheThrPheSerSerTyrProIleSerTyrAspGlyGlyAsnLysAlaArgValGlySerGlyGlyTrpThrP | 18 |
| | roAspTyrSerSerAspValGlyGlyTyrAsnTyrGluValThrSerSerTyrThrSerSerSerThrPheValV | |
| | al | |
| mAb101 | GlyPheThrPheSerSerTyrAlaIleSerTyrAspGlySerAsnLysAlaArgAspArgGlyValGluGlyAla | 19 |
| | TyrGlyMetAspValGlnArgValArgSerSerTyrGlyAlaSerGlnGlnTyrGlySerSerProProArgIleIl | |
| | e | |
| mAb102 | GlyTyrThrPheThrGlyTyrTyrIleAsnProAsnSerGlyGlyThrAlaArgGlyGlyAspCysSerSerThr | 20 |
| | SerCysTyrAspProAspTyrGlyGlySerIleAlaSerAsnTyrLysAspAsnGlnSerTyrGlySerGlyAsn | |
| | ValVal | |
| mAb103 | GlyTyrThrPheThrSerTyrTyrIleAsnProSerGlyGlySerThrAlaArgGluAspHisAspTyrSerAsn | 21 |
| | GlnGlyGlyPheAspTyrGlnSerValThrSerAsnTyrGlyAlaSerGlnGlnTyrGlySerSerProThr | |
| mAb104 | GlyAspSerValSerSerAsnSerAlaAlaThrTyrTyrArgSerLysTrpTyrAsnAlaArgGluLysIleAlaV | 22 |
| | alAlaGlyTyrTyrTyrGlyMetAspValLysLeuGlyAspLysTyrGlnAsnAsnGlnAlaTrpAspSerSer | |
| | AlaValVal | |
| mAb105 | GlyAspSerValSerSerAsnSerAlaAlaThrTyrTyrSerSerLysTrpTyrAsnAlaArgGlyGlySerSerG | 23 |
| | luPheTyrTyrTyrGlyMetAspValLysLeuGlyAsnLysTyrGluAsnAsnGlnAlaTrpAspSerSerThr | |
| | AlaVal | |
| mAb106 | GlyPheThrPheAspAspTyrAlaIleSerTrpAsnSerGlySerIleAlaLysAspIleAlaAlaGlyGlyLeuAs | 24 |
| | pSerGlnSerlIeSerSerTyrAlaAlaSerGlnGlnSerTyrSerThrSerTrpThr | |
| mAb107 | GlyTyrThrPheThrSerTyrGlyIleSerAlaTyrAsnGlyAsnThrAlaArgGlyLeuGlyAspSerSerSerS | 25 |
| | erTyrThrSerAsnIleGlyAlaAsnHisThrLysAsnAlaAlaTrpAspAspSerLeuArgGlyTrpThr | |
| mAb108 | GlyTyrSerPheThrSerTyrTrpIleTyrProGlyAspSerAspThrAlaSerGlyAlaSerProTyrTyrPheA | 26 |
| | spTyrSerLeuArgSerTyrTyrGlyLysAsnAsnSerArgAspSerSerGlyAsnHisTrpVal | |
| mAb109 | GlyTyrThrPheThrSerTyrGlyIleSerAlaTyrAsnGlyAsnThrAlaArgAspProValTyrSerSerSerT | 27 |
| | rpGlyGlyTyrAlaPheAspIleGlnGlyValAsnSerAspGlyAlaSerGlnGlnTyrAsnAsnTrpProTrpT | |
| | hr | |
| mAb110 | GlyPheThrPheSerSerTyrProIleSerTyrAspGlySerAsnLysThrArgValGlySerGlyGlyTrpThr | 28 |
| | ProAspTyrSerSerAspValGlyGlyTyrAsnTyrGluValSerSerSerTyrThrSerSerSerThrLeuVal | |
| | Val | |
| mAb111 | GlyPheThrValSerSerAsnTyrIleTyrSerGlyGlySerThrAlaArgAspThrAlaSerGlyGlyMetAsp | 29 |
| | ValGlnSerValSerSerAsnGlyAlaSerGlnGlnTyrGlyTyrSerGlnIleThr | |
| mAb112 | GlyPheThrPheSerSerTyrGlyIleTrpTyrAspGlySerAsnLysAlaArgGluValValGlySerTyrTyrL | 30 |
| | euAspTyrSerSerAspIleGlyGlyTyrLysTyrAspValThrGlySerTyrSerSerSerSerSerHisTyrVal | |
| mAb113 | GlyPheThrPheSerSerTyrTrplIeLysGlnAspGlySerGluLysAlaArgAspLeuHisCysGlySerSer | 31 |
| | CysGlyProGluAlaGlnThrlIeSerSerTyrGlyAlaSerGlnGlnSerTyrSerThrProGlnThr | |

TABLE 2
VH AND VL ELISA REACTIVITY

| | | | huEGFR |
|---|---|---|---|
| | | | ELISA |
| mAb | Seq ID | huLGALS3BP | reactivity |
| ID | No: | ELISA reactivity (OD) | (OD) |
| mAb1 | | 1.5794 | 0.0948 |
| mAb2 | | 2.559 | 0.0944 |
| mAb3 | | 2.5552 | 0.0936 |
| mAb4 | | 2.5288 | 0.0898 |
| mAb5 | | 0.8091 | 0.0856 |
| mAb6 | | 2.5542 | 0.0797 |
| mAb7 | | 1.6491 | 0.1006 |
| mAb8 | | 0.128 | 0.0899 |
| mAb9 | | 2.5658 | 0.0984 |
| mAb10 | | 2.4879 | 0.096 |
| mAb11 | | 2.5157 | 0.0978 |
| mAb12 | | 2.5803 | 0.0939 |
| mAb13 | | 2.5866 | 0.084 |
| mAb14 | | 0.203 | 0.0901 |
| mAb15 | | 0.8852 | 0.0785 |
| mAb16 | | 2.549 | 0.0844 |
| mAb22 | | 2.47 | 0.0925 |
| mAb101 | | Full dose response in graph | |
| mAb102 | | Full dose response in graph | |
| mAb103 | | Full dose response in graph | |
| mAb104 | | Full dose response in graph | |
| mAb105 | | Full dose response in graph | |
| mAb106 | | Full dose response in graph | |
| mAb107 | | Full dose response in graph | |
| mAb108 | | Full dose response in graph | |
| mAb109 | | Full dose response in graph | |
| mAb110 | | Full dose response in graph | |
| mAb111 | | Full dose response in graph | |
| mAb112 | | Full dose response in graph | |
| mAb113 | | Full dose response in graph | |

TABLE 3
DISCRETE CDR5 FOR VH AND VL SEQUENCES

| mAb | HCDR1 | HCDR2 | HCDR3 | LCDR1 | LCDR2 | LCDR3 |
|---|---|---|---|---|---|---|
| mAb1 | GlyPheT | IleSerTyrAs | AlaLysGlySe | GlnSerValS | GlyAlaSer | GlnGlnTyrAsnThrTrpProProV |
| | hrPheSer | pGlySerAsn | rSerProTyrT | erThrAsn | (SEQ ID | alArg |
| | SerTyrGl | Lys (SEQ ID | yrTyrTyrGly | (SEQ ID | NO: 36) | (SEQ ID NO: 37) |
| | y (SEQ | NO: 33) | MetAspVal | NO: 35) | | |
| | ID NO: | | (SEQ ID NO: | | | |
| | 32) | | 34) | | | |
| mAb2 | GlyPheT | IleTyrSerGl | AlaArgAspT | GlnSerValS | GlyAlaSer | GlnGlnTyrGlyTyrSerGlnIleThr |
| | hrValSer | yGlySerThr | hrAlaSerGly | erSerAsn | (SEQ ID | (SEQ ID NO: 43) |
| | SerAsnTy | (SEQ ID NO: | GlyMetAsp | (SEQ ID | NO: 42) | |
| | r (SEQ ID | 39) | Val (SEQ ID | NO: 41) | | |
| | NO: 38) | | NO: 40) | | | |
| mAb3 | GlyPheT | IleSerGlySe | AlaLysAlaT | GlnSerValS | GlyAlaSer | GlnGlnTyrGlySerSerProLeuT |
| | hrPheSer | rGlyGlySerT | hrGlyTyrSer | erSerSerTyr | (SEQ ID | hr |
| | SerTyrGl | hr | SerGlyTrpT | (SEQ ID | NO: 48) | (SEQ ID NO: 49) |
| | y | (SEQ ID NO: | yrGlyAlaTyr | NO: 47) | | |
| | (SEQ ID | 45) | PheAspTyr | | | |
| | NO: 44) | | (SEQ ID NO: | | | |
| | | | 46) | | | |
| mAb4 | GlyAspSe | ThrTyrTyrA | AlaArgGluP | SerSerAspV | AspValSer | SerSerTyrAlaGlySerSerValVal |
| | rValSerS | rgSerLysTrp | heGlnAspS | alGlyGlyTyr | (SEQ ID | (SEQ ID NO: 55) |
| | erAsnSer | TyrAsn | erSerSerTrp | AsnTyr | NO: 54) | |
| | AlaAla | (SEQ ID NO: | TyrGluGlyA | (SEQ ID | | |
| | (SEQ ID | 51) | rgAlaPheAs | NO: 53) | | |
| | NO: 50) | | pIle (SEQ ID | | | |
| | | | NO: 52) | | | |
| mAb5 | GlyAspSe | ThrTyrTyrA | AlaArgGlyG | LysLeuGlyA | GlnAspSer | GlnThrTrpAspSerSerThrValV |
| | rValSerS | rgSerLysTrp | lyValGlyAla | spLysTyr | (SEQ ID | al |
| | erAsnSer | TyrAsn | ThrTrpTyrT | (SEQ ID | NO: 60) | (SEQ ID NO: 61) |
| | AlaAla | (SEQ ID NO: | yrGlyMetAs | NO: 59) | | |
| | (SEQ ID | 57) | pVal (SEQ | | | |
| | NO: 56) | | ID NO: 58) | | | |
| mAb6 | GlyPheT | IleTrpTyrAs | AlaArgLeuG | SerSerAspV | AspValAsn | SerSerTyrThrSerSerAsnThrLe |
| | hrPheSer | pGlySerAsn | lySerGlyTrp | alGlyGlyTyr | (SEQ ID | uValVal (SEQ ID NO: 67) |
| | SerTyrSe | Lys (SEQ ID | SerLeuAspT | AsnTyr | NO: 66) | |
| | r (SEQ ID | NO: 63) | yr (SEQ ID | (SEQ ID | | |
| | NO: 62) | | NO: 64) | NO: 65) | | |
| mAb7 | GlyPheT | IleSerTyrAs | AlaArgValGl | SerSerAspV | AspValSer | SerSerTyrThrSerSerSerThrLe |
| | hrPheSer | pGlySerAsn | ySerGlyGly | alGlyGlyTyr | (SEQ ID | uValVal (SEQ ID NO: 73) |
| | SerTyrPr | Lys (SEQ ID | TrpThrProA | AsnTyr | NO: 72) | |
| | o (SEQ ID | NO: 69) | spTyr (SEQ | (SEQ ID | | |
| | NO: 68) | | ID NO: 70) | NO: 71) | | |
| mAb8 | GlyPheT | IleLysSerLys | ThrThrAlaP | SerSerTyrIl | SerAspSer | AlaAlaTrpAspAspSerLeuAsnA |
| | hrPheSer | AsnAspGly | roSerLeuM | eAlaThrAsn | (SEQ ID | laTyrVal (SEQ ID NO: 79) |
| | AsnAlaTr | GlyThrThr | etAspVal | Ser | NO: 78) | |
| | p (SEQ ID | (SEQ ID NO: | (SEQ ID NO: | (SEQ ID | | |
| | NO: 74) | 75) | 76) | NO: 77) | | |
| mAb9 | GlyPheT | IleSerTyrAs | AlaArgValGl | SerSerAspIl | GluValSer | SerSerTyrThrSerSerSerThrLe |
| | hrPheSer | pGlySerAsn | ySerGlyGly | eGlyGlyTyr | (SEQ ID | uValVal (SEQ ID NO: 85) |
| | SerTyrPr | Lys (SEQ ID | TrpThrProA | AsnTyr | NO: 84) | |
| | o (SEQ ID | NO: 81) | spTyr (SEQ | (SEQ ID | | |
| | NO: 80) | | ID NO: 82) | NO: 83) | | |
| mAb10 | GlyPheT | IleSerTyrAs | AlaArgValGl | SerSerAspV | GluValSer | SerSerTyrThrSerSerSerThrLe |
| | hrPheSer | pGlySerAsn | ySerGlyGly | alGlyGlyTyr | (SEQ ID | uValVal (SEQ ID NO: 91) |
| | SerTyrPr | Lys (SEQ ID | TrpThrProA | AsnTyr | NO: 90) | |
| | o (SEQ ID | NO: 87) | spTyr (SEQ | (SEQ ID | | |
| | NO: 86) | | ID NO: 88) | NO: 89) | | |
| mAb11 | GlyPheT | IleSerTyrAs | AlaArgValGl | SerSerAspV | AspValSer | SerSerTyrThrSerSerSerThrLe |
| | hrPheSer | pGlySerAsn | ySerGlyGly | alGlyGlyTyr | (SEQ ID | uValVal (SEQ ID NO: 97) |
| | SerTyrPr | Lys (SEQ ID | TrpThrProA | AsnTyr | NO: 96) | |
| | o (SEQ ID | NO: 93) | spTyr (SEQ | (SEQ ID | | |
| | NO: 92) | | ID NO: 94) | NO: 95) | | |
| mAb12 | GlyPheT | IleTyrSerGl | AlaArgAspL | GlyAsnAsnT | GluAsnAsn | GlyThrTrpAspSerSerLeuAsnV |
| | hrValSer | yGlySerThr | euHisSerAl | yr (SEQ ID | (SEQ ID | alGlyVal (SEQ ID NO: 103) |
| | SerAsnTy | (SEQ ID NO: | aAlaGlyPhe | NO: 101) | NO: 102) | |
| | r (SEQ ID | 99) | AspTyr | | | |
| | NO: 98) | | (SEQ ID NO: | | | |
| | | | 100) | | | |
| mAb13 | GlyPheT | IleTyrSerGl | AlaArgAspP | AsnIleGlyAs | TyrAspThr | GlnValTrpAspThrAspThrAsn |
| | hrValSer | yGlySerThr | heGluGlySe | pLysArg | (SEQ ID | HisAlaVal (SEQ ID NO: 109) |
| | SerAsnTy | (SEQ ID NO: | rGlyAlaLeu | (SEQ ID | NO: 108) | |
| | r (SEQ ID | 105) | AspVal | NO: 107) | | |
| | NO: 104) | | (SEQ ID NO: | | | |
| | | | 106) | | | |
| mAb14 | GlyPheT | IleLysSerLys | ThrThrAlaP | IleLeuGlyHi | GlyLysAspA | AsnSerArgAspArgSerGlyThrG |
| | hrPheSer | AsnAspGly | roSerLeuM | sTyrHis | sn | lnValLeu |
| | AsnAlaTr | GlyThrThr | etAspVal | (SEQ ID | (SEQ ID | (SEQ ID NO: 115) |
| | p (SEQ ID | (SEQ ID NO: | (SEQ ID NO: | NO: 113) | NO: 114) | |
| | NO: 110) | 111) | 112) | | | |
| mAb15 | GlyPheT | IleTyrSerGl | AlaArgAspL | SerSerAsnIl | AspAsnAsp | GlyThrTrpAspAsnSerLeuSerA |
| | hrValSer | yGlySerThr | euSerTyrSe | eGlyAsnAs | (SEQ ID | laValVal (SEQ ID NO: 121) |
| | SerAsnTy | (SEQ ID NO: | rAspAlaPhe | nTyr | NO: 120) | |
| | r (SEQ ID | 117) | AspIle (SEQ | (SEQ ID | | |
| | NO: 116) | | ID NO: 118) | NO: 119) | | |
| mAb16 | GlyPheT | IleTrpTyrAs | AlaArgAspA | SerSerAspV | GluValSer | SerSerTyrSerGlySerAsnAsnLe |
| | hrPheSer | pGlyAsnAs | snSerGlySe | alGlyGlyTyr | (SEQ ID | uValVal (SEQ ID NO: 127) |
| | SerTyrGl | nLys (SEQ | rTyrAsnTrp | AsnTyr | NO: 126) | |
| | y (SEQ ID | ID NO: 123) | PheAsnPro | (SEQ ID | | |
| | NO: 122) | | (SEQ ID NO: | NO: 125) | | |
| | | | 124) | | | |
| mAb22 | GlyPheT | IleSerTyrAs | AlaArgValGl | SerSerAspV | GluValThr | SerSerTyrThrSerSerSerThrPh |
| | hrPheSer | pGlyGlyAsn | ySerGlyGly | alGlyGlyTyr | (SEQ ID | eValVal (SEQ ID NO: 133) |
| | SerTyrPr | Lys (SEQ ID | TrpThrProA | AsnTyr | NO: 132) | |
| | o (SEQ ID | NO: 129) | spTyr (SEQ | (SEQ ID | | |
| | NO: 128) | | ID NO: 130) | NO: 131) | | |
| mAb101 | GlyPheT | IleSerTyrAs | AlaArgAspA | GlnArgValA | GlyAlaSer | GlnGlnTyrGlySerSerProProAr |
| | hrPheSer | pGlySerAsn | rgGlyValGlu | rgSerSerTyr | (SEQ ID | gIleIle |
| | SerTyrAl | Lys (SEQ ID | GlyAlaTyrGl | (SEQ ID | NO: 138) | (SEQ ID NO: 139) |
| | a (SEQ ID | NO: 135) | yMetAspVa | NO: 137) | | |
| | NO: 134) | | l (SEQ ID | | | |
| | | | NO: 136) | | | |
| mAb102 | GlyTyrTh | IleAsnProA | AlaArgGlyG | GlyGlySerOl | LysAspAsn | GlnSerTyrGlySerGlyAsnValVa |
| | rPheThr | snSerGlyGl | IyAspCysSe | eAlaSerAsn | (SEQ ID | l |
| | GlyTyrTy | yThr (SEQ | rSerThrSer | Tyr (SEQ ID | NO: 144) | (SEQ ID NO: 145) |
| | r (SEQ ID | ID NO: 141) | CysTyrAspP | NO: 143) | | |
| | NO: 140) | | roAspTyr | | | |
| | | | (SEQ ID NO: | | | |
| | | | 142) | | | |
| mAb103 | GlyTyrTh | IleAsnProS | AlaArgGluA | GlnSerValT | GlyAlaSer | GlnGlnTyrGlySerSerProThr |
| | rPheThrS | erGlyGlySer | spHisAspTy | hrSerAsnTy | (SEQ ID | (SEQ ID NO: 151) |
| | erTyrTyr | Thr (SEQ ID | rSerAsnGln | r (SEQ ID | NO: 150) | |
| | (SEQ ID | NO: 147) | GlyGlyPheA | NO: 149) | | |
| | NO: 146) | | spTyr (SEQ | | | |
| | | | ID NO: 148) | | | |
| mAb104 | GlyAspSe | ThrTyrTyrA | AlaArgGluL | LysLeuGlyA | GlnAsnAsn | GlnAlaTrpAspSerSerAlaValVa |
| | rValSerS | rgSerLysTrp | ysIleAlaVal | spLysTyr | (SEQ ID | l |
| | erAsnSer | TyrAsn | AlaGlyTyrT | (SEQ ID | NO: 156) | (SEQ ID NO: 157) |
| | AlaAla | (SEQ ID NO: | yrTyrGlyMe | NO: 155) | | |
| | (SEQ ID | 153) | tAspVal | | | |
| | NO: 152) | | (SEQ ID NO: | | | |
| | | | 154) | | | |
| mAb105 | GlyAspSe | ThrTyrTyrS | AlaArgGlyG | LysLeuGlyA | GluAsnAsn | GlnAlaTrpAspSerSerThrAlaV |
| | rValSerS | erSerLysTrp | lySerSerGlu | snLysTyr | (SEQ ID | al (SEQ ID NO: 163) |
| | erAsnSer | TyrAsn | PheTyrTyrT | (SEQ ID | NO: 162) | |
| | AlaAla | (SEQ ID NO: | yrGlyMetAs | NO: 161) | | |
| | (SEQ ID | 159) | pVal (SEQ | | | |
| | NO: 158) | | ID NO: 160) | | | |
| mAb106 | GlyPheT | IleSerTrpAs | AlaLysAspIl | GlnSerIleSe | AlaAlaSer | GlnGlnSerTyrSerThrSerTrpTh |
| | hrPheAs | nSerGlySerI | eAlaAlaGly | rSerTyr | (SEQ ID | r |
| | pAspTyr | le (SEQ ID | GlyLeuAspS | (SEQ ID | NO: 168) | (SEQ ID NO: 169) |
| | Ala (SEQ | NO: 165) | er (SEQ ID | NO: 167) | | |
| | ID NO: | | NO: 166) | | | |
| | 164) | | | | | |
| mAb107 | GlyTyrTh | IleSerAlaTy | AlaArgGlyL | ThrSerAsnIl | ThrLysAsn | AlaAlaTrpAspAspSerLeuArgG |
| | rPheThrS | rAsnGlyAsn | euGlyAspSe | eGlyAlaAsn | (SEQ ID | lyTrpThr (SEQ ID NO: 175) |
| | erTyrGly | Thr (SEQ ID | rSerSerSerT | His (SEQ ID | NO: 174) | |
| | (SEQ ID | NO: 171) | yr (SEQ ID | NO: 173) | | |
| | NO: 170) | | NO: 172) | | | |
| mAb108 | GlyTyrSe | IleTyrProGl | AlaSerGlyAl | SerLeuArgS | GlyLysAsn | AsnSerArgAspSerSerGlyAsnH |
| | rPheThrS | yAspSerAsp | aSerProTyr | erTyrTyr | (SEQ ID | isTrpVal |
| | erTyrTrp | Thr (SEQ ID | TyrPheAspT | (SEQ ID | NO: 180) | (SEQ ID NO: 181) |
| | (SEQ ID | NO: 177) | yr (SEQ ID | NO: 179) | | |
| | NO: 176) | | NO: 178) | | | |
| mAb109 | GlyTyrTh | IleSerAlaTy | AlaArgAspP | GlnGlyValA | GlyAlaSer | GlnGlnTyrAsnAsnTrpProTrpT |
| | rPheThrS | rAsnGlyAsn | roValTyrSer | snSerAsp | (SEQ ID | hr (SEQ ID NO: 187) |
| | erTyrGly | Thr | SerSerTrpG | (SEQ ID | NO: 186) | |
| | (SEQ ID | (SEQ ID NO: | lyGlyTyrAla | NO: 185) | | |
| | NO: 182) | 183) | PheAspIle | | | |
| | | | (SEQ ID NO: | | | |
| | | | 184) | | | |
| mAb110 | GlyPheT | IleSerTyrAs | ThrArgValG | SerSerAspV | GluValSer | SerSerTyrThrSerSerSerThrLe |
| | hrPheSer | pGlySerAsn | lySerGlyGly | alGlyGlyTyr | (SEQ ID | uValVal (SEQ ID NO: 193) |
| | SerTyrPr | Lys (SEQ ID | TrpThrProA | AsnTyr | NO: 192) | |
| | o (SEQ ID | NO: 189) | spTyr (SEQ | (SEQ ID | | |
| | NO: 188) | | ID NO: 190) | NO: 191) | | |
| mAb111 | GlyPheT | IleTyrSerGl | AlaArgAspT | GlnSerValS | GlyAlaSer | GlnGlnTyrGlyTyrSerGlnIleThr |
| | hrValSer | yGlySerThr | hrAlaSerGly | erSerAsn | (SEQ ID | (SEQ ID NO: 199) |
| | SerAsnTy | (SEQ ID NO: | GlyMetAsp | (SEQ ID | NO: 198) | |
| | r (SEQ ID | 195) | Val (SEQ ID | NO: 197) | | |
| | NO: 194) | | NO: 196) | | | |
| mAb112 | GlyPheT | IleTrpTyrAs | AlaArgGluV | SerSerAspIl | AspValThr | GlySerTyrSerSerSerSerSerHis |
| | hrPheSer | pGlySerAsn | alValGlySer | eGlyGlyTyr | (SEQ ID | TyrVal |
| | SerTyrGl | Lys (SEQ ID | TyrTyrLeuA | LysTyr (SEQ | NO: 204) | (SEQ ID NO: 205) |
| | y (SEQ ID | NO: 201) | spTyr (SEQ | ID NO: 203) | | |
| | NO: 200) | | ID NO: 202) | | | |
| mAb113 | GlyPheT | IleLysGlnAs | AlaArgAspL | GlnThrIleSe | GlyAlaSer | GlnGlnSerTyrSerThrProGlnT |
| | hrPheSer | pGlySerGlu | euHisCysGl | rSerTyr | (SEQ ID | hr |
| | SerTyrTr | Lys (SEQ ID | ySerSerCys | (SEQ ID | NO: 210) | (SEQ ID NO: 211) |
| | p (SEQ ID | NO: 207) | GlyProGluA | NO: 209) | | |
| | NO: 206) | | la (SEQ ID | | | |
| | | | NO: 208) | | | |

TABLE 4
DISCRETE CDR5 FOR LH SEQUENCES

| | SEQ | | SEQ | | SEQ | |
|---|---|---|---|---|---|---|
| | ID | | ID | | ID | |
| VH_ID | NO: | VH_CDR1 | NO: | VH_CDR2 | NO: | VH_CDR3 |
| VH_1 | 212 | GlyAspSerIleSerSerG | 382 | IleSerTyrAspGlySerAs | 552 | AlaArgValGlySerGlyGlyTrpThrPr |
| | | lyTyrTrp | | nLys | | oAspTyr |
| VH_2 | 213 | GlyAspSerValSerSer | 383 | IleAsnProAsnSerGlyGl | 553 | AlaArgGluValAlaThrIleProAlaHi |
| | | AsnSerAlaAla | | yThr | | sPheAspTyr |
| VH_3 | 214 | GlyAspSerValSerSer | 384 | IleSerAlaTyrAsnGlyAs | 554 | AlaArgAspTyrAspIleLeuThrGlyL |
| | | AsnSerAlaAla | | nThr | | euAspTyr |
| VH_4 | 215 | GlyAspSerValSerSer | 385 | IleSerGlySerGlyGlyArg | 555 | AlaLysAspTrpAlaGlyTyrIleAsnGl |
| | | AsnSerAlaAla | | Thr | | yTrpTyrGlyAsn |
| VH_5 | 216 | GlyAspSerValSerSer | 386 | IleSerGlySerGlyGlySer | 556 | AlaLysAspTrpAlaGlyTyrValAsnG |
| | | AsnSerAlaAla | | Thr | | lyTrpTyrGlyAsn |
| VH_6 | 217 | GlyAspSerValSerSer | 387 | IleSerGlySerGlyGlySer | 557 | AlaLysAspTrpGlyThrSerLeuLeuT |
| | | AsnSerAlaAla | | Thr | | yrGlyTyrPheAspTyr |
| VH_7 | 218 | GlyAspSerValSerSer | 388 | IleSerTyrAspGlySerAs | 558 | AlaArgValGlySerGlyGlyTrpThrPr |
| | | AsnSerAlaAla | | nLys | | oAspTyr |
| VH_8 | 219 | GlyAspSerValSerSer | 389 | IleTyrSerGlyGlySerThr | 559 | AlaArgAspPheGluGlySerGlyAlaL |
| | | AsnSerAlaAla | | | | euAspVal |
| VH_9 | 220 | GlyAspSerValSerSer | 390 | ThrTyrTyrSerSerLysTr | 560 | AlaArgGlyGlySerSerGluPheTyrT |
| | | AsnSerAlaAla | | pTyrAsn | | yrTyrGlyMetAspVal |
| VH_10 | 221 | GlyAspSerValSerSer | 391 | IleSerGlySerGlyGlyIleT | 561 | AlaLysAspTrpAlaGlyTyrThrAsnG |
| | | AspSerAlaSer | | hr | | lyTrpTyrGlySer |
| VH_11 | 222 | GlyGlySerIleSerGlyS | 392 | IleSerGlySerGlyGlyIleT | 562 | AlaLysAspTrpAlaGlyTyrThrAsnG |
| | | erAsnTyrTyr | | hr | | lyTrpTyrGlySer |
| VH_12 | 223 | GlyGlySerIleSerSerS | 393 | IleSerGlySerGlyGlySer | 563 | AlaLysAspArgSerArgArgAlaProT |
| | | erAsnTrp | | Thr | | yrTyrPheAspTyr |
| VH_13 | 224 | GlyGlySerIleSerSerS | 394 | IleSerGlySerGlyGlySer | 564 | AlaLysValTyrArgGlyTyrAspAlaP |
| | | erAsnTrp | | Thr | | heAspIle |
| VH_14 | 225 | GlyGlySerIleSerSerS | 395 | IleTyrProGlyAspSerAs | 565 | AlaArgHisAlaGlyAspGlyGlnIleAs |
| | | erAsnTrp | | pThr | | pTyr |
| VH_15 | 226 | GlyGlySerIleSerSerS | 396 | ThrTyrTyrArgSerLysTr | 566 | AlaArgGluGlySerGlyLeuTyrTyrT |
| | | erAsnTrp | | pTyrAsn | | yrTyrGlyMetAspVal |
| VH_16 | 227 | GlyGlySerValSerSer | 397 | IleSerGlySerGlyGlySer | 567 | AlaArgGlyGlySerGlyTrpTyrHisTy |
| | | AsnSerAlaAla | | Thr | | rPheAspTyr |
| VH_17 | 228 | GlyGlyThrPheSerSer | 398 | IleSerGlyThrGlyGlyArg | 568 | AlaLysAspTrpAlaGlyTyrIleAsnGl |
| | | TyrAla | | Thr | | yTrpTyrGlySer |
| VH_18 | 229 | GlyGlyThrPheSerSer | 399 | IleSerTyrAspGlySerAs | 569 | AlaArgValGlySerGlyGlyTrpThrPr |
| | | TyrAla | | nLys | | oAspTyr |
| VH_19 | 230 | GlyGlyThrPheSerSer | 400 | IleTrpTyrAspGlySerAs | 570 | AlaArgLeuGlySerGlyTrpSerLeuA |
| | | TyrAla | | nLys | | spTyr |
| VH_20 | 231 | GlyPheThrPheAsnTh | 401 | IleSerGlySerGlyAspArg | 571 | AlaLysAspTrpAlaGlyTyrIleAsnGl |
| | | rTyrAla | | Thr | | yTrpPheGlyAsn |
| VH_21 | 232 | GlyPheThrPheAsnTh | 402 | IleSerGlySerGlyAspIle | 572 | AlaLysAspTrpAlaGlyTyrValAsnG |
| | | rTyrAla | | Thr | | lyTrpTyrGlyAsn |
| VH_22 | 233 | GlyPheThrPheAsnTh | 403 | IleSerTyrAspGlySerAs | 573 | AlaArgValGlySerGlyGlyTrpThrPr |
| | | rTyrAla | | nLys | | oAspTyr |
| VH_23 | 234 | GlyPheThrPheAspAs | 404 | IleAsnAlaGlyAsnGlyAs | 574 | AlaArgGlyGlyTyrCysSerSerThrS |
| | | pTyrAla | | nThr | | erCysTyrProAspTyrAsnTrpPheA |
| | | | | | | spPro |
| VH_24 | 235 | GlyPheThrPheAspAs | 405 | IleSerGlySerGlyAspArg | 575 | AlaLysAspTrpAlaGlyTyrIleAsnGl |
| | | pTyrAla | | Thr | | yTrpTyrAlaAsn |
| VH_25 | 236 | GlyPheThrPheAspAs | 406 | IleTyrSerGlyGlySerThr | 576 | AlaArgAspArgArgGlyGlyAsnTrp |
| | | pTyrAla | | | | TyrGluPheAspTyr |
| VH_26 | 237 | GlyPheThrPheAspAs | 407 | IleTyrSerGlyGlySerThr | 577 | AlaArgGluGlyLeuAlaMetAlaGly |
| | | pTyrAla | | | | TyrPheAspTyr |
| VH_27 | 238 | GlyPheThrPheGlyAs | 408 | IleLysHisAspGlySerGlu | 578 | AlaArgValAlaValGlyAlaAsnLeuA |
| | | nHisGly | | Gln | | laPheAspIle |
| VH_28 | 239 | GlyPheThrPheSerAr | 409 | IleSerGlySerGlyAspArg | 579 | AlaLysAspTrpAlaGlyTyrIleAsnGl |
| | | gTyrGly | | Thr | | yTrpTyrGlyAsn |
| VH_29 | 240 | GlyPheThrPheSerAs | 410 | IleIleProIlePheGlyThrA | 580 | AlaArgGlyMetAlaGlnSerProAla |
| | | nAlaTrp | | la | | PheAspTyr |
| VH_30 | 241 | GlyPheThrPheSerAs | 411 | IleSerGlySerGlyGlyArg | 581 | AlaLysAspTrpAlaGlyTyrIleAsnGl |
| | | nAlaTrp | | Thr | | yTrpTyrGlyAsn |
| VH_31 | 242 | GlyPheThrPheSerAs | 412 | ThrTyrTyrAsnSerLysTr | 582 | AlaArgGluThrGlyGlyPheAspTyr |
| | | nAlaTrp | | pTyrAsn | | |
| VH_32 | 243 | GlyPheThrPheSerAs | 413 | IleAsnThrAspGlyGlyAs | 583 | AlaArgAspProValArgGlyAspGly |
| | | nTyrAla | | nThr | | TyrAsnPheAspTyr |
| VH_33 | 244 | GlyPheThrPheSerAs | 414 | IleSerGlySerGlyAspIle | 584 | AlaLysAspTrpAlaGlyTyrValAsnG |
| | | nTyrAla | | Thr | | lyTrpTyrGlyAsn |
| VH_34 | 245 | GlyPheThrPheSerAs | 415 | IleSerGlySerGlyGlySer | 585 | AlaLysAlaThrGlyTyrSerSerGlyTr |
| | | nTyrAla | | Thr | | pTyrGlyAlaTyrPheAspTyr |
| VH_35 | 246 | GlyPheThrPheSerAs | 416 | IleTyrHisSerGlySerThr | 586 | AlaArgAspArgGlySerMetAspVal |
| | | nTyrAla | | | | |
| VH_36 | 247 | GlyPheThrPheSerAs | 417 | IleTyrProGlyAspSerAs | 587 | AlaArgLeuGlyArgThrSerHisGlnS |
| | | nTyrAla | | pThr | | erTrpAspLeuGlyTyr |
| VH_37 | 248 | GlyPheThrPheSerAs | 418 | IleTyrProGlyAspSerAs | 588 | AlaSerGlyAlaSerProTyrTyrPheA |
| | | nTyrAla | | pThr | | spTyr |
| VH_38 | 249 | GlyPheThrPheSerAs | 419 | IleTyrSerGlyGlySerThr | 589 | AlaArgGluSerAsnThrAlaAsnThr |
| | | nTyrAla | | | | HisPheAspTyr |
| VH_39 | 250 | GlyPheThrPheSerAs | 420 | ThrTyrTyrArgSerLysTr | 590 | AlaArgGlyGlyValGlyAlaThrTrpT |
| | | nTyrAla | | pTyrAsn | | yrTyrGlyMetAspVal |
| VH_40 | 251 | GlyPheThrPheSerAs | 421 | IleSerTyrAspGlySerAs | 591 | AlaLysGlnGlnTrpLeuGlyThrTrpT |
| | | nTyrGly | | nLys | | yrPheAspLeu |
| VH_41 | 252 | GlyPheThrPheSerAs | 422 | IleSerTyrAspGlySerAs | 592 | AlaLysGlyLeuLeuValAlaSerIleTy |
| | | nTyrGly | | nLys | | rAspAlaPheAspIle |
| VH_42 | 253 | GlyPheThrPheSerAs | 423 | IleSerTrpAsnSerGlySer | 593 | AlaLysAspIleAlaAlaGlyGlyLeuAs |
| | | pTyrAla | | Ile | | pSer |
| VH_43 | 254 | GlyPheThrPheSerAs | 424 | ValSerGlySerGlyThrSe | 594 | AlaLysAspTrpAlaGlyTyrIleAsnGl |
| | | pTyrTyr | | rThr | | yTrpTyrGlyAsn |
| VH_44 | 255 | GlyPheThrPheSerSe | 425 | IleAsnProAsnSerGlyAs | 595 | AlaArgGluGlnTrpLeuGlyProAla |
| | | rTyrAla | | pThr | | HisPheAspTyr |
| VH_45 | 256 | GlyPheThrPheSerSe | 426 | IleAsnProAsnSerGlyGl | 596 | AlaArgGluArgAsnArgAlaGlyGlu |
| | | rTyrAla | | yThr | | PheSerAlaPheAspIle |
| VH_46 | 257 | GlyPheThrPheSerSe | 427 | IleGluProGlyAsnGlyAs | 597 | AlaArgGlyAlaSerGlyLeuAspPhe |
| | | rTyrAla | | pThr | | |
| VH_47 | 258 | GlyPheThrPheSerSe | 428 | IleLysGlnAspGlySerGlu | 598 | AlaArgAspLeuHisCysGlySerSerC |
| | | rTyrAla | | Lys | | ysGlyProGluAla |
| VH_48 | 259 | GlyPheThrPheSerSe | 429 | IleSerAlaTyrAsnGlyAs | 599 | AlaArgAspProValTyrSerSerSerT |
| | | rTyrAla | | nThr | | rpGlyGlyTyrAlaPheAspIle |
| VH_49 | 260 | GlyPheThrPheSerSe | 430 | IleSerAlaTyrAsnGlyAs | 600 | AlaArgAspThrPheGlyGlyGlySer |
| | | rTyrAla | | nThr | | TyrTyrGlyHisGlyTyr |
| VH_50 | 261 | GlyPheThrPheSerSe | 431 | IleSerAsnAspGlyValAs | 601 | AlaArgGluAsnSerAsnAlaTrpLys |
| | | rTyrAla | | nAsn | | ValMetAspVal |
| VH_51 | 262 | GlyPheThrPheSerSe | 432 | IleSerGlySerGlyAspArg | 602 | AlaLysAspTrpAlaGlyTyrIleAsnGl |
| | | rTyrAla | | Thr | | yTrpTyrGlyAsn |
| VH_52 | 263 | GlyPheThrPheSerSe | 433 | IleSerGlySerGlyGlyArg | 603 | AlaLysAspTrpAlaGlyTyrIleAsnGl |
| | | rTyrAla | | Thr | | yTrpTyrGlyAsn |
| VH_53 | 264 | GlyPheThrPheSerSe | 434 | IleSerGlySerGlyGlyArg | 604 | AlaLysAspTrpAlaGlyTyrIleAspGl |
| | | rTyrAla | | Thr | | yTrpTyrGlyAsn |
| VH_54 | 265 | GlyPheThrPheSerSe | 435 | IleSerGlySerGlyGlyArg | 605 | AlaLysAspTrpGlyAlaTyrSerSerGl |
| | | rTyrAla | | Thr | | yTrpTyrGlyAsp |
| VH_55 | 266 | GlyPheThrPheSerSe | 436 | IleSerGlySerGlyGlyAsn | 606 | AlaLysAspTrpAlaGlyTyrSerAsnG |
| | | rTyrAla | | Ile | | lyTrpTyrGlySer |
| VH_56 | 267 | GlyPheThrPheSerSe | 437 | IleSerGlySerGlyGlyIleT | 607 | AlaLysAspTrpAlaGlyTyrSerAsnG |
| | | rTyrAla | | hr | | lyTrpPheGlySer |
| VH_57 | 268 | GlyPheThrPheSerSe | 438 | IleSerTyrAspGlyGlyAs | 608 | AlaArgValGlySerGlyGlyTrpThrPr |
| | | rTyrAla | | nLys | | oAspTyr |
| VH_58 | 269 | GlyPheThrPheSerSe | 439 | IleSerTyrAspGlySerAs | 609 | AlaValGlyValGlyPheIleThrAspGl |
| | | rTyrAla | | nGln | | yTyrPheGlnHis |
| VH_59 | 270 | GlyPheThrPheSerSe | 440 | IleSerTyrAspGlySerAs | 610 | AlaArgValGlySerGlyGlyTrpThrPr |
| | | rTyrAla | | nLys | | oAspTyr |
| VH_60 | 271 | GlyPheThrPheSerSe | 441 | IleSerTyrAspGlySerAs | 611 | AlaArgValGlySerGlyGlyTrpThrPr |
| | | rTyrAla | | nLys | | oAspTyr |
| VH_61 | 272 | GlyPheThrPheSerSe | 442 | IleSerTyrAspGlySerAs | 612 | AlaLysGlnGlnTrpLeuGlyThrTrpT |
| | | rTyrAla | | nLys | | yrPheAspLeu |
| VH_62 | 273 | GlyPheThrPheSerSe | 443 | IleSerTyrAspGlySerAs | 613 | AlaLysGluTrpGlyGlyGlyAspSerP |
| | | rTyrAla | | nLys | | roThrAspMetGlyLeuPheAspTyr |
| VH_63 | 274 | GlyPheThrPheSerSe | 444 | IleSerTyrAspGlySerAs | 614 | ThrArgValGlySerGlyGlyTrpThrP |
| | | rTyrAla | | nLys | | roAspTyr |
| VH_64 | 275 | GlyPheThrPheSerSe | 445 | IleTrpTyrAspGlyAsnAs | 615 | AlaArgAspAsnSerGlySerTyrAsn |
| | | rTyrAla | | nLys | | TrpPheAsnPro |
| VH_65 | 276 | GlyPheThrPheSerSe | 446 | IleTyrProGlyAspSerAs | 616 | AlaArgSerHisGlyGlySerAsnTrpP |
| | | rTyrAla | | pThr | | heAspPro |
| VH_66 | 277 | GlyPheThrPheSerSe | 447 | IleTyrProGlyAspSerAs | 617 | AlaThrSerLeuGlyAspAspAlaPhe |
| | | rTyrAla | | pThr | | AspIle |
| VH_67 | 278 | GlyPheThrPheSerSe | 448 | IleTyrProGlyAspSerGl | 618 | AlaArgLeuGlyHisSerGlySerTrpT |
| | | rTyrAla | | uThr | | yrPheAspLeu |
| VH_68 | 279 | GlyPheThrPheSerSe | 449 | IleTyrSerGlyGlySerThr | 619 | AlaArgAspLeuSerTyrSerAspAla |
| | | rTyrAla | | | | PheAspIle |
| VH_69 | 280 | GlyPheThrPheSerSe | 450 | IleTyrSerGlyGlySerThr | 620 | AlaArgAspMetThrThrValAspAla |
| | | rTyrAla | | | | PheAspIle |
| VH_70 | 281 | GlyPheThrPheSerSe | 451 | IleTyrSerGlyGlySerThr | 621 | AlaArgAspThrAlaSerGlyGlyMet |
| | | rTyrAla | | | | AspVal |
| VH_71 | 282 | GlyPheThrPheSerSe | 452 | PheTyrSerGlyGlySerTh | 622 | AlaArgGluProTyrProGlyGlyProP |
| | | rTyrAla | | r | | heAspIle |
| VH_72 | 283 | GlyPheThrPheSerSe | 453 | IleSerAlaSerGlyGlySer | 623 | AlaAsnLeuTyrGlyAspTyrAsnAla |
| | | rTyrGly | | Thr | | Tyr |
| VH_73 | 284 | GlyPheThrPheSerSe | 454 | IleSerGlySerGlyAspArg | 624 | AlaLysAspTrpAlaGlyTyrIleAsnGl |
| | | rTyrGly | | Thr | | yTrpTyrGlyAsn |
| VH_74 | 285 | GlyPheThrPheSerSe | 455 | IleSerGlySerGlyGlyArg | 625 | AlaLysAspTrpAlaGlyTyrIleAsnGl |
| | | rTyrGly | | Thr | | yTrpTyrGlyAsn |
| VH_75 | 286 | GlyPheThrPheSerSe | 456 | IleSerGlySerGlyGlyIleT | 626 | AlaLysAspTrpAlaGlyTyrThrAsnG |
| | | rTyrGly | | hr | | lyTrpTyrGlySer |
| VH_76 | 287 | GlyPheThrPheSerSe | 457 | IleSerGlySerGlyGlySer | 627 | AlaLysAspLeuValLeuGly |
| | | rTyrGly | | Thr | | |
| VH_77 | 288 | GlyPheThrPheSerSe | 458 | IleSerTrpAsnSerGlySer | 628 | AlaLysAspTrpAspSerSerGlyTyrT |
| | | rTyrGly | | Ile | | rpProLeuPheAspTyr |
| VH_78 | 289 | GlyPheThrPheSerSe | 459 | IleSerTyrAspGlySerAs | 629 | AlaArgValGlySerGlyGlyTrpThrPr |
| | | rTyrGly | | nLys | | oAspTyr |
| VH_79 | 290 | GlyPheThrPheSerSe | 460 | IleSerTyrAspGlySerAs | 630 | AlaArgValGlySerGlyGlyTrpThrPr |
| | | rTyrGly | | nLys | | oAspTyr |
| VH_80 | 291 | GlyPheThrPheSerSe | 461 | IleTrpTyrAspGlySerAs | 631 | AlaArgGluValValGlySerTyrTyrLe |
| | | rTyrGly | | nLys | | uAspTyr |
| VH_81 | 292 | GlyPheThrPheSerSe | 462 | IleAsnProAsnSerGlyGl | 632 | AlaArgGlyGlyAspCysSerSerThrS |
| | | rTyrPro | | yThr | | erCysTyrAspProAspTyr |
| VH_82 | 293 | GlyPheThrPheSerSe | 463 | IleLysGlnAspGlySerGlu | 633 | AlaArgIleGlyArgPheGlyArgLysT |
| | | rTyrPro | | Lys | | yrGlyMetAspVal |
| VH_83 | 294 | GlyPheThrPheSerSe | 464 | IleSerAlaTyrAsnGlyAs | 634 | AlaArgGlyLeuGlyAspSerSerSerS |
| | | rTyrPro | | nThr | | erTyr |
| VH_84 | 295 | GlyPheThrPheSerSe | 465 | IleSerGlySerGlyAspIle | 635 | AlaLysAspTrpAlaGlyTyrValAsnG |
| | | rTyrPro | | Thr | | lyTrpTyrGlyAsn |
| VH_85 | 296 | GlyPheThrPheSerSe | 466 | IleSerGlySerGlyAspIle | 636 | AlaLysAspTrpAlaGlyTyrValAsnG |
| | | rTyrPro | | Thr | | lyTrpTyrGlyAsn |
| VH_86 | 297 | GlyPheThrPheSerSe | 467 | IleSerGlySerGlyGlyArg | 637 | AlaLysAspTrpAlaGlyTyrIleAsnGl |
| | | rTyrPro | | Thr | | yTrpTyrGlyAsn |
| VH_87 | 298 | GlyPheThrPheSerSe | 468 | IleSerGlySerGlyGlyArg | 638 | AlaLysAspTrpGlyAlaTyrSerSerGl |
| | | rTyrPro | | Thr | | yTrpTyrGlyAsp |
| VH_88 | 299 | GlyPheThrPheSerSe | 469 | IleSerGlySerGlyGlyIleT | 639 | AlaLysAspTrpAlaGlyTyrThrAsnG |
| | | rTyrPro | | hr | | lyTrpTyrGlySer |
| VH_89 | 300 | GlyPheThrPheSerSe | 470 | IleSerGlyThrGlyGlyArg | 640 | AlaLysAspTrpAlaGlyTyrIleAsnGl |
| | | rTyrPro | | Thr | | yTrpTyrGlySer |
| VH_90 | 301 | GlyPheThrPheSerSe | 471 | IleSerTyrAspAlaThrAs | 641 | AlaLysGluArgPheThrGlyGlyTyrT |
| | | rTyrPro | | nAsn | | yrThrTyrPheAspTyr |
| VH_91 | 302 | GlyPheThrPheSerSe | 472 | IleTyrHisSerGlySerThr | 642 | AlaArgAlaGlyGlyLeuHisLeuAspT |
| | | rTyrPro | | | | yr |
| VH_92 | 303 | GlyPheThrPheSerSe | 473 | IleTyrProGlyAspSerAs | 643 | AlaArgGlyAsnGlyAspGlyGlyPhe |
| | | rTyrPro | | pThr | | AspTyr |
| VH_93 | 304 | GlyPheThrPheSerSe | 474 | IleSerGlySerGlyGlyArg | 644 | AlaLysAspTrpAlaGlyTyrIleAsnGl |
| | | rTyrSer | | Thr | | yTrpTyrGlyAsn |
| VH_94 | 305 | GlyPheThrPheSerSe | 475 | IleSerGlySerGlyAspIle | 645 | AlaLysAspTrpAlaGlyTyrValAsnG |
| | | rTyrTrp | | Thr | | lyTrpTyrGlyAsn |
| VH_95 | 306 | GlyPheThrPheSerSe | 476 | IleSerTyrAspGlySerAs | 646 | AlaArgAspArgGlyValGluGlyAlaT |
| | | rTyrTrp | | nLys | | yrGlyMetAspVal |
| VH_96 | 307 | GlyPheThrPheSerSe | 477 | IleSerTyrAspGlySerAs | 647 | AlaLysGlyLeuLeuValAlaSerIleTy |
| | | rTyrTrp | | nLys | | rAspAlaPheAspIle |
| VH_97 | 308 | GlyPheThrPheSerSe | 478 | IleTyrHisSerGlySerThr | 648 | AlaArgGlySerAsnIlePheAspIle |
| | | rTyrTrp | | | | |
| VH_98 | 309 | GlyPheThrPheSerTh | 479 | IleLysSerLysAsnAspGly | 649 | ThrThrAlaProSerLeuMetAspVal |
| | | rTyrAla | | GlyThrThr | | |
| VH_99 | 310 | GlyPheThrPheSerTh | 480 | IleSerAlaTyrAsnGlyAs | 650 | AlaArgAspLeuThrPheGlySerGly |
| | | rTyrAla | | nThr | | ProThrArgAspTyr |
| VH_100 | 311 | GlyPheThrPheSerTh | 481 | IleSerGlySerGlyAspIle | 651 | AlaLysAspTrpAlaGlyTyrThrAsnG |
| | | rTyrAla | | Thr | | lyTrpTyrGlySer |
| VH_101 | 312 | GlyPheThrPheSerTh | 482 | IleSerGlySerGlyAspIle | 652 | AlaLysAspTrpAlaGlyTyrValAsnG |
| | | rTyrAla | | Thr | | lyTrpTyrGlyAsn |
| VH_102 | 313 | GlyPheThrPheSerTh | 483 | IleSerGlySerGlyGlyArg | 653 | AlaLysAspTrpGlyAlaTyrSerSerGl |
| | | rTyrAla | | Thr | | yTrpTyrGlyAsp |
| VH_103 | 314 | GlyPheThrPheSerTh | 484 | IleSerGlySerGlyGlySer | 654 | AlaLysAspTrpAlaGlyTyrIleAsnGl |
| | | rTyrAla | | Thr | | yTrpTyrGlyAsn |
| VH_104 | 315 | GlyPheThrPheSerTh | 485 | IleSerGlySerGlyGlySer | 655 | AlaLysAspTrpThrAsnGlnTrpLeu |
| | | rTyrAla | | Thr | | AspAlaTyrPheAspTyr |
| VH_105 | 316 | GlyPheThrPheSerTh | 486 | IleSerGlySerGlyGlySer | 656 | AlaLysGluThrIleLeuTyrAspIleLe |
| | | rTyrAla | | Thr | | uThrGlyTyrTyrAsnGluGlyAlaPhe |
| | | | | | | AspIle |
| VH_106 | 317 | GlyPheThrPheSerTh | 487 | IleSerTyrAspGlySerAs | 657 | AlaLysAspTrpGlyArgPheGlyGluL |
| | | rTyrAla | | nLys | | euLeuGluGlySerProTyr |
| VH_107 | 318 | GlyPheThrPheSerTh | 488 | ThrTyrTyrArgSerLysTr | 658 | AlaArgGluPheGlnAspSerSerSer |
| | | rTyrAla | | pTyrAsn | | TrpTyrGluGlyArgAlaPheAspIle |
| VH_108 | 319 | GlyPheThrValSerSer | 489 | IleAsnProAsnSerGlyGl | 659 | AlaArgAspTrpGlyArgGlyValGlyA |
| | | AsnTyr | | yThr | | spSerGlyPheValAspTyr |
| VH_109 | 320 | GlyPheThrValSerSer | 490 | IleAsnProLysSerGlyGly | 660 | AlaArgAspPheValGlyAlaSerLeu |
| | | AsnTyr | | Ala | | AspTyr |
| VH_110 | 321 | GlyPheThrValSerSer | 491 | IleSerGlySerGlyAspArg | 661 | AlaLysAspTrpAlaGlyTyrIleAsnGl |
| | | AsnTyr | | Thr | | yTrpTyrGlyAsn |
| VH_111 | 322 | GlyPheThrValSerSer | 492 | IleSerSerSerGlySerThrI | 662 | AlaArgGlyTyrLeuGlyAlaTrpAsnP |
| | | AsnTyr | | le | | roAspPheTyrAspTyr |
| VH_112 | 323 | GlyPheThrValSerSer | 493 | IleSerTyrAspGlySerAs | 663 | AlaArgValGlySerGlyGlyTrpThrPr |
| | | AsnTyr | | nLys | | oAspTyr |
| VH_113 | 324 | GlyPheThrValSerSer | 494 | IleThrGlySerGlyGlyThr | 664 | AlaLysAspTrpAlaGlyTyrIleAsnGl |
| | | AsnTyr | | | | yTrpPheGlySer |
| VH_114 | 325 | GlyPheThrValSerSer | 495 | IleTyrProGlyAspSerAs | 665 | AlaArgLeuGlyAspGlySerAsnPhe |
| | | AsnTyr | | pThr | | AspTyr |
| VH_115 | 326 | GlyPheThrValSerSer | 496 | ThrTyrTyrArgSerLysTr | 666 | AlaArgGluLysIleAlaValAlaGlyTyr |
| | | AsnTyr | | pTyrAsn | | TyrTyrGlyMetAspVal |
| VH_116 | 327 | GlyPheThrValSerSer | 497 | ThrTyrTyrAsnArgLysTr | 667 | AlaArgAspGlyGlyTrpSerGlySerA |
| | | AsnTyr | | pIleAsn | | laLeuAspVal |
| VH_117 | 328 | GlyTyrArgPheThrSer | 498 | IleTyrSerGlyGlySerThr | 668 | AlaArgAspLeuHisSerAlaAlaGlyP |
| | | TyrTrp | | | | heAspTyr |
| VH_118 | 329 | GlyTyrSerPheThrArg | 499 | IleLysSerLysAsnAspGly | 669 | ThrThrAlaProSerLeuMetAspVal |
| | | TyrTrp | | GlyThrThr | | |
| VH_119 | 330 | GlyTyrSerPheThrSer | 500 | IleSerGlySerGlyAspArg | 670 | AlaLysAspTrpAlaGlyTyrIleAsnGl |
| | | TyrTrp | | Thr | | yTrpTyrGlyAsn |
| VH_120 | 331 | GlyTyrSerPheThrSer | 501 | IleSerGlySerGlyAspArg | 671 | AlaLysAspTrpAlaGlyTyrIleAsnGl |
| | | TyrTrp | | Thr | | yTrpTyrGlyAsn |
| VH_121 | 332 | GlyTyrSerPheThrSer | 502 | IleSerTyrAspGlySerAs | 672 | AlaLysGlySerSerProTyrTyrTyrTy |
| | | TyrTrp | | nLys | | rGlyMetAspVal |
| VH_122 | 333 | GlyTyrSerPheThrSer | 503 | IleTyrHisSerGlySerThr | 673 | AlaArgAspGlyGlySerGlyTrpTyrA |
| | | TyrTrp | | | | spTyr |
| VH_123 | 334 | GlyTyrSerPheThrSer | 504 | IleTyrSerGlyGlySerThr | 674 | AlaArgAspThrAlaSerGlyGlyMet |
| | | TyrTrp | | | | AspVal |
| VH_124 | 335 | GlyTyrSerPheThrSer | 505 | ThrTyrTyrArgSerLysTr | 675 | AlaArgGlyValThrValProTyrTyrT |
| | | TyrTrp | | pTyrAsn | | yrTyrGlyMetAspVal |
| VH_125 | 336 | GlyTyrSerPheThrSer | 506 | ThrTyrTyrArgSerLysTr | 676 | AlaArgSerSerGlySerTyrGlyTyrP |
| | | TyrTrp | | pTyrAsn | | heGlnHis |
| VH_126 | 337 | GlyTyrThrPheThrArg | 507 | ThrTyrTyrArgSerLysTr | 677 | AlaArgGluGlyThrAspIleTyrTyrTy |
| | | AsnAla | | pTyrAsn | | rTyrGlyMetAspVal |
| VH_127 | 338 | GlyTyrThrPheThrGly | 508 | IleAspTyrSerGlySerThr | 678 | AlaArgAspGlyTrpIleArgLysGluAl |
| | | TyrTyr | | | | aPheAspPro |
| VH_128 | 339 | GlyTyrThrPheThrGly | 509 | IleLysSerLysAsnAspGly | 679 | ThrThrAlaProSerLeuMetAspVal |
| | | TyrTyr | | GlyThrThr | | |
| VH_129 | 340 | GlyTyrThrPheThrGly | 510 | IleSerAlaTyrAsnGlyAs | 680 | AlaArgAspProGlyGlyTyrTyrTyrT |
| | | TyrTyr | | nThr | | yrTyrGlyMetAspVal |
| VH_130 | 341 | GlyTyrThrPheThrGly | 511 | IleSerTyrAspGlySerAs | 681 | AlaArgValGlySerGlyGlyTrpThrPr |
| | | TyrTyr | | nLys | | oAspTyr |
| VH_131 | 342 | GlyTyrThrPheThrGly | 512 | IleSerTyrAspGlySerAs | 682 | AlaLysLeuGlyGlySerTyrSerIleTyr |
| | | TyrTyr | | nLys | | TyrGlyMetAspVal |
| VH_132 | 343 | GlyTyrThrPheThrGly | 513 | IleTyrProGlyAspSerGl | 683 | AlaArgAspGlyGlyAsnTyrGlnPhe |
| | | TyrTyr | | uThr | | AspTyr |
| VH_133 | 344 | GlyTyrThrPheThrSer | 514 | IleIleProIlePheGlyThrA | 684 | AlaArgThrGlyArgSerGlySerTyrT |
| | | TyrAla | | la | | yrSerAspAlaPheAspIle |
| VH_134 | 345 | GlyTyrThrPheThrSer | 515 | IleAsnProSerGlyGlySer | 685 | AlaArgGluAspHisAspTyrSerAsn |
| | | TyrGly | | Thr | | GlnGlyGlyPheAspTyr |
| VH_135 | 346 | GlyTyrThrPheThrSer | 516 | IleIleProIlePheGlyThrA | 686 | AlaAlaArgAlaProGlyGlySerSerT |
| | | TyrGly | | la | | yrTyrTyrTyrGlyMetAspVal |
| VH_136 | 347 | GlyTyrThrPheThrSer | 517 | IleSerAlaTyrAsnGlyAs | 687 | AlaArgAspProGlyTyrAspPheTrp |
| | | TyrGly | | nThr | | SerGlyTyrSerAspVal |
| VH_137 | 348 | GlyTyrThrPheThrSer | 518 | IleSerGlySerGlyGlyArg | 688 | AlaLysAspTrpAlaGlyTyrIleAsnGl |
| | | TyrGly | | Thr | | yTrpTyrGlyAsn |
| VH_138 | 349 | GlyTyrThrPheThrSer | 519 | IleSerTrpAsnSerGlySer | 689 | AlaLysAspMetTrpGlySerLeuSerl |
| | | TyrGly | | Ile | | leValGlyAlaThrArgAlaPheAspTy |
| | | | | | | r |
| VH_139 | 350 | GlyTyrThrPheThrSer | 520 | IleThrGlySerGlyGlyThr | 690 | AlaLysAspTrpAlaGlyTyrIleAsnGl |
| | | TyrGly | | | | yTrpPheGlySer |
| VH_140 | 351 | GlyTyrThrPheThrSer | 521 | IleTyrHisSerGlySerThr | 691 | AlaArgGlyProLeuLeuIleAlaAlaAl |
| | | TyrGly | | | | aGlyThrAspTyrTyrTyrGlyMetAs |
| | | | | | | pVal |
| VH_141 | 352 | GlyTyrThrPheThrSer | 522 | IleSerGlySerGlyGlySer | 692 | AlaSerSerTyrGlyGlyAsnProLeuA |
| | | TyrTyr | | Thr | | spAlaPheAspIle |
| VH_142 | 353 | GlyAspSerValSerSer | 523 | ThrTyrTyrArgSerLysTr | 693 | AlaArgGluLysIleAlaValAlaGlyTyr |
| | | AsnSerAlaAla | | pTyrAsn | | TyrTyrGlyMetAspVal |
| VH_143 | 354 | GlyAspSerValSerSer | 524 | ThrTyrTyrArgSerLysTr | 694 | AlaArgGluPheGlnAspSerSerSer |
| | | AsnSerAlaAla | | pTyrAsn | | TrpTyrGluGlyArgAlaPheAspIle |
| VH_144 | 355 | GlyAspSerValSerSer | 525 | ThrTyrTyrArgSerLysTr | 695 | AlaArgGlyGlyValGlyAlaThrTrpT |
| | | AsnSerAlaAla | | pTyrAsn | | yrTyrGlyMetAspVal |
| VH_145 | 356 | GlyPheThrPheAspAs | 526 | IleSerTrpAsnSerGlySer | 696 | AlaLysAspIleAlaAlaGlyGlyLeuAs |
| | | pTyrAla | | Ile | | pSer |
| VH_146 | 357 | GlyPheThrPheSerAs | 527 | IleLysSerLysAsnAspGly | 697 | ThrThrAlaProSerLeuMetAspVal |
| | | nAlaTrp | | GlyThrThr | | |
| VH_147 | 358 | GlyPheThrPheSerAs | 528 | IleLysSerLysAsnAspGly | 698 | ThrThrAlaProSerLeuMetAspVal |
| | | nAlaTrp | | GlyThrThr | | |
| VH_148 | 359 | GlyPheThrPheSerSe | 529 | IleSerTyrAspGlySerAs | 699 | AlaArgAspArgGlyValGluGlyAlaT |
| | | rTyrAla | | nLys | | yrGlyMetAspVal |
| VH_149 | 360 | GlyPheThrPheSerSe | 530 | IleSerGlySerGlyGlySer | 700 | AlaLysAlaThrGlyTyrSerSerGlyTr |
| | | rTyrGly | | Thr | | pTyrGlyAlaTyrPheAspTyr |
| VH_150 | 361 | GlyPheThrPheSerSe | 531 | IleSerTyrAspGlySerAs | 701 | AlaLysGlySerSerProTyrTyrTyrTy |
| | | rTyrGly | | nLys | | rGlyMetAspVal |
| VH_151 | 362 | GlyPheThrPheSerSe | 532 | IleTrpTyrAspGlyAsnAs | 702 | AlaArgAspAsnSerGlySerTyrAsn |
| | | rTyrGly | | nLys | | TrpPheAsnPro |
| VH_152 | 363 | GlyPheThrPheSerSe | 533 | IleTrpTyrAspGlySerAs | 703 | AlaArgGluValValGlySerTyrTyrLe |
| | | rTyrGly | | nLys | | uAspTyr |
| VH_153 | 364 | GlyPheThrPheSerSe | 534 | IleSerTyrAspGlyGlyAs | 704 | AlaArgValGlySerGlyGlyTrpThrPr |
| | | rTyrPro | | nLys | | oAspTyr |
| VH_154 | 365 | GlyPheThrPheSerSe | 535 | IleSerTyrAspGlySerAs | 705 | AlaArgValGlySerGlyGlyTrpThrPr |
| | | rTyrPro | | nLys | | oAspTyr |
| VH_155 | 366 | GlyPheThrPheSerSe | 536 | IleSerTyrAspGlySerAs | 706 | AlaArgValGlySerGlyGlyTrpThrPr |
| | | rTyrPro | | nLys | | oAspTyr |
| VH_156 | 367 | GlyPheThrPheSerSe | 537 | IleSerTyrAspGlySerAs | 707 | AlaArgValGlySerGlyGlyTrpThrPr |
| | | rTyrPro | | nLys | | oAspTyr |
| VH_157 | 368 | GlyPheThrPheSerSe | 538 | IleSerTyrAspGlySerAs | 708 | AlaArgValGlySerGlyGlyTrpThrPr |
| | | rTyrPro | | nLys | | oAspTyr |
| VH_158 | 369 | GlyPheThrPheSerSe | 539 | IleSerTyrAspGlySerAs | 709 | ThrArgValGlySerGlyGlyTrpThrP |
| | | rTyrPro | | nLys | | roAspTyr |
| VH_159 | 370 | GlyPheThrPheSerSe | 540 | IleTrpTyrAspGlySerAs | 710 | AlaArgLeuGlySerGlyTrpSerLeuA |
| | | rTyrSer | | nLys | | spTyr |
| VH_160 | 371 | GlyPheThrPheSerSe | 541 | IleLysGlnAspGlySerGlu | 711 | AlaArgAspLeuHisCysGlySerSerC |
| | | rTyrTrp | | Lys | | ysGlyProGluAla |
| VH_161 | 372 | GlyPheThrValSerSer | 542 | IleTyrSerGlyGlySerThr | 712 | AlaArgAspLeuHisSerAlaAlaGlyP |
| | | AsnTyr | | | | heAspTyr |
| VH_162 | 373 | GlyPheThrValSerSer | 543 | IleTyrSerGlyGlySerThr | 713 | AlaArgAspLeuSerTyrSerAspAla |
| | | AsnTyr | | | | PheAspIle |
| VH_163 | 374 | GlyPheThrValSerSer | 544 | IleTyrSerGlyGlySerThr | 714 | AlaArgAspPheGluGlySerGlyAlaL |
| | | AsnTyr | | | | euAspVal |
| VH_164 | 375 | GlyPheThrValSerSer | 545 | IleTyrSerGlyGlySerThr | 715 | AlaArgAspThrAlaSerGlyGlyMet |
| | | AsnTyr | | | | AspVal |
| VH_165 | 376 | GlyPheThrValSerSer | 546 | IleTyrSerGlyGlySerThr | 716 | AlaArgAspThrAlaSerGlyGlyMet |
| | | AsnTyr | | | | AspVal |
| VH_166 | 377 | GlyTyrSerPheThrSer | 547 | IleTyrProGlyAspSerAs | 717 | AlaSerGlyAlaSerProTyrTyrPheA |
| | | TyrTrp | | pThr | | spTyr |
| VH_167 | 378 | GlyTyrThrPheThrGly | 548 | IleAsnProAsnSerGlyGl | 718 | AlaArgGlyGlyAspCysSerSerThrS |
| | | TyrTyr | | yThr | | erCysTyrAspProAspTyr |
| VH_168 | 379 | GlyTyrThrPheThrSer | 549 | IleSerAlaTyrAsnGlyAs | 719 | AlaArgAspProValTyrSerSerSerT |
| | | TyrGly | | nThr | | rpGlyGlyTyrAlaPheAspIle |
| VH_169 | 380 | GlyTyrThrPheThrSer | 550 | IleSerAlaTyrAsnGlyAs | 720 | AlaArgGlyLeuGlyAspSerSerSerS |
| | | TyrGly | | nThr | | erTyr |
| VH_170 | 381 | GlyTyrThrPheThrSer | 551 | IleAsnProSerGlyGlySer | 721 | AlaArgGluAspHisAspTyrSerAsn |
| | | TyrTyr | | Thr | | GlnGlyGlyPheAspTyr |

TABLE 5
VL CDR SEQUENCES COMBINED

| mAb ID | VL_CDR1/2/3 | SEQ ID NO: |
|---|---|---|
| VL_1 | ThrSerAsnIleGlyAlaAsnHisThrLysAsnAlaAlaTrpAspAspSerLeuArgGlyTrpThr | 722 |
| VL_2 | SerSerAspIleGlyGlyTyrLysTyrAspValThrGlySerTyrSerSerSerSerSerHisTyrVal | 723 |
| VL_3 | GlnSerIleSerSerPheAlaAlaSerGlnGlnSerTyrSerThrProTrpThr | 724 |
| VL_4 | GlnSerValSerSerAsnGlyAlaSerGlnHisTyrAsnAsnTrpProProGlnIleThr | 725 |
| VL_5 | GlnSerValSerSerAsnGlyAlaSerGlnGlnTyrGlyTyrSerGlnIleThr | 726 |
| VL_6 | AsnIleGlySerLysSerAspAspSerGlnValTrpAspSerSerSerAspHisValVal | 727 |
| VL_7 | AsnIleGlySerLysSerAspAspSerGlnValTrpAspSerSerSerAspHisValVal | 728 |
| VL_8 | SerSerAsnIleGlyAlaGlyTyrAspSerSerAsnGlnSerPheAspProSerLeuSerAspSerTrpVal | 729 |
| VL_9 | SerGlySerIleThrAspAspTyrGluAspHisGlnSerTyrAspAlaGluSerTrpVal | 730 |
| VL_10 | GlnSerValSerSerAsnGlyAlaSerGlnGlnTyrGlyTyrSerGlnIleThr | 731 |
| VL_11 | AsnIleGlySerLysSerAspAspSerGlnValTrpAspSerSerSerAspLeuLeuTyrVal | 732 |
| VL_12 | GlnSerValSerSerSerTyrGlyAlaSerGlnGlnTyrGlyArgSerProPheThr | 733 |
| VL_13 | GlnSerValThrSerAsnTyrGlyAlaSerGlnGlnTyrGlySerSerProThr | 734 |
| VL_14 | ThrGlyAlaValThrSerGlyPheTyrSerAlaThrLeuLeuTyrTyrGlyGlyAlaGlnProTrpVal | 735 |
| VL_15 | AsnIleGlySerLysSerAspAspSerGlnLeuTrpAspGlyAlaSerAspLeuValIle | 736 |
| VL_16 | GlnThrIleSerSerTyrGlyAlaSerGlnGlnSerTyrSerThrProGlnThr | 737 |
| VL_17 | AsnIleGlySerLysSerAspAspSerGlnValTrpAspSerSerSerAspHisValVal | 738 |
| VL_18 | AsnIleGlySerLysSerAspAspSerGlnValTrpAspSerSerSerAspHisValVal | 739 |
| VL_19 | GlnArgValArgSerSerTyrGlyAlaSerGlnGlnTyrGlySerSerProProArgIleIle | 740 |
| VL_20 | GlnThrValSerAsnAsnAspAlaSerGlnGlnTyrGlySerSerProLeuThr | 741 |
| VL_21 | AsnIleGlySerLysSerAspAspSerGlnValTrpAspSerSerSerAspHisValVal | 742 |
| VL_22 | AspIleGluSerLysSerAspAspSerGlnValTrpAspGlyIleIleAsnGlnValVal | 743 |
| VL_23 | GlnGlyValArgAlaSerSerAlaAlaSerGlnGlnTyrGlyArgSerProThr | 744 |
| VL_24 | GlnSerIleSerSerTyrAlaAlaSerGlnGlnSerTyrSerThrProProTyrThr | 745 |
| VL_25 | GlnSerValSerSerSerTyrGlyAlaSerGlnGlnTyrGlySerSerProGlnTyrThr | 746 |
| VL_26 | AsnIleGlySerLysSerAspAspSerGlnValTrpGlySerSerAsnAspProValVal | 747 |
| VL_27 | AsnIleGlySerLysSerAspAspSerGlnValTrpAspSerSerSerAspHisValVal | 748 |
| VL_28 | SerSerAsnIleGlyAsnAsnTyrAspAsnAsnGlyThrTrpAspSerSerLeuSerAlaValVal | 749 |
| VL_29 | AsnIleGlyAlaLysSerAspAspSerGlnValTrpAspAsnThrGlyAspHisProArgValIle | 750 |
| VL_30 | GlnSerLeuValTyrSerAspGlyAsnThrTyrLysValSerMetGlnGlyLysHisTrpProProThr | 751 |
| VL_31 | SerLeuArgSerTyrTyrGlyLysAsnAsnSerArgAspSerSerGlyAsnHisTrpVal | 752 |
| VL_32 | AsnIleGlySerLysSerAspAspSerGlnValTrpAspSerSerSerAspHisSerValVal | 753 |
| VL_33 | AsnIleGlySerTyrSerAspAspSerGlnValTrpAspSerSerSerAspHisValIle | 754 |
| VL_34 | AsnIleGlySerLysSerAspAspSerGlnValTrpAspSerSerSerAspHisValVal | 755 |
| VL_35 | AsnLeuGlyGlyArgTyrGlnAspLeuGlnAlaTrpAspThrTyrThrValVal | 756 |
| VL_36 | AsnIleGlySerLysSerAspAspSerGlnValTrpAspSerSerSerAspHisValVal | 757 |
| VL_37 | SerLeuArgSerTyrTyrGlyLysAsnAsnSerArgAspSerSerGlyAsnHisValVal | 758 |
| VL_38 | LysLeuGlyAspLysTyrGlnAspThrGlnAlaTrpAspSerSerThrAsnTyrVal | 759 |
| VL_39 | GlnSerIleAsnSerAsnGlyAlaSerGlnGlnPheGluGlnTrpProLeuThr | 760 |
| VL_40 | GlnArgIleSerLysTyrGlySerSerGlnGlnSerAspSerValProIleThr | 761 |
| VL_41 | SerSerAsnIleGlyAlaGlyTyrArgGlyAspAsnGlnSerHisAspGluSerLeuAsnSerLysVal | 762 |
| VL_42 | GlnSerValSerSerAsnGlyAlaSerGlnGlnTyrGlySerSerProLeuThr | 763 |
| VL_43 | AsnIleGlySerLysSerAspAspSerGlnLeuTrpAspGlyAlaSerAspLeuValIle | 764 |
| VL_44 | AsnIleGlySerLysSerAspAspSerGlnValTrpAspSerSerSerAspHisValVal | 765 |
| VL_45 | GlnSerValSerSerAsnGlyAlaSerGlnGlnTyrAsnAsnTrpProProGlnTyrThr | 766 |
| VL_46 | AsnIleGlySerLysSerAspAspSerGlnValTrpAspSerSerSerAspTyrValVal | 767 |
| VL_47 | AsnIleGlySerLysSerAspAspSerGlnValTrpAspSerLeuSerAspHisValIle | 768 |
| VL_48 | AsnIleGlyThrLysSerAspAspSerGlnValTrpAspHisSerAsnAspHisValVal | 769 |
| VL_49 | AsnIleGlySerLysSerAspAspSerSerAlaTrpAspSerSerLeuThrAlaValVal | 770 |
| VL_50 | AsnIleGlySerLysGlyAspAspArgGlnValTrpAspThrAsnSerGlnHisValVal | 771 |
| VL_51 | SerSerAsnIleGlyAsnAsnGlyTyrAspAspAlaThrTrpAspAspArgLeuLysGlyTyrVal | 772 |
| VL_52 | AsnIleGlySerLysSerAspAspSerGlnValTrpAspSerSerSerAspGlnGlyVal | 773 |
| VL_53 | GlyGlySerLeuAlaSerAsnTyrGluAspLysGlnSerTyrAspSerAlaAsnProLeuValVal | 774 |
| VL_54 | AsnLeuGlyGlyTyrSerAspAspSerGlnValTrpAspSerSerSerAspLeuValVal | 775 |
| VL_55 | SerGlySerIleAlaSerAsnTyrGluAspAsnGlnSerTyrAspThrSerAsnLeuValVal | 776 |
| VL_56 | AsnIleGlySerLysAsnAspAspThrGlnValTrpAspArgAsnThrGlyHisValVal | 777 |
| VL_57 | SerSerAspValGlyGlyTyrAsnTyrGluValSerSerSerTyrThrSerSerSerThrLeuValVal | 778 |
| VL_58 | AsnIleGlyAsnLysAsnAspAspLysGlnValTrpAspThrSerGluTyrGlnAsnArgVal | 779 |
| VL_59 | SerGlySerIleAlaSerAsnTyrGluHisAsnGlnSerTyrAspAsnSerAsnProHisValVal | 780 |
| VL_60 | SerSerAsnIleGlyAlaGlyTyrAspGlyAsnSerGlnSerTyrAspSerSerLeuSerGlyPheTyrVal | 781 |
| VL_61 | AsnIleGlyAsnLysAsnAspAspSerGlnValTrpAspSerSerSerAspHisValVal | 782 |
| VL_62 | GlnGlyIleSerSerTrpGlyAlaSerGlnGlnAlaAsnSerPheProIleThr | 783 |
| VL_63 | SerGlySerIleAlaSerAsnTyrGluAspAsnGlnSerTyrAspSerSerAsnHisValVal | 784 |
| VL_64 | GlnGlyValAsnSerAspGlyAlaSerGlnGlnTyrAsnAsnTrpProTrpThr | 785 |
| VL_65 | LysLeuGlyAspLysTyrGluAspThrGlnAlaTrpAspThrSerAlaValVal | 786 |
| VL_66 | AsnIleGlySerLysSerAspAspSerGlnLeuTrpAspAspSerSerAspHisValVal | 787 |
| VL_67 | AsnIleGlySerLysSerAspAspSerGlnValTrpAspSerSerSerAspHisValVal | 788 |
| VL_68 | SerLeuArgAspTyrTyrGlyLysAsnAsnSerArgAspSerSerGlyAsnHisValVal | 789 |
| VL_69 | AsnIleGlyArgLysSerAspAspThrGlnLeuTyrAspSerAspSerAspAsnValVal | 790 |
| VL_70 | AsnIleGlySerLysSerAspAspSerGlnValTrpAspSerSerSerAspHisProVal | 791 |
| VL_71 | SerLeuArgSerTyrTyrGlyLysAsnAsnSerArgAspSerSerGlyAsnLeuGlyVal | 792 |
| VL_72 | GlnAsnIleLeuThrAsnAlaAlaSerGlnGlnSerTyrSerIleProTrpThr | 793 |
| VL_73 | LysLeuGlyAsnLysTyrGluAsnAsnGlnAlaTrpAspSerSerThrAlaVal | 794 |
| VL_74 | GlnSerIleSerSerTyrAlaAlaSerGlnGlnSerTyrSerThrSerTrpThr | 795 |
| VL_75 | AsnIleGlySerLysSerAspAspSerAlaAlaTrpAspAspSerLeuAsnGlyGlnValVal | 796 |
| VL_76 | AsnIleGlySerLysSerAspAspSerGlnValTrpAspSerSerSerAspHisValVal | 797 |
| VL_77 | AsnValGlyThrThrSerAspAspThrGlnValTrpAspSerSerSerAspHisValIle | 798 |
| VL_78 | LysIleGlySerTyrSerAspAspSerGlnValTrpAspThrTyrGlyAspGlnValVal | 799 |
| VL_79 | AsnIleGlySerLysSerAspAspSerGlnValTrpAspSerSerSerAspHisProVal | 800 |
| VL_80 | AsnIleGlySerLysSerAspAspSerGlnValTrpAspSerGlySerAspPheValVal | 801 |
| VL_81 | AsnIleGlySerLysSerAspAspSerGlnValTrpAspSerSerSerAspHisProVal | 802 |
| VL_82 | AsnIleGlySerGlnSerAspAspSerGlnValTrpAspGlySerAsnAspHisValVal | 803 |
| VL_83 | AsnIleGlyArgGluSerAspAspSerGlnValTrpAspSerSerIleAspHisValVal | 804 |
| VL_84 | AsnIleGlySerLysSerAspAspSerGlnValTrpAspSerSerSerAspHisValVal | 805 |
| VL_85 | AsnIleGlySerLysSerAspAspSerGlnValTrpAspSerSerSerAspHisValVal | 806 |
| VL_86 | AsnIleGlySerLysGlyAspAspSerGlnValTrpAspAsnSerSerAspSerValVal | 807 |
| VL_87 | GlyGlySerIleAlaSerAsnTyrLysAspAsnGlnSerTyrGlySerGlyAsnValVal | 808 |
| VL_88 | SerGlySerIleAlaSerAsnTyrGluHisAsnGlnSerPheAspArgAsnAsnProLysTrpVal | 809 |
| VL_89 | AsnIleGlySerLysSerAspAspSerGlnValTrpAspSerSerSerAspHisLeuValVal | 810 |
| VL_90 | LysLeuGlyAspLysTyrHisAspThrGlnValTrpAspGlyThrThrAspHisPheLeu | 811 |
| VL_91 | AsnIleGlySerLysSerTyrAspSerGlnValTrpAspSerValSerAspProValMet | 812 |
| VL_92 | SerSerAspValGlyGlyTyrAsnTyrGluValSerSerSerTyrAlaGlySerAsnAsnLeuVal | 813 |
| VL_93 | LysLeuGlyAspLysTyrGlnAsnAsnGlnAlaTrpAspSerSerAlaValVal | 814 |
| VL_94 | AsnIleGlySerLysSerAspAspSerGlnValTrpAspSerThrSerAspHisProGluValVal | 815 |
| VL_95 | AsnIleGlySerLysSerAspAspAspGlnValTrpAspSerGlySerAspHisValVal | 816 |
| VL_96 | AsnIleGlySerLysSerAspAspSerGlnValTrpAspSerSerSerAspHisValVal | 817 |
| VL_97 | AsnIleGlySerLysSerAspAspSerGlnValTrpAspSerSerSerAspHisValVal | 818 |
| VL_98 | SerSerAsnIleGlyAsnAsnTyrGluAsnAsnGlyThrTrpAspSerSerLeuSerAlaGlyVal | 819 |
| VL_99 | SerSerAsnIleGlyAlaGlyTyrAspGlyAsnSerGlnSerTyrAspSerSerLeuSerTrpVal | 820 |
| VL_100 | SerSerAspValGlyGlyTyrAsnPheGlyValSerSerSerTyrArgIleArgAspSerLeuVal | 821 |
| VL_101 | AsnIleGlySerLysSerAspAspSerGlnValTrpAspSerSerSerAspHisValVal | 822 |
| VL_102 | GlyGlyGlyIleAlaAspAsnTyrAspAspAspGlnSerTyrAspSerAlaValProValVal | 823 |
| VL_103 | AsnIleGlySerLysSerAspAspSerGlnValTrpAspSerAspAsnAspAsnSerGluValIle | 824 |
| VL_104 | AsnIleGlySerLysAsnAspAspAsnGlnValTrpAspSerSerSerGluHisValVal | 825 |
| VL_105 | AsnIleGlySerAsnSerAspAspSerGlnValTrpAspSerSerSerAspHisValVal | 826 |
| VL_106 | IleLeuGlyHisTyrHisGlyLysAspAsnAsnSerArgAspArgSerGlyThrGlnValLeu | 827 |
| VL_107 | SerSerAspValGlyGlyTyrAsnTyrGluValSerSerSerTyrThrSerSerSerThrLeuValVal | 828 |
| VL_108 | GlnSerValSerThrAsnGlyAlaSerGlnGlnTyrAsnThrTrpProProValArg | 829 |
| VL_109 | SerSerAspValGlyGlyTyrAsnTyrAspValSerSerSerTyrThrSerSerSerThrLeuValVal | 830 |
| VL_110 | SerSerAspValGlyGlyTyrAsnTyrAspValSerSerSerTyrThrSerSerSerThrLeuValVal | 831 |
| VL_111 | LysIleGlySerLysIleHisAspSerGlnValTrpAspValAsnThrAspHisValVal | 832 |
| VL_112 | SerSerAspValGlyGlyTyrAsnTyrGluValThrSerSerTyrThrSerSerSerThrPheValVal | 833 |
| VL_113 | SerGlySerIleValSerAsnTyrGluAspAsnGlnSerTyrAspSerGlyAsnValVal | 834 |
| VL_114 | GlnSerValSerSerSerTyrGlyAlaSerGlnGlnTyrGlySerSerProLeuThr | 835 |
| VL_115 | SerGlySerIleAlaThrAsnTyrGluAspAsnGlnSerTyrAspSerSerThrGlyVal | 836 |
| VL_116 | SerSerAspValGlyGlyTyrAsnTyrAspValSerSerSerTyrThrSerSerSerThrLeuValVal | 837 |
| VL_117 | AsnIleGluSerLysSerAspAspSerGlnValTrpAspSerGlyHisGlnVal | 838 |
| VL_118 | SerSerTyrIleAlaThrAsnSerSerAspSerAlaAlaTrpAspAspSerLeuAsnAlaTyrVal | 839 |
| VL_119 | SerSerAspIleGlyGlyTyrAsnTyrGluValSerSerSerTyrThrSerSerSerThrLeuValVal | 840 |
| VL_120 | SerSerAspValGlyGlyTyrAsnTyrAspValSerSerSerTyrThrSerSerSerThrLeuValVal | 841 |
| VL_121 | SerSerAsnIleGlyAlaGlyTyrAspGlyAsnAsnAlaThrTrpAspAspSerLeuAsnAlaProTyrVal | 842 |
| VL_122 | LysLeuGlyAsnLysTyrGlnAspAspGlnAlaTrpAspSerThrTyrValVal | 843 |
| VL_123 | LysLeuGlyAspLysTyrGlnAspThrGlnAlaTrpAspSerThrThrLeuVal | 844 |
| VL_124 | GlyGlySerIleAlaSerAsnTyrLysAspAsnGlnSerTyrGlySerGlyAsnValVal | 845 |
| VL_125 | SerSerAsnIleAlaSerAsnThrSerAsnAsnSerAlaTrpAspAspSerLeuHisThrTyrVal | 846 |
| VL_126 | SerSerAspValGlyGlyTyrAsnTyrGluValSerSerSerTyrAlaGlySerAspThrValVal | 847 |
| VL_127 | SerSerAsnIleGlyAsnAsnTyrAspAsnAspGlyThrTrpAspAsnSerLeuSerAlaValVal | 848 |
| VL_128 | AsnIleGlySerLysSerAspAspSerGlnValTrpAspSerSerSerAspHisValVal | 849 |
| VL_129 | SerSerAspValGlyGlyTyrAsnTyrAspValSerSerSerTyrThrSerSerSerThrLeuValVal | 850 |
| VL_130 | SerSerAsnIleGlyAsnAsnTyrGluAsnAsnGlyThrTrpAspSerSerLeuSerAlaValVal | 851 |
| VL_131 | SerSerAspValGlyGlyTyrAspTyrGluValSerSerSerTyrThrSerSerSerThrLeuValVal | 852 |
| VL_132 | AsnIleGlySerLysSerAlaAspSerGlnValTrpAspSerSerPheAspValAla | 853 |
| VL_133 | AsnIleGlyAspLysArgTyrAspThrGlnValTrpAspThrAspThrAsnHisAlaVal | 854 |
| VL_134 | SerSerAspValGlyAlaTyrAsnTyrAspValSerSerSerTyrThrThrSerSerThrLeuVal | 855 |
| VL_135 | LysLeuGlyAspLysTyrGlnAspSerGlnThrTrpAspSerSerThrValVal | 856 |
| VL_136 | LysLeuGlyAspLysTyrGlnAspIleGlnAlaTrpAspArgSerSerTyrVal | 857 |
| VL_137 | SerSerAspValGlyGlyTyrAsnTyrGluValSerSerSerTyrSerGlySerAsnAsnLeuValVal | 858 |
| VL_138 | SerSerAspValGlyGlyTyrAsnTyrAspValAsnSerSerTyrThrSerSerAsnThrLeuValVal | 859 |
| VL_139 | SerSerAsnIleGlyAlaGlyTyrAspGlyAsnSerGlnSerTyrAspSerSerLeuSerGlySerGlyTyrVal | 860 |
| VL_140 | SerSerAspValGlyGlyTyrAsnTyrGluValSerSerSerTyrThrSerSerSerThrLeuValVal | 861 |
| VL_141 | SerSerAspValGlyGlyTyrAsnTyrAspValSerSerSerTyrThrSerSerSerThrLeuValVal | 862 |
| VL_142 | AsnIleGlySerLysSerAspAspSerGlnValTrpAspSerGlyAsnIleHisProValVal | 863 |
| VL_143 | GlyAsnAsnTyrGluAsnAsnGlyThrTrpAspSerSerLeuAsnValGlyVal | 864 |
| VL_144 | LysLeuGlyAsnLysTyrGlnAspAsnGlnAlaTrpAspSerSerThrAlaVal | 865 |
| VL_145 | SerSerAspValGlyGlyTyrAsnTyrAspValSerSerSerTyrAlaGlySerSerValVal | 866 |
| VL_146 | SerSerAspValGlyGlyTyrAsnTyrGluValSerSerSerTyrThrSerSerSerThrLeuValVal | 867 |
| VL_147 | GlySerAsnIleGlyAlaGlyTyrAspGlyAsnIleAlaAlaTrpAspAspSerLeuAsnGlyLeuTyrVal | 868 |
| VL_148 | SerSerAspValGlyGlyTyrAsnTyrAspValSerSerSerTyrThrSerSerSerThrPheValVal | 869 |
| VL_149 | SerSerAsnIleGlyIleAsnThrArgAsnAsnAlaAlaTrpAspAspSerLeuSerGlyTrpVal | 870 |
| VL_150 | GlySerAspIleGlyAspTyrLysTyrAspValThrSerProHisThrProSerArgValIle | 871 |
| VL_151 | SerSerAsnIleGlyAlaGlyTyrAspGlyAsnSerAlaAlaTrpAspAspGlyProSerGlyTyrVal | 872 |
| VL_152 | LysLeuGlyAspLysTyrArgAspAsnGlnAlaTrpAspSerSerThrValVal | 873 |
| VL_153 | GlnSerIleAspThrSerAlaAlaSerGlnGlnSerTyrSerThrProGlnTyrThr | 874 |
| VL_154 | GlnSerIleSerSerTrpLysAlaSerGlnGlnTyrAsnThrTyrPheProThr | 875 |

TABLE 6
DISCRETE CDR5 FOR VL SEQUENCES

| | SEQ | | SEQ | | SEQ | |
|---|---|---|---|---|---|---|
| | ID | | ID | | ID | |
| VL_ID | NO: | VL_CDR1 | NO: | VL_CDR2 | NO: | VL_CDR3 |
| VL_1 | 876 | ThrSerAsnIleGlyAlaAsnHi | 1030 | ThrLysAsn | 1184 | AlaAlaTrpAspAspSerLeuArgGlyTrpT |
| | | s | | | | hr |
| VL_2 | 877 | SerSerAspIleGlyGlyTyrLys | 1031 | AspValThr | 1185 | GlySerTyrSerSerSerSerSerHisTyrVal |
| | | Tyr | | | | |
| VL_3 | 878 | GlnSerIleSerSerPhe | 1032 | AlaAlaSer | 1186 | GlnGlnSerTyrSerThrProTrpThr |
| VL_4 | 879 | GlnSerValSerSerAsn | 1033 | GlyAlaSer | 1187 | GlnHisTyrAsnAsnTrpProProGlnIleTh |
| | | | | | | r |
| VL_5 | 880 | GlnSerValSerSerAsn | 1034 | GlyAlaSer | 1188 | GlnGlnTyrGlyTyrSerGlnIleThr |
| VL_6 | 881 | AsnIleGlySerLysSer | 1035 | AspAspSer | 1189 | GlnValTrpAspSerSerSerAspHisValVa |
| | | | | | | l |
| VL_7 | 882 | AsnIleGlySerLysSer | 1036 | AspAspSer | 1190 | GlnValTrpAspSerSerSerAspHisValVa |
| | | | | | | l |
| VL_8 | 883 | SerSerAsnIleGlyAlaGlyTyr | 1037 | SerSerAsn | 1191 | GlnSerPheAspProSerLeuSerAspSerT |
| | | Asp | | | | rpVal |
| VL_9 | 884 | SerGlySerIleThrAspAspTy | 1038 | GluAspHis | 1192 | GlnSerTyrAspAlaGluSerTrpVal |
| | | r | | | | |
| VL_10 | 885 | GlnSerValSerSerAsn | 1039 | GlyAlaSer | 1193 | GlnGlnTyrGlyTyrSerGlnIleThr |
| VL_11 | 886 | AsnIleGlySerLysSer | 1040 | AspAspSer | 1194 | GlnValTrpAspSerSerSerAspLeuLeuT |
| | | | | | | yrVal |
| VL_12 | 887 | GlnSerValSerSerSerTyr | 1041 | GlyAlaSer | 1195 | GlnGlnTyrGlyArgSerProPheThr |
| VL_13 | 888 | GlnSerValThrSerAsnTyr | 1042 | GlyAlaSer | 1196 | GlnGlnTyrGlySerSerProThr |
| VL_14 | 889 | ThrGlyAlaValThrSerGlyPh | 1043 | SerAlaThr | 1197 | LeuLeuTyrTyrGlyGlyAlaGlnProTrpVa |
| | | eTyr | | | | l |
| VL_15 | 890 | AsnIleGlySerLysSer | 1044 | AspAspSer | 1198 | GlnLeuTrpAspGlyAlaSerAspLeuValIl |
| | | | | | | e |
| VL_16 | 891 | GlnThrIleSerSerTyr | 1045 | GlyAlaSer | 1199 | GlnGlnSerTyrSerThrProGlnThr |
| VL_17 | 892 | AsnIleGlySerLysSer | 1046 | AspAspSer | 1200 | GlnValTrpAspSerSerSerAspHisValVa |
| | | | | | | l |
| VL_18 | 893 | AsnIleGlySerLysSer | 1047 | AspAspSer | 1201 | GlnValTrpAspSerSerSerAspHisValVa |
| | | | | | | l |
| VL_19 | 894 | GlnArgValArgSerSerTyr | 1048 | GlyAlaSer | 1202 | GlnGlnTyrGlySerSerProProArgIleIle |
| VL_20 | 895 | GlnThrValSerAsnAsn | 1049 | AspAlaSer | 1203 | GlnGlnTyrGlySerSerProLeuThr |
| VL_21 | 896 | AsnIleGlySerLysSer | 1050 | AspAspSer | 1204 | GlnValTrpAspSerSerSerAspHisValVa |
| | | | | | | l |
| VL_22 | 897 | AspIleGluSerLysSer | 1051 | AspAspSer | 1205 | GlnValTrpAspGlyIleIleAsnGlnValVal |
| VL_23 | 898 | GlnGlyValArgAlaSerSer | 1052 | AlaAlaSer | 1206 | GlnGlnTyrGlyArgSerProThr |
| VL_24 | 899 | GlnSerIleSerSerTyr | 1053 | AlaAlaSer | 1207 | GlnGlnSerTyrSerThrProProTyrThr |
| VL_25 | 900 | GlnSerValSerSerSerTyr | 1054 | GlyAlaSer | 1208 | GlnGlnTyrGlySerSerProGlnTyrThr |
| VL_26 | 901 | AsnIleGlySerLysSer | 1055 | AspAspSer | 1209 | GlnValTrpGlySerSerAsnAspProValV |
| | | | | | | al |
| VL_27 | 902 | AsnIleGlySerLysSer | 1056 | AspAspSer | 1210 | GlnValTrpAspSerSerSerAspHisValVa |
| | | | | | | l |
| VL_28 | 903 | SerSerAsnIleGlyAsnAsnTy | 1057 | AspAsnAsn | 1211 | GlyThrTrpAspSerSerLeuSerAlaValVa |
| | | r | | | | l |
| VL_29 | 904 | AsnIleGlyAlaLysSer | 1058 | AspAspSer | 1212 | GlnValTrpAspAsnThrGlyAspHisProA |
| | | | | | | rgValIle |
| VL_30 | 905 | GlnSerLeuValTyrSerAspGl | 1059 | LysValSer | 1213 | MetGlnGlyLysHisTrpProProThr |
| | | yAsnThrTyr | | | | |
| VL_31 | 906 | SerLeuArgSerTyrTyr | 1060 | GlyLysAsn | 1214 | AsnSerArgAspSerSerGlyAsnHisTrpV |
| | | | | | | al |
| VL_32 | 907 | AsnIleGlySerLysSer | 1061 | AspAspSer | 1215 | GlnValTrpAspSerSerSerAspHisSerVa |
| | | | | | | lVal |
| VL_33 | 908 | AsnIleGlySerTyrSer | 1062 | AspAspSer | 1216 | GlnValTrpAspSerSerSerAspHisValIle |
| VL_34 | 909 | AsnIleGlySerLysSer | 1063 | AspAspSer | 1217 | GlnValTrpAspSerSerSerAspHisValVa |
| | | | | | | l |
| VL_35 | 910 | AsnLeuGlyGlyArgTyr | 1064 | GlnAspLeu | 1218 | GlnAlaTrpAspThrTyrThrValVal |
| VL_36 | 911 | AsnIleGlySerLysSer | 1065 | AspAspSer | 1219 | GlnValTrpAspSerSerSerAspHisValVa |
| | | | | | | l |
| VL_37 | 912 | SerLeuArgSerTyrTyr | 1066 | GlyLysAsn | 1220 | AsnSerArgAspSerSerGlyAsnHisValV |
| | | | | | | al |
| VL_38 | 913 | LysLeuGlyAspLysTyr | 1067 | GlnAspThr | 1221 | GlnAlaTrpAspSerSerThrAsnTyrVal |
| VL_39 | 914 | GlnSerIleAsnSerAsn | 1068 | GlyAlaSer | 1222 | GlnGlnPheGluGlnTrpProLeuThr |
| VL_40 | 915 | GlnArgIleSerLysTyr | 1069 | GlySerSer | 1223 | GlnGlnSerAspSerValProIleThr |
| VL_41 | 916 | SerSerAsnIleGlyAlaGlyTyr | 1070 | GlyAspAsn | 1224 | GlnSerHisAspGluSerLeuAsnSerLysV |
| | | Arg | | | | al |
| VL_42 | 917 | GlnSerValSerSerAsn | 1071 | GlyAlaSer | 1225 | GlnGlnTyrGlySerSerProLeuThr |
| VL_43 | 918 | AsnIleGlySerLysSer | 1072 | AspAspSer | 1226 | GlnLeuTrpAspGlyAlaSerAspLeuValIl |
| | | | | | | e |
| VL_44 | 919 | AsnIleGlySerLysSer | 1073 | AspAspSer | 1227 | GlnValTrpAspSerSerSerAspHisValVa |
| | | | | | | l |
| VL_45 | 920 | GlnSerValSerSerAsn | 1074 | GlyAlaSer | 1228 | GlnGlnTyrAsnAsnTrpProProGlnTyrT |
| | | | | | | hr |
| VL_46 | 921 | AsnIleGlySerLysSer | 1075 | AspAspSer | 1229 | GlnValTrpAspSerSerSerAspTyrValVa |
| | | | | | | l |
| VL_47 | 922 | AsnIleGlySerLysSer | 1076 | AspAspSer | 1230 | GlnValTrpAspSerLeuSerAspHisValIle |
| VL_48 | 923 | AsnIleGlyThrLysSer | 1077 | AspAspSer | 1231 | GlnValTrpAspHisSerAsnAspHisValV |
| | | | | | | al |
| VL_49 | 924 | AsnIleGlySerLysSer | 1078 | AspAspSer | 1232 | SerAlaTrpAspSerSerLeuThrAlaValVa |
| | | | | | | l |
| VL_50 | 925 | AsnIleGlySerLysGly | 1079 | AspAspArg | 1233 | GlnValTrpAspThrAsnSerGlnHisValV |
| | | | | | | al |
| VL_51 | 926 | SerSerAsnIleGlyAsnAsnGl | 1080 | TyrAspAsp | 1234 | AlaThrTrpAspAspArgLeuLysGlyTyrV |
| | | y | | | | al |
| VL_52 | 927 | AsnIleGlySerLysSer | 1081 | AspAspSer | 1235 | GlnValTrpAspSerSerSerAspGlnGlyV |
| | | | | | | al |
| VL_53 | 928 | GlyGlySerLeuAlaSerAsnT | 1082 | GluAspLys | 1236 | GlnSerTyrAspSerAlaAsnProLeuValV |
| | | yr | | | | al |
| VL_54 | 929 | AsnLeuGlyGlyTyrSer | 1083 | AspAspSer | 1237 | GlnValTrpAspSerSerSerAspLeuValV |
| | | | | | | al |
| VL_55 | 930 | SerGlySerIleAlaSerAsnTyr | 1084 | GluAspAsn | 1238 | GlnSerTyrAspThrSerAsnLeuValVal |
| VL_56 | 931 | AsnIleGlySerLysAsn | 1085 | AspAspThr | 1239 | GlnValTrpAspArgAsnThrGlyHisValV |
| | | | | | | al |
| VL_57 | 932 | SerSerAspValGlyGlyTyrAs | 1086 | GluValSer | 1240 | SerSerTyrThrSerSerSerThrLeuValVal |
| | | nTyr | | | | |
| VL_58 | 933 | AsnIleGlyAsnLysAsn | 1087 | AspAspLys | 1241 | GlnValTrpAspThrSerGluTyrGlnAsnA |
| | | | | | | rgVal |
| VL_59 | 934 | SerGlySerIleAlaSerAsnTyr | 1088 | GluHisAsn | 1242 | GlnSerTyrAspAsnSerAsnProHisValV |
| | | | | | | al |
| VL_60 | 935 | SerSerAsnIleGlyAlaGlyTyr | 1089 | GlyAsnSer | 1243 | GlnSerTyrAspSerSerLeuSerGlyPheT |
| | | Asp | | | | yrVal |
| VL_61 | 936 | AsnIleGlyAsnLysAsn | 1090 | AspAspSer | 1244 | GlnValTrpAspSerSerSerAspHisValVa |
| | | | | | | l |
| VL_62 | 937 | GlnGlyIleSerSerTrp | 1091 | GlyAlaSer | 1245 | GlnGlnAlaAsnSerPheProIleThr |
| VL_63 | 938 | SerGlySerIleAlaSerAsnTyr | 1092 | GluAspAsn | 1246 | GlnSerTyrAspSerSerAsnHisValVal |
| VL_64 | 939 | GlnGlyValAsnSerAsp | 1093 | GlyAlaSer | 1247 | GlnGlnTyrAsnAsnTrpProTrpThr |
| VL_65 | 940 | LysLeuGlyAspLysTyr | 1094 | GluAspThr | 1248 | GlnAlaTrpAspThrSerAlaValVal |
| VL_66 | 941 | AsnIleGlySerLysSer | 1095 | AspAspSer | 1249 | GlnLeuTrpAspAspSerSerAspHisValV |
| | | | | | | al |
| VL_67 | 942 | AsnIleGlySerLysSer | 1096 | AspAspSer | 1250 | GlnValTrpAspSerSerSerAspHisValVa |
| | | | | | | l |
| VL_68 | 943 | SerLeuArgAspTyrTyr | 1097 | GlyLysAsn | 1251 | AsnSerArgAspSerSerGlyAsnHisValV |
| | | | | | | al |
| VL_69 | 944 | AsnIleGlyArgLysSer | 1098 | AspAspThr | 1252 | GlnLeuTyrAspSerAspSerAspAsnValV |
| | | | | | | al |
| VL_70 | 945 | AsnIleGlySerLysSer | 1099 | AspAspSer | 1253 | GlnValTrpAspSerSerSerAspHisProV |
| | | | | | | al |
| VL_71 | 946 | SerLeuArgSerTyrTyr | 1100 | GlyLysAsn | 1254 | AsnSerArgAspSerSerGlyAsnLeuGlyV |
| | | | | | | al |
| VL_72 | 947 | GlnAsnIleLeuThrAsn | 1101 | AlaAlaSer | 1255 | GlnGlnSerTyrSerIleProTrpThr |
| VL_73 | 948 | LysLeuGlyAsnLysTyr | 1102 | GluAsnAsn | 1256 | GlnAlaTrpAspSerSerThrAlaVal |
| VL_74 | 949 | GlnSerIleSerSerTyr | 1103 | AlaAlaSer | 1257 | GlnGlnSerTyrSerThrSerTrpThr |
| VL_75 | 950 | AsnIleGlySerLysSer | 1104 | AspAspSer | 1258 | AlaAlaTrpAspAspSerLeuAsnGlyGlnV |
| | | | | | | alVal |
| VL_76 | 951 | AsnIleGlySerLysSer | 1105 | AspAspSer | 1259 | GlnValTrpAspSerSerSerAspHisValVa |
| | | | | | | l |
| VL_77 | 952 | AsnValGlyThrThrSer | 1106 | AspAspThr | 1260 | GlnValTrpAspSerSerSerAspHisValIle |
| VL_78 | 953 | LysIleGlySerTyrSer | 1107 | AspAspSer | 1261 | GlnValTrpAspThrTyrGlyAspGlnValV |
| | | | | | | al |
| VL_79 | 954 | AsnIleGlySerLysSer | 1108 | AspAspSer | 1262 | GlnValTrpAspSerSerSerAspHisProV |
| | | | | | | al |
| VL_80 | 955 | AsnIleGlySerLysSer | 1109 | AspAspSer | 1263 | GlnValTrpAspSerGlySerAspPheValV |
| | | | | | | al |
| VL_81 | 956 | AsnIleGlySerLysSer | 1110 | AspAspSer | 1264 | GlnValTrpAspSerSerSerAspHisProV |
| | | | | | | al |
| VL_82 | 957 | AsnIleGlySerGlnSer | 1111 | AspAspSer | 1265 | GlnValTrpAspGlySerAsnAspHisValV |
| | | | | | | al |
| VL_83 | 958 | AsnIleGlyArgGluSer | 1112 | AspAspSer | 1266 | GlnValTrpAspSerSerIleAspHisValVal |
| VL_84 | 959 | AsnIleGlySerLysSer | 1113 | AspAspSer | 1267 | GlnValTrpAspSerSerSerAspHisValVa |
| | | | | | | l |
| VL_85 | 960 | AsnIleGlySerLysSer | 1114 | AspAspSer | 1268 | GlnValTrpAspSerSerSerAspHisValVa |
| | | | | | | l |
| VL_86 | 961 | AsnIleGlySerLysGly | 1115 | AspAspSer | 1269 | GlnValTrpAspAsnSerSerAspSerValV |
| | | | | | | al |
| VL_87 | 962 | GlyGlySerIleAlaSerAsnTyr | 1116 | LysAspAsn | 1270 | GlnSerTyrGlySerGlyAsnValVal |
| VL_88 | 963 | SerGlySerIleAlaSerAsnTyr | 1117 | GluHisAsn | 1271 | GlnSerPheAspArgAsnAsnProLysTrp |
| | | | | | | Val |
| VL_89 | 964 | AsnIleGlySerLysSer | 1118 | AspAspSer | 1272 | GlnValTrpAspSerSerSerAspHisLeuV |
| | | | | | | alVal |
| VL_90 | 965 | LysLeuGlyAspLysTyr | 1119 | HisAspThr | 1273 | GlnValTrpAspGlyThrThrAspHisPheL |
| | | | | | | eu |
| VL_91 | 966 | AsnIleGlySerLysSer | 1120 | TyrAspSer | 1274 | GlnValTrpAspSerValSerAspProValM |
| | | | | | | et |
| VL_92 | 967 | SerSerAspValGlyGlyTyrAs | 1121 | GluValSer | 1275 | SerSerTyrAlaGlySerAsnAsnLeuVal |
| | | nTyr | | | | |
| VL_93 | 968 | LysLeuGlyAspLysTyr | 1122 | GlnAsnAsn | 1276 | GlnAlaTrpAspSerSerAlaValVal |
| VL_94 | 969 | AsnIleGlySerLysSer | 1123 | AspAspSer | 1277 | GlnValTrpAspSerThrSerAspHisProGl |
| | | | | | | uValVal |
| VL_95 | 970 | AsnIleGlySerLysSer | 1124 | AspAspAsp | 1278 | GlnValTrpAspSerGlySerAspHisValVa |
| | | | | | | l |
| VL_96 | 971 | AsnIleGlySerLysSer | 1125 | AspAspSer | 1279 | GlnValTrpAspSerSerSerAspHisValVa |
| | | | | | | l |
| VL_97 | 972 | AsnIleGlySerLysSer | 1126 | AspAspSer | 1280 | GlnValTrpAspSerSerSerAspHisValVa |
| | | | | | | l |
| VL_98 | 973 | SerSerAsnIleGlyAsnAsnTy | 1127 | GluAsnAsn | 1281 | GlyThrTrpAspSerSerLeuSerAlaGlyVa |
| | | r | | | | l |
| VL_99 | 974 | SerSerAsnIleGlyAlaGlyTyr | 1128 | GlyAsnSer | 1282 | GlnSerTyrAspSerSerLeuSerTrpVal |
| | | Asp | | | | |
| VL_100 | 975 | SerSerAspValGlyGlyTyrAs | 1129 | GlyValSer | 1283 | SerSerTyrArgIleArgAspSerLeuVal |
| | | nPhe | | | | |
| VL_101 | 976 | AsnIleGlySerLysSer | 1130 | AspAspSer | 1284 | GlnValTrpAspSerSerSerAspHisValVa |
| | | | | | | l |
| VL_102 | 977 | GlyGlyGlyIleAlaAspAsnTy | 1131 | AspAspAsp | 1285 | GlnSerTyrAspSerAlaValProValVal |
| | | r | | | | |
| VL_103 | 978 | AsnIleGlySerLysSer | 1132 | AspAspSer | 1286 | GlnValTrpAspSerAspAsnAspAsnSer |
| | | | | | | GluValIle |
| VL_104 | 979 | AsnIleGlySerLysAsn | 1133 | AspAspAsn | 1287 | GlnValTrpAspSerSerSerGluHisValVa |
| | | | | | | l |
| VL_105 | 980 | AsnIleGlySerAsnSer | 1134 | AspAspSer | 1288 | GlnValTrpAspSerSerSerAspHisValVa |
| | | | | | | l |
| VL_106 | 981 | IleLeuGlyHisTyrHis | 1135 | GlyLysAsp | 1289 | AsnSerArgAspArgSerGlyThrGlnValL |
| | | | | Asn | | eu |
| VL_107 | 982 | SerSerAspValGlyGlyTyrAs | 1136 | GluValSer | 1290 | SerSerTyrThrSerSerSerThrLeuValVal |
| | | nTyr | | | | |
| VL_108 | 983 | GlnSerValSerThrAsn | 1137 | GlyAlaSer | 1291 | GlnGlnTyrAsnThrTrpProProValArg |
| VL_109 | 984 | SerSerAspValGlyGlyTyrAs | 1138 | AspValSer | 1292 | SerSerTyrThrSerSerSerThrLeuValVal |
| | | nTyr | | | | |
| VL_110 | 985 | SerSerAspValGlyGlyTyrAs | 1139 | AspValSer | 1293 | SerSerTyrThrSerSerSerThrLeuValVal |
| | | nTyr | | | | |
| VL_111 | 986 | LysIleGlySerLysIle | 1140 | HisAspSer | 1294 | GlnValTrpAspValAsnThrAspHisValV |
| | | | | | | al |
| VL_112 | 987 | SerSerAspValGlyGlyTyrAs | 1141 | GluValThr | 1295 | SerSerTyrThrSerSerSerThrPheValVa |
| | | nTyr | | | | l |
| VL_113 | 988 | SerGlySerIleValSerAsnTyr | 1142 | GluAspAsn | 1296 | GlnSerTyrAspSerGlyAsnValVal |
| VL_114 | 989 | GlnSerValSerSerSerTyr | 1143 | GlyAlaSer | 1297 | GlnGlnTyrGlySerSerProLeuThr |
| VL_115 | 990 | SerGlySerIleAlaThrAsnTyr | 1144 | GluAspAsn | 1298 | GlnSerTyrAspSerSerThrGlyVal |
| VL_116 | 991 | SerSerAspValGlyGlyTyrAs | 1145 | AspValSer | 1299 | SerSerTyrThrSerSerSerThrLeuValVal |
| | | nTyr | | | | |
| VL_117 | 992 | AsnIleGluSerLysSer | 1146 | AspAspSer | 1300 | GlnValTrpAspSerGlyHisGlnVal |
| VL_118 | 993 | SerSerTyrIleAlaThrAsnSer | 1147 | SerAspSer | 1301 | AlaAlaTrpAspAspSerLeuAsnAlaTyrV |
| | | | | | | al |
| VL_119 | 994 | SerSerAspIleGlyGlyTyrAs | 1148 | GluValSer | 1302 | SerSerTyrThrSerSerSerThrLeuValVal |
| | | nTyr | | | | |
| VL_120 | 995 | SerSerAspValGlyGlyTyrAs | 1149 | AspValSer | 1303 | SerSerTyrThrSerSerSerThrLeuValVal |
| | | nTyr | | | | |
| VL_121 | 996 | SerSerAsnIleGlyAlaGlyTyr | 1150 | GlyAsnAsn | 1304 | AlaThrTrpAspAspSerLeuAsnAlaProT |
| | | Asp | | | | yrVal |
| VL_122 | 997 | LysLeuGlyAsnLysTyr | 1151 | GlnAspAsp | 1305 | GlnAlaTrpAspSerThrTyrValVal |
| VL_123 | 998 | LysLeuGlyAspLysTyr | 1152 | GlnAspThr | 1306 | GlnAlaTrpAspSerThrThrLeuVal |
| VL_124 | 999 | GlyGlySerIleAlaSerAsnTyr | 1153 | LysAspAsn | 1307 | GlnSerTyrGlySerGlyAsnValVal |
| VL_125 | 1000 | SerSerAsnIleAlaSerAsnTh | 1154 | SerAsnAsn | 1308 | SerAlaTrpAspAspSerLeuHisThrTyrV |
| | | r | | | | al |
| VL_126 | 1001 | SerSerAspValGlyGlyTyrAs | 1155 | GluValSer | 1309 | SerSerTyrAlaGlySerAspThrValVal |
| | | nTyr | | | | |
| VL_127 | 1002 | SerSerAsnIleGlyAsnAsnTy | 1156 | AspAsnAsp | 1310 | GlyThrTrpAspAsnSerLeuSerAlaValV |
| | | r | | | | al |
| VL_128 | 1003 | AsnIleGlySerLysSer | 1157 | AspAspSer | 1311 | GlnValTrpAspSerSerSerAspHisValVa |
| | | | | | | l |
| VL_129 | 1004 | SerSerAspValGlyGlyTyrAs | 1158 | AspValSer | 1312 | SerSerTyrThrSerSerSerThrLeuValVal |
| | | nTyr | | | | |
| VL_130 | 1005 | SerSerAsnIleGlyAsnAsnTy | 1159 | GluAsnAsn | 1313 | GlyThrTrpAspSerSerLeuSerAlaValVa |
| | | r | | | | l |
| VL_131 | 1006 | SerSerAspValGlyGlyTyrAs | 1160 | GluValSer | 1314 | SerSerTyrThrSerSerSerThrLeuValVal |
| | | pTyr | | | | |
| VL_132 | 1007 | AsnIleGlySerLysSer | 1161 | AlaAspSer | 1315 | GlnValTrpAspSerSerPheAspValAla |
| VL_133 | 1008 | AsnIleGlyAspLysArg | 1162 | TyrAspThr | 1316 | GlnValTrpAspThrAspThrAsnHisAlaV |
| | | | | | | al |
| VL_134 | 1009 | SerSerAspValGlyAlaTyrAs | 1163 | AspValSer | 1317 | SerSerTyrThrThrSerSerThrLeuVal |
| | | nTyr | | | | |
| VL_135 | 1010 | LysLeuGlyAspLysTyr | 1164 | GlnAspSer | 1318 | GlnThrTrpAspSerSerThrValVal |
| VL_136 | 1011 | LysLeuGlyAspLysTyr | 1165 | GlnAspIle | 1319 | GlnAlaTrpAspArgSerSerTyrVal |
| VL_137 | 1012 | SerSerAspValGlyGlyTyrAs | 1166 | GluValSer | 1320 | SerSerTyrSerGlySerAsnAsnLeuValVa |
| | | nTyr | | | | l |
| VL_138 | 1013 | SerSerAspValGlyGlyTyrAs | 1167 | AspValAsn | 1321 | SerSerTyrThrSerSerAsnThrLeuValVa |
| | | nTyr | | | | l |
| VL_139 | 1014 | SerSerAsnIleGlyAlaGlyTyr | 1168 | GlyAsnSer | 1322 | GlnSerTyrAspSerSerLeuSerGlySerGl |
| | | Asp | | | | yTyrVal |
| VL_140 | 1015 | SerSerAspValGlyGlyTyrAs | 1169 | GluValSer | 1323 | SerSerTyrThrSerSerSerThrLeuValVal |
| | | nTyr | | | | |
| VL_141 | 1016 | SerSerAspValGlyGlyTyrAs | 1170 | AspValSer | 1324 | SerSerTyrThrSerSerSerThrLeuValVal |
| | | nTyr | | | | |
| VL_142 | 1017 | AsnIleGlySerLysSer | 1171 | AspAspSer | 1325 | GlnValTrpAspSerGlyAsnIleHisProVal |
| | | | | | | Val |
| VL_143 | 1018 | GlyAsnAsnTyr | 1172 | GluAsnAsn | 1326 | GlyThrTrpAspSerSerLeuAsnValGlyV |
| | | | | | | al |
| VL_144 | 1019 | LysLeuGlyAsnLysTyr | 1173 | GlnAspAsn | 1327 | GlnAlaTrpAspSerSerThrAlaVal |
| VL_145 | 1020 | SerSerAspValGlyGlyTyrAs | 1174 | AspValSer | 1328 | SerSerTyrAlaGlySerSerValVal |
| | | nTyr | | | | |
| VL_146 | 1021 | SerSerAspValGlyGlyTyrAs | 1175 | GluValSer | 1329 | SerSerTyrThrSerSerSerThrLeuValVal |
| | | nTyr | | | | |
| VL_147 | 1022 | GlySerAsnIleGlyAlaGlyTyr | 1176 | GlyAsnIle | 1330 | AlaAlaTrpAspAspSerLeuAsnGlyLeuT |
| | | Asp | | | | yrVal |
| VL_148 | 1023 | SerSerAspValGlyGlyTyrAs | 1177 | AspValSer | 1331 | SerSerTyrThrSerSerSerThrPheValVa |
| | | nTyr | | | | l |
| VL_149 | 1024 | SerSerAsnIleGlyIleAsnThr | 1178 | ArgAsnAsn | 1332 | AlaAlaTrpAspAspSerLeuSerGlyTrpV |
| | | | | | | al |
| VL_150 | 1025 | GlySerAspIleGlyAspTyrLy | 1179 | AspValThr | 1333 | SerProHisThrProSerArgValIle |
| | | sTyr | | | | |
| VL_151 | 1026 | SerSerAsnIleGlyAlaGlyTyr | 1180 | GlyAsnSer | 1334 | AlaAlaTrpAspAspGlyProSerGlyTyrVa |
| | | Asp | | | | l |
| VL_152 | 1027 | LysLeuGlyAspLysTyr | 1181 | ArgAspAsn | 1335 | GlnAlaTrpAspSerSerThrValVal |
| VL_153 | 1028 | GlnSerIleAspThrSer | 1182 | AlaAlaSer | 1336 | GlnGlnSerTyrSerThrProGlnTyrThr |
| VL_154 | 1029 | GlnSerIleSerSerTrp | 1183 | LysAlaSer | 1337 | GlnGlnTyrAsnThrTyrPheProThr |

TABLE 7
VH CDR SEQUENCES COMBINED

| | | SEQ ID |
|---|---|---|
| mAb ID | VH_CDR1/2/3 | NO: |
| VH_1 | GlyAspSerIleSerSerGlyTyrTrpIleSerTyrAspGlySerAsnLysAlaArgValGlySerGlyGlyTrpThrP | 1338 |
| | roAspTyr | |
| VH_2 | GlyAspSerValSerSerAsnSerAlaAlaIleAsnProAsnSerGlyGlyThrAlaArgGluValAlaThrIlePro | 1339 |
| | AlaHisPheAspTyr | |
| VH_3 | GlyAspSerValSerSerAsnSerAlaAlaIleSerAlaTyrAsnGlyAsnThrAlaArgAspTyrAspIleLeuThr | 1340 |
| | GlyLeuAspTyr | |
| VH_4 | GlyAspSerValSerSerAsnSerAlaAlaIleSerGlySerGlyGlyArgThrAlaLysAspTrpAlaGlyTyrIleA | 1341 |
| | snGlyTrpTyrGlyAsn | |
| VH_5 | GlyAspSerValSerSerAsnSerAlaAlaIleSerGlySerGlyGlySerThrAlaLysAspTrpAlaGlyTyrValA | 1342 |
| | snGlyTrpTyrGlyAsn | |
| VH_6 | GlyAspSerValSerSerAsnSerAlaAlaIleSerGlySerGlyGlySerThrAlaLysAspTrpGlyThrSerLeuL | 1343 |
| | euTyrGlyTyrPheAspTyr | |
| VH_7 | GlyAspSerValSerSerAsnSerAlaAlaIleSerTyrAspGlySerAsnLysAlaArgValGlySerGlyGlyTrp | 1344 |
| | ThrProAspTyr | |
| VH_8 | GlyAspSerValSerSerAsnSerAlaAlaIleTyrSerGlyGlySerThrAlaArgAspPheGluGlySerGlyAla | 1345 |
| | LeuAspVal | |
| VH_9 | GlyAspSerValSerSerAsnSerAlaAlaThrTyrTyrSerSerLysTrpTyrAsnAlaArgGlyGlySerSerGlu | 1346 |
| | PheTyrTyrTyrGlyMetAspVal | |
| VH_10 | GlyAspSerValSerSerAspSerAlaSerIleSerGlySerGlyGlyIleThrAlaLysAspTrpAlaGlyTyrThrA | 1347 |
| | snGlyTrpTyrGlySer | |
| VH_11 | GlyGlySerIleSerGlySerAsnTyrTyrIleSerGlySerGlyGlyIleThrAlaLysAspTrpAlaGlyTyrThrAs | 1348 |
| | nGlyTrpTyrGlySer | |
| VH_12 | GlyGlySerIleSerSerSerAsnTrpIleSerGlySerGlyGlySerThrAlaLysAspArgSerArgArgAlaProT | 1349 |
| | yrTyrPheAspTyr | |
| VH_13 | GlyGlySerIleSerSerSerAsnTrpIleSerGlySerGlyGlySerThrAlaLysValTyrArgGlyTyrAspAlaPh | 1350 |
| | eAspIle | |
| VH_14 | GlyGlySerIleSerSerSerAsnTrpIleTyrProGlyAspSerAspThrAlaArgHisAlaGlyAspGlyGlnIleA | 1351 |
| | spTyr | |
| VH_15 | GlyGlySerIleSerSerSerAsnTrpThrTyrTyrArgSerLysTrpTyrAsnAlaArgGluGlySerGlyLeuTyr | 1352 |
| | TyrTyrTyrGlyMetAspVal | |
| VH_16 | GlyGlySerValSerSerAsnSerAlaAlaIleSerGlySerGlyGlySerThrAlaArgGlyGlySerGlyTrpTyrHi | 1353 |
| | sTyrPheAspTyr | |
| VH_17 | GlyGlyThrPheSerSerTyrAlaIleSerGlyThrGlyGlyArgThrAlaLysAspTrpAlaGlyTyrIleAsnGlyT | 1354 |
| | rpTyrGlySer | |
| VH_18 | GlyGlyThrPheSerSerTyrAlaIleSerTyrAspGlySerAsnLysAlaArgValGlySerGlyGlyTrpThrPro | 1355 |
| | AspTyr | |
| VH_19 | GlyGlyThrPheSerSerTyrAlaIleTrpTyrAspGlySerAsnLysAlaArgLeuGlySerGlyTrpSerLeuAs | 1356 |
| | pTyr | |
| VH_20 | GlyPheThrPheAsnThrTyrAlaIleSerGlySerGlyAspArgThrAlaLysAspTrpAlaGlyTyrIleAsnGly | 1357 |
| | TrpPheGlyAsn | |
| VH_21 | GlyPheThrPheAsnThrTyrAlaIleSerGlySerGlyAspIleThrAlaLysAspTrpAlaGlyTyrValAsnGly | 1358 |
| | TrpTyrGlyAsn | |
| VH_22 | GlyPheThrPheAsnThrTyrAlaIleSerTyrAspGlySerAsnLysAlaArgValGlySerGlyGlyTrpThrPr | 1359 |
| | oAspTyr | |
| VH_23 | GlyPheThrPheAspAspTyrAlaIleAsnAlaGlyAsnGlyAsnThrAlaArgGlyGlyTyrCysSerSerThrS | 1360 |
| | erCysTyrProAspTyrAsnTrpPheAspPro | |
| VH_24 | GlyPheThrPheAspAspTyrAlaIleSerGlySerGlyAspArgThrAlaLysAspTrpAlaGlyTyrIleAsnGl | 1361 |
| | yTrpTyrAlaAsn | |
| VH_25 | GlyPheThrPheAspAspTyrAlaIleTyrSerGlyGlySerThrAlaArgAspArgArgGlyGlyAsnTrpTyrGl | 1362 |
| | uPheAspTyr | |
| VH_26 | GlyPheThrPheAspAspTyrAlaIleTyrSerGlyGlySerThrAlaArgGluGlyLeuAlaMetAlaGlyTyrP | 1363 |
| | heAspTyr | |
| VH_27 | GlyPheThrPheGlyAsnHisGlyIleLysHisAspGlySerGluGlnAlaArgValAlaValGlyAlaAsnLeuAla | 1364 |
| | PheAspIle | |
| VH_28 | GlyPheThrPheSerArgTyrGlyIleSerGlySerGlyAspArgThrAlaLysAspTrpAlaGlyTyrIleAsnGly | 1365 |
| | TrpTyrGlyAsn | |
| VH_29 | GlyPheThrPheSerAsnAlaTrpIleIleProIlePheGlyThrAlaAlaArgGlyMetAlaGlnSerProAlaPh | 1366 |
| | eAspTyr | |
| VH_30 | GlyPheThrPheSerAsnAlaTrpIleSerGlySerGlyGlyArgThrAlaLysAspTrpAlaGlyTyrIleAsnGly | 1367 |
| | TrpTyrGlyAsn | |
| VH_31 | GlyPheThrPheSerAsnAlaTrpThrTyrTyrAsnSerLysTrpTyrAsnAlaArgGluThrGlyGlyPheAsp | 1368 |
| | Tyr | |
| VH_32 | GlyPheThrPheSerAsnTyrAlaIleAsnThrAspGlyGlyAsnThrAlaArgAspProValArgGlyAspGlyT | 1369 |
| | yrAsnPheAspTyr | |
| VH_33 | GlyPheThrPheSerAsnTyrAlaIleSerGlySerGlyAspIleThrAlaLysAspTrpAlaGlyTyrValAsnGly | 1370 |
| | TrpTyrGlyAsn | |
| VH_34 | GlyPheThrPheSerAsnTyrAlaIleSerGlySerGlyGlySerThrAlaLysAlaThrGlyTyrSerSerGlyTrp | 1371 |
| | TyrGlyAlaTyrPheAspTyr | |
| VH_35 | GlyPheThrPheSerAsnTyrAlaIleTyrHisSerGlySerThrAlaArgAspArgGlySerMetAspVal | 1372 |
| VH_36 | GlyPheThrPheSerAsnTyrAlaIleTyrProGlyAspSerAspThrAlaArgLeuGlyArgThrSerHisGlnS | 1373 |
| | erTrpAspLeuGlyTyr | |
| VH_37 | GlyPheThrPheSerAsnTyrAlaIleTyrProGlyAspSerAspThrAlaSerGlyAlaSerProTyrTyrPheAs | 1374 |
| | pTyr | |
| VH_38 | GlyPheThrPheSerAsnTyrAlaIleTyrSerGlyGlySerThrAlaArgGluSerAsnThrAlaAsnThrHisPh | 1375 |
| | eAspTyr | |
| VH_39 | GlyPheThrPheSerAsnTyrAlaThrTyrTyrArgSerLysTrpTyrAsnAlaArgGlyGlyValGlyAlaThrTr | 1376 |
| | pTyrTyrGlyMetAspVal | |
| VH_40 | GlyPheThrPheSerAsnTyrGlyIleSerTyrAspGlySerAsnLysAlaLysGlnGlnTrpLeuGlyThrTrpTy | 1377 |
| | rPheAspLeu | |
| VH_41 | GlyPheThrPheSerAsnTyrGlyIleSerTyrAspGlySerAsnLysAlaLysGlyLeuLeuValAlaSerIleTyr | 1378 |
| | AspAlaPheAspIle | |
| VH_42 | GlyPheThrPheSerAspTyrAlaIleSerTrpAsnSerGlySerIleAlaLysAspIleAlaAlaGlyGlyLeuAspS | 1379 |
| | er | |
| VH_43 | GlyPheThrPheSerAspTyrTyrValSerGlySerGlyThrSerThrAlaLysAspTrpAlaGlyTyrIleAsnGly | 1380 |
| | TrpTyrGlyAsn | |
| VH_44 | GlyPheThrPheSerSerTyrAlaIleAsnProAsnSerGlyAspThrAlaArgGluGlnTrpLeuGlyProAlaH | 1381 |
| | isPheAspTyr | |
| VH_45 | GlyPheThrPheSerSerTyrAlaIleAsnProAsnSerGlyGlyThrAlaArgGluArgAsnArgAlaGlyGluP | 1382 |
| | heSerAlaPheAspIle | |
| VH_46 | GlyPheThrPheSerSerTyrAlaIleGluProGlyAsnGlyAspThrAlaArgGlyAlaSerGlyLeuAspPhe | 1383 |
| VH_47 | GlyPheThrPheSerSerTyrAlaIleLysGlnAspGlySerGluLysAlaArgAspLeuHisCysGlySerSerCy | 1384 |
| | sGlyProGluAla | |
| VH_48 | GlyPheThrPheSerSerTyrAlaIleSerAlaTyrAsnGlyAsnThrAlaArgAspProValTyrSerSerSerTr | 1385 |
| | pGlyGlyTyrAlaPheAspIle | |
| VH_49 | GlyPheThrPheSerSerTyrAlaIleSerAlaTyrAsnGlyAsnThrAlaArgAspThrPheGlyGlyGlySerTy | 1386 |
| | rTyrGlyHisGlyTyr | |
| VH_50 | GlyPheThrPheSerSerTyrAlaIleSerAsnAspGlyValAsnAsnAlaArgGluAsnSerAsnAlaTrpLysV | 1387 |
| | alMetAspVal | |
| VH_51 | GlyPheThrPheSerSerTyrAlaIleSerGlySerGlyAspArgThrAlaLysAspTrpAlaGlyTyrIleAsnGly | 1388 |
| | TrpTyrGlyAsn | |
| VH_52 | GlyPheThrPheSerSerTyrAlaIleSerGlySerGlyGlyArgThrAlaLysAspTrpAlaGlyTyrIleAsnGlyT | 1389 |
| | rpTyrGlyAsn | |
| VH_53 | GlyPheThrPheSerSerTyrAlaIleSerGlySerGlyGlyArgThrAlaLysAspTrpAlaGlyTyrIleAspGlyT | 1390 |
| | rpTyrGlyAsn | |
| VH_54 | GlyPheThrPheSerSerTyrAlaIleSerGlySerGlyGlyArgThrAlaLysAspTrpGlyAlaTyrSerSerGly | 1391 |
| | TrpTyrGlyAsp | |
| VH_55 | GlyPheThrPheSerSerTyrAlaIleSerGlySerGlyGlyAsnIleAlaLysAspTrpAlaGlyTyrSerAsnGlyT | 1392 |
| | rpTyrGlySer | |
| VH_56 | GlyPheThrPheSerSerTyrAlaIleSerGlySerGlyGlyIleThrAlaLysAspTrpAlaGlyTyrSerAsnGlyT | 1393 |
| | rpPheGlySer | |
| VH_57 | GlyPheThrPheSerSerTyrAlaIleSerTyrAspGlyGlyAsnLysAlaArgValGlySerGlyGlyTrpThrPro | 1394 |
| | AspTyr | |
| VH_58 | GlyPheThrPheSerSerTyrAlaIleSerTyrAspGlySerAsnGlnAlaValGlyValGlyPheIleThrAspGly | 1395 |
| | TyrPheGlnHis | |
| VH_59 | GlyPheThrPheSerSerTyrAlaIleSerTyrAspGlySerAsnLysAlaArgValGlySerGlyGlyTrpThrPro | 1396 |
| | AspTyr | |
| VH_60 | GlyPheThrPheSerSerTyrAlaIleSerTyrAspGlySerAsnLysAlaArgValGlySerGlyGlyTrpThrPro | 1397 |
| | AspTyr | |
| VH_61 | GlyPheThrPheSerSerTyrAlaIleSerTyrAspGlySerAsnLysAlaLysGlnGlnTrpLeuGlyThrTrpTyr | 1398 |
| | PheAspLeu | |
| VH_62 | GlyPheThrPheSerSerTyrAlaIleSerTyrAspGlySerAsnLysAlaLysGluTrpGlyGlyGlyAspSerPro | 1399 |
| | ThrAspMetGlyLeuPheAspTyr | |
| VH_63 | GlyPheThrPheSerSerTyrAlaIleSerTyrAspGlySerAsnLysThrArgValGlySerGlyGlyTrpThrPro | 1400 |
| | AspTyr | |
| VH_64 | GlyPheThrPheSerSerTyrAlaIleTrpTyrAspGlyAsnAsnLysAlaArgAspAsnSerGlySerTyrAsnT | 1401 |
| | rpPheAsnPro | |
| VH_65 | GlyPheThrPheSerSerTyrAlaIleTyrProGlyAspSerAspThrAlaArgSerHisGlyGlySerAsnTrpPh | 1402 |
| | eAspPro | |
| VH_66 | GlyPheThrPheSerSerTyrAlaIleTyrProGlyAspSerAspThrAlaThrSerLeuGlyAspAspAlaPheA | 1403 |
| | spIle | |
| VH_67 | GlyPheThrPheSerSerTyrAlaIleTyrProGlyAspSerGluThrAlaArgLeuGlyHisSerGlySerTrpTyr | 1404 |
| | PheAspLeu | |
| VH_68 | GlyPheThrPheSerSerTyrAlaIleTyrSerGlyGlySerThrAlaArgAspLeuSerTyrSerAspAlaPheAs | 1405 |
| | pIle | |
| VH_69 | GlyPheThrPheSerSerTyrAlaIleTyrSerGlyGlySerThrAlaArgAspMetThrThrValAspAlaPheA | 1406 |
| | spIle | |
| VH_70 | GlyPheThrPheSerSerTyrAlaIleTyrSerGlyGlySerThrAlaArgAspThrAlaSerGlyGlyMetAspVa | 1407 |
| | l | |
| VH_71 | GlyPheThrPheSerSerTyrAlaPheTyrSerGlyGlySerThrAlaArgGluProTyrProGlyGlyProPheA | 1408 |
| | spIle | |
| VH_72 | GlyPheThrPheSerSerTyrGlyIleSerAlaSerGlyGlySerThrAlaAsnLeuTyrGlyAspTyrAsnAlaTyr | 1409 |
| VH_73 | GlyPheThrPheSerSerTyrGlyIleSerGlySerGlyAspArgThrAlaLysAspTrpAlaGlyTyrIleAsnGly | 1410 |
| | TrpTyrGlyAsn | |
| VH_74 | GlyPheThrPheSerSerTyrGlyIleSerGlySerGlyGlyArgThrAlaLysAspTrpAlaGlyTyrIleAsnGlyT | 1411 |
| | rpTyrGlyAsn | |
| VH_75 | GlyPheThrPheSerSerTyrGlyIleSerGlySerGlyGlyIleThrAlaLysAspTrpAlaGlyTyrThrAsnGlyT | 1412 |
| | rpTyrGlySer | |
| VH_76 | GlyPheThrPheSerSerTyrGlyIleSerGlySerGlyGlySerThrAlaLysAspLeuValLeuGly | 1413 |
| VH_77 | GlyPheThrPheSerSerTyrGlyIleSerTrpAsnSerGlySerIleAlaLysAspTrpAspSerSerGlyTyrTrp | 1414 |
| | ProLeuPheAspTyr | |
| VH_78 | GlyPheThrPheSerSerTyrGlyIleSerTyrAspGlySerAsnLysAlaArgValGlySerGlyGlyTrpThrPro | 1415 |
| | AspTyr | |
| VH_79 | GlyPheThrPheSerSerTyrGlyIleSerTyrAspGlySerAsnLysAlaArgValGlySerGlyGlyTrpThrPro | 1416 |
| | AspTyr | |
| VH_80 | GlyPheThrPheSerSerTyrGlyIleTrpTyrAspGlySerAsnLysAlaArgGluValValGlySerTyrTyrLeu | 1417 |
| | AspTyr | |
| VH_81 | GlyPheThrPheSerSerTyrProIleAsnProAsnSerGlyGlyThrAlaArgGlyGlyAspCysSerSerThrSe | 1418 |
| | rCysTyrAspProAspTyr | |
| VH_82 | GlyPheThrPheSerSerTyrProIleLysGlnAspGlySerGluLysAlaArgIleGlyArgPheGlyArgLysTyr | 1419 |
| | GlyMetAspVal | |
| VH_83 | GlyPheThrPheSerSerTyrProIleSerAlaTyrAsnGlyAsnThrAlaArgGlyLeuGlyAspSerSerSerSe | 1420 |
| | rTyr | |
| VH_84 | GlyPheThrPheSerSerTyrProIleSerGlySerGlyAspIleThrAlaLysAspTrpAlaGlyTyrValAsnGly | 1421 |
| | TrpTyrGlyAsn | |
| VH_85 | GlyPheThrPheSerSerTyrProIleSerGlySerGlyAspIleThrAlaLysAspTrpAlaGlyTyrValAsnGly | 1422 |
| | TrpTyrGlyAsn | |
| VH_86 | GlyPheThrPheSerSerTyrProIleSerGlySerGlyGlyArgThrAlaLysAspTrpAlaGlyTyrIleAsnGly | 1423 |
| | TrpTyrGlyAsn | |
| VH_87 | GlyPheThrPheSerSerTyrProIleSerGlySerGlyGlyArgThrAlaLysAspTrpGlyAlaTyrSerSerGly | 1424 |
| | TrpTyrGlyAsp | |
| VH_88 | GlyPheThrPheSerSerTyrProIleSerGlySerGlyGlyIleThrAlaLysAspTrpAlaGlyTyrThrAsnGly | 1425 |
| | TrpTyrGlySer | |
| VH_89 | GlyPheThrPheSerSerTyrProIleSerGlyThrGlyGlyArgThrAlaLysAspTrpAlaGlyTyrIleAsnGly | 1426 |
| | TrpTyrGlySer | |
| VH_90 | GlyPheThrPheSerSerTyrProIleSerTyrAspAlaThrAsnAsnAlaLysGluArgPheThrGlyGlyTyrT | 1427 |
| | yrThrTyrPheAspTyr | |
| VH_91 | GlyPheThrPheSerSerTyrProIleTyrHisSerGlySerThrAlaArgAlaGlyGlyLeuHisLeuAspTyr | 1428 |
| VH_92 | GlyPheThrPheSerSerTyrProIleTyrProGlyAspSerAspThrAlaArgGlyAsnGlyAspGlyGlyPheA | 1429 |
| | spTyr | |
| VH_93 | GlyPheThrPheSerSerTyrSerIleSerGlySerGlyGlyArgThrAlaLysAspTrpAlaGlyTyrIleAsnGlyT | 1430 |
| | rpTyrGlyAsn | |
| VH_94 | GlyPheThrPheSerSerTyrTrpIleSerGlySerGlyAspIleThrAlaLysAspTrpAlaGlyTyrValAsnGly | 1431 |
| | TrpTyrGlyAsn | |
| VH_95 | GlyPheThrPheSerSerTyrTrpIleSerTyrAspGlySerAsnLysAlaArgAspArgGlyValGluGlyAlaTy | 1432 |
| | rGlyMetAspVal | |
| VH_96 | GlyPheThrPheSerSerTyrTrpIleSerTyrAspGlySerAsnLysAlaLysGlyLeuLeuValAlaSerIleTyr | 1433 |
| | AspAlaPheAspIle | |
| VH_97 | GlyPheThrPheSerSerTyrTrpIleTyrHisSerGlySerThrAlaArgGlySerAsnIlePheAspIle | 1434 |
| VH_98 | GlyPheThrPheSerThrTyrAlaIleLysSerLysAsnAspGlyGlyThrThrThrThrAlaProSerLeuMetA | 1435 |
| | spVal | |
| VH_99 | GlyPheThrPheSerThrTyrAlaIleSerAlaTyrAsnGlyAsnThrAlaArgAspLeuThrPheGlySerGlyP | 1436 |
| | roThrArgAspTyr | |
| VH_100 | GlyPheThrPheSerThrTyrAlaIleSerGlySerGlyAspIleThrAlaLysAspTrpAlaGlyTyrThrAsnGly | 1437 |
| | TrpTyrGlySer | |
| VH_101 | GlyPheThrPheSerThrTyrAlaIleSerGlySerGlyAspIleThrAlaLysAspTrpAlaGlyTyrValAsnGly | 1438 |
| | TrpTyrGlyAsn | |
| VH_102 | GlyPheThrPheSerThrTyrAlaIleSerGlySerGlyGlyArgThrAlaLysAspTrpGlyAlaTyrSerSerGly | 1439 |
| | TrpTyrGlyAsp | |
| VH_103 | GlyPheThrPheSerThrTyrAlaIleSerGlySerGlyGlySerThrAlaLysAspTrpAlaGlyTyrIleAsnGlyT | 1440 |
| | rpTyrGlyAsn | |
| VH_104 | GlyPheThrPheSerThrTyrAlaIleSerGlySerGlyGlySerThrAlaLysAspTrpThrAsnGlnTrpLeuAs | 1441 |
| | pAlaTyrPheAspTyr | |
| VH_105 | GlyPheThrPheSerThrTyrAlaIleSerGlySerGlyGlySerThrAlaLysGluThrIleLeuTyrAspIleLeuT | 1442 |
| | hrGlyTyrTyrAsnGluGlyAlaPheAspIle | |
| VH_106 | GlyPheThrPheSerThrTyrAlaIleSerTyrAspGlySerAsnLysAlaLysAspTrpGlyArgPheGlyGluLe | 1443 |
| | uLeuGluGlySerProTyr | |
| VH_107 | GlyPheThrPheSerThrTyrAlaThrTyrTyrArgSerLysTrpTyrAsnAlaArgGluPheGlnAspSerSerS | 1444 |
| | erTrpTyrGluGlyArgAlaPheAspIle | |
| VH_108 | GlyPheThrValSerSerAsnTyrIleAsnProAsnSerGlyGlyThrAlaArgAspTrpGlyArgGlyValGlyAs | 1445 |
| | pSerGlyPheValAspTyr | |
| VH_109 | GlyPheThrValSerSerAsnTyrIleAsnProLysSerGlyGlyAlaAlaArgAspPheValGlyAlaSerLeuAs | 1446 |
| | pTyr | |
| VH_110 | GlyPheThrValSerSerAsnTyrIleSerGlySerGlyAspArgThrAlaLysAspTrpAlaGlyTyrIleAsnGly | 1447 |
| | TrpTyrGlyAsn | |
| VH_111 | GlyPheThrValSerSerAsnTyrIleSerSerSerGlySerThrIleAlaArgGlyTyrLeuGlyAlaTrpAsnPro | 1448 |
| | AspPheTyrAspTyr | |
| VH_112 | GlyPheThrValSerSerAsnTyrIleSerTyrAspGlySerAsnLysAlaArgValGlySerGlyGlyTrpThrPro | 1449 |
| | AspTyr | |
| VH_113 | GlyPheThrValSerSerAsnTyrIleThrGlySerGlyGlyThrAlaLysAspTrpAlaGlyTyrIleAsnGlyTrpP | 1450 |
| | heGlySer | |
| VH_114 | GlyPheThrValSerSerAsnTyrIleTyrProGlyAspSerAspThrAlaArgLeuGlyAspGlySerAsnPheA | 1451 |
| | spTyr | |
| VH_115 | GlyPheThrValSerSerAsnTyrThrTyrTyrArgSerLysTrpTyrAsnAlaArgGluLysIleAlaValAlaGly | 1452 |
| | TyrTyrTyrGlyMetAspVal | |
| VH_116 | GlyPheThrValSerSerAsnTyrThrTyrTyrAsnArgLysTrpIleAsnAlaArgAspGlyGlyTrpSerGlySe | 1453 |
| | rAlaLeuAspVal | |
| VH_117 | GlyTyrArgPheThrSerTyrTrpIleTyrSerGlyGlySerThrAlaArgAspLeuHisSerAlaAlaGlyPheAsp | 1454 |
| | Tyr | |
| VH_118 | GlyTyrSerPheThrArgTyrTrpIleLysSerLysAsnAspGlyGlyThrThrThrThrAlaProSerLeuMetAs | 1455 |
| | pVal | |
| VH_119 | GlyTyrSerPheThrSerTyrTrpIleSerGlySerGlyAspArgThrAlaLysAspTrpAlaGlyTyrIleAsnGlyT | 1456 |
| | rpTyrGlyAsn | |
| VH_120 | GlyTyrSerPheThrSerTyrTrpIleSerGlySerGlyAspArgThrAlaLysAspTrpAlaGlyTyrIleAsnGlyT | 1457 |
| | rpTyrGlyAsn | |
| VH_121 | GlyTyrSerPheThrSerTyrTrpIleSerTyrAspGlySerAsnLysAlaLysGlySerSerProTyrTyrTyrTyrG | 1458 |
| | lyMetAspVal | |
| VH_122 | GlyTyrSerPheThrSerTyrTrpIleTyrHisSerGlySerThrAlaArgAspGlyGlySerGlyTrpTyrAspTyr | 1459 |
| VH_123 | GlyTyrSerPheThrSerTyrTrpIleTyrSerGlyGlySerThrAlaArgAspThrAlaSerGlyGlyMetAspVal | 1460 |
| VH_124 | GlyTyrSerPheThrSerTyrTrpThrTyrTyrArgSerLysTrpTyrAsnAlaArgGlyValThrValProTyrTyr | 1461 |
| | TyrTyrGlyMetAspVal | |
| VH_125 | GlyTyrSerPheThrSerTyrTrpThrTyrTyrArgSerLysTrpTyrAsnAlaArgSerSerGlySerTyrGlyTyr | 1462 |
| | PheGlnHis | |
| VH_126 | GlyTyrThrPheThrArgAsnAlaThrTyrTyrArgSerLysTrpTyrAsnAlaArgGluGlyThrAspIleTyrTy | 1463 |
| | rTyrTyrGlyMetAspVal | |
| VH_127 | GlyTyrThrPheThrGlyTyrTyrIleAspTyrSerGlySerThrAlaArgAspGlyTrpIleArgLysGluAlaPhe | 1464 |
| | AspPro | |
| VH_128 | GlyTyrThrPheThrGlyTyrTyrIleLysSerLysAsnAspGlyGlyThrThrThrThrAlaProSerLeuMetAs | 1465 |
| | pVal | |
| VH_129 | GlyTyrThrPheThrGlyTyrTyrIleSerAlaTyrAsnGlyAsnThrAlaArgAspProGlyGlyTyrTyrTyrTyr | 1466 |
| | TyrGlyMetAspVal | |
| VH_130 | GlyTyrThrPheThrGlyTyrTyrIleSerTyrAspGlySerAsnLysAlaArgValGlySerGlyGlyTrpThrPro | 1467 |
| | AspTyr | |
| VH_131 | GlyTyrThrPheThrGlyTyrTyrIleSerTyrAspGlySerAsnLysAlaLysLeuGlyGlySerTyrSerIleTyrT | 1468 |
| | yrGlyMetAspVal | |
| VH_132 | GlyTyrThrPheThrGlyTyrTyrIleTyrProGlyAspSerGluThrAlaArgAspGlyGlyAsnTyrGlnPheAs | 1469 |
| | pTyr | |
| VH_133 | GlyTyrThrPheThrSerTyrAlaIleIleProIlePheGlyThrAlaAlaArgThrGlyArgSerGlySerTyrTyrSe | 1470 |
| | rAspAlaPheAspIle | |
| VH_134 | GlyTyrThrPheThrSerTyrGlyIleAsnProSerGlyGlySerThrAlaArgGluAspHisAspTyrSerAsnGl | 1471 |
| | nGlyGlyPheAspTyr | |
| VH_135 | GlyTyrThrPheThrSerTyrGlyIleIleProIlePheGlyThrAlaAlaAlaArgAlaProGlyGlySerSerTyrTy | 1472 |
| | rTyrTyrGlyMetAspVal | |
| VH_136 | GlyTyrThrPheThrSerTyrGlyIleSerAlaTyrAsnGlyAsnThrAlaArgAspProGlyTyrAspPheTrpSe | 1473 |
| | rGlyTyrSerAspVal | |
| VH_137 | GlyTyrThrPheThrSerTyrGlyIleSerGlySerGlyGlyArgThrAlaLysAspTrpAlaGlyTyrIleAsnGlyT | 1474 |
| | rpTyrGlyAsn | |
| VH_138 | GlyTyrThrPheThrSerTyrGlyIleSerTrpAsnSerGlySerIleAlaLysAspMetTrpGlySerLeuSerIleV | 1475 |
| | alGlyAlaThrArgAlaPheAspTyr | |
| VH_139 | GlyTyrThrPheThrSerTyrGlyIleThrGlySerGlyGlyThrAlaLysAspTrpAlaGlyTyrIleAsnGlyTrpP | 1476 |
| | heGlySer | |
| VH_140 | GlyTyrThrPheThrSerTyrGlyIleTyrHisSerGlySerThrAlaArgGlyProLeuLeuIleAlaAlaAlaGlyT | 1477 |
| | hrAspTyrTyrTyrGlyMetAspVal | |
| VH_141 | GlyTyrThrPheThrSerTyrTyrIleSerGlySerGlyGlySerThrAlaSerSerTyrGlyGlyAsnProLeuAsp | 1478 |
| | AlaPheAspIle | |
| VH_142 | GlyAspSerValSerSerAsnSerAlaAlaThrTyrTyrArgSerLysTrpTyrAsnAlaArgGluLysIleAlaVal | 1479 |
| | AlaGlyTyrTyrTyrGlyMetAspVal | |
| VH_143 | GlyAspSerValSerSerAsnSerAlaAlaThrTyrTyrArgSerLysTrpTyrAsnAlaArgGluPheGlnAspS | 1480 |
| | erSerSerTrpTyrGluGlyArgAlaPheAspIle | |
| VH_144 | GlyAspSerValSerSerAsnSerAlaAlaThrTyrTyrArgSerLysTrpTyrAsnAlaArgGlyGlyValGlyAla | 1481 |
| | ThrTrpTyrTyrGlyMetAspVal | |
| VH_145 | GlyPheThrPheAspAspTyrAlaIleSerTrpAsnSerGlySerIleAlaLysAspIleAlaAlaGlyGlyLeuAsp | 1482 |
| | Ser | |
| VH_146 | GlyPheThrPheSerAsnAlaTrpIleLysSerLysAsnAspGlyGlyThrThrThrThrAlaProSerLeuMetA | 1483 |
| | spVal | |
| VH_147 | GlyPheThrPheSerAsnAlaTrpIleLysSerLysAsnAspGlyGlyThrThrThrThrAlaProSerLeuMetA | 1484 |
| | spVal | |
| VH_148 | GlyPheThrPheSerSerTyrAlaIleSerTyrAspGlySerAsnLysAlaArgAspArgGlyValGluGlyAlaTyr | 1485 |
| | GlyMetAspVal | |
| VH_149 | GlyPheThrPheSerSerTyrGlyIleSerGlySerGlyGlySerThrAlaLysAlaThrGlyTyrSerSerGlyTrpT | 1486 |
| | yrGlyAlaTyrPheAspTyr | |
| VH_150 | GlyPheThrPheSerSerTyrGlyIleSerTyrAspGlySerAsnLysAlaLysGlySerSerProTyrTyrTyrTyr | 1487 |
| | GlyMetAspVal | |
| VH_151 | GlyPheThrPheSerSerTyrGlyIleTrpTyrAspGlyAsnAsnLysAlaArgAspAsnSerGlySerTyrAsnT | 1488 |
| | rpPheAsnPro | |
| VH_152 | GlyPheThrPheSerSerTyrGlyIleTrpTyrAspGlySerAsnLysAlaArgGluValValGlySerTyrTyrLeu | 1489 |
| | AspTyr | |
| VH_153 | GlyPheThrPheSerSerTyrProIleSerTyrAspGlyGlyAsnLysAlaArgValGlySerGlyGlyTrpThrPro | 1490 |
| | AspTyr | |
| VH_154 | GlyPheThrPheSerSerTyrProIleSerTyrAspGlySerAsnLysAlaArgValGlySerGlyGlyTrpThrPro | 1491 |
| | AspTyr | |
| VH_155 | GlyPheThrPheSerSerTyrProIleSerTyrAspGlySerAsnLysAlaArgValGlySerGlyGlyTrpThrPro | 1492 |
| | AspTyr | |
| VH_156 | GlyPheThrPheSerSerTyrProIleSerTyrAspGlySerAsnLysAlaArgValGlySerGlyGlyTrpThrPro | 1493 |
| | AspTyr | |
| VH_157 | GlyPheThrPheSerSerTyrProIleSerTyrAspGlySerAsnLysAlaArgValGlySerGlyGlyTrpThrPro | 1494 |
| | AspTyr | |
| VH_158 | GlyPheThrPheSerSerTyrProIleSerTyrAspGlySerAsnLysThrArgValGlySerGlyGlyTrpThrPr | 1495 |
| | oAspTyr | |
| VH_159 | GlyPheThrPheSerSerTyrSerIleTrpTyrAspGlySerAsnLysAlaArgLeuGlySerGlyTrpSerLeuAs | 1496 |
| | pTyr | |
| VH_160 | GlyPheThrPheSerSerTyrTrpIleLysGlnAspGlySerGluLysAlaArgAspLeuHisCysGlySerSerCy | 1497 |
| | sGlyProGluAla | |
| VH_161 | GlyPheThrValSerSerAsnTyrIleTyrSerGlyGlySerThrAlaArgAspLeuHisSerAlaAlaGlyPheAsp | 1498 |
| | Tyr | |
| VH_162 | GlyPheThrValSerSerAsnTyrIleTyrSerGlyGlySerThrAlaArgAspLeuSerTyrSerAspAlaPheAs | 1499 |
| | pIle | |
| VH_163 | GlyPheThrValSerSerAsnTyrIleTyrSerGlyGlySerThrAlaArgAspPheGluGlySerGlyAlaLeuAs | 1500 |
| | pVal | |
| VH_164 | GlyPheThrValSerSerAsnTyrIleTyrSerGlyGlySerThrAlaArgAspThrAlaSerGlyGlyMetAspVa | 1501 |
| | l | |
| VH_165 | GlyPheThrValSerSerAsnTyrIleTyrSerGlyGlySerThrAlaArgAspThrAlaSerGlyGlyMetAspVa | 1502 |
| | l | |
| VH_166 | GlyTyrSerPheThrSerTyrTrpIleTyrProGlyAspSerAspThrAlaSerGlyAlaSerProTyrTyrPheAs | 1503 |
| | pTyr | |
| VH_167 | GlyTyrThrPheThrGlyTyrTyrIleAsnProAsnSerGlyGlyThrAlaArgGlyGlyAspCysSerSerThrSe | 1504 |
| | rCysTyrAspProAspTyr | |
| VH_168 | GlyTyrThrPheThrSerTyrGlyIleSerAlaTyrAsnGlyAsnThrAlaArgAspProValTyrSerSerSerTrp | 1505 |
| | GlyGlyTyrAlaPheAspIle | |
| VH_169 | GlyTyrThrPheThrSerTyrGlyIleSerAlaTyrAsnGlyAsnThrAlaArgGlyLeuGlyAspSerSerSerSer | 1506 |
| | Tyr | |
| VH_170 | GlyTyrThrPheThrSerTyrTyrIleAsnProSerGlyGlySerThrAlaArgGluAspHisAspTyrSerAsnGl | 1507 |
| | nGlyGlyPheAspTyr | |

### LGALS3BP Detection Assay and Kit

In one embodiment of the present invention is a kit. This Human uG3BP ELISA kit is used for the non-radioactive quantification of human G3BP (galectin-3-binding protein, LGALS3BP, lectin galactoside-binding soluble 3 binding protein, M2BP; Mac-2 BP; 90K/Mac-2-binding protein) in urine samples. One kit is sufficient to measure 38 unknown samples in duplicate.

### PRINCIPLES OF ASSAY

This assay is a Sandwich ELISA based, sequentially, on: 1) capture of human G3BP molecules from samples to the wells of a microtiter plate coated with an anti-human G3BP monoclonal antibody, 2) washing of unbound materials from samples, 3) binding of a second biotinylated anti-human G3BP monoclonal antibody to the captured molecules, 4) washing of unbound materials from samples, 5) binding of streptavidin-horseradish peroxidase (HRP) conjugate to the immobilized biotinylated antibodies, 6) washing of excess free enzyme conjugates, and 7) quantification of immobilized antibody-enzyme conjugates by monitoring horseradish peroxidase activities in the presence of the substrate 3,3′,5,5′-tetramethylbenzidine (TMB). The enzyme activity is measured spectrophotometrically by the increased absorbance at 450 nm-590 nm after acidification of formed products. Since the increase in absorbance is directly proportional to the amount of captured human G3BP in the unknown sample, the latter can be derived by interpolation from a reference curve generated in the same assay with reference standards of known concentrations of human G3BP. It will be appreciated to one of skill in the art that the anti-human G3BP monoclonal antibodies described by SEQ ID Nos: 2-31 may be incorporated into the instant assay.

### REAGENTS SUPPLIED

Each kit is sufficient to run one 96-well plate and contains the following reagents: (store all reagents at 2-8° C.).

| Reagents Supplied | Volume | Quantity |
|---|---|---|
| Microtiter Plate with 2 plate sealers | — | 1 plate |
| | | 2 sealers |
| Human G3BP Standard | lyophilized | 2 vials |
| Human G3BP Quality Controls 1 and 2 | lyophilized | 2 vials |
| Assay Buffer | 40 mL | 1 bottle |
| 10X Wash Buffer | 50 mL | 2 bottles |
| Human G3BP Detection Antibody | 12 mL | 1 bottle |
| Enzyme Solution | 12 mL | 1 bottle |
| Substrate Solution | 12 mL | 1 bottle |
| Stop Solution | 12 mL | 1 bottle |

### STORAGE AND STABILITY

All components are shipped and stored at 2-8° C. Reconstituted standards and controls can be frozen for future use but repeated freeze/thaw cycles should be avoided. Refer to expiration dates on all reagents prior to use. Do not mix reagents from different kits unless they have the same lot numbers.

### MATERIALS REQUIRED BUT NOT PROVIDED

1. Multi-channel Pipettes and pipette tips: 5-50 μL and 50-300 μL
2. Pipettes and pipette tips: 10 μL-20 μL or 20 μL-100 μL
3. Reagent Reservoirs
4. Polypropylene Microfuge Tubes
5. Vortex Mixer
6. De-ionized water
7. Microtiter Plate Reader capable of reading absorbency at 450 nm and 590 nm
8. Orbital Microtiter Plate Shaker
9. Absorbent Paper or Cloth
10.

[symbol]

### SAMPLE COLLECTION AND STORAGE

### Preparation of Urine Samples:

Centrifuge the sample at 4° C. to remove debris and assay immediately or aliquot and store samples at ≤−20° C.
Avoid repeated freeze/thaw cycles.
Urine samples may require a 1:10 dilution with assay buffer prior to assay.

### Note:

A maximum of 100 μL per well of diluted or neat urine sample can be used.
All samples must be stored in polypropylene tubes. DO NOT STORE SAMPLES IN GLASS.

### REAGENT PREPARATION

### Human G3BP Standard Preparation

1. Using a pipette, reconstitute the Human G3BP Standard with 500 μL distilled or de-ionized water. Invert and mix gently, let sit for 5 minutes then mix well.
2. Label seven polypropylene microfuge tubes as 1, 2, 3, 4, 5, 6 and 7. Add 200 μL of Assay Buffer to tubes 1, 2, 3, 4, 5 and 6. Prepare serial dilutions by adding 500 μL of the reconstituted standard to the Tube 7, mix well and transfer 100 μL of Tube 7 to Tube 6, mix well and transfer 100 μL of Tube 6 to Tube 5, mix well and transfer 100 μL of Tube 5 to Tube 4, mix well and transfer 100 μL of the Tube 4 to Tube 3, mix well and transfer 100 μL of Tube 3 to Tube 2, mix well and transfer 100 μL of Tube 2 to Tube 1, mix well. The 0 ng/mL standard (Background) will be Assay Buffer.
Note: Change tip for every dilution. Wet tip with standard before dispensing. Unused portions of reconstituted standard should be stored in small aliquots at ≤−20° C. Avoid multiple freeze/thaw cycles.

| | Volume of | Volume of | Standard |
|---|---|---|---|
| | Deionized | Standard | Stock |
| Tube # | Water to Add | to Add | Concentration |
| Reconstituted | 500 μL | 0 | 200 ng/mL |
| standard | | | |

| | Volume | Volume of | Standard |
|---|---|---|---|
| | of Assay | Standard to | Concentration |
| Tube # | Buffer to Add | Add | (ng/mL) |
| Tube 7 | 0 | 500 μL of reconstituted | 200 |
| | | standard | |
| Tube 6 | 200 μL | 100 μL of Tube 7 | 66.67 |
| Tube 5 | 200 μL | 100 μL of Tube 6 | 22.22 |
| Tube 4 | 200 μL | 100 μL of Tube 5 | 7.41 |
| Tube 3 | 200 μL | 100 μL of Tube 4 | 2.47 |
| Tube 2 | 200 μL | 100 μL of Tube 3 | 0.82 |
| Tube 1 | 200 μL | 100 μL of Tube 2 | 0.27 |

### REAGENT PREPARATION (CONTINUED)

### B. Human G3BP Quality Control 1 and 2 Preparation

Reconstitute each Human G3BP Quality Control 1 and Quality Control 2 with 500 μL distilled or de-ionized water and gently invert to ensure complete hydration (mix gently, let sit for 5 minutes then mix well). Unused portions of the reconstituted Quality Controls should be stored in small aliquots at ≤−20° C. Avoid further freeze/thaw cycles.

### C. Preparation of Wash Buffer

Bring the 10× Wash Buffer to room temperature and mix to bring all salts into solution. Dilute 50 mL of 10× Wash Buffer with 450 mL deionized water. Store unused portion at 2-8° C. for up to one month.

### HUMAN uG3BP ELISA ASSAY PROCEDURE

Warm All Reagents to Room Temperature before Setting Up the Assay.
1. Remove the required number of strips from the Microtiter Assay Plate. Unused strips should be resealed in the foil pouch and stored at 2-8° C. Assemble the strips in an empty plate holder. Add 300 μL diluted Wash Buffer to each well of the plate. Decant Wash Buffer and remove the residual volume by inverting the plate and tapping it smartly onto absorbent towels several times. Repeat wash procedure two additional times. Do not let wells dry before proceeding to the next step. If an automated machine is used for the assay, follow the manufacturer's instructions for all washing steps described in this protocol.
2. Add 50 uL Assay Buffer to all wells.
3. Add 50 μL Assay Buffer to each of the Blank wells.
4. Add 50 μL of Standards and Quality Controls to the appropriate wells (refer to Microtiter Plate Arrangement section for suggested sample order placement).
5. Add 50 μL of diluted urine sample to the appropriate wells.
6. Cover the plate with plate sealer and incubate at room temperature for 2 hours on an orbital microtiter plate shaker set to rotate at moderate speed, about 400 to 500 rpm.
7. Remove plate sealer and decant reagents from the plate. Tap as before to remove residual volume in well. Wash wells 3 times with diluted Wash Buffer, 300 μL per well per wash. Decant and tap after each wash to remove residual buffer. (add an agitating/soaking step is recommended between each wash if using the automatic plate washer.)
8. Add 100 μL Detection Antibody to each well. Re-cover plate with sealer and incubate at room temperature for 1 hour on an orbital microtiter plate shaker set to rotate at moderate speed, approximately 400-500 rpm.
9. Remove plate sealer and decant reagents from the plate. Tap as before to remove residual volume in well. Wash wells 3 times with diluted Wash Buffer, 300 μL per well per wash. Decant and tap after each wash to remove residual buffer.
10. Add 100 μL Enzyme Solution to each well. Cover plate with sealer and incubate with moderate shaking at room temperature for 30 minutes on the microtiter plate shaker.
11. Remove sealer, decant reagents from the plate and tap plate to remove the residual volume. Wash wells 4 times with diluted Wash Buffer, 300 μL per well per wash. Decant and tap after each wash to remove residual buffer.
12. Add 100 μL of Substrate Solution to each well, cover plate with sealer and shake on the plate shaker for approximately 5-20 minutes. Blue color should be formed in wells of the Human G3BP standards with intensity proportional to increasing concentrations of Human G3BP.
Note: The color may develop more quickly or more slowly than the recommended incubation time depending on the localized room temperature. Please visually monitor the color development to optimize the incubation time.
13. Remove sealer and add 100 μL Stop Solution and gently shake plate by hand to ensure complete mixing of solution in all wells. The blue color should turn to yellow after acidification. Wipe the bottom of the microtiter plate to remove any residue prior to reading on plate reader. Read absorbance at 450 nm (signal) and 590 nm (background) in a plate reader within 5 minutes and ensure that there are no air bubbles in any well. Record the difference of absorbance units. The absorbance of the highest Human G3BP standard should be approximately 2.5-3.5, or not to exceed the capability of the plate reader used.
Note: If urine samples are diluted 1:10, final results, ng/mL concentrations of G3BP in samples, should be multiplied by a dilution factor of 10.

### ASSAY CHARACTERISTICS

### A. Sensitivity

The Minimum Detectable Concentration (MinDC) of Human G3BP is 0.08 ng/mL. It is calculated by using MILLIPLEX® Analyst 5.1. It measures the true limits of detection for an assay by mathematically determining what the empirical MinDC would be if an infinite number of standard concentrations were run for the assay under the same conditions. This reported value is the mean plus 2 standard deviations of the MinDC of multiple assays (n=8).

### B. Specificity

The antibody pair used in this assay is specific to human G3BP.

### C. Precision

Intra-Assay Variation

| | Mean | Intra-Assay |
|---|---|---|
| | Levels (ng/mL) | % CV |
| 1 | 219 | 5.9 |
| 2 | 636 | 5.6 |

Inter-Assay Variation

| | Mean Levels | Inter-Assay |
|---|---|---|
| | (ng/mL) | % CV |
| 1 | 380 | 8.3 |
| 2 | 607 | 8.1 |

The assay variations of this uG3BP ELISA kit was studied on urine samples at two levels on the uG3BP standard curve. The mean intra-assay variation was calculated from results of eight determinations of the indicated samples. The mean inter-assay variations of each sample were calculated from results of 8 separate assays with duplicate samples in each assay. (The urine samples were diluted with assay buffer prior to assay.)

### D. Spike Recovery of G3BP in Assay Samples

The average recovery of human G3BP in eight urine samples is 103%. Three concentrations of human G3BP were added to individual urine samples (n=8) and the resulting G3BP content of each sample was assayed by Human uG3BP ELISA. The recovery =[(observed G3BP/(spiked G3BP concentration+basal G3BP]×100%. (The urine samples were diluted with assay buffer prior to assay.)

### E. Linearity of Sample Dilution

The average % of expected linearity in eight urine samples is 96%. Required amounts of Assay Buffer were added for resulting dilution factors of 1, 2, 4 and 8 assayed, respectively. % expected=(observed/expected)×100%. (The urine samples were diluted with assay buffer prior to assay.)

### EXPERIMENTAL EXAMPLES

The following examples are intended for illustration only and should not be construed to limit the scope of the claimed invention.

### Example 1: LGALS3BP Expression is Increased in PBMCs From LN Patients and Correlates with their Interferon Status

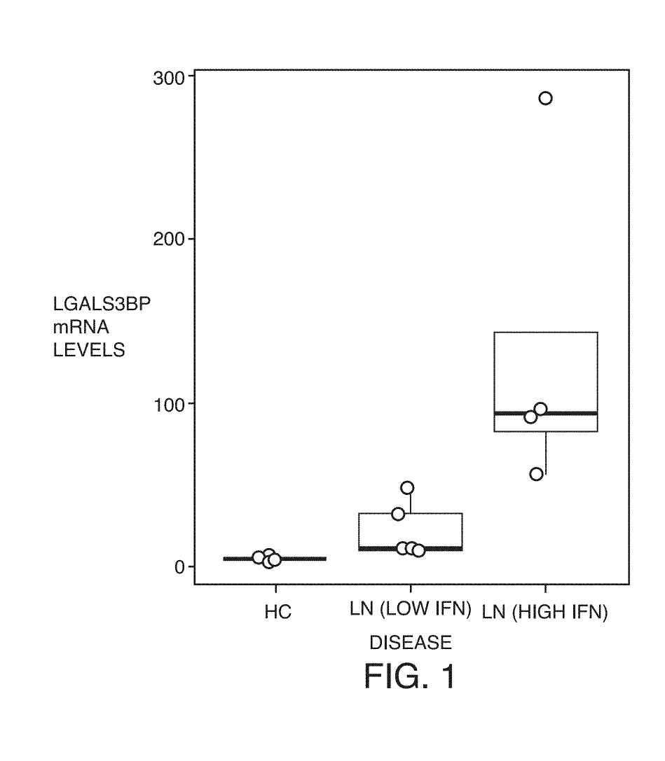

In order to find predictive markers of disease activity in LN patients, the mRNA profiles of PBMCs isolated from LN patients were assessed and compared these profiles to those of healthy controls (HC). PBMCs were isolated from whole blood of HC (n=4) and LN donors (n=9) by Ficoll gradient. Gene expression profiling was performed by RNA-seq. FPKM values are shown. LN patients were grouped into Low interferon (IFN) or High IFN based on the median average z-score of four IFN-inducible genes, IFI44L, RSAD2, MX1, and OAS2 (Hagberg N and Rönnblom L, Scand J Immunol 2015 September; 82(3):199-20). LGALS3BP mRNA levels were significantly higher in the LN (High IFN) group vs the LN (Low IFN) group (p=0.044) and the HC group (p=0.028). From the profiling described above it was found that LGALS3BP mRNA expression was one of the best genes whose levels could be used to distinguish between LN and HC PBMCs (FIG. 1). It was also observed there was significant variability in the levels of LGALS3BP among the LN patients. LN patients are often grouped based on their type I interferon levels as measured by the levels of interferon-inducible genes (Scand J Immunol. 2015 September; 82(3):199-20). A subsequent evaluation determined if the interferon levels between the LN samples could explain the large variability observed in LGALS3BP. In the lupus nephritis patients, a bimodal distribution in the type I interferon-inducible genes was found indicating that some patients had a high interferon signature while others had a low interferon signature. In order to further sort the lupus nephritis patients into these two groups, the expression levels of four known interferon-inducible genes, IFI44L, RSAD2, OAS2, and MX1 were combined by taking the average z-score of the four genes across all the samples. Samples with interferon signature scores equal to or below the median levels were assigned to the low interferon group. Those samples with interferon scores above the median were assigned to the high interferon group. After classifying the donors into these two groups, it was found that LGALS3BP levels were 5-fold higher in the low interferon group as compared to healthy controls, and 30-fold higher in the high interferon group compared to healthy controls (p=0.028; FIG. 1). Additionally, LGALS3BP levels were 6-fold higher in the high interferon group as compared to the low interferon group (p=0.044). These data demonstrate that LGALS3BP expression is increased in LN patients and that LGALS3BP expression is likely regulated by type I interferon.

### Example 2: LGALS3BP Expression can be Induced by IFNα and Other Inflammatory Stimuli

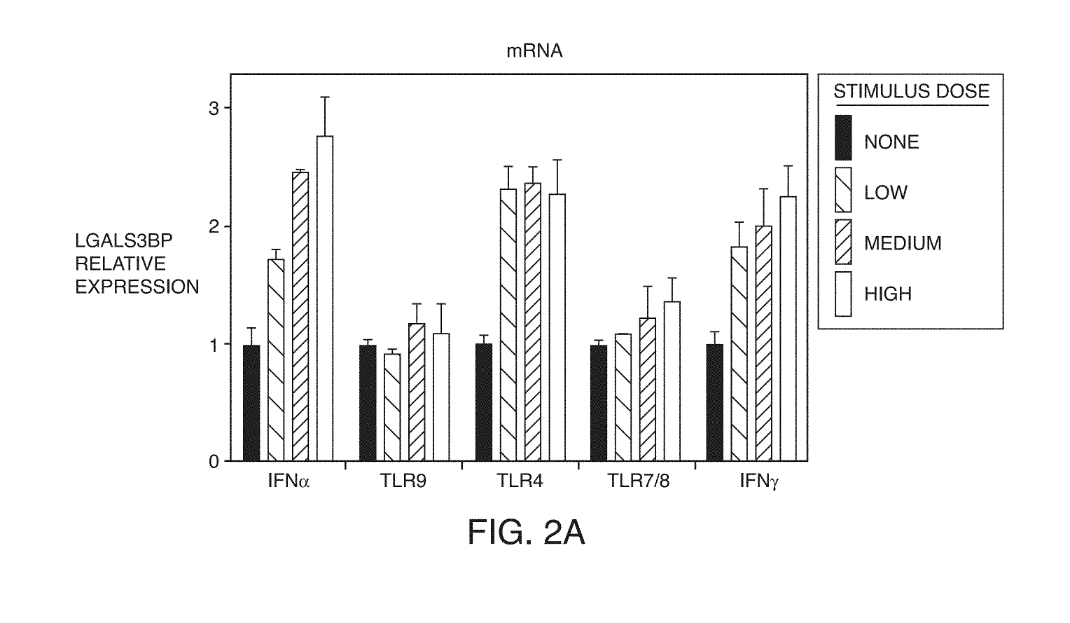

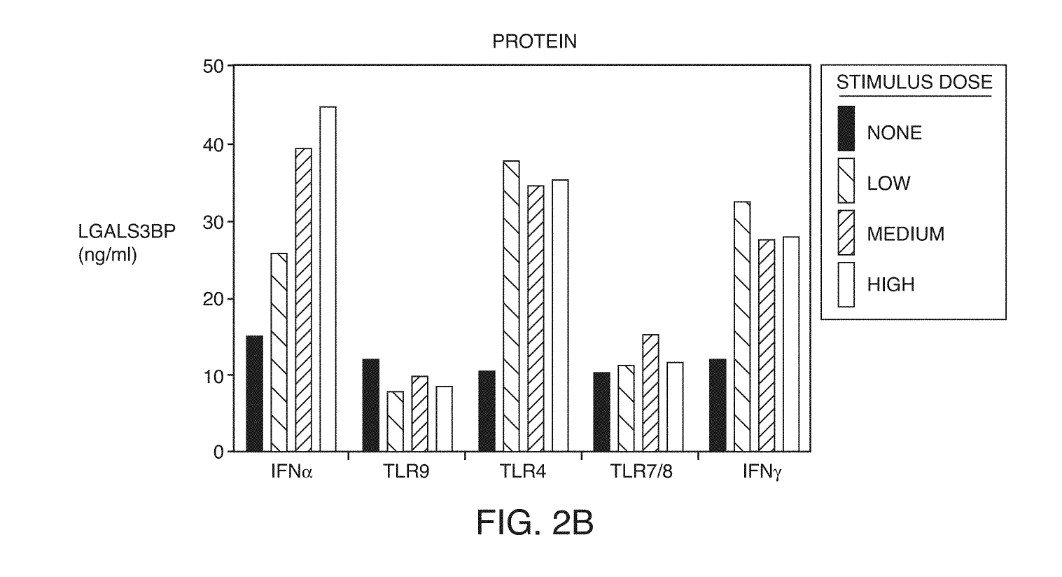

LGALS3BP has an IRF7 binding site consistent with regulation by type I interferons. In order to discover which pathways can induce LGALS3BP expression, primary human monocytes were differentiated into macrophages in vitro and were subsequently stimulated with IFNα, IFNγ, TLR4 agonist (LPS), TLR7/8 agonist (resiquimod) and TLR9 agonist (CpG). IFNα, IFNγ, and LPS induced LGALS3BP mRNA expression (FIG. 2a) and increased secretion of the protein (FIG. 2b). All stimuli induced secretion of IL-6. These data indicated that not only type I interferons can drive LGALS3BP expression but also IFNγ and other innate triggers. Based on location of histone acetylation sites, LGALS3BP expression is likely regulated by factors binding to four different regions in the LGALS3BP gene: at the promoter start site, in an upstream enhancer (region 5 K upstream), in an intronic site, or in the 3′ UTR. Motif scanning by three different methods identified immune-relevant transcriptional regulators. IRFs, AP-1, and STATs as well as other important factors such as NF-KB were found in and around the LGALS3BP gene locus. Prediction of transcription factor binding indicates that LGALS3BP expression is regulated by interferons through interferon regulatory factors (IRFs) as well as other immune stimuli that activate STATs, NF-kB, and AP-1.

### Example 3: LGALS3BP Protein is Increased in Urine From LN Patients but not in Plasma

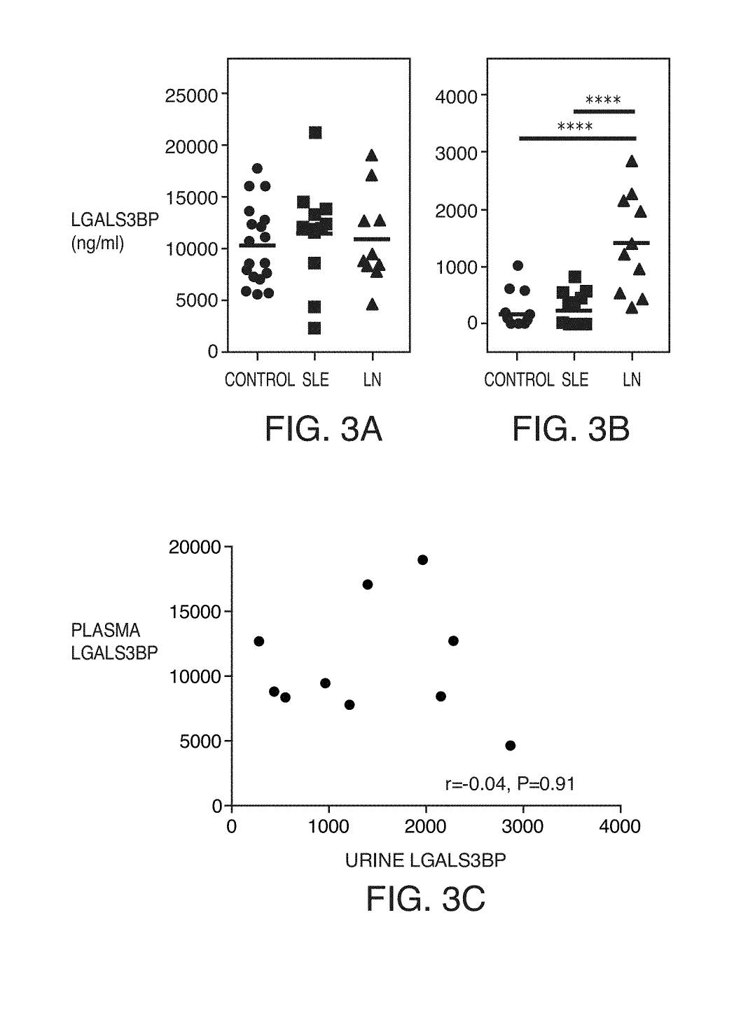

To determine if increased mRNA levels in PBMCs led to increased levels of LGALS3BP protein in patient blood, LGALS3BP was measured by ELISA in plasma from LN patients, SLE patients and healthy control (HC) donors. No significant difference in plasma LGALS3BP levels between these three groups were found despite the upregulated mRNA in PBMCs (FIG. 3). It has been demonstrated that PBMCs only contributed minor amounts of total plasma LGALS3BP. Nonetheless, significantly higher LGALS3BP levels were found in urine from LN patients compared to SLE patients and healthy controls.

### Example 4: LGALS3BP Expression is Elevated in LN Patient Kidneys

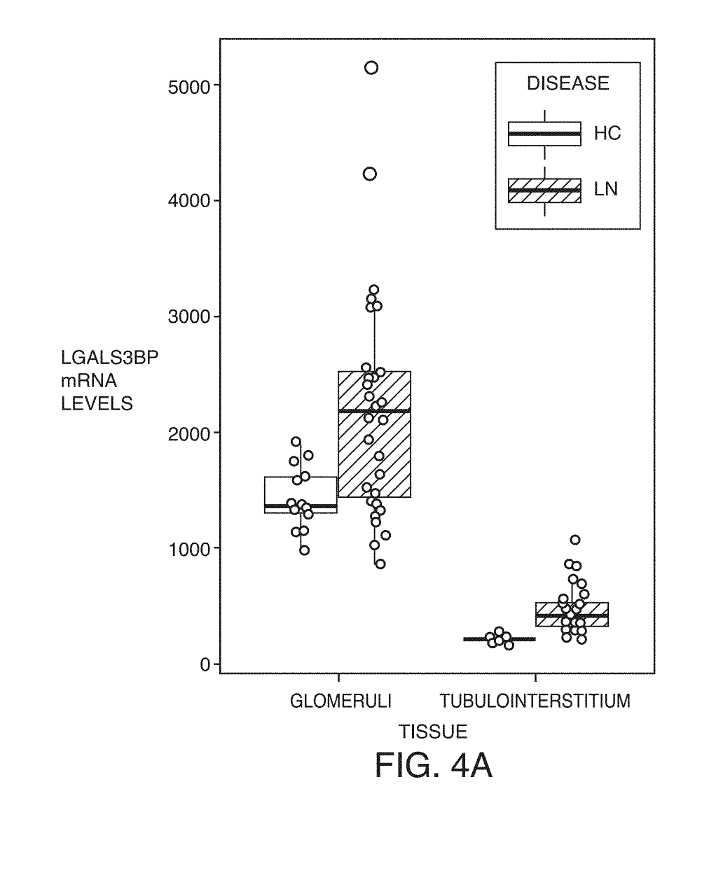

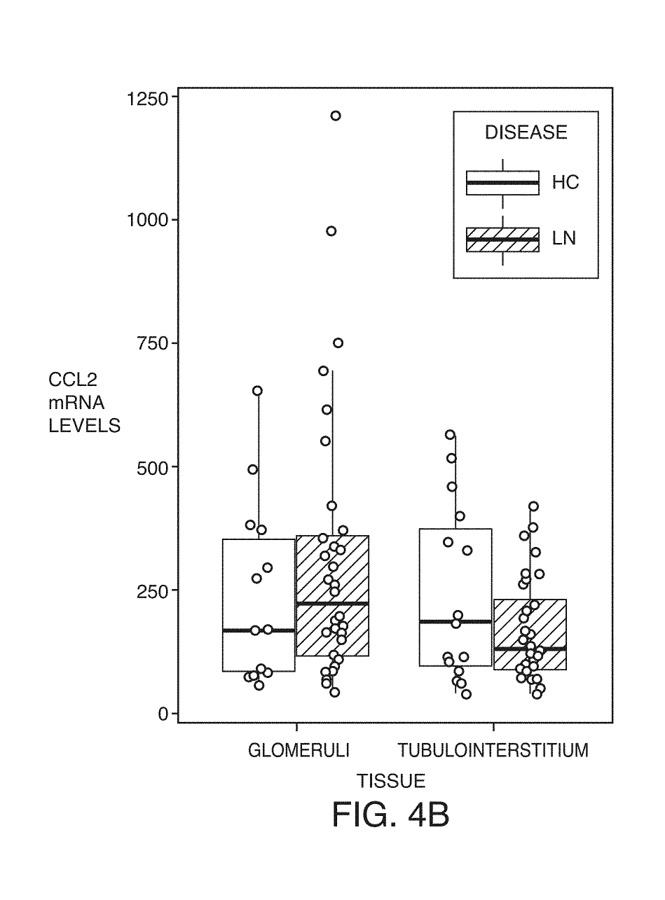

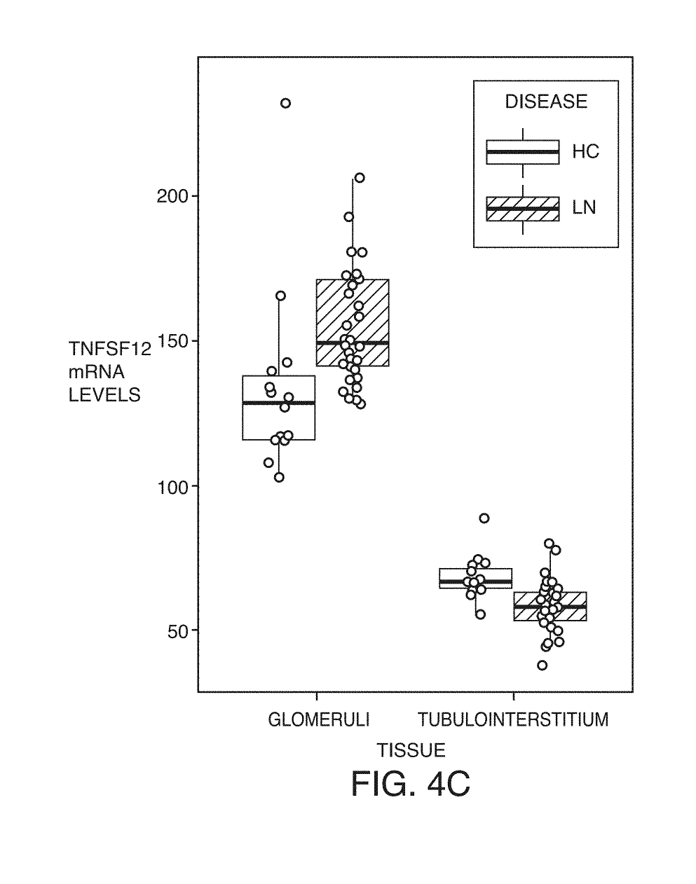

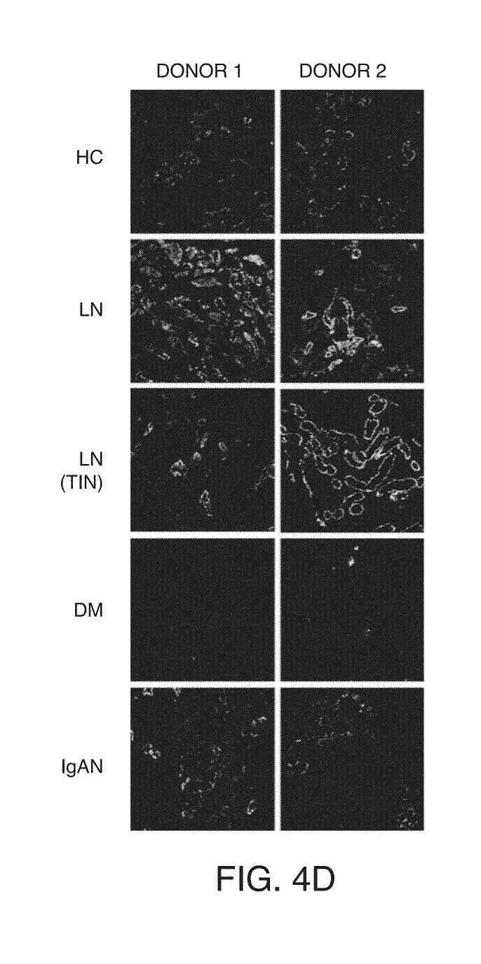

LN is characterized by kidney inflammation. Current tests to monitor disease activity measure kidney function in blood and urine but not causal inflammation. LGALS3BP is induced by inflammatory stimuli and its elevated presence in urine could reflect kidney inflammation. In order to determine if increased urinary LGALS3BP is relevant as a urinary protein measurement to monitor inflammation in lupus nephritis, LGALS3BP's mRNA expression profile was examined in kidney biopsies. GEO dataset (GSE32592) that contained a total of 46 kidney biopsy samples (n=14 HC and 32 LN) that were collected from the European Renal cDNA Bank was used. The glomeruli and tubulointerstitium were isolated by microdissection and expression profiling was performed using Affymetrix GeneChip arrays. After initial quality control assessments and normalization, the expression level of LGALS3BP was found to be significantly higher in both the glomeruli (1.5-fold, p=9.2e-12) and tubulointerstitium (2.2-fold, p=1.5e-4) of LN patients compared to healthy controls (FIG. 4a). The expression profile of two additional genes, CCL2 (MCP-1) and TNFSF12 (TWEAK), both of which have been proposed as potential urinary biomarkers (Schwartz et al. Ann N Y Acad Sci. 2007 August; 1109:265-74) was then evaluated. In that dataset, CCL2 (MCP-1) (FIG. 4b) expression levels were found to be equivalent between LN and HC samples in both the glomeruli (1.3-fold, p=0.392) and tubulointerstitium (0.7-fold, p=0.33). Expression levels of TNFSF12 (FIG. 4C) was significantly higher in the glomeruli of LN samples (1.2-fold, p=9.1e-5), but significantly lower in the tubulointerstitium of LN samples (0.85-fold, p=0.017). These data suggest that LGALS3BP may be a more suitable urinary predictive marker than CCL2 (MCP-1) and TNFSF12 to distinguish between HC and LN samples.

Global differential expression was also evaluated in order to elucidate all the genes that were significantly modulated in LN patients. Using the R package limma, a model was constructed to perform the differential expression calculations while controlling for tissue differences. This allowed for the utilization of data from both the glomeruli and tubulointerstitium together. Of the 12,030 total genes included in the analysis, only 166 genes had a p-value less than 0 01 and a fold change of at least 2. The genes significantly upregulated in LN numbered 137 while 29 genes were downregulated in LN. In this analysis, LGALS3BP had a p-value of 2.11e-8 and was in the top 3% of genes with the lowest p-values. These data confirm that LGALS3BP is one of the few genes significantly upregulated in both the glomeruli and tubulointerstitium of LN kidney biopsies and, thereby, is a good predictive marker.

Staining of LN kidney biopsies with anti-LGALS3BP antibodies showed increased levels and punctate patterns in certain areas, specifically around tubules in patients with and without tubolointerstitial nephritis (FIG. 4d). LGALS3BP signal in a healthy control sample was less intense, more diffuse and mostly due to background staining of the secondary antibody (FITC anti-rabbit). Samples from diabetes mellitus (DM) and IgA nephropathy (IgAN) patients showed some but weaker LGALS3BP staining than LN.

### Example 5: LGALS3BP Expression is Increased in a Mouse Model of LN Only when Kidney Damage is Detected

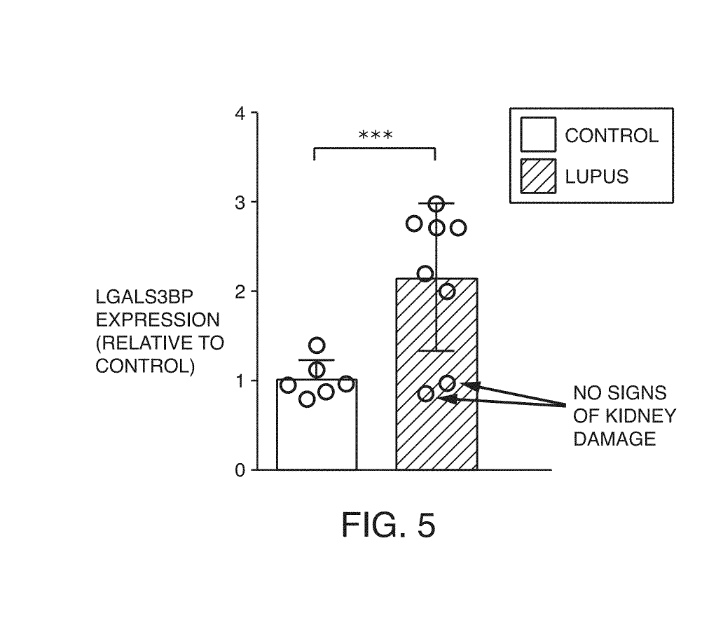

To further investigate if increased LGALS3BP kidney expression is induced by local inflammation its expression in BXSB-Yaa lupus mice was measured. These mice spontaneously develop systemic symptoms of SLE and LN-like inflammation and damage of the kidneys. The model is based on a duplication of the Yaa locus, which encompasses the TLR7 gene and results in increased TLR7 expression and type I interferon inflammation. Measuring the murine homolog of LGALS3BP elevated levels in mice were found with disease only when kidney damage and inflammation were detected by histology evaluating glomerular crescents, protein casts, interstitial inflammation, and vasculitis (FIG. 5). These results further indicate that LGALS3BP is expressed locally during an inflammatory process in the kidney.

### Example 6: LGALS3BP Protein is Elevated in LN Patient Urine

The following experiment was designed to determine if increased LGALS3BP expression in patient kidneys translated into a measurable difference in urine protein levels, which could distinguish between LN patients, SLE patients, and healthy control donors. LGALS3BP protein was measured by ELISA in urine from LN patients, SLE patients and healthy controls. After normalizing the data to urine creatinine levels, it was found that LGALS3BP (FIG. 3A) was significantly higher in LN patients than SLE (6.8-fold, p<0.001) and HC donors (17.7-fold, p<0.001). There was also a trend for higher levels of LGALS3BP found in SLE patients versus HC donors, but this trend was not statistically significant (2.6-fold, p=0.59).

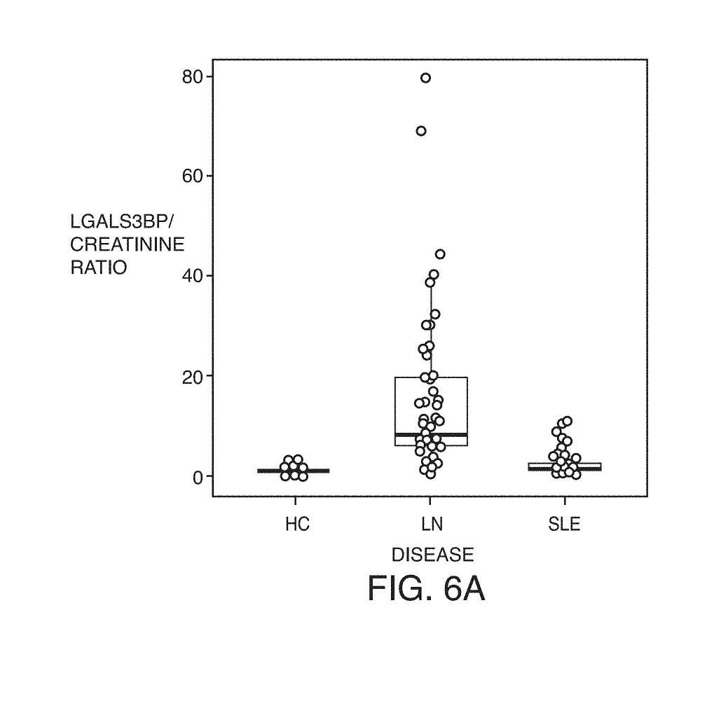

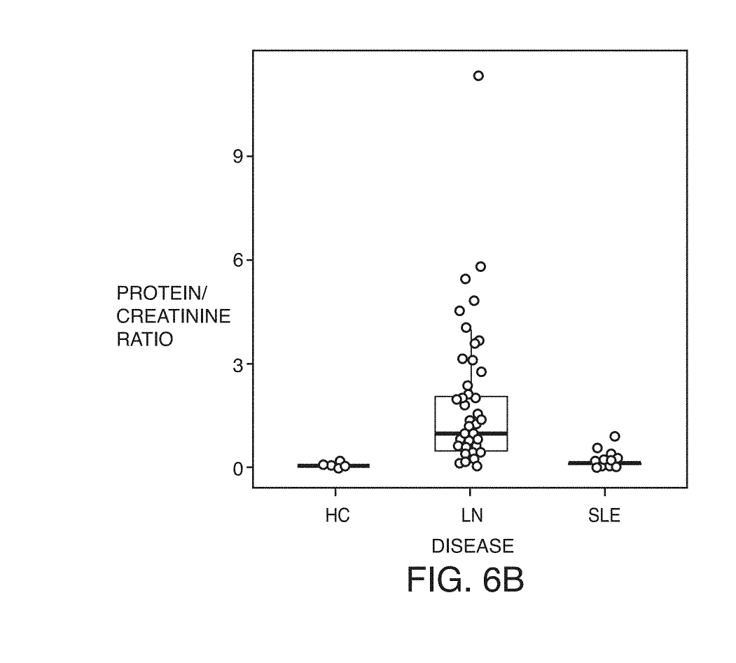

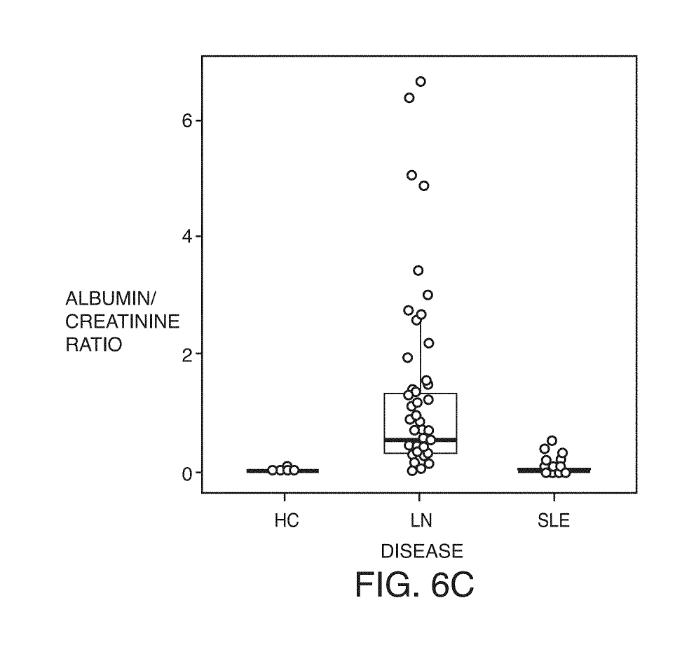

How the urine protein levels of LGALS3BP compared to other common urinalysis readouts, such as total protein levels or albumin levels was next considered. After normalizing all values to urine creatinine levels, total protein levels or albumin levels were found to perform as well to distinguish LN patients from SLE and HC donors. Both total protein levels (FIG. 6B) and albumin (FIG. 6C) levels were significantly higher in LN patients than SLE or HC donors (p<0.001 for both).

In order to apply these data to the construction of a diagnostic test, values associated with renal inflammation needed to be defined. In order to arrive at these values, the maximum value from the healthy control samples was set as the cutoff, meaning that any sample with a value higher than the maximum healthy control sample would likely have kidney inflammation. The rationale for this is based upon the assumption that healthy control donors should not have any inflammation and therefore, the values found in healthy controls should represent the normal range. For LGALS3BP/creatinine ratios, protein/creatinine ratios, and albumin/creatinine ratios, the cutoff values were 3.133, 0.166, and 0.457, respectively. Using these values, it was found that for LGALS3BP, 50 LN and 12 SLE samples were above the cutoff (FIG. 6A). For total protein, 53 LN and 18 SLE samples were above the cutoff (FIG. 6B). For albumin, 56 LN and 9 SLE samples were above the cutoff (FIG. 6C). These data suggest that LGALS3BP is more conservative in the identification of samples that are likely to have inflammation in the kidneys. For the SLE samples with LGALS3BP levels above the cutoff, these may be patients most at risk of developing lupus nephritis or SLE patients with undiagnosed LN.

### Example 7: LGALS3BP Urine Levels are not a Reflection of Kidney Function and Filtering Capacity

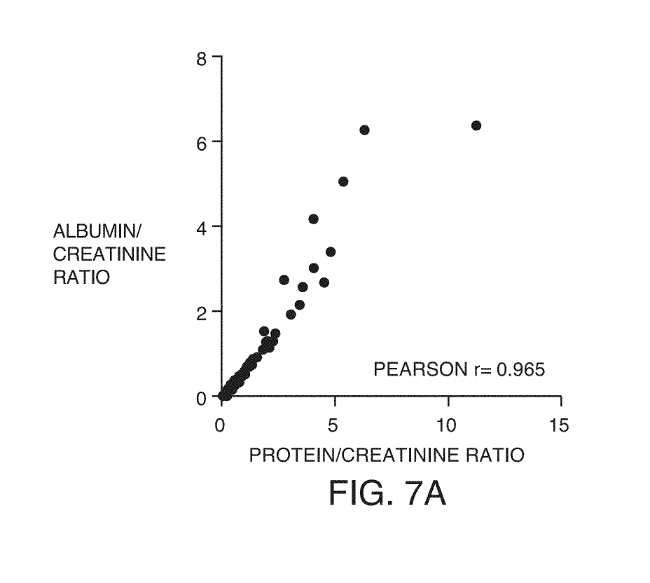

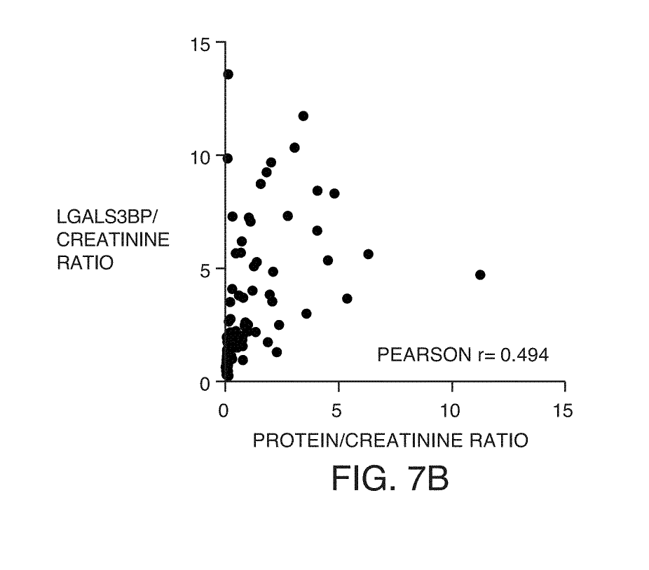

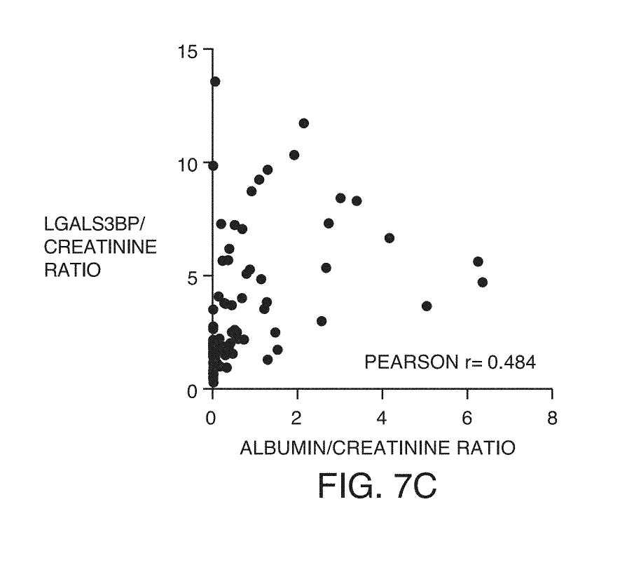

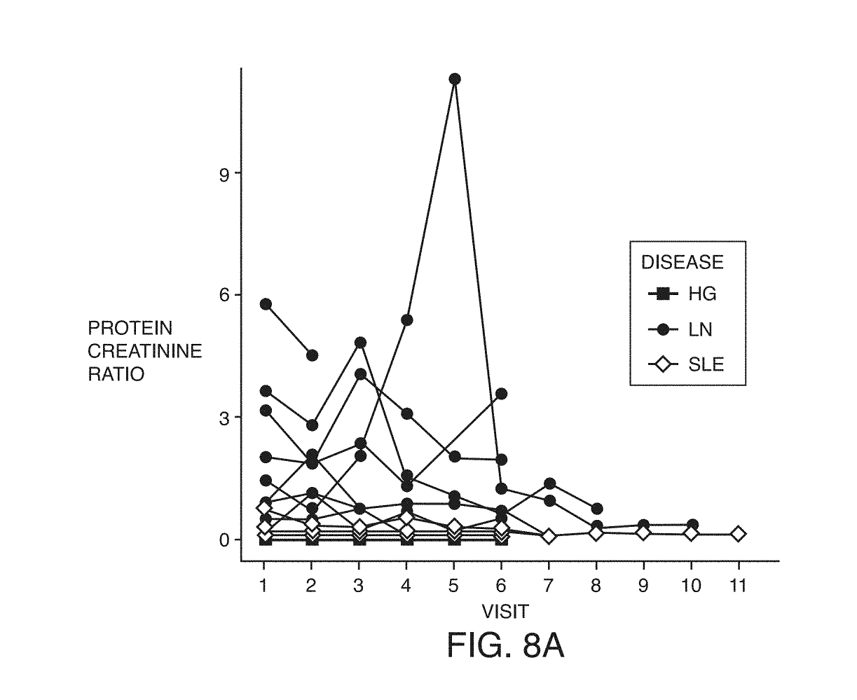

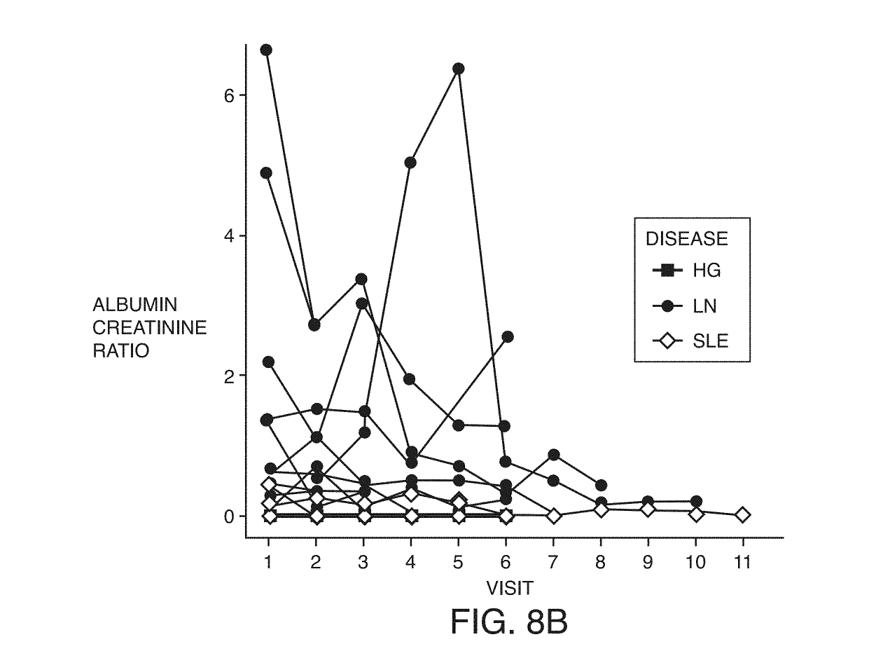

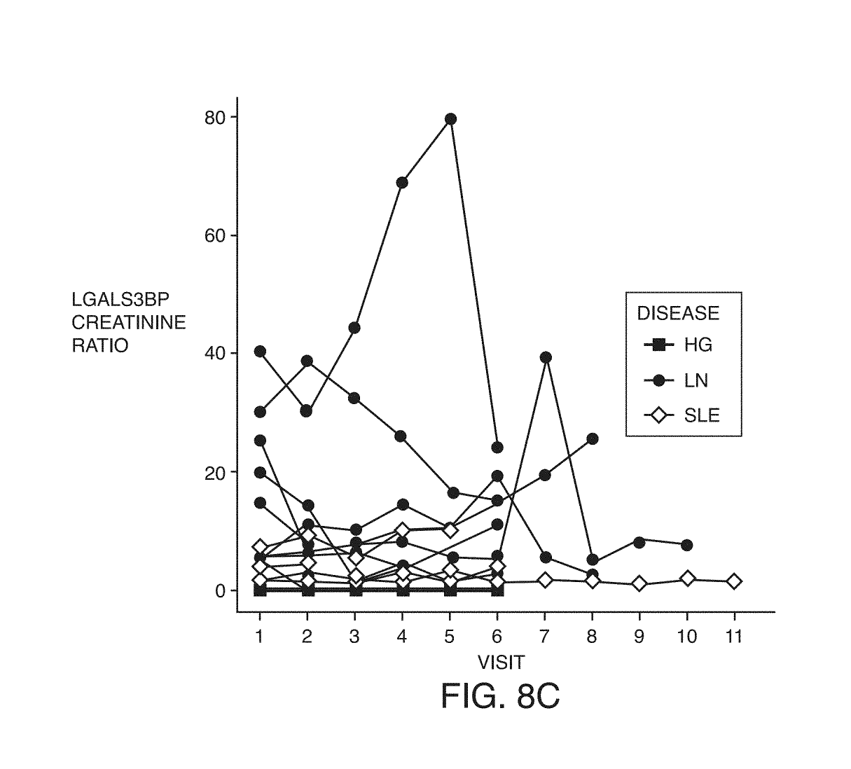

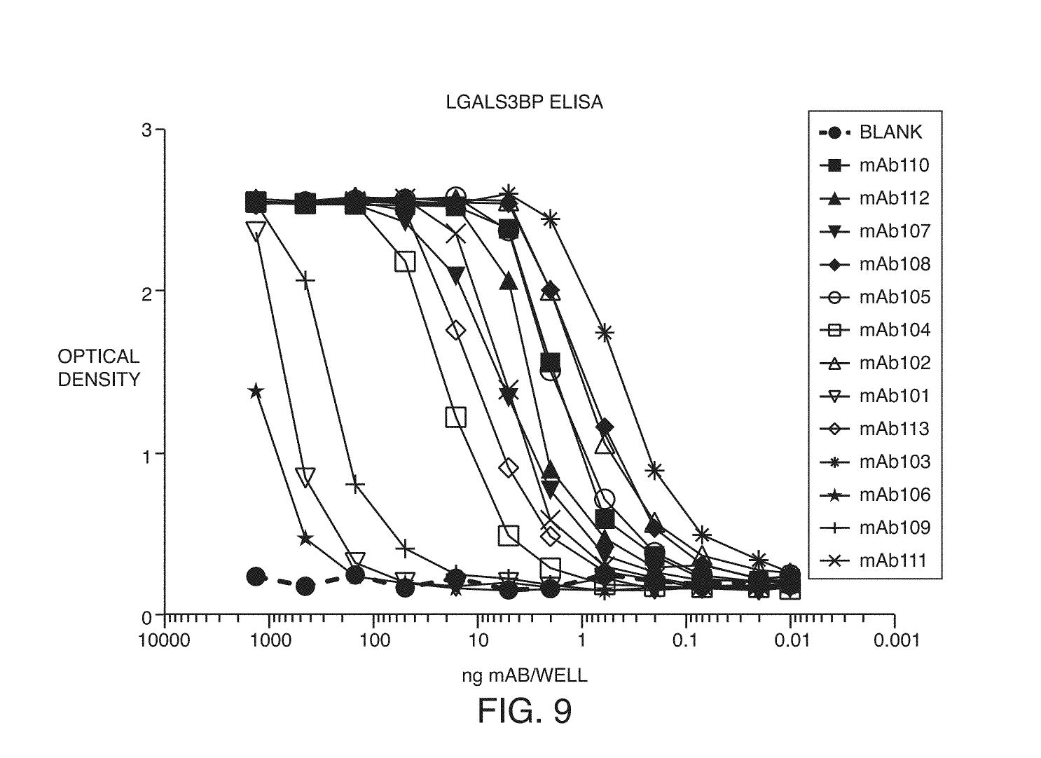

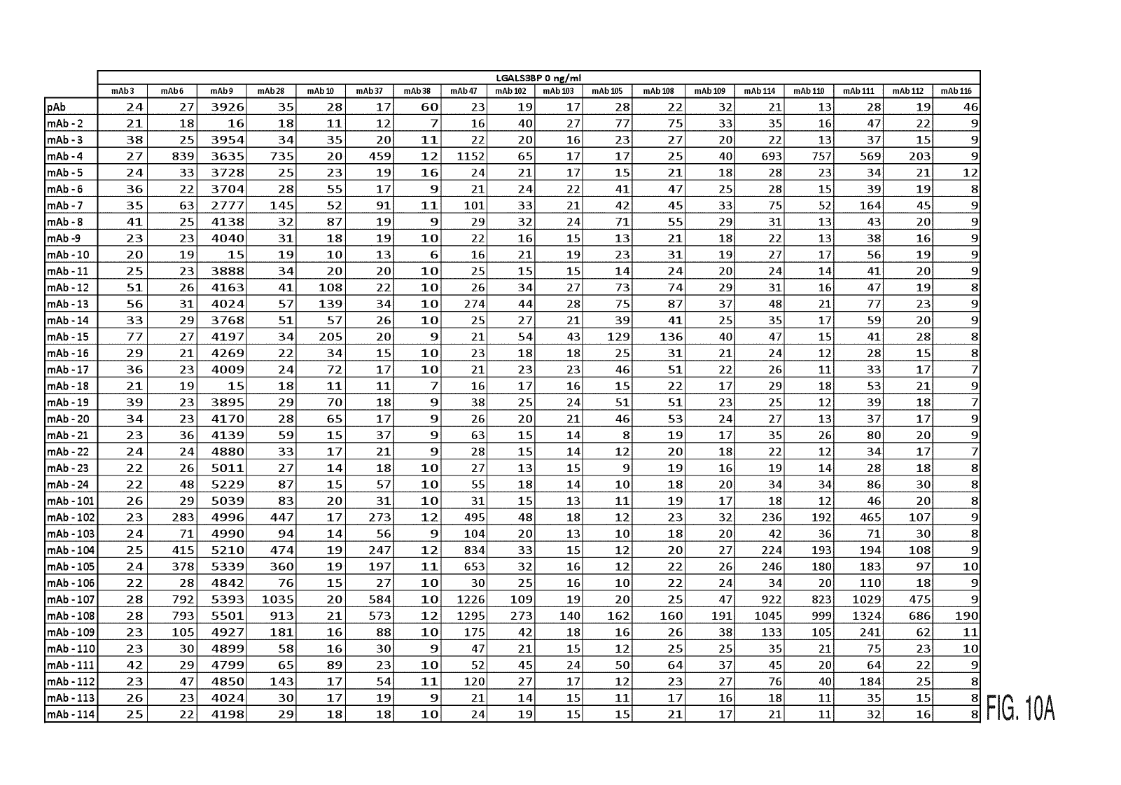

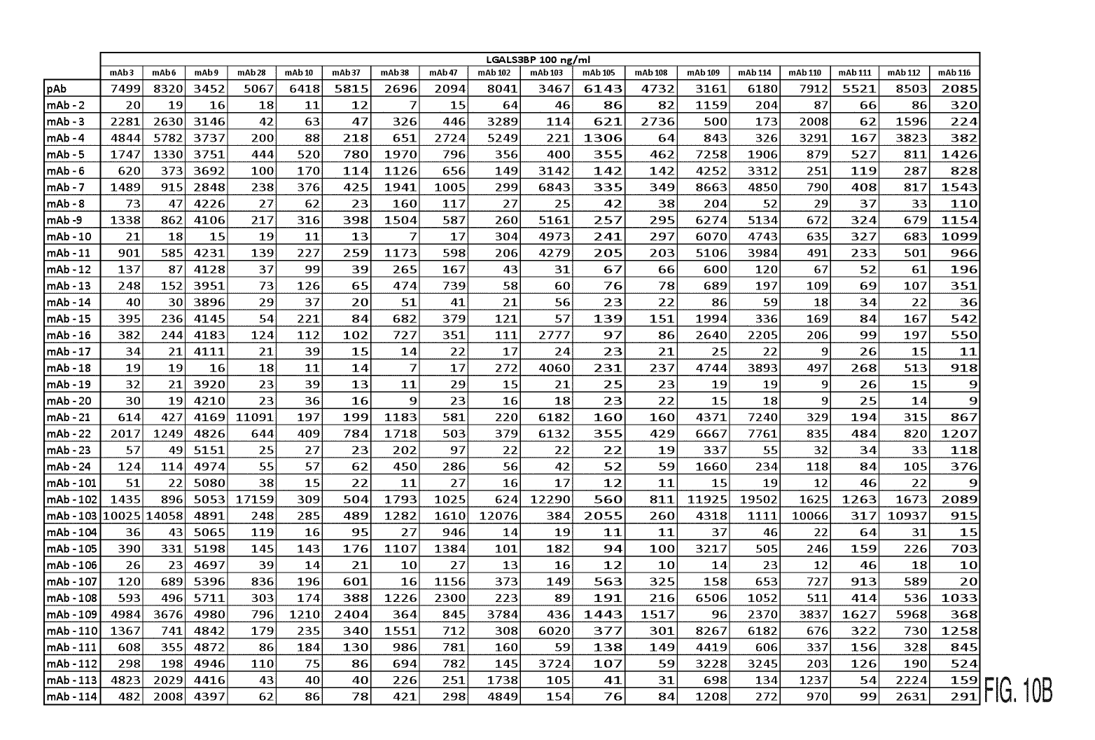

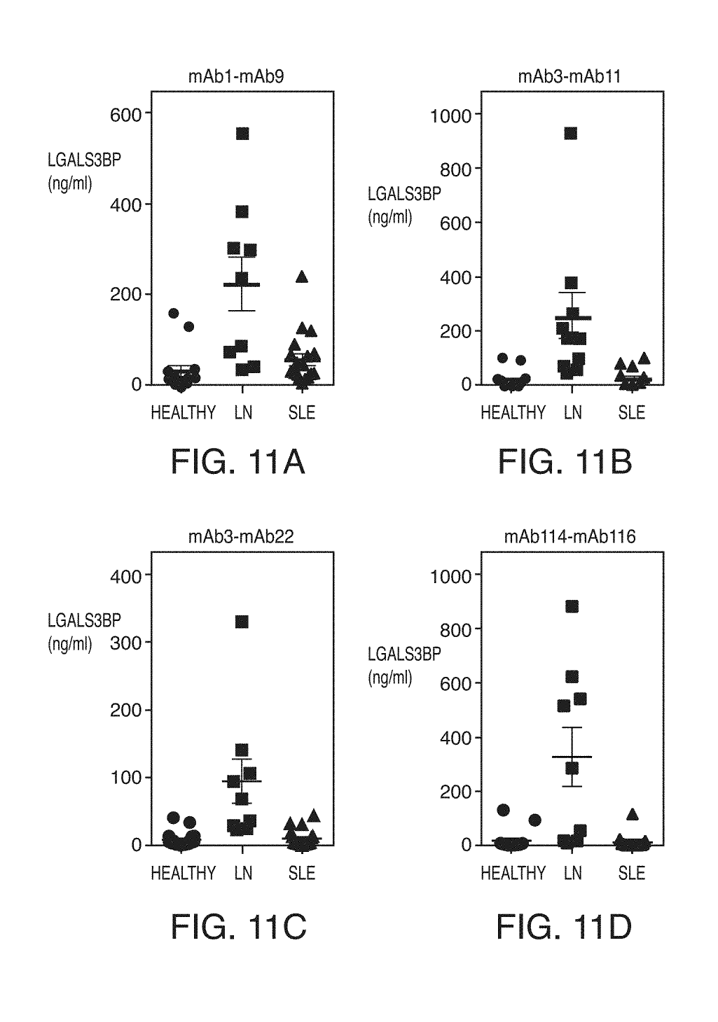

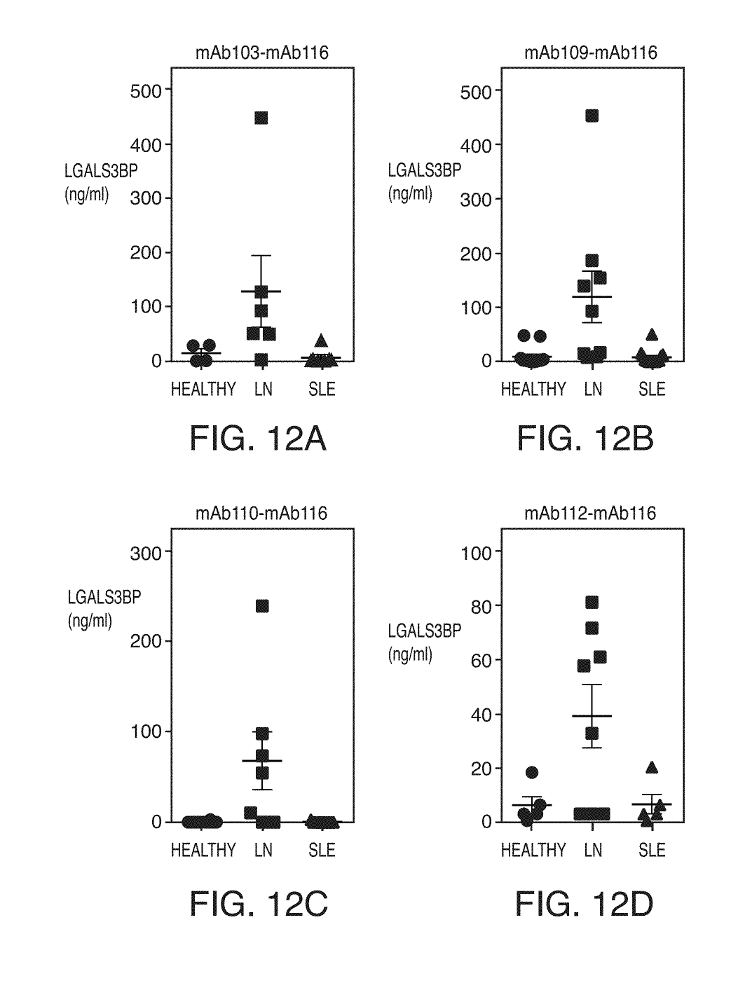

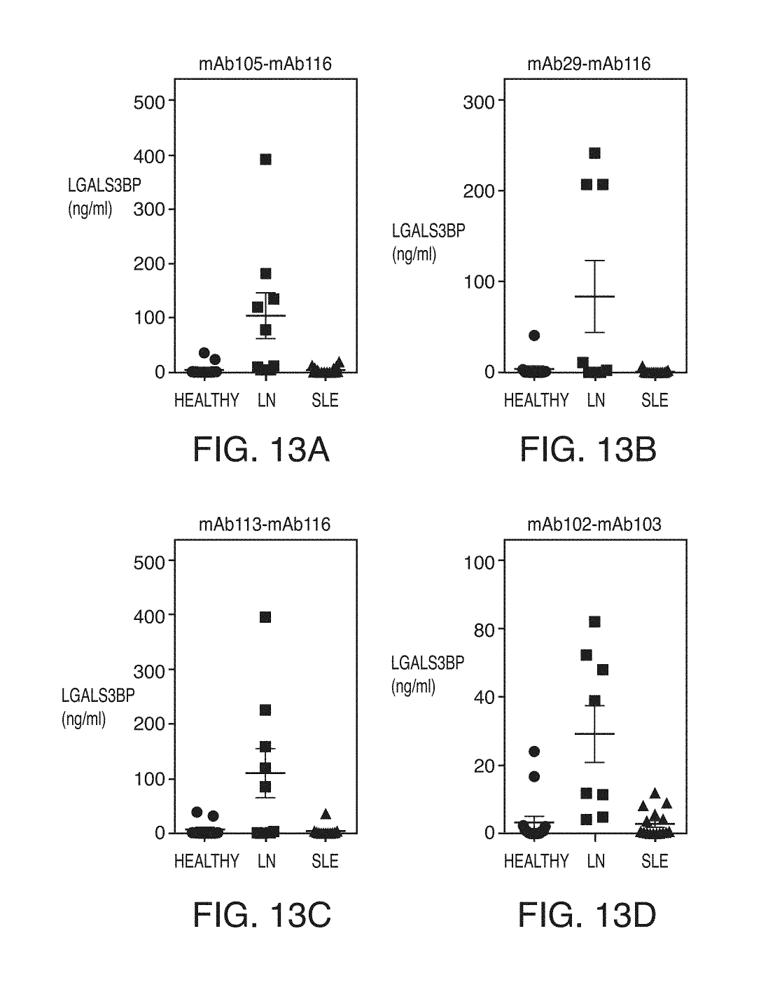

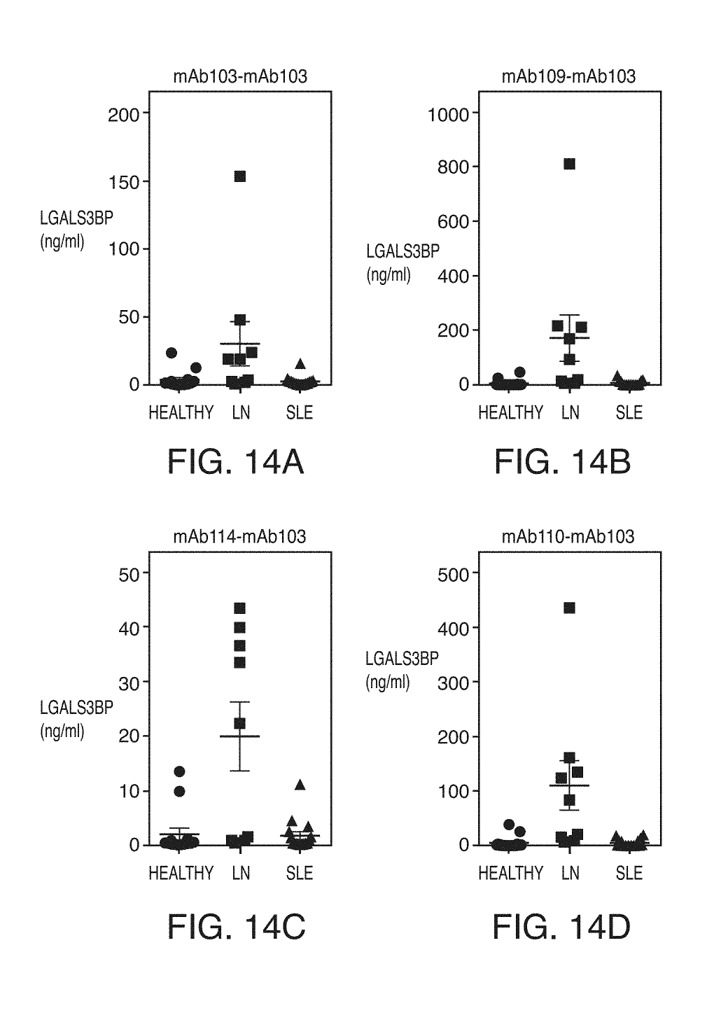

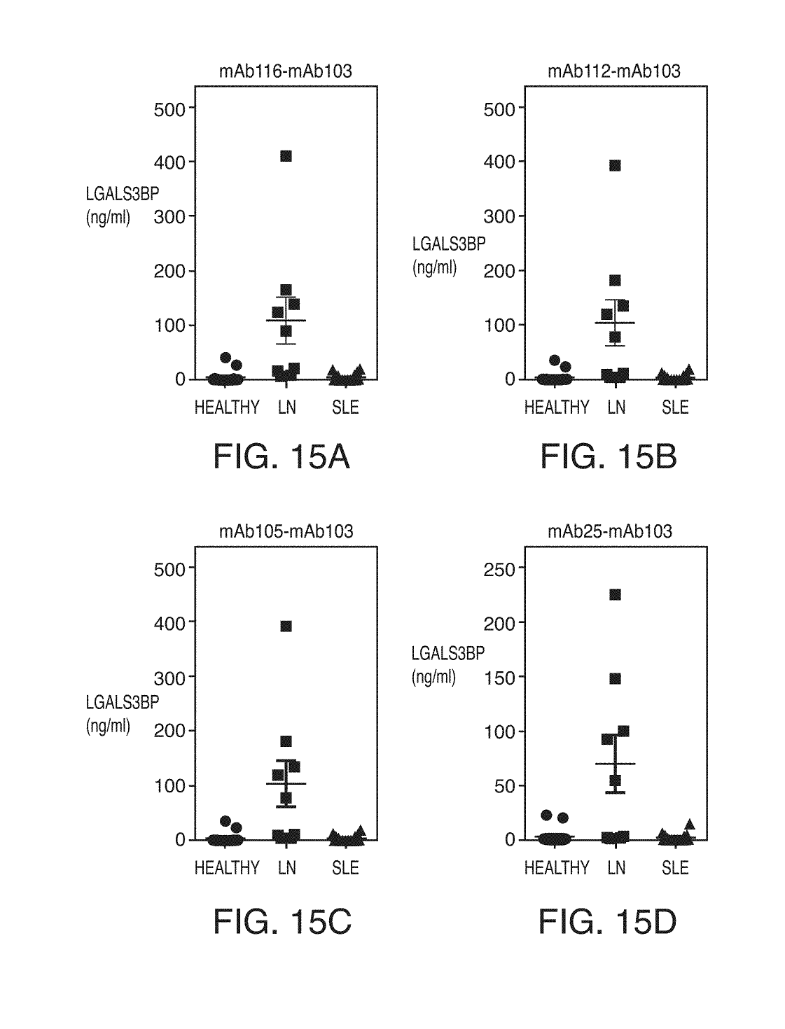

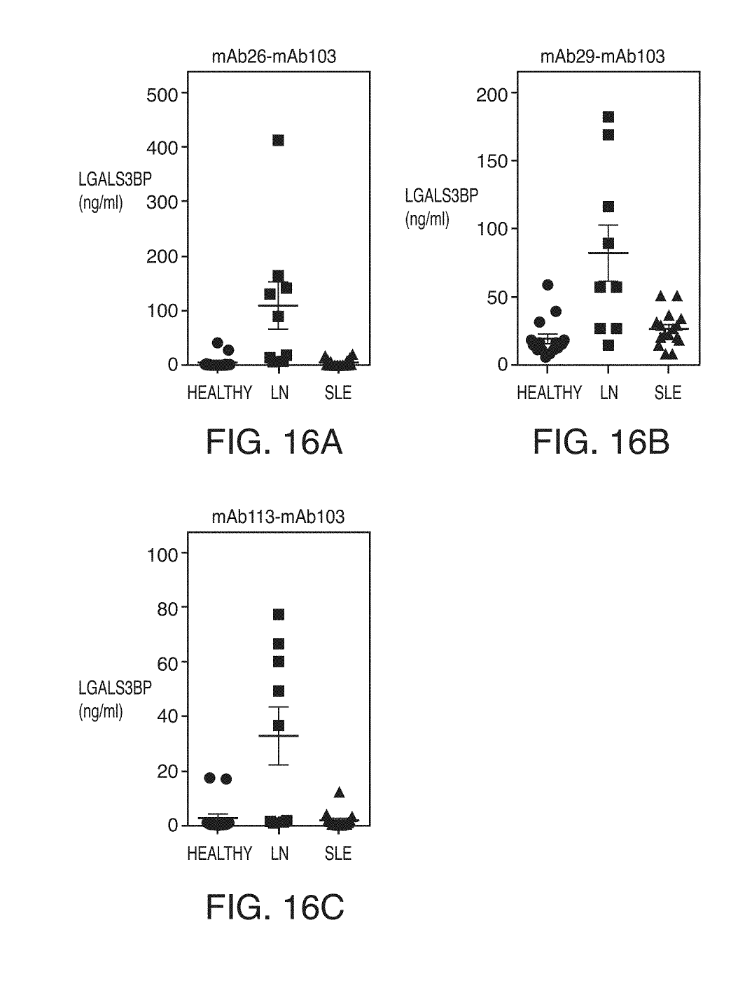

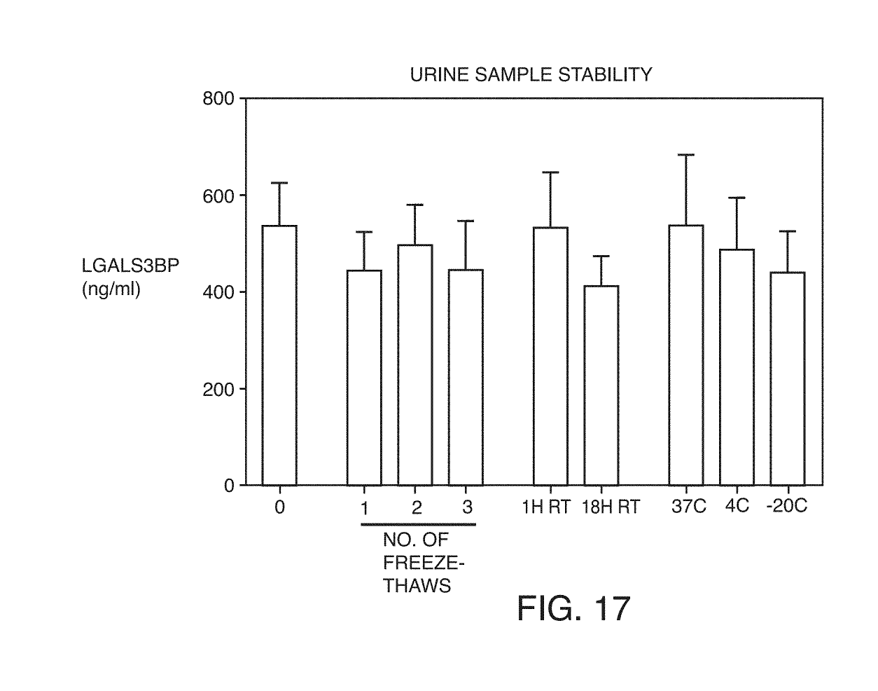

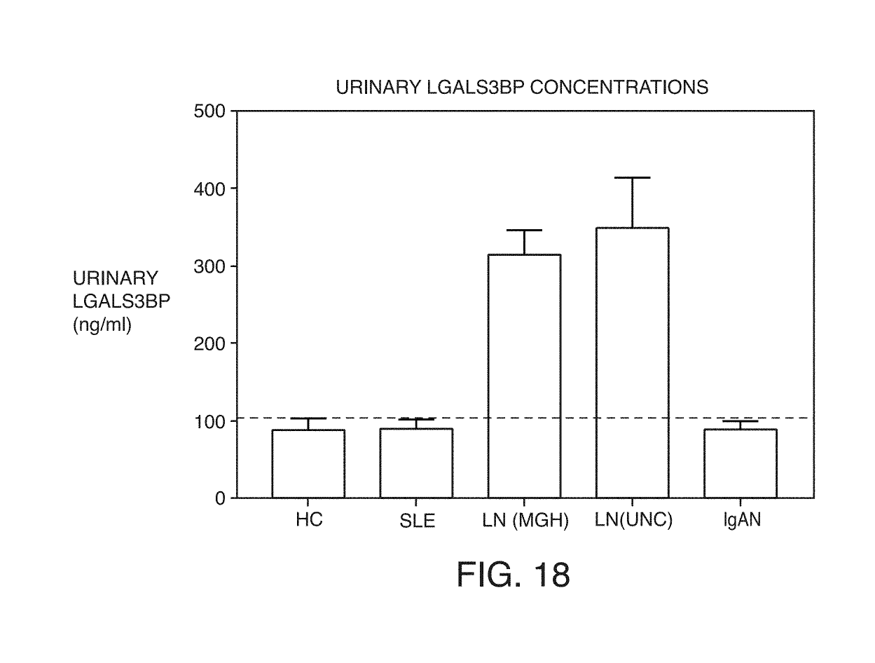

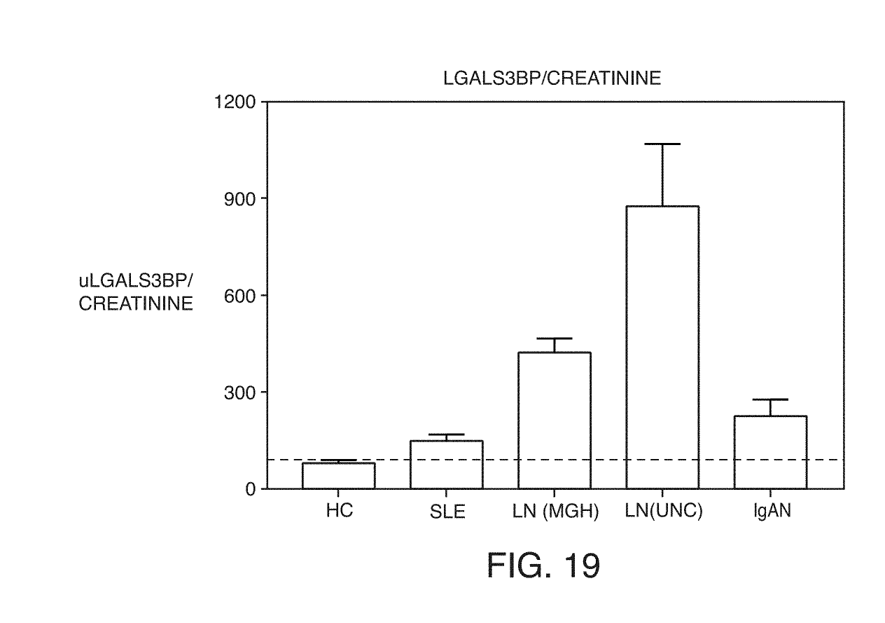

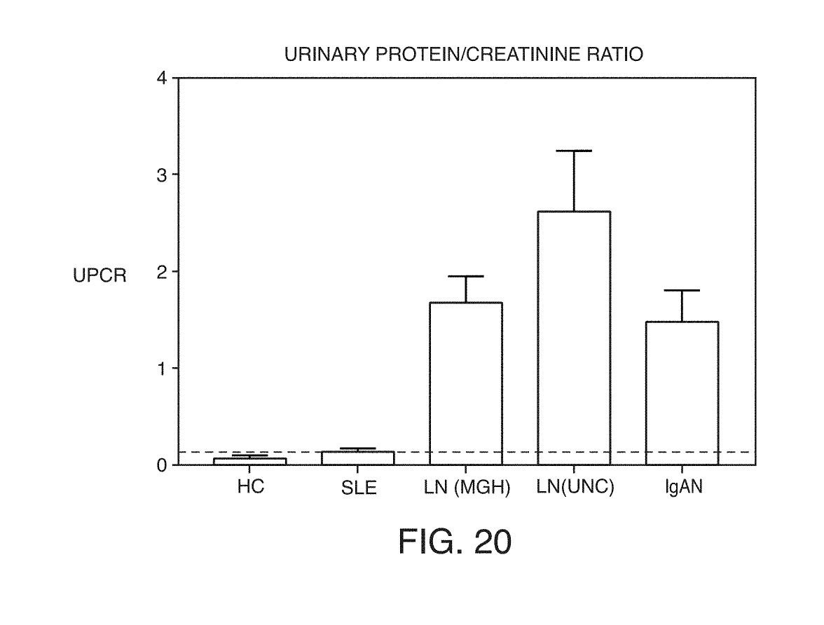

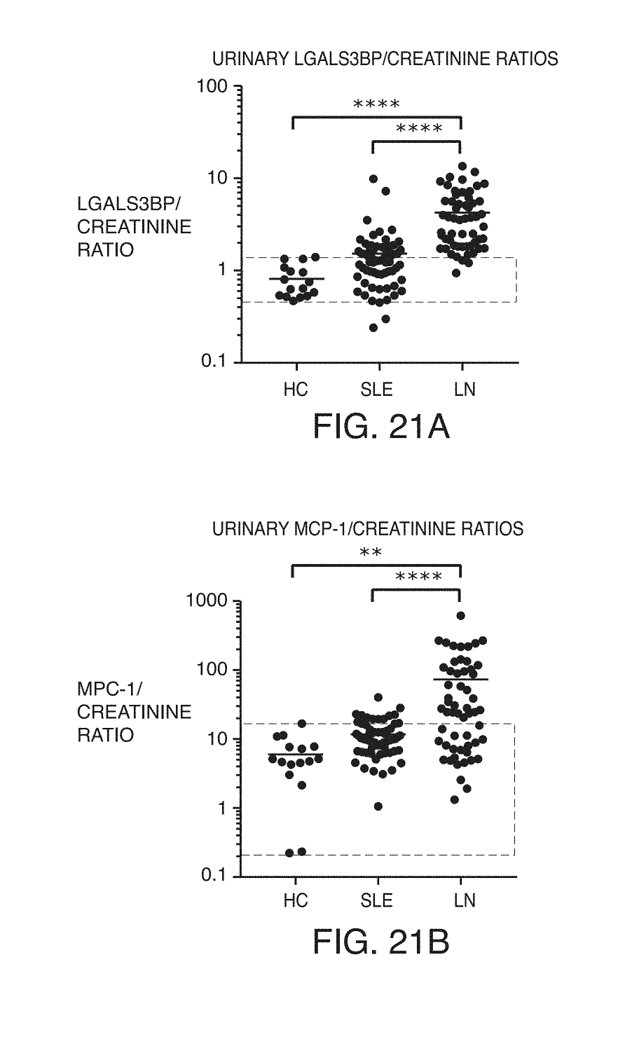

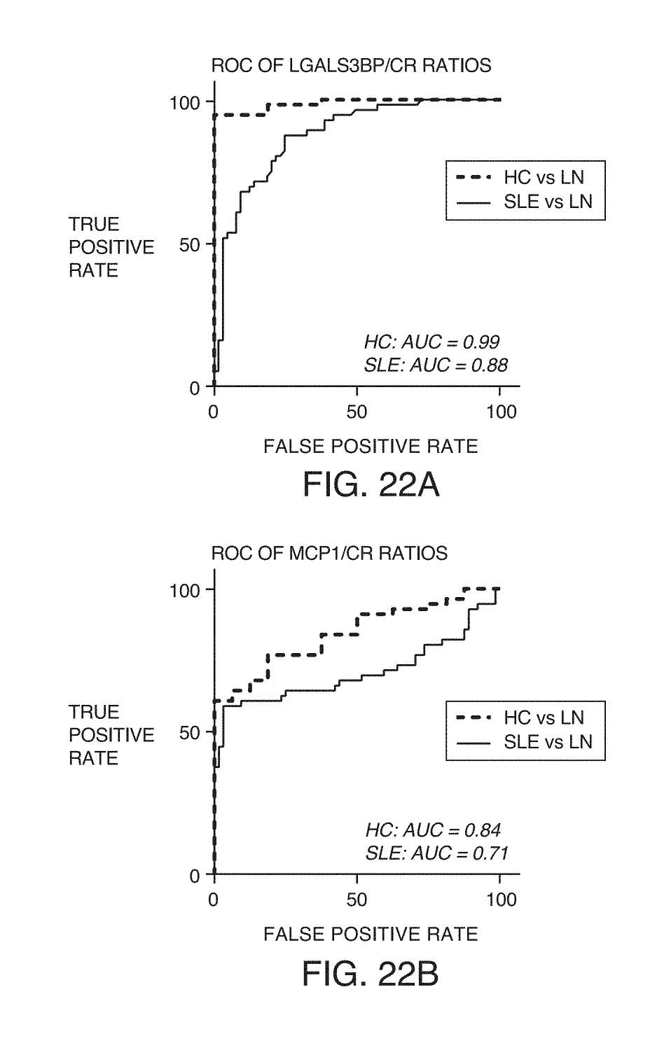

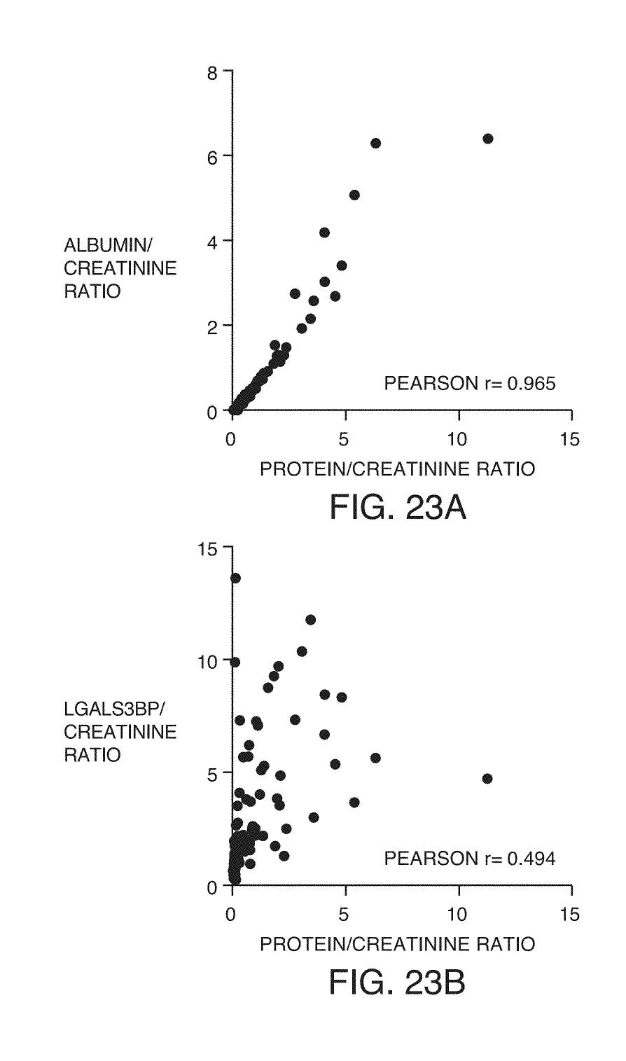

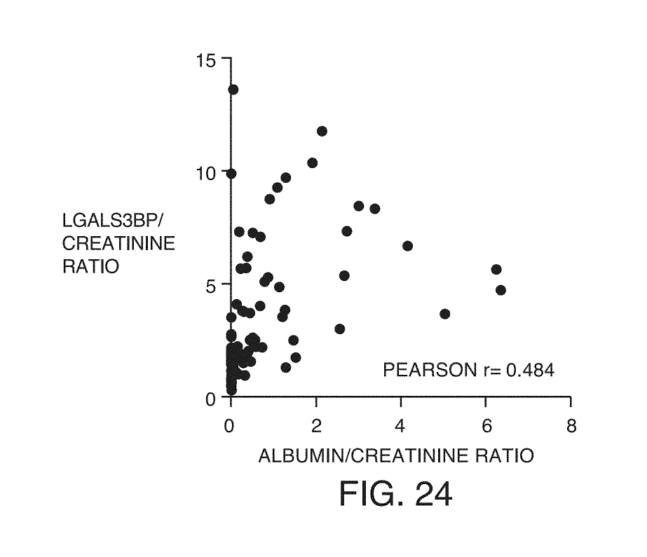

To validate LGALS3BP as a predictive marker for LN, we further examined detected LGALS3BP in terms of total protein or albumin levels. To determine this, the Pearson correlation coefficients were assessed comparing these three measurements to one another after normalizing to urine creatinine levels. Through this empirical inquiry a very strong correlation between total protein and albumin levels was found (R=0.95; FIG. 7A). We also found positive correlations between LGALS3BP and total protein (R=0.513; FIG. 7B) and LGALS3BP and albumin levels (R=0.507; FIG. 7C). Based on these correlation coefficients, these data demonstrate that measured LGALS3BP provides a differential read-out as compared to measured total protein or albumin More specifically, in patient samples which had high levels of LGALS3BP and low levels of total protein this expression profile is consistent with patients having high levels of inflammation in their kidneys, but relatively low levels of kidney damage; consistent with a pathophysiology in LN of early stage LN. In patient samples presenting low levels of LGALS3BP and high total protein levels that expression profile is consistent with patients having low levels of kidney inflammation but a high level of kidney damage; consistent with a pathophysiology in LN of class V late-stage kidney disease with risk of kidney failure. These data demonstrate that, urinary measurements of LGALS3BP provide different and more nuanced diagnostic information concerning the severity and progression of LN as compared to measuring total protein or albumin levels in the urine.

### Example 8: Urine LGALS3BP Levels Fluctuate over Time

LN patients have higher levels of total protein, albumin and LGALS3BP as compared to SLE and HC donors. In most sample donors these values remained fairly constant, especially in the HC and SLE groups over the course of time. In some LN patients, however, spikes were observed in the total protein (FIG. 5A) and albumin (FIG. 5B) and LGALS3BP (FIG. 5C). These metrics are not only in and of themselves (i.e., monitoring renal inflammation in LN patients) but are also useful in evaluating the effectiveness of certain immunosuppressive treatments in LN patients.

For all purposes in the United States of America, each and every publication and patent document cited herein is incorporated by reference for all purposes as if each such publication or document was specifically and individually indicated to be incorporated, herein, by reference.

While the invention has been described with reference to the specific embodiments, changes can be made and equivalents can be substituted to adapt to a particular context or intended use, thereby achieving benefits of the invention without departing from the scope of the claims that follow.

### Example 9: Urinary LGALS3BP/Creatinine Ratios in Different Kidney Disease Groups

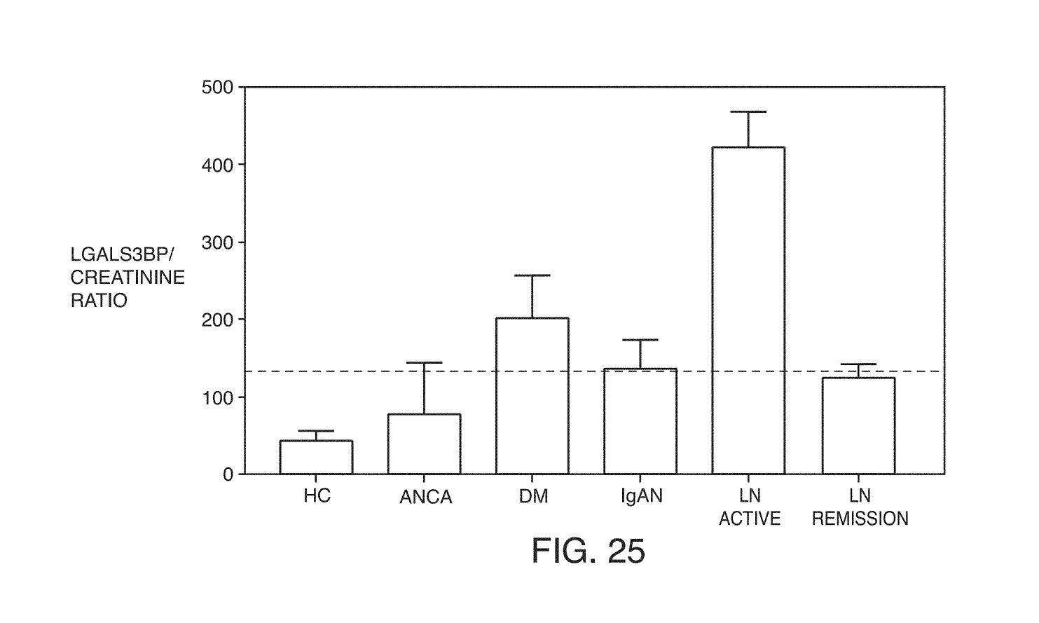

As show in FIG. 25, increased levels of urinary LGALS3BP preferentially in LN when active (flaring). This shows a disease-specific pattern in urinary LGALS3BP expression and a trend that is mainly driven by active inflammation in the context of LN. Diabetic Nephropathy (DM), IgAN and ANCA show low urinary LGALS3BP levels. Considering that ANCA, DM are characterized by chronic low-grade inflammation, the data show that urinary LGALS3BP levels are disease specific and are not increased by non-LN-specific kidney inflammatory states.

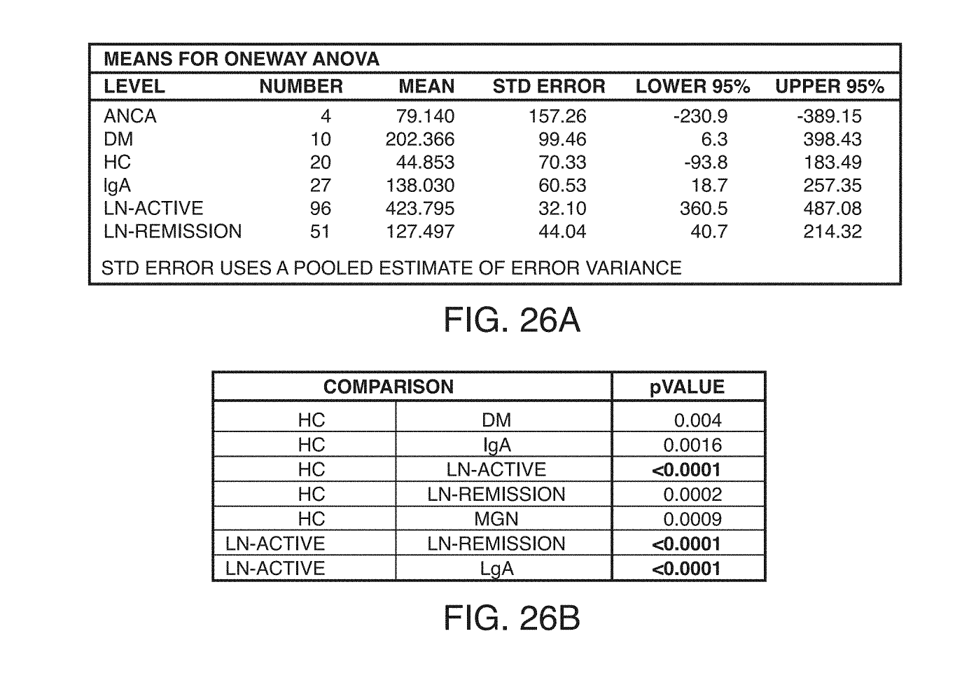

Active LN vs. remitting LN shows striking differences. This is significant in view of the advantages of the urinary LGALS3BP assay described in the instant application: to differentiate between active vs. chronic disease. As shown in FIGS. 26A and 26B, urine LGALS3BP data were normalized to creatinine concentration, natural log transformed and outliers were excluded for data analysis. Also, JMP pro v12 were used including ANOVA and Wilcoxon non parametric multiple comparison showing average LGALS3BP/creatinine ratios and standard error mean. Dotted line indicates average+2 standard deviations for healthy control (132.95).

### Example 10: Urinary LGALS3BP/Creatinine and Urinary Protein/Creatinine Ratios do not Correlate in LN

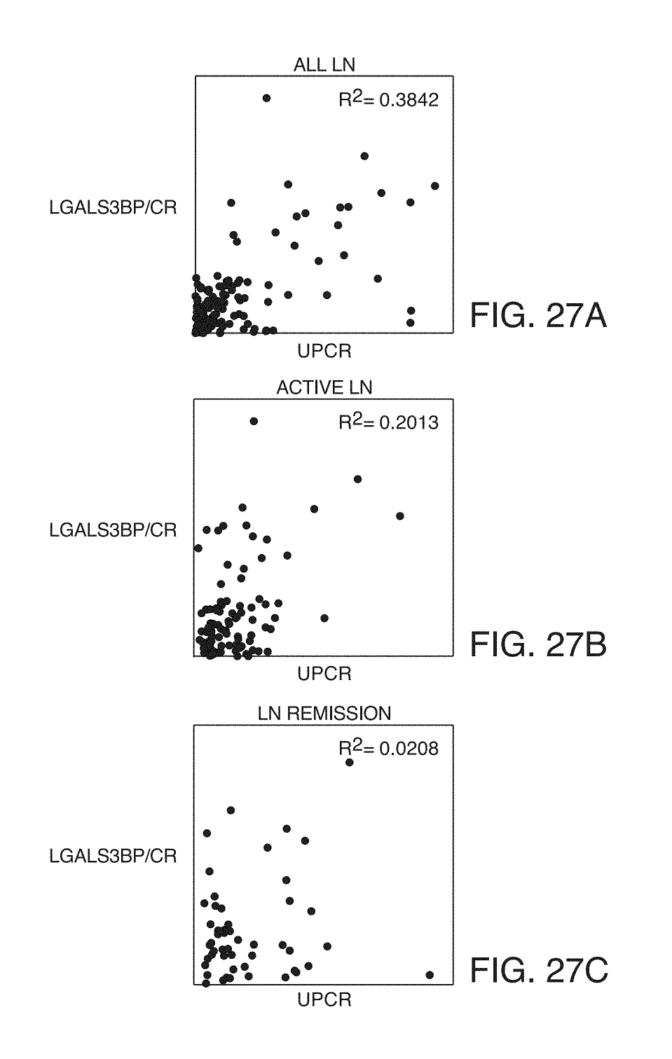

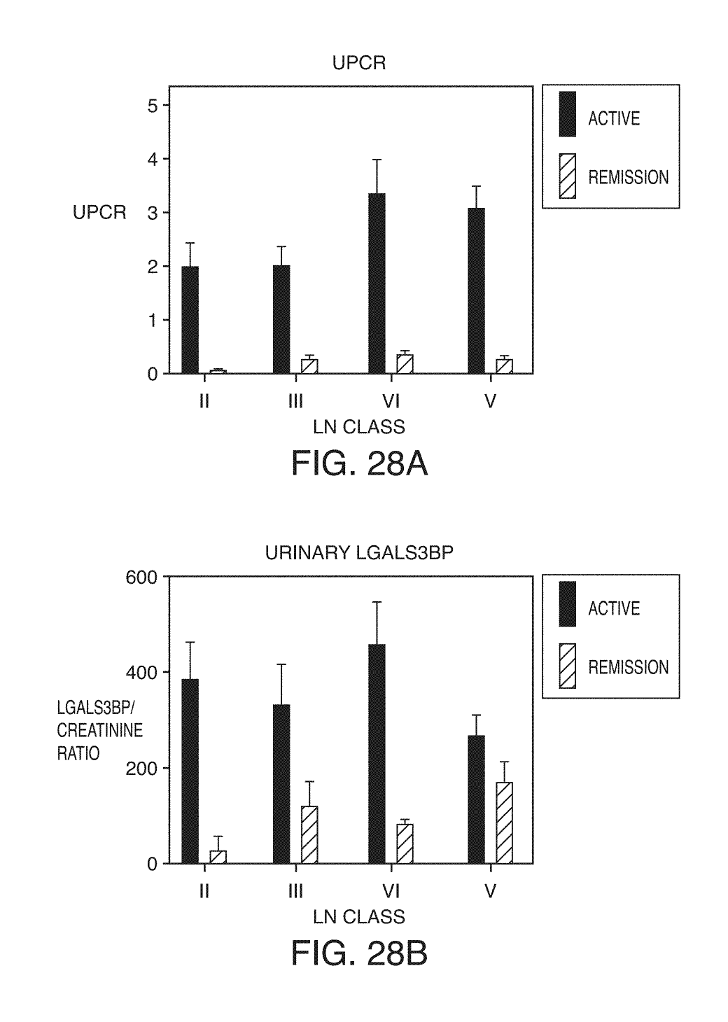

As show in FIGS. 27A, 27B and 27C, patient urine samples were compared for LGALS3BP/Creatinine and urinary total protein/Creatinine (UPCR) levels. These data demonstrate that LGALS3BP/creatinine reports on something else (i.e., inflammation) rather than UPCR (i.e. damage) in active LN kidney disease. The fact that LGALS3BP/Cr is elevated without UPCR being up in active LN demonstrates that this metric reports on active inflammation. The same is true for more samples having elevated UPCR but low LGALS3BP/Cr in remission indicating that inflammation has resolved but kidney damage persists. Patients in remission who, nonetheless, present elevated LGALS3BP/Cr but low UPCR are at risk for a flare of LN. In the aforementioned figures, R² are Pearson correlation coefficients.

### Example 11: Fluctuation of Urinary LGAL3BP/Creatinine Levels in LN Patients

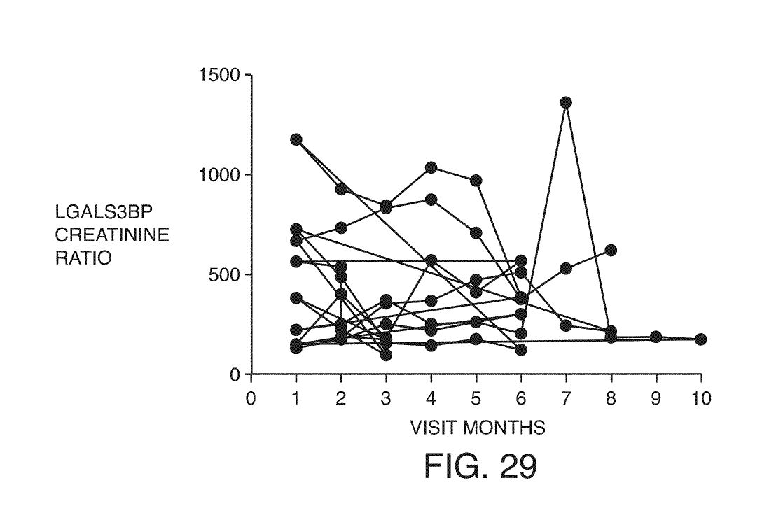

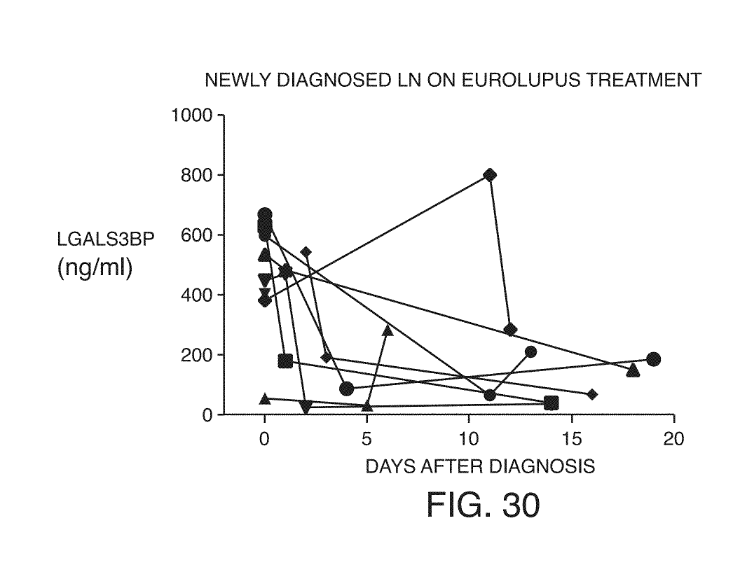

As shown in FIG. 29, there is a fluctuation, over time, of urinary LGALS3BP/creatinine levels in LN patients. More specifically, LN patient urine was monitored monthly. These data indicate that urinary LGALS3BP levels change over time correlate as an early indicator of inflammation.

It is understood that in light of the teachings of this invention to one of ordinary skill in the art that certain changes and modifications may be made thereto without departing from the spirit and scope of the invention.

1. A method for generating data dispositive in diagnosing and non-invasively monitoring renal pathology using samples obtained from a mammalian subject, comprising:
(i) obtaining a dataset associated with the samples, wherein the dataset comprises protein expression levels for at least two markers selected from the group consisting of: urinary LGALS3BP, urinary creatinine and proteinuria expressed as a ratio of urine protein:creatine (uPCR); and
(ii) inputting the dataset into an analytical process that uses the data to generate a result useful in diagnosing and monitoring the renal pathology.

2. The method of claim 2, wherein the renal pathology comprises one or more of: glomerular diseases; systemic lupus erythematosus (SLE) disease; interstitial inflammation in lupus nephritis (LN); interstitial fibrosis in lupus nephritis (LN); renal-interstitial inflammation (INF); crescentic glomerulonephritis; membranous glomerulopathy and glomerular basement membrane abnormalities.

3. An in vitro method for prediction and/or diagnosis of lupus nephritis in a subject affected or potentially affected by systemic lupus erythematosus comprising the following steps: a) providing a sample of urine from said subject: b) measuring the levels of LGALS3BP, creatinine and total protein in said urine; c) expressing the measured levels of LGALS3BP and creatinine (c), as measured in step b), as the ratio: LGALS3BP/c and d) comparing said LGALS3BP/c ratio to said total protein with a control value, wherein an increase of the ratio of LGALS3BP/c to total protein with respect to said control value indicates a development of lupus nephritis.

4. The method according to claim 3, wherein the measurement of said LGALS3BP and creatinine levels is carried out by ELISA or Western-Blot.

5. An in vitro method for monitoring progression of lupus nephritis in a patient affected by systemic lupus erythematosus comprising the following steps: a) providing a sample of urine from said subject: b) measuring the levels of galectin 3 binding protein, creatinine and total protein in said urine; c) expressing the measured levels of LGALS3BP and creatinine (c), as measured in step b), as the ratio: LGALS3BP/c to said total protein in at least a first and at least a second urine sample of said subject, wherein said at least a first and a second urine samples obtained at different times; and d) comparing said measured LGALS3BP/c ratio to said total protein concentration obtained for said first and second urine samples.

6. The method according to claim 5, wherein said at least a first and second sample are respectively obtained before starting a therapy and during and/or after said therapy.

7. The method according to claim 6, wherein said therapy comprises treatment with steroid drugs, immunosuppressant, Rituximab, or inhibitors of angiotensin converting enzyme.

8. An in vitro method for diagnosis of systemic lupus erythematosus and lupus nephritis in a subject and discriminating them from other rheumatologic conditions and primary glomerular nephritis, said method comprising: a) providing a sample of urine from said subject: b) measuring the levels of LGALS3BP, creatinine and total protein in said urine; c) expressing the measured levels LGALS3BP and creatinine (c), as measured in step b), as the ratio: LGALS3BP/c and d) comparing said LGALS3BP/c ratio to said total protein with a control value, wherein an increase of the ratio of LGALS3BP/c to total protein with respect to said control value indicates development of lupus nephritis.


SEQUENCE LISTING (1507 sequences)

SEQ ID NO: 1 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 2 - protein, 49 aa, Artificial Sequence
  source: /note=“Description of Artificial Sequence Synthetic polypeptide”
GFTFSSYGISYDGSNKAKGSSPYYYYGMDVQSVSTNGASQQYNTWPPVR

SEQ ID NO: 3 - protein, 44 aa, Artificial Sequence
  source: /note=“Description of Artificial Sequence Synthetic polypeptide”
GFTVSSNYIYSGGSTARDTASGGMDVQSVSSNGASQQYGYSQIT

SEQ ID NO: 4 - protein, 52 aa, Artificial Sequence
  source: /note=“Description of Artificial Sequence Synthetic polypeptide”
GFTFSSYGISGSGGSTAKATGYSSGWYGAYFDYQSVSSSYGASQQYGSSPLT

SEQ ID NO: 5 - protein, 58 aa, Artificial Sequence
  source: /note=“Description of Artificial Sequence Synthetic polypeptide”
GDSVSSNSAATYYRSKWYNAREFQDSSSWYEGRAFDISSDVGGYNYDVSSSYAGSSVV

SEQ ID NO: 6 - protein, 52 aa, Artificial Sequence
  source: /note=“Description of Artificial Sequence Synthetic polypeptide”
GDSVSSNSAATYYRSKWYNARGGVGATWYYGMDVKLGDKYQDSQTWDSSTVV

SEQ ID NO: 7 - protein, 50 aa, Artificial Sequence
  source: /note=“Description of Artificial Sequence Synthetic polypeptide”
GFTFSSYSIWYDGSNKARLGSGWSLDYSSDVGGYNYDVNSSYTSSNTLVV

SEQ ID NO: 8 - protein, 51 aa, Artificial Sequence
  source: /note=“Description of Artificial Sequence Synthetic polypeptide”
GFTFSSYPISYDGSNKARVGSGGWTPDYSSDVGGYNYDVSSSYTSSSTLVV

SEQ ID NO: 9 - protein, 49 aa, Artificial Sequence
  source: /note=“Description of Artificial Sequence Synthetic polypeptide”
GFTFSNAWIKSKNDGGTTTTAPSLMDVSSYIATNSSDSAAWDDSLNAYV

SEQ ID NO: 10 - protein, 51 aa, Artificial Sequence
  source: /note=“Description of Artificial Sequence Synthetic polypeptide”
GFTFSSYPISYDGSNKARVGSGGWTPDYSSDIGGYNYEVSSSYTSSSTLVV

SEQ ID NO: 11 - protein, 51 aa, Artificial Sequence
  source: /note=“Description of Artificial Sequence Synthetic polypeptide”
GFTFSSYPISYDGSNKARVGSGGWTPDYSSDVGGYNYEVSSSYTSSSTLVV

SEQ ID NO: 12 - protein, 51 aa, Artificial Sequence; identical to SEQ ID NO: 8
  source: /note=“Description of Artificial Sequence Synthetic polypeptide”

SEQ ID NO: 13 - protein, 45 aa, Artificial Sequence
  source: /note=“Description of Artificial Sequence Synthetic polypeptide”
GFTVSSNYIYSGGSTARDLHSAAGFDYGNNYENNGTWDSSLNVGV

SEQ ID NO: 14 - protein, 47 aa, Artificial Sequence
  source: /note=“Description of Artificial Sequence Synthetic polypeptide”
GFTVSSNYIYSGGSTARDFEGSGALDVNIGDKRYDTQVWDTDTNHAV

SEQ ID NO: 15 - protein, 48 aa, Artificial Sequence
  source: /note=“Description of Artificial Sequence Synthetic polypeptide”
GFTFSNAWIKSKNDGGTTTTAPSLMDVILGHYHGKDNNSRDRSGTQVL

SEQ ID NO: 16 - protein, 49 aa, Artificial Sequence
  source: /note=“Description of Artificial Sequence Synthetic polypeptide”
GFTVSSNYIYSGGSTARDLSYSDAFDISSNIGNNYDNDGTWDNSLSAVV

SEQ ID NO: 17 - protein, 52 aa, Artificial Sequence
  source: /note=“Description of Artificial Sequence Synthetic polypeptide”
GFTFSSYGIWYDGNNKARDNSGSYNWFNPSSDVGGYNYEVSSSYSGSNNLVV

SEQ ID NO: 18 - protein, 51 aa, Artificial Sequence
  source: /note=“Description of Artificial Sequence Synthetic polypeptide”
GFTFSSYPISYDGGNKARVGSGGWTPDYSSDVGGYNYEVTSSYTSSSTFVV

SEQ ID NO: 19 - protein, 51 aa, Artificial Sequence
  source: /note=“Description of Artificial Sequence Synthetic polypeptide”
GFTFSSYAISYDGSNKARDRGVEGAYGMDVQRVRSSYGASQQYGSSPPRII

SEQ ID NO: 20 - protein, 52 aa, Artificial Sequence
  source: /note=“Description of Artificial Sequence Synthetic polypeptide”
GYTFTGYYINPNSGGTARGGDCSSTSCYDPDYGGSIASNYKDNQSYGSGNVV

SEQ ID NO: 21 - protein, 49 aa, Artificial Sequence
  source: /note=“Description of Artificial Sequence Synthetic polypeptide”
GYTFTSYYINPSGGSTAREDHDYSNQGGFDYQSVTSNYGASQQYGSSPT

SEQ ID NO: 22 - protein, 53 aa, Artificial Sequence
  source: /note=“Description of Artificial Sequence Synthetic polypeptide”
GDSVSSNSAATYYRSKWYNAREKIAVAGYYYGMDVKLGDKYQNNQAWDSSAVV

SEQ ID NO: 23 - protein, 52 aa, Artificial Sequence
  source: /note=“Description of Artificial Sequence Synthetic polypeptide”
GDSVSSNSAATYYSSKWYNARGGSSEFYYYGMDVKLGNKYENNQAWDSSTAV

SEQ ID NO: 24 - protein, 45 aa, Artificial Sequence
  source: /note=“Description of Artificial Sequence Synthetic polypeptide”
GFTFDDYAISWNSGSIAKDIAAGGLDSQSISSYAASQQSYSTSWT

SEQ ID NO: 25 - protein, 49 aa, Artificial Sequence
  source: /note=“Description of Artificial Sequence Synthetic polypeptide”
GYTFTSYGISAYNGNTARGLGDSSSSYTSNIGANHTKNAAWDDSLRGWT

SEQ ID NO: 26 - protein, 47 aa, Artificial Sequence
  source: /note=“Description of Artificial Sequence Synthetic polypeptide”
GYSFTSYWIYPGDSDTASGASPYYFDYSLRSYYGKNNSRDSSGNHWV

SEQ ID NO: 27 - protein, 51 aa, Artificial Sequence
  source: /note=“Description of Artificial Sequence Synthetic polypeptide”
GYTFTSYGISAYNGNTARDPVYSSSWGGYAFDIQGVNSDGASQQYNNWPWT

SEQ ID NO: 28 - protein, 51 aa, Artificial Sequence
  source: /note=“Description of Artificial Sequence Synthetic polypeptide”
GFTFSSYPISYDGSNKTRVGSGGWTPDYSSDVGGYNYEVSSSYTSSSTLVV

SEQ ID NO: 29 - protein, 44 aa, Artificial Sequence; identical to SEQ ID NO: 3
  source: /note=“Description of Artificial Sequence Synthetic polypeptide”

SEQ ID NO: 30 - protein, 51 aa, Artificial Sequence
  source: /note=“Description of Artificial Sequence Synthetic polypeptide”
GFTFSSYGIWYDGSNKAREVVGSYYLDYSSDIGGYKYDVTGSYSSSSSHYV

SEQ ID NO: 31 - protein, 48 aa, Artificial Sequence
  source: /note=“Description of Artificial Sequence Synthetic polypeptide”
GFTFSSYWIKQDGSEKARDLHCGSSCGPEAQTISSYGASQQSYSTPQT

SEQ ID NO: 32 - protein, 8 aa, Artificial Sequence
  source: /note=“Description of Artificial Sequence Synthetic peptide”
GFTFSSYG

SEQ ID NO: 33 - protein, 8 aa, Artificial Sequence
  source: /note=“Description of Artificial Sequence Synthetic peptide”
ISYDGSNK

SEQ ID NO: 34 - protein, 14 aa, Artificial Sequence
  source: /note=“Description of Artificial Sequence Synthetic peptide”
AKGSSPYYYYGMDV

SEQ ID NO: 35 - protein, 6 aa, Artificial Sequence
  source: /note=“Description of Artificial Sequence Synthetic peptide”
QSVSTN

SEQ ID NO: 36 - protein, 3 aa, Artificial Sequence
  source: /note=“Description of Artificial Sequence Synthetic peptide”
GAS

SEQ ID NO: 37 - protein, 10 aa, Artificial Sequence
  source: /note=“Description of Artificial Sequence Synthetic peptide”
QQYNTWPPVR

SEQ ID NO: 38 - protein, 8 aa, Artificial Sequence
  source: /note=“Description of Artificial Sequence Synthetic peptide”
GFTVSSNY

SEQ ID NO: 39 - protein, 7 aa, Artificial Sequence
  source: /note=“Description of Artificial Sequence Synthetic peptide”
IYSGGST

SEQ ID NO: 40 - protein, 11 aa, Artificial Sequence
  source: /note=“Description of Artificial Sequence Synthetic peptide”
ARDTASGGMDV

SEQ ID NO: 41 - protein, 6 aa, Artificial Sequence
  source: /note=“Description of Artificial Sequence Synthetic peptide”
QSVSSN

SEQ ID NO: 42 - protein, 3 aa, Artificial Sequence
  source: /note=“Description of Artificial Sequence Synthetic peptide”
GAS

SEQ ID NO: 43 - protein, 9 aa, Artificial Sequence
  source: /note=“Description of Artificial Sequence Synthetic peptide”
QQYGYSQIT

SEQ ID NO: 44 - protein, 8 aa, Artificial Sequence
  source: /note=“Description of Artificial Sequence Synthetic peptide”
GFTFSSYG

SEQ ID NO: 45 - protein, 8 aa, Artificial Sequence
  source: /note=“Description of Artificial Sequence Synthetic peptide”
ISGSGGST

SEQ ID NO: 46 - protein, 17 aa, Artificial Sequence
  source: /note=“Description of Artificial Sequence Synthetic peptide”
AKATGYSSGWYGAYFDY

SEQ ID NO: 47 - protein, 7 aa, Artificial Sequence
  source: /note=“Description of Artificial Sequence Synthetic peptide”
QSVSSSY

SEQ ID NO: 48 - protein, 3 aa, Artificial Sequence
  source: /note=“Description of Artificial Sequence Synthetic peptide”
GAS

SEQ ID NO: 49 - protein, 9 aa, Artificial Sequence
  source: /note=“Description of Artificial Sequence Synthetic peptide”
QQYGSSPLT

SEQ ID NO: 50 - protein, 10 aa, Artificial Sequence
  source: /note=“Description of Artificial Sequence Synthetic peptide”
GDSVSSNSAA

SEQ ID NO: 51 - protein, 9 aa, Artificial Sequence
  source: /note=“Description of Artificial Sequence Synthetic peptide”
TYYRSKWYN

SEQ ID NO: 52 - protein, 18 aa, Artificial Sequence
  source: /note=“Description of Artificial Sequence Synthetic peptide”
AREFQDSSSWYEGRAFDI

SEQ ID NO: 53 - protein, 9 aa, Artificial Sequence
  source: /note=“Description of Artificial Sequence Synthetic peptide”
SSDVGGYNY

SEQ ID NO: 54 - protein, 3 aa, Artificial Sequence
  source: /note=“Description of Artificial Sequence Synthetic peptide”
DVS

SEQ ID NO: 55 - protein, 9 aa, Artificial Sequence
  source: /note=“Description of Artificial Sequence Synthetic peptide”
SSYAGSSVV

SEQ ID NO: 56 - protein, 10 aa, Artificial Sequence
  source: /note=“Description of Artificial Sequence Synthetic peptide”
GDSVSSNSAA

SEQ ID NO: 57 - protein, 9 aa, Artificial Sequence
  source: /note=“Description of Artificial Sequence Synthetic peptide”
TYYRSKWYN

SEQ ID NO: 58 - protein, 15 aa, Artificial Sequence
  source: /note=“Description of Artificial Sequence Synthetic peptide”
ARGGVGATWYYGMDV

SEQ ID NO: 59 - protein, 6 aa, Artificial Sequence
  source: /note=“Description of Artificial Sequence Synthetic peptide”
KLGDKY

SEQ ID NO: 60 - protein, 3 aa, Artificial Sequence
  source: /note=“Description of Artificial Sequence Synthetic peptide”
QDS

SEQ ID NO: 61 - protein, 9 aa, Artificial Sequence
  source: /note=“Description of Artificial Sequence Synthetic peptide”
QTWDSSTVV

SEQ ID NO: 62 - protein, 8 aa, Artificial Sequence
  source: /note=“Description of Artificial Sequence Synthetic peptide”
GFTFSSYS

SEQ ID NO: 63 - protein, 8 aa, Artificial Sequence
  source: /note=“Description of Artificial Sequence Synthetic peptide”
IWYDGSNK

SEQ ID NO: 64 - protein, 11 aa, Artificial Sequence
  source: /note=“Description of Artificial Sequence Synthetic peptide”
ARLGSGWSLDY

SEQ ID NO: 65 - protein, 9 aa, Artificial Sequence
  source: /note=“Description of Artificial Sequence Synthetic peptide”
SSDVGGYNY

SEQ ID NO: 66 - protein, 3 aa, Artificial Sequence
  source: /note=“Description of Artificial Sequence Synthetic peptide”
DVN

SEQ ID NO: 67 - protein, 11 aa, Artificial Sequence
  source: /note=“Description of Artificial Sequence Synthetic peptide”
SSYTSSNTLVV

SEQ ID NO: 68 - protein, 8 aa, Artificial Sequence
  source: /note=“Description of Artificial Sequence Synthetic peptide”
GFTFSSYP

SEQ ID NO: 69 - protein, 8 aa, Artificial Sequence
  source: /note=“Description of Artificial Sequence Synthetic peptide”
ISYDGSNK

SEQ ID NO: 70 - protein, 12 aa, Artificial Sequence
  source: /note=“Description of Artificial Sequence Synthetic peptide”
ARVGSGGWTPDY

SEQ ID NO: 71 - protein, 9 aa, Artificial Sequence
  source: /note=“Description of Artificial Sequence Synthetic peptide”
SSDVGGYNY

SEQ ID NO: 72 - protein, 3 aa, Artificial Sequence
  source: /note=“Description of Artificial Sequence Synthetic peptide”
DVS

SEQ ID NO: 73 - protein, 11 aa, Artificial Sequence
  source: /note=“Description of Artificial Sequence Synthetic peptide”
SSYTSSSTLVV

SEQ ID NO: 74 - protein, 8 aa, Artificial Sequence
  source: /note=“Description of Artificial Sequence Synthetic peptide”
GFTFSNAW

SEQ ID NO: 75 - protein, 10 aa, Artificial Sequence
  source: /note=“Description of Artificial Sequence Synthetic peptide”
IKSKNDGGTT

SEQ ID NO: 76 - protein, 9 aa, Artificial Sequence
  source: /note=“Description of Artificial Sequence Synthetic peptide”
TTAPSLMDV

SEQ ID NO: 77 - protein, 8 aa, Artificial Sequence
  source: /note=“Description of Artificial Sequence Synthetic peptide”
SSYIATNS

SEQ ID NO: 78 - protein, 3 aa, Artificial Sequence
  source: /note=“Description of Artificial Sequence Synthetic peptide”
SDS

SEQ ID NO: 79 - protein, 11 aa, Artificial Sequence
  source: /note=“Description of Artificial Sequence Synthetic peptide”
AAWDDSLNAYV

SEQ ID NO: 80 - protein, 8 aa, Artificial Sequence
  source: /note=“Description of Artificial Sequence Synthetic peptide”
GFTFSSYP

SEQ ID NO: 81 - protein, 8 aa, Artificial Sequence
  source: /note=“Description of Artificial Sequence Synthetic peptide”
ISYDGSNK

SEQ ID NO: 82 - protein, 12 aa, Artificial Sequence
  source: /note=“Description of Artificial Sequence Synthetic peptide”
ARVGSGGWTPDY

SEQ ID NO: 83 - protein, 9 aa, Artificial Sequence
  source: /note=“Description of Artificial Sequence Synthetic peptide”
SSDIGGYNY

SEQ ID NO: 84 - protein, 3 aa, Artificial Sequence
  source: /note=“Description of Artificial Sequence Synthetic peptide”
EVS

SEQ ID NO: 85 - protein, 11 aa, Artificial Sequence
  source: /note=“Description of Artificial Sequence Synthetic peptide”
SSYTSSSTLVV

SEQ ID NO: 86 - protein, 8 aa, Artificial Sequence
  source: /note=“Description of Artificial Sequence Synthetic peptide”
GFTFSSYP

SEQ ID NO: 87 - protein, 8 aa, Artificial Sequence
  source: /note=“Description of Artificial Sequence Synthetic peptide”
ISYDGSNK

SEQ ID NO: 88 - protein, 12 aa, Artificial Sequence
  source: /note=“Description of Artificial Sequence Synthetic peptide”
ARVGSGGWTPDY

SEQ ID NO: 89 - protein, 9 aa, Artificial Sequence
  source: /note=“Description of Artificial Sequence Synthetic peptide”
SSDVGGYNY

SEQ ID NO: 90 - protein, 3 aa, Artificial Sequence
  source: /note=“Description of Artificial Sequence Synthetic peptide”
EVS

SEQ ID NO: 91 - protein, 11 aa, Artificial Sequence
  source: /note=“Description of Artificial Sequence Synthetic peptide”
SSYTSSSTLVV

SEQ ID NO: 92 - protein, 8 aa, Artificial Sequence
  source: /note=“Description of Artificial Sequence Synthetic peptide”
GFTFSSYP

SEQ ID NO: 93 - protein, 8 aa, Artificial Sequence
  source: /note=“Description of Artificial Sequence Synthetic peptide”
ISYDGSNK

SEQ ID NO: 94 - protein, 12 aa, Artificial Sequence
  source: /note=“Description of Artificial Sequence Synthetic peptide”
ARVGSGGWTPDY

SEQ ID NO: 95 - protein, 9 aa, Artificial Sequence
  source: /note=“Description of Artificial Sequence Synthetic peptide”
SSDVGGYNY

SEQ ID NO: 96 - protein, 3 aa, Artificial Sequence
  source: /note=“Description of Artificial Sequence Synthetic peptide”
DVS

SEQ ID NO: 97 - protein, 11 aa, Artificial Sequence
  source: /note=“Description of Artificial Sequence Synthetic peptide”
SSYTSSSTLVV

SEQ ID NO: 98 - protein, 8 aa, Artificial Sequence
  source: /note=“Description of Artificial Sequence Synthetic peptide”
GFTVSSNY

SEQ ID NO: 99 - protein, 7 aa, Artificial Sequence
  source: /note=“Description of Artificial Sequence Synthetic peptide”
IYSGGST

SEQ ID NO: 100 - protein, 12 aa, Artificial Sequence
  source: /note=“Description of Artificial Sequence Synthetic peptide”
ARDLHSAAGFDY

SEQ ID NO: 101 - protein, 4 aa, Artificial Sequence
  source: /note=“Description of Artificial Sequence Synthetic peptide”
GNNY

SEQ ID NO: 102 - protein, 3 aa, Artificial Sequence
  source: /note=“Description of Artificial Sequence Synthetic peptide”
ENN

SEQ ID NO: 103 - protein, 11 aa, Artificial Sequence
  source: /note=“Description of Artificial Sequence Synthetic peptide”
GTWDSSLNVGV

SEQ ID NO: 104 - protein, 8 aa, Artificial Sequence
  source: /note=“Description of Artificial Sequence Synthetic peptide”
GFTVSSNY

SEQ ID NO: 105 - protein, 7 aa, Artificial Sequence
  source: /note=“Description of Artificial Sequence Synthetic peptide”
IYSGGST

SEQ ID NO: 106 - protein, 12 aa, Artificial Sequence
  source: /note=“Description of Artificial Sequence Synthetic peptide”
ARDFEGSGALDV

SEQ ID NO: 107 - protein, 6 aa, Artificial Sequence
  source: /note=“Description of Artificial Sequence Synthetic peptide”
NIGDKR

SEQ ID NO: 108 - protein, 3 aa, Artificial Sequence
  source: /note=“Description of Artificial Sequence Synthetic peptide”
YDT

SEQ ID NO: 109 - protein, 11 aa, Artificial Sequence
  source: /note=“Description of Artificial Sequence Synthetic peptide”
QVWDTDTNHAV

SEQ ID NO: 110 - protein, 8 aa, Artificial Sequence
  source: /note=“Description of Artificial Sequence Synthetic peptide”
GFTFSNAW

SEQ ID NO: 111 - protein, 10 aa, Artificial Sequence
  source: /note=“Description of Artificial Sequence Synthetic peptide”
IKSKNDGGTT

SEQ ID NO: 112 - protein, 9 aa, Artificial Sequence
  source: /note=“Description of Artificial Sequence Synthetic peptide”
TTAPSLMDV

SEQ ID NO: 113 - protein, 6 aa, Artificial Sequence
  source: /note=“Description of Artificial Sequence Synthetic peptide”
ILGHYH

SEQ ID NO: 114 - protein, 4 aa, Artificial Sequence
  source: /note=“Description of Artificial Sequence Synthetic peptide”
GKDN

SEQ ID NO: 115 - protein, 11 aa, Artificial Sequence
  source: /note=“Description of Artificial Sequence Synthetic peptide”
NSRDRSGTQVL

SEQ ID NO: 116 - protein, 8 aa, Artificial Sequence
  source: /note=“Description of Artificial Sequence Synthetic peptide”
GFTVSSNY

SEQ ID NO: 117 - protein, 7 aa, Artificial Sequence
  source: /note=“Description of Artificial Sequence Synthetic peptide”
IYSGGST

SEQ ID NO: 118 - protein, 12 aa, Artificial Sequence
  source: /note=“Description of Artificial Sequence Synthetic peptide”
ARDLSYSDAFDI

SEQ ID NO: 119 - protein, 8 aa, Artificial Sequence
  source: /note=“Description of Artificial Sequence Synthetic peptide”
SSNIGNNY

SEQ ID NO: 120 - protein, 3 aa, Artificial Sequence
  source: /note=“Description of Artificial Sequence Synthetic peptide”
DND

SEQ ID NO: 121 - protein, 11 aa, Artificial Sequence
  source: /note=“Description of Artificial Sequence Synthetic peptide”
GTWDNSLSAVV

SEQ ID NO: 122 - protein, 8 aa, Artificial Sequence
  source: /note=“Description of Artificial Sequence Synthetic peptide”
GFTFSSYG

SEQ ID NO: 123 - protein, 8 aa, Artificial Sequence
  source: /note=“Description of Artificial Sequence Synthetic peptide”
IWYDGNNK

SEQ ID NO: 124 - protein, 13 aa, Artificial Sequence
  source: /note=“Description of Artificial Sequence Synthetic peptide”
ARDNSGSYNWFNP

SEQ ID NO: 125 - protein, 9 aa, Artificial Sequence
  source: /note=“Description of Artificial Sequence Synthetic peptide”
SSDVGGYNY

SEQ ID NO: 126 - protein, 3 aa, Artificial Sequence
  source: /note=“Description of Artificial Sequence Synthetic peptide”
EVS

SEQ ID NO: 127 - protein, 11 aa, Artificial Sequence
  source: /note=“Description of Artificial Sequence Synthetic peptide”
SSYSGSNNLVV

SEQ ID NO: 128 - protein, 8 aa, Artificial Sequence
  source: /note=“Description of Artificial Sequence Synthetic peptide”
GFTFSSYP

SEQ ID NO: 129 - protein, 8 aa, Artificial Sequence
  source: /note=“Description of Artificial Sequence Synthetic peptide”
ISYDGGNK

SEQ ID NO: 130 - protein, 12 aa, Artificial Sequence
  source: /note=“Description of Artificial Sequence Synthetic peptide”
ARVGSGGWTPDY

SEQ ID NO: 131 - protein, 9 aa, Artificial Sequence
  source: /note=“Description of Artificial Sequence Synthetic peptide”
SSDVGGYNY

SEQ ID NO: 132 - protein, 3 aa, Artificial Sequence
  source: /note=“Description of Artificial Sequence Synthetic peptide”
EVT

SEQ ID NO: 133 - protein, 11 aa, Artificial Sequence
  source: /note=“Description of Artificial Sequence Synthetic peptide”
SSYTSSSTFVV

SEQ ID NO: 134 - protein, 8 aa, Artificial Sequence
  source: /note=“Description of Artificial Sequence Synthetic peptide”
GFTFSSYA

SEQ ID NO: 135 - protein, 8 aa, Artificial Sequence
  source: /note=“Description of Artificial Sequence Synthetic peptide”
ISYDGSNK

SEQ ID NO: 136 - protein, 14 aa, Artificial Sequence
  source: /note=“Description of Artificial Sequence Synthetic peptide”
ARDRGVEGAYGMDV

SEQ ID NO: 137 - protein, 7 aa, Artificial Sequence
  source: /note=“Description of Artificial Sequence Synthetic peptide”
QRVRSSY

SEQ ID NO: 138 - protein, 3 aa, Artificial Sequence
  source: /note=“Description of Artificial Sequence Synthetic peptide”
GAS

SEQ ID NO: 139 - protein, 11 aa, Artificial Sequence
  source: /note=“Description of Artificial Sequence Synthetic peptide”
QQYGSSPPRII

SEQ ID NO: 140 - protein, 8 aa, Artificial Sequence
  source: /note=“Description of Artificial Sequence Synthetic peptide”
GYTFTGYY

SEQ ID NO: 141 - protein, 8 aa, Artificial Sequence
  source: /note=“Description of Artificial Sequence Synthetic peptide”
INPNSGGT

SEQ ID NO: 142 - protein, 16 aa, Artificial Sequence
  source: /note=“Description of Artificial Sequence Synthetic peptide”
ARGGDCSSTSCYDPDY

SEQ ID NO: 143 - protein, 8 aa, Artificial Sequence
  source: /note=“Description of Artificial Sequence Synthetic peptide”
GGSIASNY

SEQ ID NO: 144 - protein, 3 aa, Artificial Sequence
  source: /note=“Description of Artificial Sequence Synthetic peptide”
KDN

SEQ ID NO: 145 - protein, 9 aa, Artificial Sequence
  source: /note=“Description of Artificial Sequence Synthetic peptide”
QSYGSGNVV

SEQ ID NO: 146 - protein, 8 aa, Artificial Sequence
  source: /note=“Description of Artificial Sequence Synthetic peptide”
GYTFTSYY

SEQ ID NO: 147 - protein, 8 aa, Artificial Sequence
  source: /note=“Description of Artificial Sequence Synthetic peptide”
INPSGGST

SEQ ID NO: 148 - protein, 15 aa, Artificial Sequence
  source: /note=“Description of Artificial Sequence Synthetic peptide”
AREDHDYSNQGGFDY

SEQ ID NO: 149 - protein, 7 aa, Artificial Sequence
  source: /note=“Description of Artificial Sequence Synthetic peptide”
QSVTSNY

SEQ ID NO: 150 - protein, 3 aa, Artificial Sequence
  source: /note=“Description of Artificial Sequence Synthetic peptide”
GAS

SEQ ID NO: 151 - protein, 8 aa, Artificial Sequence
  source: /note=“Description of Artificial Sequence Synthetic peptide”
QQYGSSPT

SEQ ID NO: 152 - protein, 10 aa, Artificial Sequence
  source: /note=“Description of Artificial Sequence Synthetic peptide”
GDSVSSNSAA

SEQ ID NO: 153 - protein, 9 aa, Artificial Sequence
  source: /note=“Description of Artificial Sequence Synthetic peptide”
TYYRSKWYN

SEQ ID NO: 154 - protein, 16 aa, Artificial Sequence
  source: /note=“Description of Artificial Sequence Synthetic peptide”
AREKIAVAGYYYGMDV

SEQ ID NO: 155 - protein, 6 aa, Artificial Sequence
  source: /note=“Description of Artificial Sequence Synthetic peptide”
KLGDKY

SEQ ID NO: 156 - protein, 3 aa, Artificial Sequence
  source: /note=“Description of Artificial Sequence Synthetic peptide”
QNN

SEQ ID NO: 157 - protein, 9 aa, Artificial Sequence
  source: /note=“Description of Artificial Sequence Synthetic peptide”
QAWDSSAVV

SEQ ID NO: 158 - protein, 10 aa, Artificial Sequence
  source: /note=“Description of Artificial Sequence Synthetic peptide”
GDSVSSNSAA

SEQ ID NO: 159 - protein, 9 aa, Artificial Sequence
  source: /note=“Description of Artificial Sequence Synthetic peptide”
TYYSSKWYN

SEQ ID NO: 160 - protein, 15 aa, Artificial Sequence
  source: /note=“Description of Artificial Sequence Synthetic peptide”
ARGGSSEFYYYGMDV

SEQ ID NO: 161 - protein, 6 aa, Artificial Sequence
  source: /note=“Description of Artificial Sequence Synthetic peptide”
KLGNKY

SEQ ID NO: 162 - protein, 3 aa, Artificial Sequence
  source: /note=“Description of Artificial Sequence Synthetic peptide”
ENN

SEQ ID NO: 163 - protein, 9 aa, Artificial Sequence
  source: /note=“Description of Artificial Sequence Synthetic peptide”
QAWDSSTAV

SEQ ID NO: 164 - protein, 8 aa, Artificial Sequence
  source: /note=“Description of Artificial Sequence Synthetic peptide”
GFTFDDYA

SEQ ID NO: 165 - protein, 8 aa, Artificial Sequence
  source: /note=“Description of Artificial Sequence Synthetic peptide”
ISWNSGSI

SEQ ID NO: 166 - protein, 11 aa, Artificial Sequence
  source: /note=“Description of Artificial Sequence Synthetic peptide”
AKDIAAGGLDS

SEQ ID NO: 167 - protein, 6 aa, Artificial Sequence
  source: /note=“Description of Artificial Sequence Synthetic peptide”
QSISSY

SEQ ID NO: 168 - protein, 3 aa, Artificial Sequence
  source: /note=“Description of Artificial Sequence Synthetic peptide”
AAS

SEQ ID NO: 169 - protein, 9 aa, Artificial Sequence
  source: /note=“Description of Artificial Sequence Synthetic peptide”
QQSYSTSWT

SEQ ID NO: 170 - protein, 8 aa, Artificial Sequence
  source: /note=“Description of Artificial Sequence Synthetic peptide”
GYTFTSYG

SEQ ID NO: 171 - protein, 8 aa, Artificial Sequence
  source: /note=“Description of Artificial Sequence Synthetic peptide”
ISAYNGNT

SEQ ID NO: 172 - protein, 11 aa, Artificial Sequence
  source: /note=“Description of Artificial Sequence Synthetic peptide”
ARGLGDSSSSY

SEQ ID NO: 173 - protein, 8 aa, Artificial Sequence
  source: /note=“Description of Artificial Sequence Synthetic peptide”
TSNIGANH

SEQ ID NO: 174 - protein, 3 aa, Artificial Sequence
  source: /note=“Description of Artificial Sequence Synthetic peptide”
TKN

SEQ ID NO: 175 - protein, 11 aa, Artificial Sequence
  source: /note=“Description of Artificial Sequence Synthetic peptide”
AAWDDSLRGWT

SEQ ID NO: 176 - protein, 8 aa, Artificial Sequence
  source: /note=“Description of Artificial Sequence Synthetic peptide”
GYSFTSYW

SEQ ID NO: 177 - protein, 8 aa, Artificial Sequence
  source: /note=“Description of Artificial Sequence Synthetic peptide”
IYPGDSDT

SEQ ID NO: 178 - protein, 11 aa, Artificial Sequence
  source: /note=“Description of Artificial Sequence Synthetic peptide”
ASGASPYYFDY

SEQ ID NO: 179 - protein, 6 aa, Artificial Sequence
  source: /note=“Description of Artificial Sequence Synthetic peptide”
SLRSYY

SEQ ID NO: 180 - protein, 3 aa, Artificial Sequence
  source: /note=“Description of Artificial Sequence Synthetic peptide”
GKN

SEQ ID NO: 181 - protein, 11 aa, Artificial Sequence
  source: /note=“Description of Artificial Sequence Synthetic peptide”
NSRDSSGNHWV

SEQ ID NO: 182 - protein, 8 aa, Artificial Sequence
  source: /note=“Description of Artificial Sequence Synthetic peptide”
GYTFTSYG

SEQ ID NO: 183 - protein, 8 aa, Artificial Sequence
  source: /note=“Description of Artificial Sequence Synthetic peptide”
ISAYNGNT

SEQ ID NO: 184 - protein, 17 aa, Artificial Sequence
  source: /note=“Description of Artificial Sequence Synthetic peptide”
ARDPVYSSSWGGYAFDI

SEQ ID NO: 185 - protein, 6 aa, Artificial Sequence
  source: /note=“Description of Artificial Sequence Synthetic peptide”
QGVNSD

SEQ ID NO: 186 - protein, 3 aa, Artificial Sequence
  source: /note=“Description of Artificial Sequence Synthetic peptide”
GAS

SEQ ID NO: 187 - protein, 9 aa, Artificial Sequence
  source: /note=“Description of Artificial Sequence Synthetic peptide”
QQYNNWPWT

SEQ ID NO: 188 - protein, 8 aa, Artificial Sequence
  source: /note=“Description of Artificial Sequence Synthetic peptide”
GFTFSSYP

SEQ ID NO: 189 - protein, 8 aa, Artificial Sequence
  source: /note=“Description of Artificial Sequence Synthetic peptide”
ISYDGSNK

SEQ ID NO: 190 - protein, 12 aa, Artificial Sequence
  source: /note=“Description of Artificial Sequence Synthetic peptide”
TRVGSGGWTPDY

SEQ ID NO: 191 - protein, 9 aa, Artificial Sequence
  source: /note=“Description of Artificial Sequence Synthetic peptide”
SSDVGGYNY

SEQ ID NO: 192 - protein, 3 aa, Artificial Sequence
  source: /note=“Description of Artificial Sequence Synthetic peptide”
EVS

SEQ ID NO: 193 - protein, 11 aa, Artificial Sequence
  source: /note=“Description of Artificial Sequence Synthetic peptide”
SSYTSSSTLVV

SEQ ID NO: 194 - protein, 8 aa, Artificial Sequence
  source: /note=“Description of Artificial Sequence Synthetic peptide”
GFTVSSNY

SEQ ID NO: 195 - protein, 7 aa, Artificial Sequence
  source: /note=“Description of Artificial Sequence Synthetic peptide”
IYSGGST

SEQ ID NO: 196 - protein, 11 aa, Artificial Sequence
  source: /note=“Description of Artificial Sequence Synthetic peptide”
ARDTASGGMDV

SEQ ID NO: 197 - protein, 6 aa, Artificial Sequence
  source: /note=“Description of Artificial Sequence Synthetic peptide”
QSVSSN

SEQ ID NO: 198 - protein, 3 aa, Artificial Sequence
  source: /note=“Description of Artificial Sequence Synthetic peptide”
GAS

SEQ ID NO: 199 - protein, 9 aa, Artificial Sequence
  source: /note=“Description of Artificial Sequence Synthetic peptide”
QQYGYSQIT

SEQ ID NO: 200 - protein, 8 aa, Artificial Sequence
  source: /note=“Description of Artificial Sequence Synthetic peptide”
GFTFSSYG

SEQ ID NO: 201 - protein, 8 aa, Artificial Sequence
  source: /note=“Description of Artificial Sequence Synthetic peptide”
IWYDGSNK

SEQ ID NO: 202 - protein, 12 aa, Artificial Sequence
  source: /note=“Description of Artificial Sequence Synthetic peptide”
AREVVGSYYLDY

SEQ ID NO: 203 - protein, 9 aa, Artificial Sequence
  source: /note=“Description of Artificial Sequence Synthetic peptide”
SSDIGGYKY

SEQ ID NO: 204 - protein, 3 aa, Artificial Sequence
  source: /note=“Description of Artificial Sequence Synthetic peptide”
DVT

SEQ ID NO: 205 - protein, 11 aa, Artificial Sequence
  source: /note=“Description of Artificial Sequence Synthetic peptide”
GSYSSSSSHYV

SEQ ID NO: 206 - protein, 8 aa, Artificial Sequence
  source: /note=“Description of Artificial Sequence Synthetic peptide”
GFTFSSYW

SEQ ID NO: 207 - protein, 8 aa, Artificial Sequence
  source: /note=“Description of Artificial Sequence Synthetic peptide”
IKQDGSEK

SEQ ID NO: 208 - protein, 14 aa, Artificial Sequence
  source: /note=“Description of Artificial Sequence Synthetic peptide”
ARDLHCGSSCGPEA

SEQ ID NO: 209 - protein, 6 aa, Artificial Sequence
  source: /note=“Description of Artificial Sequence Synthetic peptide”
QTISSY

SEQ ID NO: 210 - protein, 3 aa, Artificial Sequence
  source: /note=“Description of Artificial Sequence Synthetic peptide”
GAS

SEQ ID NO: 211 - protein, 9 aa, Artificial Sequence
  source: /note=“Description of Artificial Sequence Synthetic peptide”
QQSYSTPQT

SEQ ID NO: 212 - protein, 9 aa, Artificial Sequence
  source: /note=“Description of Artificial Sequence Synthetic peptide”
GDSISSGYW

SEQ ID NO: 213 - protein, 10 aa, Artificial Sequence
  source: /note=“Description of Artificial Sequence Synthetic peptide”
GDSVSSNSAA

SEQ ID NO: 214 - protein, 10 aa, Artificial Sequence
  source: /note=“Description of Artificial Sequence Synthetic peptide”
GDSVSSNSAA

SEQ ID NO: 215 - protein, 10 aa, Artificial Sequence
  source: /note=“Description of Artificial Sequence Synthetic peptide”
GDSVSSNSAA

SEQ ID NO: 216 - protein, 10 aa, Artificial Sequence
  source: /note=“Description of Artificial Sequence Synthetic peptide”
GDSVSSNSAA

SEQ ID NO: 217 - protein, 10 aa, Artificial Sequence
  source: /note=“Description of Artificial Sequence Synthetic peptide”
GDSVSSNSAA

SEQ ID NO: 218 - protein, 10 aa, Artificial Sequence
  source: /note=“Description of Artificial Sequence Synthetic peptide”
GDSVSSNSAA

SEQ ID NO: 219 - protein, 10 aa, Artificial Sequence
  source: /note=“Description of Artificial Sequence Synthetic peptide”
GDSVSSNSAA

SEQ ID NO: 220 - protein, 10 aa, Artificial Sequence
  source: /note=“Description of Artificial Sequence Synthetic peptide”
GDSVSSNSAA

SEQ ID NO: 221 - protein, 10 aa, Artificial Sequence
  source: /note=“Description of Artificial Sequence Synthetic peptide”
GDSVSSDSAS

SEQ ID NO: 222 - protein, 10 aa, Artificial Sequence
  source: /note=“Description of Artificial Sequence Synthetic peptide”
GGSISGSNYY

SEQ ID NO: 223 - protein, 9 aa, Artificial Sequence
  source: /note=“Description of Artificial Sequence Synthetic peptide”
GGSISSSNW

SEQ ID NO: 224 - protein, 9 aa, Artificial Sequence
  source: /note=“Description of Artificial Sequence Synthetic peptide”
GGSISSSNW

SEQ ID NO: 225 - protein, 9 aa, Artificial Sequence
  source: /note=“Description of Artificial Sequence Synthetic peptide”
GGSISSSNW

SEQ ID NO: 226 - protein, 9 aa, Artificial Sequence
  source: /note=“Description of Artificial Sequence Synthetic peptide”
GGSISSSNW

SEQ ID NO: 227 - protein, 10 aa, Artificial Sequence
  source: /note=“Description of Artificial Sequence Synthetic peptide”
GGSVSSNSAA

SEQ ID NO: 228 - protein, 8 aa, Artificial Sequence
  source: /note=“Description of Artificial Sequence Synthetic peptide”
GGTFSSYA

SEQ ID NO: 229 - protein, 8 aa, Artificial Sequence
  source: /note=“Description of Artificial Sequence Synthetic peptide”
GGTFSSYA

SEQ ID NO: 230 - protein, 8 aa, Artificial Sequence
  source: /note=“Description of Artificial Sequence Synthetic peptide”
GGTFSSYA

SEQ ID NO: 231 - protein, 8 aa, Artificial Sequence
  source: /note=“Description of Artificial Sequence Synthetic peptide”
GFTFNTYA

SEQ ID NO: 232 - protein, 8 aa, Artificial Sequence
  source: /note=“Description of Artificial Sequence Synthetic peptide”
GFTFNTYA

SEQ ID NO: 233 - protein, 8 aa, Artificial Sequence
  source: /note=“Description of Artificial Sequence Synthetic peptide”
GFTFNTYA

SEQ ID NO: 234 - protein, 8 aa, Artificial Sequence
  source: /note=“Description of Artificial Sequence Synthetic peptide”
GFTFDDYA

SEQ ID NO: 235 - protein, 8 aa, Artificial Sequence
  source: /note=“Description of Artificial Sequence Synthetic peptide”
GFTFDDYA

SEQ ID NO: 236 - protein, 8 aa, Artificial Sequence
  source: /note=“Description of Artificial Sequence Synthetic peptide”
GFTFDDYA

SEQ ID NO: 237 - protein, 8 aa, Artificial Sequence
  source: /note=“Description of Artificial Sequence Synthetic peptide”
GFTFDDYA

SEQ ID NO: 238 - protein, 8 aa, Artificial Sequence
  source: /note=“Description of Artificial Sequence Synthetic peptide”
GFTFGNHG

SEQ ID NO: 239 - protein, 8 aa, Artificial Sequence
  source: /note=“Description of Artificial Sequence Synthetic peptide”
GFTFSRYG

SEQ ID NO: 240 - protein, 8 aa, Artificial Sequence
  source: /note=“Description of Artificial Sequence Synthetic peptide”
GFTFSNAW

SEQ ID NO: 241 - protein, 8 aa, Artificial Sequence
  source: /note=“Description of Artificial Sequence Synthetic peptide”
GFTFSNAW

SEQ ID NO: 242 - protein, 8 aa, Artificial Sequence
  source: /note=“Description of Artificial Sequence Synthetic peptide”
GFTFSNAW

SEQ ID NO: 243 - protein, 8 aa, Artificial Sequence
  source: /note=“Description of Artificial Sequence Synthetic peptide”
GFTFSNYA

SEQ ID NO: 244 - protein, 8 aa, Artificial Sequence
  source: /note=“Description of Artificial Sequence Synthetic peptide”
GFTFSNYA

SEQ ID NO: 245 - protein, 8 aa, Artificial Sequence
  source: /note=“Description of Artificial Sequence Synthetic peptide”
GFTFSNYA

SEQ ID NO: 246 - protein, 8 aa, Artificial Sequence
  source: /note=“Description of Artificial Sequence Synthetic peptide”
GFTFSNYA

SEQ ID NO: 247 - protein, 8 aa, Artificial Sequence
  source: /note=“Description of Artificial Sequence Synthetic peptide”
GFTFSNYA

SEQ ID NO: 248 - protein, 8 aa, Artificial Sequence
  source: /note=“Description of Artificial Sequence Synthetic peptide”
GFTFSNYA

SEQ ID NO: 249 - protein, 8 aa, Artificial Sequence
  source: /note=“Description of Artificial Sequence Synthetic peptide”
GFTFSNYA

SEQ ID NO: 250 - protein, 8 aa, Artificial Sequence
  source: /note=“Description of Artificial Sequence Synthetic peptide”
GFTFSNYA

SEQ ID NO: 251 - protein, 8 aa, Artificial Sequence
  source: /note=“Description of Artificial Sequence Synthetic peptide”
GFTFSNYG

SEQ ID NO: 252 - protein, 8 aa, Artificial Sequence
  source: /note=“Description of Artificial Sequence Synthetic peptide”
GFTFSNYG

SEQ ID NO: 253 - protein, 8 aa, Artificial Sequence
  source: /note=“Description of Artificial Sequence Synthetic peptide”
GFTFSDYA

SEQ ID NO: 254 - protein, 8 aa, Artificial Sequence
  source: /note=“Description of Artificial Sequence Synthetic peptide”
GFTFSDYY

SEQ ID NO: 255 - protein, 8 aa, Artificial Sequence
  source: /note=“Description of Artificial Sequence Synthetic peptide”
GFTFSSYA

SEQ ID NO: 256 - protein, 8 aa, Artificial Sequence
  source: /note=“Description of Artificial Sequence Synthetic peptide”
GFTFSSYA

SEQ ID NO: 257 - protein, 8 aa, Artificial Sequence
  source: /note=“Description of Artificial Sequence Synthetic peptide”
GFTFSSYA

SEQ ID NO: 258 - protein, 8 aa, Artificial Sequence
  source: /note=“Description of Artificial Sequence Synthetic peptide”
GFTFSSYA

SEQ ID NO: 259 - protein, 8 aa, Artificial Sequence
  source: /note=“Description of Artificial Sequence Synthetic peptide”
GFTFSSYA

SEQ ID NO: 260 - protein, 8 aa, Artificial Sequence
  source: /note=“Description of Artificial Sequence Synthetic peptide”
GFTFSSYA

SEQ ID NO: 261 - protein, 8 aa, Artificial Sequence
  source: /note=“Description of Artificial Sequence Synthetic peptide”
GFTFSSYA

SEQ ID NO: 262 - protein, 8 aa, Artificial Sequence
  source: /note=“Description of Artificial Sequence Synthetic peptide”
GFTFSSYA

SEQ ID NO: 263 - protein, 8 aa, Artificial Sequence
  source: /note=“Description of Artificial Sequence Synthetic peptide”
GFTFSSYA

SEQ ID NO: 264 - protein, 8 aa, Artificial Sequence
  source: /note=“Description of Artificial Sequence Synthetic peptide”
GFTFSSYA

SEQ ID NO: 265 - protein, 8 aa, Artificial Sequence
  source: /note=“Description of Artificial Sequence Synthetic peptide”
GFTFSSYA

SEQ ID NO: 266 - protein, 8 aa, Artificial Sequence
  source: /note=“Description of Artificial Sequence Synthetic peptide”
GFTFSSYA

SEQ ID NO: 267 - protein, 8 aa, Artificial Sequence
  source: /note=“Description of Artificial Sequence Synthetic peptide”
GFTFSSYA

SEQ ID NO: 268 - protein, 8 aa, Artificial Sequence
  source: /note=“Description of Artificial Sequence Synthetic peptide”
GFTFSSYA

SEQ ID NO: 269 - protein, 8 aa, Artificial Sequence
  source: /note=“Description of Artificial Sequence Synthetic peptide”
GFTFSSYA

SEQ ID NO: 270 - protein, 8 aa, Artificial Sequence
  source: /note=“Description of Artificial Sequence Synthetic peptide”
GFTFSSYA

SEQ ID NO: 271 - protein, 8 aa, Artificial Sequence
  source: /note=“Description of Artificial Sequence Synthetic peptide”
GFTFSSYA

SEQ ID NO: 272 - protein, 8 aa, Artificial Sequence
  source: /note=“Description of Artificial Sequence Synthetic peptide”
GFTFSSYA

SEQ ID NO: 273 - protein, 8 aa, Artificial Sequence
  source: /note=“Description of Artificial Sequence Synthetic peptide”
GFTFSSYA

SEQ ID NO: 274 - protein, 8 aa, Artificial Sequence
  source: /note=“Description of Artificial Sequence Synthetic peptide”
GFTFSSYA

SEQ ID NO: 275 - protein, 8 aa, Artificial Sequence
  source: /note=“Description of Artificial Sequence Synthetic peptide”
GFTFSSYA

SEQ ID NO: 276 - protein, 8 aa, Artificial Sequence
  source: /note=“Description of Artificial Sequence Synthetic peptide”
GFTFSSYA

SEQ ID NO: 277 - protein, 8 aa, Artificial Sequence
  source: /note=“Description of Artificial Sequence Synthetic peptide”
GFTFSSYA

SEQ ID NO: 278 - protein, 8 aa, Artificial Sequence
  source: /note=“Description of Artificial Sequence Synthetic peptide”
GFTFSSYA

SEQ ID NO: 279 - protein, 8 aa, Artificial Sequence
  source: /note=“Description of Artificial Sequence Synthetic peptide”
GFTFSSYA

SEQ ID NO: 280 - protein, 8 aa, Artificial Sequence
  source: /note=“Description of Artificial Sequence Synthetic peptide”
GFTFSSYA

SEQ ID NO: 281 - protein, 8 aa, Artificial Sequence
  source: /note=“Description of Artificial Sequence Synthetic peptide”
GFTFSSYA

SEQ ID NO: 282 - protein, 8 aa, Artificial Sequence
  source: /note=“Description of Artificial Sequence Synthetic peptide”
GFTFSSYA

SEQ ID NO: 283 - protein, 8 aa, Artificial Sequence
  source: /note=“Description of Artificial Sequence Synthetic peptide”
GFTFSSYG

SEQ ID NO: 284 - protein, 8 aa, Artificial Sequence
  source: /note=“Description of Artificial Sequence Synthetic peptide”
GFTFSSYG

SEQ ID NO: 285 - protein, 8 aa, Artificial Sequence
  source: /note=“Description of Artificial Sequence Synthetic peptide”
GFTFSSYG

SEQ ID NO: 286 - protein, 8 aa, Artificial Sequence
  source: /note=“Description of Artificial Sequence Synthetic peptide”
GFTFSSYG

SEQ ID NO: 287 - protein, 8 aa, Artificial Sequence
  source: /note=“Description of Artificial Sequence Synthetic peptide”
GFTFSSYG

SEQ ID NO: 288 - protein, 8 aa, Artificial Sequence
  source: /note=“Description of Artificial Sequence Synthetic peptide”
GFTFSSYG

SEQ ID NO: 289 - protein, 8 aa, Artificial Sequence
  source: /note=“Description of Artificial Sequence Synthetic peptide”
GFTFSSYG

SEQ ID NO: 290 - protein, 8 aa, Artificial Sequence
  source: /note=“Description of Artificial Sequence Synthetic peptide”
GFTFSSYG

SEQ ID NO: 291 - protein, 8 aa, Artificial Sequence
  source: /note=“Description of Artificial Sequence Synthetic peptide”
GFTFSSYG

SEQ ID NO: 292 - protein, 8 aa, Artificial Sequence
  source: /note=“Description of Artificial Sequence Synthetic peptide”
GFTFSSYP

SEQ ID NO: 293 - protein, 8 aa, Artificial Sequence
  source: /note=“Description of Artificial Sequence Synthetic peptide”
GFTFSSYP

SEQ ID NO: 294 - protein, 8 aa, Artificial Sequence
  source: /note=“Description of Artificial Sequence Synthetic peptide”
GFTFSSYP

SEQ ID NO: 295 - protein, 8 aa, Artificial Sequence
  source: /note=“Description of Artificial Sequence Synthetic peptide”
GFTFSSYP

SEQ ID NO: 296 - protein, 8 aa, Artificial Sequence
  source: /note=“Description of Artificial Sequence Synthetic peptide”
GFTFSSYP

SEQ ID NO: 297 - protein, 8 aa, Artificial Sequence
  source: /note=“Description of Artificial Sequence Synthetic peptide”
GFTFSSYP

SEQ ID NO: 298 - protein, 8 aa, Artificial Sequence
  source: /note=“Description of Artificial Sequence Synthetic peptide”
GFTFSSYP

SEQ ID NO: 299 - protein, 8 aa, Artificial Sequence
  source: /note=“Description of Artificial Sequence Synthetic peptide”
GFTFSSYP

SEQ ID NO: 300 - protein, 8 aa, Artificial Sequence
  source: /note=“Description of Artificial Sequence Synthetic peptide”
GFTFSSYP

SEQ ID NO: 301 - protein, 8 aa, Artificial Sequence
  source: /note=“Description of Artificial Sequence Synthetic peptide”
GFTFSSYP

SEQ ID NO: 302 - protein, 8 aa, Artificial Sequence
  source: /note=“Description of Artificial Sequence Synthetic peptide”
GFTFSSYP

SEQ ID NO: 303 - protein, 8 aa, Artificial Sequence
  source: /note=“Description of Artificial Sequence Synthetic peptide”
GFTFSSYP

SEQ ID NO: 304 - protein, 8 aa, Artificial Sequence
  source: /note=“Description of Artificial Sequence Synthetic peptide”
GFTFSSYS

SEQ ID NO: 305 - protein, 8 aa, Artificial Sequence
  source: /note=“Description of Artificial Sequence Synthetic peptide”
GFTFSSYW

SEQ ID NO: 306 - protein, 8 aa, Artificial Sequence
  source: /note=“Description of Artificial Sequence Synthetic peptide”
GFTFSSYW

SEQ ID NO: 307 - protein, 8 aa, Artificial Sequence
  source: /note=“Description of Artificial Sequence Synthetic peptide”
GFTFSSYW

SEQ ID NO: 308 - protein, 8 aa, Artificial Sequence
  source: /note=“Description of Artificial Sequence Synthetic peptide”
GFTFSSYW

SEQ ID NO: 309 - protein, 8 aa, Artificial Sequence
  source: /note=“Description of Artificial Sequence Synthetic peptide”
GFTFSTYA

SEQ ID NO: 310 - protein, 8 aa, Artificial Sequence
  source: /note=“Description of Artificial Sequence Synthetic peptide”
GFTFSTYA

SEQ ID NO: 311 - protein, 8 aa, Artificial Sequence
  source: /note=“Description of Artificial Sequence Synthetic peptide”
GFTFSTYA

SEQ ID NO: 312 - protein, 8 aa, Artificial Sequence
  source: /note=“Description of Artificial Sequence Synthetic peptide”
GFTFSTYA

SEQ ID NO: 313 - protein, 8 aa, Artificial Sequence
  source: /note=“Description of Artificial Sequence Synthetic peptide”
GFTFSTYA

SEQ ID NO: 314 - protein, 8 aa, Artificial Sequence
  source: /note=“Description of Artificial Sequence Synthetic peptide”
GFTFSTYA

SEQ ID NO: 315 - protein, 8 aa, Artificial Sequence
  source: /note=“Description of Artificial Sequence Synthetic peptide”
GFTFSTYA

SEQ ID NO: 316 - protein, 8 aa, Artificial Sequence
  source: /note=“Description of Artificial Sequence Synthetic peptide”
GFTFSTYA

SEQ ID NO: 317 - protein, 8 aa, Artificial Sequence
  source: /note=“Description of Artificial Sequence Synthetic peptide”
GFTFSTYA

SEQ ID NO: 318 - protein, 8 aa, Artificial Sequence
  source: /note=“Description of Artificial Sequence Synthetic peptide”
GFTFSTYA

SEQ ID NO: 319 - protein, 8 aa, Artificial Sequence
  source: /note=“Description of Artificial Sequence Synthetic peptide”
GFTVSSNY

SEQ ID NO: 320 - protein, 8 aa, Artificial Sequence
  source: /note=“Description of Artificial Sequence Synthetic peptide”
GFTVSSNY

SEQ ID NO: 321 - protein, 8 aa, Artificial Sequence
  source: /note=“Description of Artificial Sequence Synthetic peptide”
GFTVSSNY

SEQ ID NO: 322 - protein, 8 aa, Artificial Sequence
  source: /note=“Description of Artificial Sequence Synthetic peptide”
GFTVSSNY

SEQ ID NO: 323 - protein, 8 aa, Artificial Sequence
  source: /note=“Description of Artificial Sequence Synthetic peptide”
GFTVSSNY

SEQ ID NO: 324 - protein, 8 aa, Artificial Sequence
  source: /note=“Description of Artificial Sequence Synthetic peptide”
GFTVSSNY

SEQ ID NO: 325 - protein, 8 aa, Artificial Sequence
  source: /note=“Description of Artificial Sequence Synthetic peptide”
GFTVSSNY

SEQ ID NO: 326 - protein, 8 aa, Artificial Sequence
  source: /note=“Description of Artificial Sequence Synthetic peptide”
GFTVSSNY

SEQ ID NO: 327 - protein, 8 aa, Artificial Sequence
  source: /note=“Description of Artificial Sequence Synthetic peptide”
GFTVSSNY

SEQ ID NO: 328 - protein, 8 aa, Artificial Sequence
  source: /note=“Description of Artificial Sequence Synthetic peptide”
GYRFTSYW

SEQ ID NO: 329 - protein, 8 aa, Artificial Sequence
  source: /note=“Description of Artificial Sequence Synthetic peptide”
GYSFTRYW

SEQ ID NO: 330 - protein, 8 aa, Artificial Sequence
  source: /note=“Description of Artificial Sequence Synthetic peptide”
GYSFTSYW

SEQ ID NO: 331 - protein, 8 aa, Artificial Sequence
  source: /note=“Description of Artificial Sequence Synthetic peptide”
GYSFTSYW

SEQ ID NO: 332 - protein, 8 aa, Artificial Sequence
  source: /note=“Description of Artificial Sequence Synthetic peptide”
GYSFTSYW

SEQ ID NO: 333 - protein, 8 aa, Artificial Sequence
  source: /note=“Description of Artificial Sequence Synthetic peptide”
GYSFTSYW

SEQ ID NO: 334 - protein, 8 aa, Artificial Sequence
  source: /note=“Description of Artificial Sequence Synthetic peptide”
GYSFTSYW

SEQ ID NO: 335 - protein, 8 aa, Artificial Sequence
  source: /note=“Description of Artificial Sequence Synthetic peptide”
GYSFTSYW

SEQ ID NO: 336 - protein, 8 aa, Artificial Sequence
  source: /note=“Description of Artificial Sequence Synthetic peptide”
GYSFTSYW

SEQ ID NO: 337 - protein, 8 aa, Artificial Sequence
  source: /note=“Description of Artificial Sequence Synthetic peptide”
GYTFTRNA

SEQ ID NO: 338 - protein, 8 aa, Artificial Sequence
  source: /note=“Description of Artificial Sequence Synthetic peptide”
GYTFTGYY

SEQ ID NO: 339 - protein, 8 aa, Artificial Sequence
  source: /note=“Description of Artificial Sequence Synthetic peptide”
GYTFTGYY

SEQ ID NO: 340 - protein, 8 aa, Artificial Sequence
  source: /note=“Description of Artificial Sequence Synthetic peptide”
GYTFTGYY

SEQ ID NO: 341 - protein, 8 aa, Artificial Sequence
  source: /note=“Description of Artificial Sequence Synthetic peptide”
GYTFTGYY

SEQ ID NO: 342 - protein, 8 aa, Artificial Sequence
  source: /note=“Description of Artificial Sequence Synthetic peptide”
GYTFTGYY

SEQ ID NO: 343 - protein, 8 aa, Artificial Sequence
  source: /note=“Description of Artificial Sequence Synthetic peptide”
GYTFTGYY

SEQ ID NO: 344 - protein, 8 aa, Artificial Sequence
  source: /note=“Description of Artificial Sequence Synthetic peptide”
GYTFTSYA

SEQ ID NO: 345 - protein, 8 aa, Artificial Sequence
  source: /note=“Description of Artificial Sequence Synthetic peptide”
GYTFTSYG

SEQ ID NO: 346 - protein, 8 aa, Artificial Sequence
  source: /note=“Description of Artificial Sequence Synthetic peptide”
GYTFTSYG

SEQ ID NO: 347 - protein, 8 aa, Artificial Sequence
  source: /note=“Description of Artificial Sequence Synthetic peptide”
GYTFTSYG

SEQ ID NO: 348 - protein, 8 aa, Artificial Sequence
  source: /note=“Description of Artificial Sequence Synthetic peptide”
GYTFTSYG

SEQ ID NO: 349 - protein, 8 aa, Artificial Sequence
  source: /note=“Description of Artificial Sequence Synthetic peptide”
GYTFTSYG

SEQ ID NO: 350 - protein, 8 aa, Artificial Sequence
  source: /note=“Description of Artificial Sequence Synthetic peptide”
GYTFTSYG

SEQ ID NO: 351 - protein, 8 aa, Artificial Sequence
  source: /note=“Description of Artificial Sequence Synthetic peptide”
GYTFTSYG

SEQ ID NO: 352 - protein, 8 aa, Artificial Sequence
  source: /note=“Description of Artificial Sequence Synthetic peptide”
GYTFTSYY

SEQ ID NO: 353 - protein, 10 aa, Artificial Sequence
  source: /note=“Description of Artificial Sequence Synthetic peptide”
GDSVSSNSAA

SEQ ID NO: 354 - protein, 10 aa, Artificial Sequence
  source: /note=“Description of Artificial Sequence Synthetic peptide”
GDSVSSNSAA

SEQ ID NO: 355 - protein, 10 aa, Artificial Sequence
  source: /note=“Description of Artificial Sequence Synthetic peptide”
GDSVSSNSAA

SEQ ID NO: 356 - protein, 8 aa, Artificial Sequence
  source: /note=“Description of Artificial Sequence Synthetic peptide”
GFTFDDYA

SEQ ID NO: 357 - protein, 8 aa, Artificial Sequence
  source: /note=“Description of Artificial Sequence Synthetic peptide”
GFTFSNAW

SEQ ID NO: 358 - protein, 8 aa, Artificial Sequence
  source: /note=“Description of Artificial Sequence Synthetic peptide”
GFTFSNAW

SEQ ID NO: 359 - protein, 8 aa, Artificial Sequence
  source: /note=“Description of Artificial Sequence Synthetic peptide”
GFTFSSYA

SEQ ID NO: 360 - protein, 8 aa, Artificial Sequence
  source: /note=“Description of Artificial Sequence Synthetic peptide”
GFTFSSYG

SEQ ID NO: 361 - protein, 8 aa, Artificial Sequence
  source: /note=“Description of Artificial Sequence Synthetic peptide”
GFTFSSYG

SEQ ID NO: 362 - protein, 8 aa, Artificial Sequence
  source: /note=“Description of Artificial Sequence Synthetic peptide”
GFTFSSYG

SEQ ID NO: 363 - protein, 8 aa, Artificial Sequence
  source: /note=“Description of Artificial Sequence Synthetic peptide”
GFTFSSYG

SEQ ID NO: 364 - protein, 8 aa, Artificial Sequence
  source: /note=“Description of Artificial Sequence Synthetic peptide”
GFTFSSYP

SEQ ID NO: 365 - protein, 8 aa, Artificial Sequence
  source: /note=“Description of Artificial Sequence Synthetic peptide”
GFTFSSYP

SEQ ID NO: 366 - protein, 8 aa, Artificial Sequence
  source: /note=“Description of Artificial Sequence Synthetic peptide”
GFTFSSYP

SEQ ID NO: 367 - protein, 8 aa, Artificial Sequence
  source: /note=“Description of Artificial Sequence Synthetic peptide”
GFTFSSYP

SEQ ID NO: 368 - protein, 8 aa, Artificial Sequence
  source: /note=“Description of Artificial Sequence Synthetic peptide”
GFTFSSYP

SEQ ID NO: 369 - protein, 8 aa, Artificial Sequence
  source: /note=“Description of Artificial Sequence Synthetic peptide”
GFTFSSYP

SEQ ID NO: 370 - protein, 8 aa, Artificial Sequence
  source: /note=“Description of Artificial Sequence Synthetic peptide”
GFTFSSYS

SEQ ID NO: 371 - protein, 8 aa, Artificial Sequence
  source: /note=“Description of Artificial Sequence Synthetic peptide”
GFTFSSYW

SEQ ID NO: 372 - protein, 8 aa, Artificial Sequence
  source: /note=“Description of Artificial Sequence Synthetic peptide”
GFTVSSNY

SEQ ID NO: 373 - protein, 8 aa, Artificial Sequence
  source: /note=“Description of Artificial Sequence Synthetic peptide”
GFTVSSNY

SEQ ID NO: 374 - protein, 8 aa, Artificial Sequence
  source: /note=“Description of Artificial Sequence Synthetic peptide”
GFTVSSNY

SEQ ID NO: 375 - protein, 8 aa, Artificial Sequence
  source: /note=“Description of Artificial Sequence Synthetic peptide”
GFTVSSNY

SEQ ID NO: 376 - protein, 8 aa, Artificial Sequence
  source: /note=“Description of Artificial Sequence Synthetic peptide”
GFTVSSNY

SEQ ID NO: 377 - protein, 8 aa, Artificial Sequence
  source: /note=“Description of Artificial Sequence Synthetic peptide”
GYSFTSYW

SEQ ID NO: 378 - protein, 8 aa, Artificial Sequence
  source: /note=“Description of Artificial Sequence Synthetic peptide”
GYTFTGYY

SEQ ID NO: 379 - protein, 8 aa, Artificial Sequence
  source: /note=“Description of Artificial Sequence Synthetic peptide”
GYTFTSYG

SEQ ID NO: 380 - protein, 8 aa, Artificial Sequence
  source: /note=“Description of Artificial Sequence Synthetic peptide”
GYTFTSYG

SEQ ID NO: 381 - protein, 8 aa, Artificial Sequence
  source: /note=“Description of Artificial Sequence Synthetic peptide”
GYTFTSYY

SEQ ID NO: 382 - protein, 8 aa, Artificial Sequence
  source: /note=“Description of Artificial Sequence Synthetic peptide”
ISYDGSNK

SEQ ID NO: 383 - protein, 8 aa, Artificial Sequence
  source: /note=“Description of Artificial Sequence Synthetic peptide”
INPNSGGT

SEQ ID NO: 384 - protein, 8 aa, Artificial Sequence
  source: /note=“Description of Artificial Sequence Synthetic peptide”
ISAYNGNT

SEQ ID NO: 385 - protein, 8 aa, Artificial Sequence
  source: /note=“Description of Artificial Sequence Synthetic peptide”
ISGSGGRT

SEQ ID NO: 386 - protein, 8 aa, Artificial Sequence
  source: /note=“Description of Artificial Sequence Synthetic peptide”
ISGSGGST

SEQ ID NO: 387 - protein, 8 aa, Artificial Sequence
  source: /note=“Description of Artificial Sequence Synthetic peptide”
ISGSGGST

SEQ ID NO: 388 - protein, 8 aa, Artificial Sequence
  source: /note=“Description of Artificial Sequence Synthetic peptide”
ISYDGSNK

SEQ ID NO: 389 - protein, 7 aa, Artificial Sequence
  source: /note=“Description of Artificial Sequence Synthetic peptide”
IYSGGST

SEQ ID NO: 390 - protein, 9 aa, Artificial Sequence
  source: /note=“Description of Artificial Sequence Synthetic peptide”
TYYSSKWYN

SEQ ID NO: 391 - protein, 8 aa, Artificial Sequence
  source: /note=“Description of Artificial Sequence Synthetic peptide”
ISGSGGIT

SEQ ID NO: 392 - protein, 8 aa, Artificial Sequence
  source: /note=“Description of Artificial Sequence Synthetic peptide”
ISGSGGIT

SEQ ID NO: 393 - protein, 8 aa, Artificial Sequence
  source: /note=“Description of Artificial Sequence Synthetic peptide”
ISGSGGST

SEQ ID NO: 394 - protein, 8 aa, Artificial Sequence
  source: /note=“Description of Artificial Sequence Synthetic peptide”
ISGSGGST

SEQ ID NO: 395 - protein, 8 aa, Artificial Sequence
  source: /note=“Description of Artificial Sequence Synthetic peptide”
IYPGDSDT

SEQ ID NO: 396 - protein, 9 aa, Artificial Sequence
  source: /note=“Description of Artificial Sequence Synthetic peptide”
TYYRSKWYN

SEQ ID NO: 397 - protein, 8 aa, Artificial Sequence
  source: /note=“Description of Artificial Sequence Synthetic peptide”
ISGSGGST

SEQ ID NO: 398 - protein, 8 aa, Artificial Sequence
  source: /note=“Description of Artificial Sequence Synthetic peptide”
ISGTGGRT

SEQ ID NO: 399 - protein, 8 aa, Artificial Sequence
  source: /note=“Description of Artificial Sequence Synthetic peptide”
ISYDGSNK

SEQ ID NO: 400 - protein, 8 aa, Artificial Sequence
  source: /note=“Description of Artificial Sequence Synthetic peptide”
IWYDGSNK

SEQ ID NO: 401 - protein, 8 aa, Artificial Sequence
  source: /note=“Description of Artificial Sequence Synthetic peptide”
ISGSGDRT

SEQ ID NO: 402 - protein, 8 aa, Artificial Sequence
  source: /note=“Description of Artificial Sequence Synthetic peptide”
ISGSGDIT

SEQ ID NO: 403 - protein, 8 aa, Artificial Sequence
  source: /note=“Description of Artificial Sequence Synthetic peptide”
ISYDGSNK

SEQ ID NO: 404 - protein, 8 aa, Artificial Sequence
  source: /note=“Description of Artificial Sequence Synthetic peptide”
INAGNGNT

SEQ ID NO: 405 - protein, 8 aa, Artificial Sequence
  source: /note=“Description of Artificial Sequence Synthetic peptide”
ISGSGDRT

SEQ ID NO: 406 - protein, 7 aa, Artificial Sequence
  source: /note=“Description of Artificial Sequence Synthetic peptide”
IYSGGST

SEQ ID NO: 407 - protein, 7 aa, Artificial Sequence
  source: /note=“Description of Artificial Sequence Synthetic peptide”
IYSGGST

SEQ ID NO: 408 - protein, 8 aa, Artificial Sequence
  source: /note=“Description of Artificial Sequence Synthetic peptide”
IKHDGSEQ

SEQ ID NO: 409 - protein, 8 aa, Artificial Sequence
  source: /note=“Description of Artificial Sequence Synthetic peptide”
ISGSGDRT

SEQ ID NO: 410 - protein, 8 aa, Artificial Sequence
  source: /note=“Description of Artificial Sequence Synthetic peptide”
IIPIFGTA

SEQ ID NO: 411 - protein, 8 aa, Artificial Sequence
  source: /note=“Description of Artificial Sequence Synthetic peptide”
ISGSGGRT

SEQ ID NO: 412 - protein, 9 aa, Artificial Sequence
  source: /note=“Description of Artificial Sequence Synthetic peptide”
TYYNSKWYN

SEQ ID NO: 413 - protein, 8 aa, Artificial Sequence
  source: /note=“Description of Artificial Sequence Synthetic peptide”
INTDGGNT

SEQ ID NO: 414 - protein, 8 aa, Artificial Sequence
  source: /note=“Description of Artificial Sequence Synthetic peptide”
ISGSGDIT

SEQ ID NO: 415 - protein, 8 aa, Artificial Sequence
  source: /note=“Description of Artificial Sequence Synthetic peptide”
ISGSGGST

SEQ ID NO: 416 - protein, 7 aa, Artificial Sequence
  source: /note=“Description of Artificial Sequence Synthetic peptide”
IYHSGST

SEQ ID NO: 417 - protein, 8 aa, Artificial Sequence
  source: /note=“Description of Artificial Sequence Synthetic peptide”
IYPGDSDT

SEQ ID NO: 418 - protein, 8 aa, Artificial Sequence
  source: /note=“Description of Artificial Sequence Synthetic peptide”
IYPGDSDT

SEQ ID NO: 419 - protein, 7 aa, Artificial Sequence
  source: /note=“Description of Artificial Sequence Synthetic peptide”
IYSGGST

SEQ ID NO: 420 - protein, 9 aa, Artificial Sequence
  source: /note=“Description of Artificial Sequence Synthetic peptide”
TYYRSKWYN

SEQ ID NO: 421 - protein, 8 aa, Artificial Sequence
  source: /note=“Description of Artificial Sequence Synthetic peptide”
ISYDGSNK

SEQ ID NO: 422 - protein, 8 aa, Artificial Sequence
  source: /note=“Description of Artificial Sequence Synthetic peptide”
ISYDGSNK

SEQ ID NO: 423 - protein, 8 aa, Artificial Sequence
  source: /note=“Description of Artificial Sequence Synthetic peptide”
ISWNSGSI

SEQ ID NO: 424 - protein, 8 aa, Artificial Sequence
  source: /note=“Description of Artificial Sequence Synthetic peptide”
VSGSGTST

SEQ ID NO: 425 - protein, 8 aa, Artificial Sequence
  source: /note=“Description of Artificial Sequence Synthetic peptide”
INPNSGDT

SEQ ID NO: 426 - protein, 8 aa, Artificial Sequence
  source: /note=“Description of Artificial Sequence Synthetic peptide”
INPNSGGT

SEQ ID NO: 427 - protein, 8 aa, Artificial Sequence
  source: /note=“Description of Artificial Sequence Synthetic peptide”
IEPGNGDT

SEQ ID NO: 428 - protein, 8 aa, Artificial Sequence
  source: /note=“Description of Artificial Sequence Synthetic peptide”
IKQDGSEK

SEQ ID NO: 429 - protein, 8 aa, Artificial Sequence
  source: /note=“Description of Artificial Sequence Synthetic peptide”
ISAYNGNT

SEQ ID NO: 430 - protein, 8 aa, Artificial Sequence
  source: /note=“Description of Artificial Sequence Synthetic peptide”
ISAYNGNT

SEQ ID NO: 431 - protein, 8 aa, Artificial Sequence
  source: /note=“Description of Artificial Sequence Synthetic peptide”
ISNDGVNN

SEQ ID NO: 432 - protein, 8 aa, Artificial Sequence
  source: /note=“Description of Artificial Sequence Synthetic peptide”
ISGSGDRT

SEQ ID NO: 433 - protein, 8 aa, Artificial Sequence
  source: /note=“Description of Artificial Sequence Synthetic peptide”
ISGSGGRT

SEQ ID NO: 434 - protein, 8 aa, Artificial Sequence
  source: /note=“Description of Artificial Sequence Synthetic peptide”
ISGSGGRT

SEQ ID NO: 435 - protein, 8 aa, Artificial Sequence
  source: /note=“Description of Artificial Sequence Synthetic peptide”
ISGSGGRT

SEQ ID NO: 436 - protein, 8 aa, Artificial Sequence
  source: /note=“Description of Artificial Sequence Synthetic peptide”
ISGSGGNI

SEQ ID NO: 437 - protein, 8 aa, Artificial Sequence
  source: /note=“Description of Artificial Sequence Synthetic peptide”
ISGSGGIT

SEQ ID NO: 438 - protein, 8 aa, Artificial Sequence
  source: /note=“Description of Artificial Sequence Synthetic peptide”
ISYDGGNK

SEQ ID NO: 439 - protein, 8 aa, Artificial Sequence
  source: /note=“Description of Artificial Sequence Synthetic peptide”
ISYDGSNQ

SEQ ID NO: 440 - protein, 8 aa, Artificial Sequence
  source: /note=“Description of Artificial Sequence Synthetic peptide”
ISYDGSNK

SEQ ID NO: 441 - protein, 8 aa, Artificial Sequence
  source: /note=“Description of Artificial Sequence Synthetic peptide”
ISYDGSNK

SEQ ID NO: 442 - protein, 8 aa, Artificial Sequence
  source: /note=“Description of Artificial Sequence Synthetic peptide”
ISYDGSNK

SEQ ID NO: 443 - protein, 8 aa, Artificial Sequence
  source: /note=“Description of Artificial Sequence Synthetic peptide”
ISYDGSNK

SEQ ID NO: 444 - protein, 8 aa, Artificial Sequence
  source: /note=“Description of Artificial Sequence Synthetic peptide”
ISYDGSNK

SEQ ID NO: 445 - protein, 8 aa, Artificial Sequence
  source: /note=“Description of Artificial Sequence Synthetic peptide”
IWYDGNNK

SEQ ID NO: 446 - protein, 8 aa, Artificial Sequence
  source: /note=“Description of Artificial Sequence Synthetic peptide”
IYPGDSDT

SEQ ID NO: 447 - protein, 8 aa, Artificial Sequence
  source: /note=“Description of Artificial Sequence Synthetic peptide”
IYPGDSDT

SEQ ID NO: 448 - protein, 8 aa, Artificial Sequence
  source: /note=“Description of Artificial Sequence Synthetic peptide”
IYPGDSET

SEQ ID NO: 449 - protein, 7 aa, Artificial Sequence
  source: /note=“Description of Artificial Sequence Synthetic peptide”
IYSGGST

SEQ ID NO: 450 - protein, 7 aa, Artificial Sequence
  source: /note=“Description of Artificial Sequence Synthetic peptide”
IYSGGST

SEQ ID NO: 451 - protein, 7 aa, Artificial Sequence
  source: /note=“Description of Artificial Sequence Synthetic peptide”
IYSGGST

SEQ ID NO: 452 - protein, 7 aa, Artificial Sequence
  source: /note=“Description of Artificial Sequence Synthetic peptide”
FYSGGST

SEQ ID NO: 453 - protein, 8 aa, Artificial Sequence
  source: /note=“Description of Artificial Sequence Synthetic peptide”
ISASGGST

SEQ ID NO: 454 - protein, 8 aa, Artificial Sequence
  source: /note=“Description of Artificial Sequence Synthetic peptide”
ISGSGDRT

SEQ ID NO: 455 - protein, 8 aa, Artificial Sequence
  source: /note=“Description of Artificial Sequence Synthetic peptide”
ISGSGGRT

SEQ ID NO: 456 - protein, 8 aa, Artificial Sequence
  source: /note=“Description of Artificial Sequence Synthetic peptide”
ISGSGGIT

SEQ ID NO: 457 - protein, 8 aa, Artificial Sequence
  source: /note=“Description of Artificial Sequence Synthetic peptide”
ISGSGGST

SEQ ID NO: 458 - protein, 8 aa, Artificial Sequence
  source: /note=“Description of Artificial Sequence Synthetic peptide”
ISWNSGSI

SEQ ID NO: 459 - protein, 8 aa, Artificial Sequence
  source: /note=“Description of Artificial Sequence Synthetic peptide”
ISYDGSNK

SEQ ID NO: 460 - protein, 8 aa, Artificial Sequence
  source: /note=“Description of Artificial Sequence Synthetic peptide”
ISYDGSNK

SEQ ID NO: 461 - protein, 8 aa, Artificial Sequence
  source: /note=“Description of Artificial Sequence Synthetic peptide”
IWYDGSNK

SEQ ID NO: 462 - protein, 8 aa, Artificial Sequence
  source: /note=“Description of Artificial Sequence Synthetic peptide”
INPNSGGT

SEQ ID NO: 463 - protein, 8 aa, Artificial Sequence
  source: /note=“Description of Artificial Sequence Synthetic peptide”
IKQDGSEK

SEQ ID NO: 464 - protein, 8 aa, Artificial Sequence
  source: /note=“Description of Artificial Sequence Synthetic peptide”
ISAYNGNT

SEQ ID NO: 465 - protein, 8 aa, Artificial Sequence
  source: /note=“Description of Artificial Sequence Synthetic peptide”
ISGSGDIT

SEQ ID NO: 466 - protein, 8 aa, Artificial Sequence
  source: /note=“Description of Artificial Sequence Synthetic peptide”
ISGSGDIT

SEQ ID NO: 467 - protein, 8 aa, Artificial Sequence
  source: /note=“Description of Artificial Sequence Synthetic peptide”
ISGSGGRT

SEQ ID NO: 468 - protein, 8 aa, Artificial Sequence
  source: /note=“Description of Artificial Sequence Synthetic peptide”
ISGSGGRT

SEQ ID NO: 469 - protein, 8 aa, Artificial Sequence
  source: /note=“Description of Artificial Sequence Synthetic peptide”
ISGSGGIT

SEQ ID NO: 470 - protein, 8 aa, Artificial Sequence
  source: /note=“Description of Artificial Sequence Synthetic peptide”
ISGTGGRT

SEQ ID NO: 471 - protein, 8 aa, Artificial Sequence
  source: /note=“Description of Artificial Sequence Synthetic peptide”
ISYDATNN

SEQ ID NO: 472 - protein, 7 aa, Artificial Sequence
  source: /note=“Description of Artificial Sequence Synthetic peptide”
IYHSGST

SEQ ID NO: 473 - protein, 8 aa, Artificial Sequence
  source: /note=“Description of Artificial Sequence Synthetic peptide”
IYPGDSDT

SEQ ID NO: 474 - protein, 8 aa, Artificial Sequence
  source: /note=“Description of Artificial Sequence Synthetic peptide”
ISGSGGRT

SEQ ID NO: 475 - protein, 8 aa, Artificial Sequence
  source: /note=“Description of Artificial Sequence Synthetic peptide”
ISGSGDIT

SEQ ID NO: 476 - protein, 8 aa, Artificial Sequence
  source: /note=“Description of Artificial Sequence Synthetic peptide”
ISYDGSNK

SEQ ID NO: 477 - protein, 8 aa, Artificial Sequence
  source: /note=“Description of Artificial Sequence Synthetic peptide”
ISYDGSNK

SEQ ID NO: 478 - protein, 7 aa, Artificial Sequence
  source: /note=“Description of Artificial Sequence Synthetic peptide”
IYHSGST

SEQ ID NO: 479 - protein, 10 aa, Artificial Sequence
  source: /note=“Description of Artificial Sequence Synthetic peptide”
IKSKNDGGTT

SEQ ID NO: 480 - protein, 8 aa, Artificial Sequence
  source: /note=“Description of Artificial Sequence Synthetic peptide”
ISAYNGNT

SEQ ID NO: 481 - protein, 8 aa, Artificial Sequence
  source: /note=“Description of Artificial Sequence Synthetic peptide”
ISGSGDIT

SEQ ID NO: 482 - protein, 8 aa, Artificial Sequence
  source: /note=“Description of Artificial Sequence Synthetic peptide”
ISGSGDIT

SEQ ID NO: 483 - protein, 8 aa, Artificial Sequence
  source: /note=“Description of Artificial Sequence Synthetic peptide”
ISGSGGRT

SEQ ID NO: 484 - protein, 8 aa, Artificial Sequence
  source: /note=“Description of Artificial Sequence Synthetic peptide”
ISGSGGST

SEQ ID NO: 485 - protein, 8 aa, Artificial Sequence
  source: /note=“Description of Artificial Sequence Synthetic peptide”
ISGSGGST

SEQ ID NO: 486 - protein, 8 aa, Artificial Sequence
  source: /note=“Description of Artificial Sequence Synthetic peptide”
ISGSGGST

SEQ ID NO: 487 - protein, 8 aa, Artificial Sequence
  source: /note=“Description of Artificial Sequence Synthetic peptide”
ISYDGSNK

SEQ ID NO: 488 - protein, 9 aa, Artificial Sequence
  source: /note=“Description of Artificial Sequence Synthetic peptide”
TYYRSKWYN

SEQ ID NO: 489 - protein, 8 aa, Artificial Sequence
  source: /note=“Description of Artificial Sequence Synthetic peptide”
INPNSGGT

SEQ ID NO: 490 - protein, 8 aa, Artificial Sequence
  source: /note=“Description of Artificial Sequence Synthetic peptide”
INPKSGGA

SEQ ID NO: 491 - protein, 8 aa, Artificial Sequence
  source: /note=“Description of Artificial Sequence Synthetic peptide”
ISGSGDRT

SEQ ID NO: 492 - protein, 8 aa, Artificial Sequence
  source: /note=“Description of Artificial Sequence Synthetic peptide”
ISSSGSTI

SEQ ID NO: 493 - protein, 8 aa, Artificial Sequence
  source: /note=“Description of Artificial Sequence Synthetic peptide”
ISYDGSNK

SEQ ID NO: 494 - protein, 7 aa, Artificial Sequence
  source: /note=“Description of Artificial Sequence Synthetic peptide”
ITGSGGT

SEQ ID NO: 495 - protein, 8 aa, Artificial Sequence
  source: /note=“Description of Artificial Sequence Synthetic peptide”
IYPGDSDT

SEQ ID NO: 496 - protein, 9 aa, Artificial Sequence
  source: /note=“Description of Artificial Sequence Synthetic peptide”
TYYRSKWYN

SEQ ID NO: 497 - protein, 9 aa, Artificial Sequence
  source: /note=“Description of Artificial Sequence Synthetic peptide”
TYYNRKWIN

SEQ ID NO: 498 - protein, 7 aa, Artificial Sequence
  source: /note=“Description of Artificial Sequence Synthetic peptide”
IYSGGST

SEQ ID NO: 499 - protein, 10 aa, Artificial Sequence
  source: /note=“Description of Artificial Sequence Synthetic peptide”
IKSKNDGGTT

SEQ ID NO: 500 - protein, 8 aa, Artificial Sequence
  source: /note=“Description of Artificial Sequence Synthetic peptide”
ISGSGDRT

SEQ ID NO: 501 - protein, 8 aa, Artificial Sequence
  source: /note=“Description of Artificial Sequence Synthetic peptide”
ISGSGDRT

SEQ ID NO: 502 - protein, 8 aa, Artificial Sequence
  source: /note=“Description of Artificial Sequence Synthetic peptide”
ISYDGSNK

SEQ ID NO: 503 - protein, 7 aa, Artificial Sequence
  source: /note=“Description of Artificial Sequence Synthetic peptide”
IYHSGST

SEQ ID NO: 504 - protein, 7 aa, Artificial Sequence
  source: /note=“Description of Artificial Sequence Synthetic peptide”
IYSGGST

SEQ ID NO: 505 - protein, 9 aa, Artificial Sequence
  source: /note=“Description of Artificial Sequence Synthetic peptide”
TYYRSKWYN

SEQ ID NO: 506 - protein, 9 aa, Artificial Sequence
  source: /note=“Description of Artificial Sequence Synthetic peptide”
TYYRSKWYN

SEQ ID NO: 507 - protein, 9 aa, Artificial Sequence
  source: /note=“Description of Artificial Sequence Synthetic peptide”
TYYRSKWYN

SEQ ID NO: 508 - protein, 7 aa, Artificial Sequence
  source: /note=“Description of Artificial Sequence Synthetic peptide”
IDYSGST

SEQ ID NO: 509 - protein, 10 aa, Artificial Sequence
  source: /note=“Description of Artificial Sequence Synthetic peptide”
IKSKNDGGTT

SEQ ID NO: 510 - protein, 8 aa, Artificial Sequence
  source: /note=“Description of Artificial Sequence Synthetic peptide”
ISAYNGNT

SEQ ID NO: 511 - protein, 8 aa, Artificial Sequence
  source: /note=“Description of Artificial Sequence Synthetic peptide”
ISYDGSNK

SEQ ID NO: 512 - protein, 8 aa, Artificial Sequence
  source: /note=“Description of Artificial Sequence Synthetic peptide”
ISYDGSNK

SEQ ID NO: 513 - protein, 8 aa, Artificial Sequence
  source: /note=“Description of Artificial Sequence Synthetic peptide”
IYPGDSET

SEQ ID NO: 514 - protein, 8 aa, Artificial Sequence
  source: /note=“Description of Artificial Sequence Synthetic peptide”
IIPIFGTA

SEQ ID NO: 515 - protein, 8 aa, Artificial Sequence
  source: /note=“Description of Artificial Sequence Synthetic peptide”
INPSGGST

SEQ ID NO: 516 - protein, 8 aa, Artificial Sequence
  source: /note=“Description of Artificial Sequence Synthetic peptide”
IIPIFGTA

SEQ ID NO: 517 - protein, 8 aa, Artificial Sequence
  source: /note=“Description of Artificial Sequence Synthetic peptide”
ISAYNGNT

SEQ ID NO: 518 - protein, 8 aa, Artificial Sequence
  source: /note=“Description of Artificial Sequence Synthetic peptide”
ISGSGGRT

SEQ ID NO: 519 - protein, 8 aa, Artificial Sequence
  source: /note=“Description of Artificial Sequence Synthetic peptide”
ISWNSGSI

SEQ ID NO: 520 - protein, 7 aa, Artificial Sequence
  source: /note=“Description of Artificial Sequence Synthetic peptide”
ITGSGGT

SEQ ID NO: 521 - protein, 7 aa, Artificial Sequence
  source: /note=“Description of Artificial Sequence Synthetic peptide”
IYHSGST

SEQ ID NO: 522 - protein, 8 aa, Artificial Sequence
  source: /note=“Description of Artificial Sequence Synthetic peptide”
ISGSGGST

SEQ ID NO: 523 - protein, 9 aa, Artificial Sequence
  source: /note=“Description of Artificial Sequence Synthetic peptide”
TYYRSKWYN

SEQ ID NO: 524 - protein, 9 aa, Artificial Sequence
  source: /note=“Description of Artificial Sequence Synthetic peptide”
TYYRSKWYN

SEQ ID NO: 525 - protein, 9 aa, Artificial Sequence
  source: /note=“Description of Artificial Sequence Synthetic peptide”
TYYRSKWYN

SEQ ID NO: 526 - protein, 8 aa, Artificial Sequence
  source: /note=“Description of Artificial Sequence Synthetic peptide”
ISWNSGSI

SEQ ID NO: 527 - protein, 10 aa, Artificial Sequence
  source: /note=“Description of Artificial Sequence Synthetic peptide”
IKSKNDGGTT

SEQ ID NO: 528 - protein, 10 aa, Artificial Sequence
  source: /note=“Description of Artificial Sequence Synthetic peptide”
IKSKNDGGTT

SEQ ID NO: 529 - protein, 8 aa, Artificial Sequence
  source: /note=“Description of Artificial Sequence Synthetic peptide”
ISYDGSNK

SEQ ID NO: 530 - protein, 8 aa, Artificial Sequence
  source: /note=“Description of Artificial Sequence Synthetic peptide”
ISGSGGST

SEQ ID NO: 531 - protein, 8 aa, Artificial Sequence
  source: /note=“Description of Artificial Sequence Synthetic peptide”
ISYDGSNK

SEQ ID NO: 532 - protein, 8 aa, Artificial Sequence
  source: /note=“Description of Artificial Sequence Synthetic peptide”
IWYDGNNK

SEQ ID NO: 533 - protein, 8 aa, Artificial Sequence
  source: /note=“Description of Artificial Sequence Synthetic peptide”
IWYDGSNK

SEQ ID NO: 534 - protein, 8 aa, Artificial Sequence
  source: /note=“Description of Artificial Sequence Synthetic peptide”
ISYDGGNK

SEQ ID NO: 535 - protein, 8 aa, Artificial Sequence
  source: /note=“Description of Artificial Sequence Synthetic peptide”
ISYDGSNK

SEQ ID NO: 536 - protein, 8 aa, Artificial Sequence
  source: /note=“Description of Artificial Sequence Synthetic peptide”
ISYDGSNK

SEQ ID NO: 537 - protein, 8 aa, Artificial Sequence
  source: /note=“Description of Artificial Sequence Synthetic peptide”
ISYDGSNK

SEQ ID NO: 538 - protein, 8 aa, Artificial Sequence
  source: /note=“Description of Artificial Sequence Synthetic peptide”
ISYDGSNK

SEQ ID NO: 539 - protein, 8 aa, Artificial Sequence
  source: /note=“Description of Artificial Sequence Synthetic peptide”
ISYDGSNK

SEQ ID NO: 540 - protein, 8 aa, Artificial Sequence
  source: /note=“Description of Artificial Sequence Synthetic peptide”
IWYDGSNK

SEQ ID NO: 541 - protein, 8 aa, Artificial Sequence
  source: /note=“Description of Artificial Sequence Synthetic peptide”
IKQDGSEK

SEQ ID NO: 542 - protein, 7 aa, Artificial Sequence
  source: /note=“Description of Artificial Sequence Synthetic peptide”
IYSGGST

SEQ ID NO: 543 - protein, 7 aa, Artificial Sequence
  source: /note=“Description of Artificial Sequence Synthetic peptide”
IYSGGST

SEQ ID NO: 544 - protein, 7 aa, Artificial Sequence
  source: /note=“Description of Artificial Sequence Synthetic peptide”
IYSGGST

SEQ ID NO: 545 - protein, 7 aa, Artificial Sequence
  source: /note=“Description of Artificial Sequence Synthetic peptide”
IYSGGST

SEQ ID NO: 546 - protein, 7 aa, Artificial Sequence
  source: /note=“Description of Artificial Sequence Synthetic peptide”
IYSGGST

SEQ ID NO: 547 - protein, 8 aa, Artificial Sequence
  source: /note=“Description of Artificial Sequence Synthetic peptide”
IYPGDSDT

SEQ ID NO: 548 - protein, 8 aa, Artificial Sequence
  source: /note=“Description of Artificial Sequence Synthetic peptide”
INPNSGGT

SEQ ID NO: 549 - protein, 8 aa, Artificial Sequence
  source: /note=“Description of Artificial Sequence Synthetic peptide”
ISAYNGNT

SEQ ID NO: 550 - protein, 8 aa, Artificial Sequence
  source: /note=“Description of Artificial Sequence Synthetic peptide”
ISAYNGNT

SEQ ID NO: 551 - protein, 8 aa, Artificial Sequence
  source: /note=“Description of Artificial Sequence Synthetic peptide”
INPSGGST

SEQ ID NO: 552 - protein, 12 aa, Artificial Sequence
  source: /note=“Description of Artificial Sequence Synthetic peptide”
ARVGSGGWTPDY

SEQ ID NO: 553 - protein, 13 aa, Artificial Sequence
  source: /note=“Description of Artificial Sequence Synthetic peptide”
AREVATIPAHFDY

SEQ ID NO: 554 - protein, 12 aa, Artificial Sequence
  source: /note=“Description of Artificial Sequence Synthetic peptide”
ARDYDILTGLDY

SEQ ID NO: 555 - protein, 14 aa, Artificial Sequence
  source: /note=“Description of Artificial Sequence Synthetic peptide”
AKDWAGYINGWYGN

SEQ ID NO: 556 - protein, 14 aa, Artificial Sequence
  source: /note=“Description of Artificial Sequence Synthetic peptide”
AKDWAGYVNGWYGN

SEQ ID NO: 557 - protein, 15 aa, Artificial Sequence
  source: /note=“Description of Artificial Sequence Synthetic peptide”
AKDWGTSLLYGYFDY

SEQ ID NO: 558 - protein, 12 aa, Artificial Sequence
  source: /note=“Description of Artificial Sequence Synthetic peptide”
ARVGSGGWTPDY

SEQ ID NO: 559 - protein, 12 aa, Artificial Sequence
  source: /note=“Description of Artificial Sequence Synthetic peptide”
ARDFEGSGALDV

SEQ ID NO: 560 - protein, 15 aa, Artificial Sequence
  source: /note=“Description of Artificial Sequence Synthetic peptide”
ARGGSSEFYYYGMDV

SEQ ID NO: 561 - protein, 14 aa, Artificial Sequence
  source: /note=“Description of Artificial Sequence Synthetic peptide”
AKDWAGYTNGWYGS

SEQ ID NO: 562 - protein, 14 aa, Artificial Sequence
  source: /note=“Description of Artificial Sequence Synthetic peptide”
AKDWAGYTNGWYGS

SEQ ID NO: 563 - protein, 14 aa, Artificial Sequence
  source: /note=“Description of Artificial Sequence Synthetic peptide”
AKDRSRRAPYYFDY

SEQ ID NO: 564 - protein, 12 aa, Artificial Sequence
  source: /note=“Description of Artificial Sequence Synthetic peptide”
AKVYRGYDAFDI

SEQ ID NO: 565 - protein, 11 aa, Artificial Sequence
  source: /note=“Description of Artificial Sequence Synthetic peptide”
ARHAGDGQIDY

SEQ ID NO: 566 - protein, 15 aa, Artificial Sequence
  source: /note=“Description of Artificial Sequence Synthetic peptide”
AREGSGLYYYYGMDV

SEQ ID NO: 567 - protein, 13 aa, Artificial Sequence
  source: /note=“Description of Artificial Sequence Synthetic peptide”
ARGGSGWYHYFDY

SEQ ID NO: 568 - protein, 14 aa, Artificial Sequence
  source: /note=“Description of Artificial Sequence Synthetic peptide”
AKDWAGYINGWYGS

SEQ ID NO: 569 - protein, 12 aa, Artificial Sequence
  source: /note=“Description of Artificial Sequence Synthetic peptide”
ARVGSGGWTPDY

SEQ ID NO: 570 - protein, 11 aa, Artificial Sequence
  source: /note=“Description of Artificial Sequence Synthetic peptide”
ARLGSGWSLDY

SEQ ID NO: 571 - protein, 14 aa, Artificial Sequence
  source: /note=“Description of Artificial Sequence Synthetic peptide”
AKDWAGYINGWFGN

SEQ ID NO: 572 - protein, 14 aa, Artificial Sequence
  source: /note=“Description of Artificial Sequence Synthetic peptide”
AKDWAGYVNGWYGN

SEQ ID NO: 573 - protein, 12 aa, Artificial Sequence
  source: /note=“Description of Artificial Sequence Synthetic peptide”
ARVGSGGWTPDY

SEQ ID NO: 574 - protein, 20 aa, Artificial Sequence
  source: /note=“Description of Artificial Sequence Synthetic peptide”
ARGGYCSSTSCYPDYNWFDP

SEQ ID NO: 575 - protein, 14 aa, Artificial Sequence
  source: /note=“Description of Artificial Sequence Synthetic peptide”
AKDWAGYINGWYAN

SEQ ID NO: 576 - protein, 14 aa, Artificial Sequence
  source: /note=“Description of Artificial Sequence Synthetic peptide”
ARDRRGGNWYEFDY

SEQ ID NO: 577 - protein, 13 aa, Artificial Sequence
  source: /note=“Description of Artificial Sequence Synthetic peptide”
AREGLAMAGYFDY

SEQ ID NO: 578 - protein, 13 aa, Artificial Sequence
  source: /note=“Description of Artificial Sequence Synthetic peptide”
ARVAVGANLAFDI

SEQ ID NO: 579 - protein, 14 aa, Artificial Sequence
  source: /note=“Description of Artificial Sequence Synthetic peptide”
AKDWAGYINGWYGN

SEQ ID NO: 580 - protein, 12 aa, Artificial Sequence
  source: /note=“Description of Artificial Sequence Synthetic peptide”
ARGMAQSPAFDY

SEQ ID NO: 581 - protein, 14 aa, Artificial Sequence
  source: /note=“Description of Artificial Sequence Synthetic peptide”
AKDWAGYINGWYGN

SEQ ID NO: 582 - protein, 9 aa, Artificial Sequence
  source: /note=“Description of Artificial Sequence Synthetic peptide”
ARETGGFDY

SEQ ID NO: 583 - protein, 14 aa, Artificial Sequence
  source: /note=“Description of Artificial Sequence Synthetic peptide”
ARDPVRGDGYNFDY

SEQ ID NO: 584 - protein, 14 aa, Artificial Sequence
  source: /note=“Description of Artificial Sequence Synthetic peptide”
AKDWAGYVNGWYGN

SEQ ID NO: 585 - protein, 17 aa, Artificial Sequence
  source: /note=“Description of Artificial Sequence Synthetic peptide”
AKATGYSSGWYGAYFDY

SEQ ID NO: 586 - protein, 9 aa, Artificial Sequence
  source: /note=“Description of Artificial Sequence Synthetic peptide”
ARDRGSMDV

SEQ ID NO: 587 - protein, 15 aa, Artificial Sequence
  source: /note=“Description of Artificial Sequence Synthetic peptide”
ARLGRTSHQSWDLGY

SEQ ID NO: 588 - protein, 11 aa, Artificial Sequence
  source: /note=“Description of Artificial Sequence Synthetic peptide”
ASGASPYYFDY

SEQ ID NO: 589 - protein, 13 aa, Artificial Sequence
  source: /note=“Description of Artificial Sequence Synthetic peptide”
ARESNTANTHFDY

SEQ ID NO: 590 - protein, 15 aa, Artificial Sequence
  source: /note=“Description of Artificial Sequence Synthetic peptide”
ARGGVGATWYYGMDV

SEQ ID NO: 591 - protein, 13 aa, Artificial Sequence
  source: /note=“Description of Artificial Sequence Synthetic peptide”
AKQQWLGTWYFDL

SEQ ID NO: 592 - protein, 15 aa, Artificial Sequence
  source: /note=“Description of Artificial Sequence Synthetic peptide”
AKGLLVASIYDAFDI

SEQ ID NO: 593 - protein, 11 aa, Artificial Sequence
  source: /note=“Description of Artificial Sequence Synthetic peptide”
AKDIAAGGLDS

SEQ ID NO: 594 - protein, 14 aa, Artificial Sequence
  source: /note=“Description of Artificial Sequence Synthetic peptide”
AKDWAGYINGWYGN

SEQ ID NO: 595 - protein, 13 aa, Artificial Sequence
  source: /note=“Description of Artificial Sequence Synthetic peptide”
AREQWLGPAHFDY

SEQ ID NO: 596 - protein, 15 aa, Artificial Sequence
  source: /note=“Description of Artificial Sequence Synthetic peptide”
ARERNRAGEFSAFDI

SEQ ID NO: 597 - protein, 9 aa, Artificial Sequence
  source: /note=“Description of Artificial Sequence Synthetic peptide”
ARGASGLDF

SEQ ID NO: 598 - protein, 14 aa, Artificial Sequence
  source: /note=“Description of Artificial Sequence Synthetic peptide”
ARDLHCGSSCGPEA

SEQ ID NO: 599 - protein, 17 aa, Artificial Sequence
  source: /note=“Description of Artificial Sequence Synthetic peptide”
ARDPVYSSSWGGYAFDI

SEQ ID NO: 600 - protein, 15 aa, Artificial Sequence
  source: /note=“Description of Artificial Sequence Synthetic peptide”
ARDTFGGGSYYGHGY

SEQ ID NO: 601 - protein, 13 aa, Artificial Sequence
  source: /note=“Description of Artificial Sequence Synthetic peptide”
ARENSNAWKVMDV

SEQ ID NO: 602 - protein, 14 aa, Artificial Sequence
  source: /note=“Description of Artificial Sequence Synthetic peptide”
AKDWAGYINGWYGN

SEQ ID NO: 603 - protein, 14 aa, Artificial Sequence
  source: /note=“Description of Artificial Sequence Synthetic peptide”
AKDWAGYINGWYGN

SEQ ID NO: 604 - protein, 14 aa, Artificial Sequence
  source: /note=“Description of Artificial Sequence Synthetic peptide”
AKDWAGYIDGWYGN

SEQ ID NO: 605 - protein, 14 aa, Artificial Sequence
  source: /note=“Description of Artificial Sequence Synthetic peptide”
AKDWGAYSSGWYGD

SEQ ID NO: 606 - protein, 14 aa, Artificial Sequence
  source: /note=“Description of Artificial Sequence Synthetic peptide”
AKDWAGYSNGWYGS

SEQ ID NO: 607 - protein, 14 aa, Artificial Sequence
  source: /note=“Description of Artificial Sequence Synthetic peptide”
AKDWAGYSNGWFGS

SEQ ID NO: 608 - protein, 12 aa, Artificial Sequence
  source: /note=“Description of Artificial Sequence Synthetic peptide”
ARVGSGGWTPDY

SEQ ID NO: 609 - protein, 14 aa, Artificial Sequence
  source: /note=“Description of Artificial Sequence Synthetic peptide”
AVGVGFITDGYFQH

SEQ ID NO: 610 - protein, 12 aa, Artificial Sequence
  source: /note=“Description of Artificial Sequence Synthetic peptide”
ARVGSGGWTPDY

SEQ ID NO: 611 - protein, 12 aa, Artificial Sequence
  source: /note=“Description of Artificial Sequence Synthetic peptide”
ARVGSGGWTPDY

SEQ ID NO: 612 - protein, 13 aa, Artificial Sequence
  source: /note=“Description of Artificial Sequence Synthetic peptide”
AKQQWLGTWYFDL

SEQ ID NO: 613 - protein, 18 aa, Artificial Sequence
  source: /note=“Description of Artificial Sequence Synthetic peptide”
AKEWGGGDSPTDMGLFDY

SEQ ID NO: 614 - protein, 12 aa, Artificial Sequence
  source: /note=“Description of Artificial Sequence Synthetic peptide”
TRVGSGGWTPDY

SEQ ID NO: 615 - protein, 13 aa, Artificial Sequence
  source: /note=“Description of Artificial Sequence Synthetic peptide”
ARDNSGSYNWFNP

SEQ ID NO: 616 - protein, 12 aa, Artificial Sequence
  source: /note=“Description of Artificial Sequence Synthetic peptide”
ARSHGGSNWFDP

SEQ ID NO: 617 - protein, 11 aa, Artificial Sequence
  source: /note=“Description of Artificial Sequence Synthetic peptide”
ATSLGDDAFDI

SEQ ID NO: 618 - protein, 13 aa, Artificial Sequence
  source: /note=“Description of Artificial Sequence Synthetic peptide”
ARLGHSGSWYFDL

SEQ ID NO: 619 - protein, 12 aa, Artificial Sequence
  source: /note=“Description of Artificial Sequence Synthetic peptide”
ARDLSYSDAFDI

SEQ ID NO: 620 - protein, 12 aa, Artificial Sequence
  source: /note=“Description of Artificial Sequence Synthetic peptide”
ARDMTTVDAFDI

SEQ ID NO: 621 - protein, 11 aa, Artificial Sequence
  source: /note=“Description of Artificial Sequence Synthetic peptide”
ARDTASGGMDV

SEQ ID NO: 622 - protein, 12 aa, Artificial Sequence
  source: /note=“Description of Artificial Sequence Synthetic peptide”
AREPYPGGPFDI

SEQ ID NO: 623 - protein, 10 aa, Artificial Sequence
  source: /note=“Description of Artificial Sequence Synthetic peptide”
ANLYGDYNAY

SEQ ID NO: 624 - protein, 14 aa, Artificial Sequence
  source: /note=“Description of Artificial Sequence Synthetic peptide”
AKDWAGYINGWYGN

SEQ ID NO: 625 - protein, 14 aa, Artificial Sequence
  source: /note=“Description of Artificial Sequence Synthetic peptide”
AKDWAGYINGWYGN

SEQ ID NO: 626 - protein, 14 aa, Artificial Sequence
  source: /note=“Description of Artificial Sequence Synthetic peptide”
AKDWAGYTNGWYGS

SEQ ID NO: 627 - protein, 7 aa, Artificial Sequence
  source: /note=“Description of Artificial Sequence Synthetic peptide”
AKDLVLG

SEQ ID NO: 628 - protein, 15 aa, Artificial Sequence
  source: /note=“Description of Artificial Sequence Synthetic peptide”
AKDWDSSGYWPLFDY

SEQ ID NO: 629 - protein, 12 aa, Artificial Sequence
  source: /note=“Description of Artificial Sequence Synthetic peptide”
ARVGSGGWTPDY

SEQ ID NO: 630 - protein, 12 aa, Artificial Sequence
  source: /note=“Description of Artificial Sequence Synthetic peptide”
ARVGSGGWTPDY

SEQ ID NO: 631 - protein, 12 aa, Artificial Sequence
  source: /note=“Description of Artificial Sequence Synthetic peptide”
AREVVGSYYLDY

SEQ ID NO: 632 - protein, 16 aa, Artificial Sequence
  source: /note=“Description of Artificial Sequence Synthetic peptide”
ARGGDCSSTSCYDPDY

SEQ ID NO: 633 - protein, 14 aa, Artificial Sequence
  source: /note=“Description of Artificial Sequence Synthetic peptide”
ARIGRFGRKYGMDV

SEQ ID NO: 634 - protein, 11 aa, Artificial Sequence
  source: /note=“Description of Artificial Sequence Synthetic peptide”
ARGLGDSSSSY

SEQ ID NO: 635 - protein, 14 aa, Artificial Sequence
  source: /note=“Description of Artificial Sequence Synthetic peptide”
AKDWAGYVNGWYGN

SEQ ID NO: 636 - protein, 14 aa, Artificial Sequence
  source: /note=“Description of Artificial Sequence Synthetic peptide”
AKDWAGYVNGWYGN

SEQ ID NO: 637 - protein, 14 aa, Artificial Sequence
  source: /note=“Description of Artificial Sequence Synthetic peptide”
AKDWAGYINGWYGN

SEQ ID NO: 638 - protein, 14 aa, Artificial Sequence
  source: /note=“Description of Artificial Sequence Synthetic peptide”
AKDWGAYSSGWYGD

SEQ ID NO: 639 - protein, 14 aa, Artificial Sequence
  source: /note=“Description of Artificial Sequence Synthetic peptide”
AKDWAGYTNGWYGS

SEQ ID NO: 640 - protein, 14 aa, Artificial Sequence
  source: /note=“Description of Artificial Sequence Synthetic peptide”
AKDWAGYINGWYGS

SEQ ID NO: 641 - protein, 15 aa, Artificial Sequence
  source: /note=“Description of Artificial Sequence Synthetic peptide”
AKERFTGGYYTYFDY

SEQ ID NO: 642 - protein, 10 aa, Artificial Sequence
  source: /note=“Description of Artificial Sequence Synthetic peptide”
ARAGGLHLDY

SEQ ID NO: 643 - protein, 11 aa, Artificial Sequence
  source: /note=“Description of Artificial Sequence Synthetic peptide”
ARGNGDGGFDY

SEQ ID NO: 644 - protein, 14 aa, Artificial Sequence
  source: /note=“Description of Artificial Sequence Synthetic peptide”
AKDWAGYINGWYGN

SEQ ID NO: 645 - protein, 14 aa, Artificial Sequence
  source: /note=“Description of Artificial Sequence Synthetic peptide”
AKDWAGYVNGWYGN

SEQ ID NO: 646 - protein, 14 aa, Artificial Sequence
  source: /note=“Description of Artificial Sequence Synthetic peptide”
ARDRGVEGAYGMDV

SEQ ID NO: 647 - protein, 15 aa, Artificial Sequence
  source: /note=“Description of Artificial Sequence Synthetic peptide”
AKGLLVASIYDAFDI

SEQ ID NO: 648 - protein, 9 aa, Artificial Sequence
  source: /note=“Description of Artificial Sequence Synthetic peptide”
ARGSNIFDI

SEQ ID NO: 649 - protein, 9 aa, Artificial Sequence
  source: /note=“Description of Artificial Sequence Synthetic peptide”
TTAPSLMDV

SEQ ID NO: 650 - protein, 14 aa, Artificial Sequence
  source: /note=“Description of Artificial Sequence Synthetic peptide”
ARDLTFGSGPTRDY

SEQ ID NO: 651 - protein, 14 aa, Artificial Sequence
  source: /note=“Description of Artificial Sequence Synthetic peptide”
AKDWAGYTNGWYGS

SEQ ID NO: 652 - protein, 14 aa, Artificial Sequence
  source: /note=“Description of Artificial Sequence Synthetic peptide”
AKDWAGYVNGWYGN

SEQ ID NO: 653 - protein, 14 aa, Artificial Sequence
  source: /note=“Description of Artificial Sequence Synthetic peptide”
AKDWGAYSSGWYGD

SEQ ID NO: 654 - protein, 14 aa, Artificial Sequence
  source: /note=“Description of Artificial Sequence Synthetic peptide”
AKDWAGYINGWYGN

SEQ ID NO: 655 - protein, 15 aa, Artificial Sequence
  source: /note=“Description of Artificial Sequence Synthetic peptide”
AKDWTNQWLDAYFDY

SEQ ID NO: 656 - protein, 21 aa, Artificial Sequence
  source: /note=“Description of Artificial Sequence Synthetic peptide”
AKETILYDILTGYYNEGAFDI

SEQ ID NO: 657 - protein, 16 aa, Artificial Sequence
  source: /note=“Description of Artificial Sequence Synthetic peptide”
AKDWGRFGELLEGSPY

SEQ ID NO: 658 - protein, 18 aa, Artificial Sequence
  source: /note=“Description of Artificial Sequence Synthetic peptide”
AREFQDSSSWYEGRAFDI

SEQ ID NO: 659 - protein, 16 aa, Artificial Sequence
  source: /note=“Description of Artificial Sequence Synthetic peptide”
ARDWGRGVGDSGFVDY

SEQ ID NO: 660 - protein, 11 aa, Artificial Sequence
  source: /note=“Description of Artificial Sequence Synthetic peptide”
ARDFVGASLDY

SEQ ID NO: 661 - protein, 14 aa, Artificial Sequence
  source: /note=“Description of Artificial Sequence Synthetic peptide”
AKDWAGYINGWYGN

SEQ ID NO: 662 - protein, 15 aa, Artificial Sequence
  source: /note=“Description of Artificial Sequence Synthetic peptide”
ARGYLGAWNPDFYDY

SEQ ID NO: 663 - protein, 12 aa, Artificial Sequence
  source: /note=“Description of Artificial Sequence Synthetic peptide”
ARVGSGGWTPDY

SEQ ID NO: 664 - protein, 14 aa, Artificial Sequence
  source: /note=“Description of Artificial Sequence Synthetic peptide”
AKDWAGYINGWFGS

SEQ ID NO: 665 - protein, 11 aa, Artificial Sequence
  source: /note=“Description of Artificial Sequence Synthetic peptide”
ARLGDGSNFDY

SEQ ID NO: 666 - protein, 16 aa, Artificial Sequence
  source: /note=“Description of Artificial Sequence Synthetic peptide”
AREKIAVAGYYYGMDV

SEQ ID NO: 667 - protein, 13 aa, Artificial Sequence
  source: /note=“Description of Artificial Sequence Synthetic peptide”
ARDGGWSGSALDV

SEQ ID NO: 668 - protein, 12 aa, Artificial Sequence
  source: /note=“Description of Artificial Sequence Synthetic peptide”
ARDLHSAAGFDY

SEQ ID NO: 669 - protein, 9 aa, Artificial Sequence
  source: /note=“Description of Artificial Sequence Synthetic peptide”
TTAPSLMDV

SEQ ID NO: 670 - protein, 14 aa, Artificial Sequence
  source: /note=“Description of Artificial Sequence Synthetic peptide”
AKDWAGYINGWYGN

SEQ ID NO: 671 - protein, 14 aa, Artificial Sequence
  source: /note=“Description of Artificial Sequence Synthetic peptide”
AKDWAGYINGWYGN

SEQ ID NO: 672 - protein, 14 aa, Artificial Sequence
  source: /note=“Description of Artificial Sequence Synthetic peptide”
AKGSSPYYYYGMDV

SEQ ID NO: 673 - protein, 11 aa, Artificial Sequence
  source: /note=“Description of Artificial Sequence Synthetic peptide”
ARDGGSGWYDY

SEQ ID NO: 674 - protein, 11 aa, Artificial Sequence
  source: /note=“Description of Artificial Sequence Synthetic peptide”
ARDTASGGMDV

SEQ ID NO: 675 - protein, 15 aa, Artificial Sequence
  source: /note=“Description of Artificial Sequence Synthetic peptide”
ARGVTVPYYYYGMDV

SEQ ID NO: 676 - protein, 12 aa, Artificial Sequence
  source: /note=“Description of Artificial Sequence Synthetic peptide”
ARSSGSYGYFQH

SEQ ID NO: 677 - protein, 15 aa, Artificial Sequence
  source: /note=“Description of Artificial Sequence Synthetic peptide”
AREGTDIYYYYGMDV

SEQ ID NO: 678 - protein, 13 aa, Artificial Sequence
  source: /note=“Description of Artificial Sequence Synthetic peptide”
ARDGWIRKEAFDP

SEQ ID NO: 679 - protein, 9 aa, Artificial Sequence
  source: /note=“Description of Artificial Sequence Synthetic peptide”
TTAPSLMDV

SEQ ID NO: 680 - protein, 15 aa, Artificial Sequence
  source: /note=“Description of Artificial Sequence Synthetic peptide”
ARDPGGYYYYYGMDV

SEQ ID NO: 681 - protein, 12 aa, Artificial Sequence
  source: /note=“Description of Artificial Sequence Synthetic peptide”
ARVGSGGWTPDY

SEQ ID NO: 682 - protein, 15 aa, Artificial Sequence
  source: /note=“Description of Artificial Sequence Synthetic peptide”
AKLGGSYSIYYGMDV

SEQ ID NO: 683 - protein, 11 aa, Artificial Sequence
  source: /note=“Description of Artificial Sequence Synthetic peptide”
ARDGGNYQFDY

SEQ ID NO: 684 - protein, 16 aa, Artificial Sequence
  source: /note=“Description of Artificial Sequence Synthetic peptide”
ARTGRSGSYYSDAFDI

SEQ ID NO: 685 - protein, 15 aa, Artificial Sequence
  source: /note=“Description of Artificial Sequence Synthetic peptide”
AREDHDYSNQGGFDY

SEQ ID NO: 686 - protein, 17 aa, Artificial Sequence
  source: /note=“Description of Artificial Sequence Synthetic peptide”
AARAPGGSSYYYYGMDV

SEQ ID NO: 687 - protein, 15 aa, Artificial Sequence
  source: /note=“Description of Artificial Sequence Synthetic peptide”
ARDPGYDFWSGYSDV

SEQ ID NO: 688 - protein, 14 aa, Artificial Sequence
  source: /note=“Description of Artificial Sequence Synthetic peptide”
AKDWAGYINGWYGN

SEQ ID NO: 689 - protein, 19 aa, Artificial Sequence
  source: /note=“Description of Artificial Sequence Synthetic peptide”
AKDMWGSLSIVGATRAFDY

SEQ ID NO: 690 - protein, 14 aa, Artificial Sequence
  source: /note=“Description of Artificial Sequence Synthetic peptide”
AKDWAGYINGWFGS

SEQ ID NO: 691 - protein, 20 aa, Artificial Sequence
  source: /note=“Description of Artificial Sequence Synthetic peptide”
ARGPLLIAAAGTDYYYGMDV

SEQ ID NO: 692 - protein, 14 aa, Artificial Sequence
  source: /note=“Description of Artificial Sequence Synthetic peptide”
ASSYGGNPLDAFDI

SEQ ID NO: 693 - protein, 16 aa, Artificial Sequence
  source: /note=“Description of Artificial Sequence Synthetic peptide”
AREKIAVAGYYYGMDV

SEQ ID NO: 694 - protein, 18 aa, Artificial Sequence
  source: /note=“Description of Artificial Sequence Synthetic peptide”
AREFQDSSSWYEGRAFDI

SEQ ID NO: 695 - protein, 15 aa, Artificial Sequence
  source: /note=“Description of Artificial Sequence Synthetic peptide”
ARGGVGATWYYGMDV

SEQ ID NO: 696 - protein, 11 aa, Artificial Sequence
  source: /note=“Description of Artificial Sequence Synthetic peptide”
AKDIAAGGLDS

SEQ ID NO: 697 - protein, 9 aa, Artificial Sequence
  source: /note=“Description of Artificial Sequence Synthetic peptide”
TTAPSLMDV

SEQ ID NO: 698 - protein, 9 aa, Artificial Sequence
  source: /note=“Description of Artificial Sequence Synthetic peptide”
TTAPSLMDV

SEQ ID NO: 699 - protein, 14 aa, Artificial Sequence
  source: /note=“Description of Artificial Sequence Synthetic peptide”
ARDRGVEGAYGMDV

SEQ ID NO: 700 - protein, 17 aa, Artificial Sequence
  source: /note=“Description of Artificial Sequence Synthetic peptide”
AKATGYSSGWYGAYFDY

SEQ ID NO: 701 - protein, 14 aa, Artificial Sequence
  source: /note=“Description of Artificial Sequence Synthetic peptide”
AKGSSPYYYYGMDV

SEQ ID NO: 702 - protein, 13 aa, Artificial Sequence
  source: /note=“Description of Artificial Sequence Synthetic peptide”
ARDNSGSYNWFNP

SEQ ID NO: 703 - protein, 12 aa, Artificial Sequence
  source: /note=“Description of Artificial Sequence Synthetic peptide”
AREVVGSYYLDY

SEQ ID NO: 704 - protein, 12 aa, Artificial Sequence
  source: /note=“Description of Artificial Sequence Synthetic peptide”
ARVGSGGWTPDY

SEQ ID NO: 705 - protein, 12 aa, Artificial Sequence
  source: /note=“Description of Artificial Sequence Synthetic peptide”
ARVGSGGWTPDY

SEQ ID NO: 706 - protein, 12 aa, Artificial Sequence
  source: /note=“Description of Artificial Sequence Synthetic peptide”
ARVGSGGWTPDY

SEQ ID NO: 707 - protein, 12 aa, Artificial Sequence
  source: /note=“Description of Artificial Sequence Synthetic peptide”
ARVGSGGWTPDY

SEQ ID NO: 708 - protein, 12 aa, Artificial Sequence
  source: /note=“Description of Artificial Sequence Synthetic peptide”
ARVGSGGWTPDY

SEQ ID NO: 709 - protein, 12 aa, Artificial Sequence
  source: /note=“Description of Artificial Sequence Synthetic peptide”
TRVGSGGWTPDY

SEQ ID NO: 710 - protein, 11 aa, Artificial Sequence
  source: /note=“Description of Artificial Sequence Synthetic peptide”
ARLGSGWSLDY

SEQ ID NO: 711 - protein, 14 aa, Artificial Sequence
  source: /note=“Description of Artificial Sequence Synthetic peptide”
ARDLHCGSSCGPEA

SEQ ID NO: 712 - protein, 12 aa, Artificial Sequence
  source: /note=“Description of Artificial Sequence Synthetic peptide”
ARDLHSAAGFDY

SEQ ID NO: 713 - protein, 12 aa, Artificial Sequence
  source: /note=“Description of Artificial Sequence Synthetic peptide”
ARDLSYSDAFDI

SEQ ID NO: 714 - protein, 12 aa, Artificial Sequence
  source: /note=“Description of Artificial Sequence Synthetic peptide”
ARDFEGSGALDV

SEQ ID NO: 715 - protein, 11 aa, Artificial Sequence
  source: /note=“Description of Artificial Sequence Synthetic peptide”
ARDTASGGMDV

SEQ ID NO: 716 - protein, 11 aa, Artificial Sequence
  source: /note=“Description of Artificial Sequence Synthetic peptide”
ARDTASGGMDV

SEQ ID NO: 717 - protein, 11 aa, Artificial Sequence
  source: /note=“Description of Artificial Sequence Synthetic peptide”
ASGASPYYFDY

SEQ ID NO: 718 - protein, 16 aa, Artificial Sequence
  source: /note=“Description of Artificial Sequence Synthetic peptide”
ARGGDCSSTSCYDPDY

SEQ ID NO: 719 - protein, 17 aa, Artificial Sequence
  source: /note=“Description of Artificial Sequence Synthetic peptide”
ARDPVYSSSWGGYAFDI

SEQ ID NO: 720 - protein, 11 aa, Artificial Sequence
  source: /note=“Description of Artificial Sequence Synthetic peptide”
ARGLGDSSSSY

SEQ ID NO: 721 - protein, 15 aa, Artificial Sequence
  source: /note=“Description of Artificial Sequence Synthetic peptide”
AREDHDYSNQGGFDY

SEQ ID NO: 722 - protein, 22 aa, Artificial Sequence
  source: /note=“Description of Artificial Sequence Synthetic peptide”
TSNIGANHTKNAAWDDSLRGWT

SEQ ID NO: 723 - protein, 23 aa, Artificial Sequence
  source: /note=“Description of Artificial Sequence Synthetic peptide”
SSDIGGYKYDVTGSYSSSSSHYV

SEQ ID NO: 724 - protein, 18 aa, Artificial Sequence
  source: /note=“Description of Artificial Sequence Synthetic peptide”
QSISSFAASQQSYSTPWT

SEQ ID NO: 725 - protein, 20 aa, Artificial Sequence
  source: /note=“Description of Artificial Sequence Synthetic peptide”
QSVSSNGASQHYNNWPPQIT

SEQ ID NO: 726 - protein, 18 aa, Artificial Sequence
  source: /note=“Description of Artificial Sequence Synthetic peptide”
QSVSSNGASQQYGYSQIT

SEQ ID NO: 727 - protein, 20 aa, Artificial Sequence
  source: /note=“Description of Artificial Sequence Synthetic peptide”
NIGSKSDDSQVWDSSSDHVV

SEQ ID NO: 728 - protein, 20 aa, Artificial Sequence
  source: /note=“Description of Artificial Sequence Synthetic peptide”
NIGSKSDDSQVWDSSSDHVV

SEQ ID NO: 729 - protein, 24 aa, Artificial Sequence
  source: /note=“Description of Artificial Sequence Synthetic peptide”
SSNIGAGYDSSNQSFDPSLSDSWV

SEQ ID NO: 730 - protein, 20 aa, Artificial Sequence
  source: /note=“Description of Artificial Sequence Synthetic peptide”
SGSITDDYEDHQSYDAESWV

SEQ ID NO: 731 - protein, 18 aa, Artificial Sequence
  source: /note=“Description of Artificial Sequence Synthetic peptide”
QSVSSNGASQQYGYSQIT

SEQ ID NO: 732 - protein, 21 aa, Artificial Sequence
  source: /note=“Description of Artificial Sequence Synthetic peptide”
NIGSKSDDSQVWDSSSDLLYV

SEQ ID NO: 733 - protein, 19 aa, Artificial Sequence
  source: /note=“Description of Artificial Sequence Synthetic peptide”
QSVSSSYGASQQYGRSPFT

SEQ ID NO: 734 - protein, 18 aa, Artificial Sequence
  source: /note=“Description of Artificial Sequence Synthetic peptide”
QSVTSNYGASQQYGSSPT

SEQ ID NO: 735 - protein, 23 aa, Artificial Sequence
  source: /note=“Description of Artificial Sequence Synthetic peptide”
TGAVTSGFYSATLLYYGGAQPWV

SEQ ID NO: 736 - protein, 20 aa, Artificial Sequence
  source: /note=“Description of Artificial Sequence Synthetic peptide”
NIGSKSDDSQLWDGASDLVI

SEQ ID NO: 737 - protein, 18 aa, Artificial Sequence
  source: /note=“Description of Artificial Sequence Synthetic peptide”
QTISSYGASQQSYSTPQT

SEQ ID NO: 738 - protein, 20 aa, Artificial Sequence
  source: /note=“Description of Artificial Sequence Synthetic peptide”
NIGSKSDDSQVWDSSSDHVV

SEQ ID NO: 739 - protein, 20 aa, Artificial Sequence
  source: /note=“Description of Artificial Sequence Synthetic peptide”
NIGSKSDDSQVWDSSSDHVV

SEQ ID NO: 740 - protein, 21 aa, Artificial Sequence
  source: /note=“Description of Artificial Sequence Synthetic peptide”
QRVRSSYGASQQYGSSPPRII

SEQ ID NO: 741 - protein, 18 aa, Artificial Sequence
  source: /note=“Description of Artificial Sequence Synthetic peptide”
QTVSNNDASQQYGSSPLT

SEQ ID NO: 742 - protein, 20 aa, Artificial Sequence
  source: /note=“Description of Artificial Sequence Synthetic peptide”
NIGSKSDDSQVWDSSSDHVV

SEQ ID NO: 743 - protein, 20 aa, Artificial Sequence
  source: /note=“Description of Artificial Sequence Synthetic peptide”
DIESKSDDSQVWDGIINQVV

SEQ ID NO: 744 - protein, 18 aa, Artificial Sequence
  source: /note=“Description of Artificial Sequence Synthetic peptide”
QGVRASSAASQQYGRSPT

SEQ ID NO: 745 - protein, 19 aa, Artificial Sequence
  source: /note=“Description of Artificial Sequence Synthetic peptide”
QSISSYAASQQSYSTPPYT

SEQ ID NO: 746 - protein, 20 aa, Artificial Sequence
  source: /note=“Description of Artificial Sequence Synthetic peptide”
QSVSSSYGASQQYGSSPQYT

SEQ ID NO: 747 - protein, 20 aa, Artificial Sequence
  source: /note=“Description of Artificial Sequence Synthetic peptide”
NIGSKSDDSQVWGSSNDPVV

SEQ ID NO: 748 - protein, 20 aa, Artificial Sequence
  source: /note=“Description of Artificial Sequence Synthetic peptide”
NIGSKSDDSQVWDSSSDHVV

SEQ ID NO: 749 - protein, 22 aa, Artificial Sequence
  source: /note=“Description of Artificial Sequence Synthetic peptide”
SSNIGNNYDNNGTWDSSLSAVV

SEQ ID NO: 750 - protein, 22 aa, Artificial Sequence
  source: /note=“Description of Artificial Sequence Synthetic peptide”
NIGAKSDDSQVWDNTGDHPRVI

SEQ ID NO: 751 - protein, 23 aa, Artificial Sequence
  source: /note=“Description of Artificial Sequence Synthetic peptide”
QSLVYSDGNTYKVSMQGKHWPPT

SEQ ID NO: 752 - protein, 20 aa, Artificial Sequence
  source: /note=“Description of Artificial Sequence Synthetic peptide”
SLRSYYGKNNSRDSSGNHWV

SEQ ID NO: 753 - protein, 21 aa, Artificial Sequence
  source: /note=“Description of Artificial Sequence Synthetic peptide”
NIGSKSDDSQVWDSSSDHSVV

SEQ ID NO: 754 - protein, 20 aa, Artificial Sequence
  source: /note=“Description of Artificial Sequence Synthetic peptide”
NIGSYSDDSQVWDSSSDHVI

SEQ ID NO: 755 - protein, 20 aa, Artificial Sequence
  source: /note=“Description of Artificial Sequence Synthetic peptide”
NIGSKSDDSQVWDSSSDHVV

SEQ ID NO: 756 - protein, 18 aa, Artificial Sequence
  source: /note=“Description of Artificial Sequence Synthetic peptide”
NLGGRYQDLQAWDTYTVV

SEQ ID NO: 757 - protein, 20 aa, Artificial Sequence
  source: /note=“Description of Artificial Sequence Synthetic peptide”
NIGSKSDDSQVWDSSSDHVV

SEQ ID NO: 758 - protein, 20 aa, Artificial Sequence
  source: /note=“Description of Artificial Sequence Synthetic peptide”
SLRSYYGKNNSRDSSGNHVV

SEQ ID NO: 759 - protein, 19 aa, Artificial Sequence
  source: /note=“Description of Artificial Sequence Synthetic peptide”
KLGDKYQDTQAWDSSTNYV

SEQ ID NO: 760 - protein, 18 aa, Artificial Sequence
  source: /note=“Description of Artificial Sequence Synthetic peptide”
QSINSNGASQQFEQWPLT

SEQ ID NO: 761 - protein, 18 aa, Artificial Sequence
  source: /note=“Description of Artificial Sequence Synthetic peptide”
QRISKYGSSQQSDSVPIT

SEQ ID NO: 762 - protein, 23 aa, Artificial Sequence
  source: /note=“Description of Artificial Sequence Synthetic peptide”
SSNIGAGYRGDNQSHDESLNSKV

SEQ ID NO: 763 - protein, 18 aa, Artificial Sequence
  source: /note=“Description of Artificial Sequence Synthetic peptide”
QSVSSNGASQQYGSSPLT

SEQ ID NO: 764 - protein, 20 aa, Artificial Sequence
  source: /note=“Description of Artificial Sequence Synthetic peptide”
NIGSKSDDSQLWDGASDLVI

SEQ ID NO: 765 - protein, 20 aa, Artificial Sequence
  source: /note=“Description of Artificial Sequence Synthetic peptide”
NIGSKSDDSQVWDSSSDHVV

SEQ ID NO: 766 - protein, 20 aa, Artificial Sequence
  source: /note=“Description of Artificial Sequence Synthetic peptide”
QSVSSNGASQQYNNWPPQYT

SEQ ID NO: 767 - protein, 20 aa, Artificial Sequence
  source: /note=“Description of Artificial Sequence Synthetic peptide”
NIGSKSDDSQVWDSSSDYVV

SEQ ID NO: 768 - protein, 20 aa, Artificial Sequence
  source: /note=“Description of Artificial Sequence Synthetic peptide”
NIGSKSDDSQVWDSLSDHVI

SEQ ID NO: 769 - protein, 20 aa, Artificial Sequence
  source: /note=“Description of Artificial Sequence Synthetic peptide”
NIGTKSDDSQVWDHSNDHVV

SEQ ID NO: 770 - protein, 20 aa, Artificial Sequence
  source: /note=“Description of Artificial Sequence Synthetic peptide”
NIGSKSDDSSAWDSSLTAVV

SEQ ID NO: 771 - protein, 20 aa, Artificial Sequence
  source: /note=“Description of Artificial Sequence Synthetic peptide”
NIGSKGDDRQVWDTNSQHVV

SEQ ID NO: 772 - protein, 22 aa, Artificial Sequence
  source: /note=“Description of Artificial Sequence Synthetic peptide”
SSNIGNNGYDDATWDDRLKGYV

SEQ ID NO: 773 - protein, 20 aa, Artificial Sequence
  source: /note=“Description of Artificial Sequence Synthetic peptide”
NIGSKSDDSQVWDSSSDQGV

SEQ ID NO: 774 - protein, 22 aa, Artificial Sequence
  source: /note=“Description of Artificial Sequence Synthetic peptide”
GGSLASNYEDKQSYDSANPLVV

SEQ ID NO: 775 - protein, 20 aa, Artificial Sequence
  source: /note=“Description of Artificial Sequence Synthetic peptide”
NLGGYSDDSQVWDSSSDLVV

SEQ ID NO: 776 - protein, 21 aa, Artificial Sequence
  source: /note=“Description of Artificial Sequence Synthetic peptide”
SGSIASNYEDNQSYDTSNLVV

SEQ ID NO: 777 - protein, 20 aa, Artificial Sequence
  source: /note=“Description of Artificial Sequence Synthetic peptide”
NIGSKNDDTQVWDRNTGHVV

SEQ ID NO: 778 - protein, 23 aa, Artificial Sequence
  source: /note=“Description of Artificial Sequence Synthetic peptide”
SSDVGGYNYEVSSSYTSSSTLVV

SEQ ID NO: 779 - protein, 21 aa, Artificial Sequence
  source: /note=“Description of Artificial Sequence Synthetic peptide”
NIGNKNDDKQVWDTSEYQNRV

SEQ ID NO: 780 - protein, 22 aa, Artificial Sequence
  source: /note=“Description of Artificial Sequence Synthetic peptide”
SGSIASNYEHNQSYDNSNPHVV

SEQ ID NO: 781 - protein, 24 aa, Artificial Sequence
  source: /note=“Description of Artificial Sequence Synthetic peptide”
SSNIGAGYDGNSQSYDSSLSGFYV

SEQ ID NO: 782 - protein, 20 aa, Artificial Sequence
  source: /note=“Description of Artificial Sequence Synthetic peptide”
NIGNKNDDSQVWDSSSDHVV

SEQ ID NO: 783 - protein, 18 aa, Artificial Sequence
  source: /note=“Description of Artificial Sequence Synthetic peptide”
QGISSWGASQQANSFPIT

SEQ ID NO: 784 - protein, 21 aa, Artificial Sequence
  source: /note=“Description of Artificial Sequence Synthetic peptide”
SGSIASNYEDNQSYDSSNHVV

SEQ ID NO: 785 - protein, 18 aa, Artificial Sequence
  source: /note=“Description of Artificial Sequence Synthetic peptide”
QGVNSDGASQQYNNWPWT

SEQ ID NO: 786 - protein, 18 aa, Artificial Sequence
  source: /note=“Description of Artificial Sequence Synthetic peptide”
KLGDKYEDTQAWDTSAVV

SEQ ID NO: 787 - protein, 20 aa, Artificial Sequence
  source: /note=“Description of Artificial Sequence Synthetic peptide”
NIGSKSDDSQLWDDSSDHVV

SEQ ID NO: 788 - protein, 20 aa, Artificial Sequence
  source: /note=“Description of Artificial Sequence Synthetic peptide”
NIGSKSDDSQVWDSSSDHVV

SEQ ID NO: 789 - protein, 20 aa, Artificial Sequence
  source: /note=“Description of Artificial Sequence Synthetic peptide”
SLRDYYGKNNSRDSSGNHVV

SEQ ID NO: 790 - protein, 20 aa, Artificial Sequence
  source: /note=“Description of Artificial Sequence Synthetic peptide”
NIGRKSDDTQLYDSDSDNVV

SEQ ID NO: 791 - protein, 20 aa, Artificial Sequence
  source: /note=“Description of Artificial Sequence Synthetic peptide”
NIGSKSDDSQVWDSSSDHPV

SEQ ID NO: 792 - protein, 20 aa, Artificial Sequence
  source: /note=“Description of Artificial Sequence Synthetic peptide”
SLRSYYGKNNSRDSSGNLGV

SEQ ID NO: 793 - protein, 18 aa, Artificial Sequence
  source: /note=“Description of Artificial Sequence Synthetic peptide”
QNILTNAASQQSYSIPWT

SEQ ID NO: 794 - protein, 18 aa, Artificial Sequence
  source: /note=“Description of Artificial Sequence Synthetic peptide”
KLGNKYENNQAWDSSTAV

SEQ ID NO: 795 - protein, 18 aa, Artificial Sequence
  source: /note=“Description of Artificial Sequence Synthetic peptide”
QSISSYAASQQSYSTSWT

SEQ ID NO: 796 - protein, 21 aa, Artificial Sequence
  source: /note=“Description of Artificial Sequence Synthetic peptide”
NIGSKSDDSAAWDDSLNGQVV

SEQ ID NO: 797 - protein, 20 aa, Artificial Sequence
  source: /note=“Description of Artificial Sequence Synthetic peptide”
NIGSKSDDSQVWDSSSDHVV

SEQ ID NO: 798 - protein, 20 aa, Artificial Sequence
  source: /note=“Description of Artificial Sequence Synthetic peptide”
NVGTTSDDTQVWDSSSDHVI

SEQ ID NO: 799 - protein, 20 aa, Artificial Sequence
  source: /note=“Description of Artificial Sequence Synthetic peptide”
KIGSYSDDSQVWDTYGDQVV

SEQ ID NO: 800 - protein, 20 aa, Artificial Sequence
  source: /note=“Description of Artificial Sequence Synthetic peptide”
NIGSKSDDSQVWDSSSDHPV

SEQ ID NO: 801 - protein, 20 aa, Artificial Sequence
  source: /note=“Description of Artificial Sequence Synthetic peptide”
NIGSKSDDSQVWDSGSDFVV

SEQ ID NO: 802 - protein, 20 aa, Artificial Sequence
  source: /note=“Description of Artificial Sequence Synthetic peptide”
NIGSKSDDSQVWDSSSDHPV

SEQ ID NO: 803 - protein, 20 aa, Artificial Sequence
  source: /note=“Description of Artificial Sequence Synthetic peptide”
NIGSQSDDSQVWDGSNDHVV

SEQ ID NO: 804 - protein, 20 aa, Artificial Sequence
  source: /note=“Description of Artificial Sequence Synthetic peptide”
NIGRESDDSQVWDSSIDHVV

SEQ ID NO: 805 - protein, 20 aa, Artificial Sequence
  source: /note=“Description of Artificial Sequence Synthetic peptide”
NIGSKSDDSQVWDSSSDHVV

SEQ ID NO: 806 - protein, 20 aa, Artificial Sequence
  source: /note=“Description of Artificial Sequence Synthetic peptide”
NIGSKSDDSQVWDSSSDHVV

SEQ ID NO: 807 - protein, 20 aa, Artificial Sequence
  source: /note=“Description of Artificial Sequence Synthetic peptide”
NIGSKGDDSQVWDNSSDSVV

SEQ ID NO: 808 - protein, 20 aa, Artificial Sequence
  source: /note=“Description of Artificial Sequence Synthetic peptide”
GGSIASNYKDNQSYGSGNVV

SEQ ID NO: 809 - protein, 22 aa, Artificial Sequence
  source: /note=“Description of Artificial Sequence Synthetic peptide”
SGSIASNYEHNQSFDRNNPKWV

SEQ ID NO: 810 - protein, 21 aa, Artificial Sequence
  source: /note=“Description of Artificial Sequence Synthetic peptide”
NIGSKSDDSQVWDSSSDHLVV

SEQ ID NO: 811 - protein, 20 aa, Artificial Sequence
  source: /note=“Description of Artificial Sequence Synthetic peptide”
KLGDKYHDTQVWDGTTDHFL

SEQ ID NO: 812 - protein, 20 aa, Artificial Sequence
  source: /note=“Description of Artificial Sequence Synthetic peptide”
NIGSKSYDSQVWDSVSDPVM

SEQ ID NO: 813 - protein, 22 aa, Artificial Sequence
  source: /note=“Description of Artificial Sequence Synthetic peptide”
SSDVGGYNYEVSSSYAGSNNLV

SEQ ID NO: 814 - protein, 18 aa, Artificial Sequence
  source: /note=“Description of Artificial Sequence Synthetic peptide”
KLGDKYQNNQAWDSSAVV

SEQ ID NO: 815 - protein, 22 aa, Artificial Sequence
  source: /note=“Description of Artificial Sequence Synthetic peptide”
NIGSKSDDSQVWDSTSDHPEVV

SEQ ID NO: 816 - protein, 20 aa, Artificial Sequence
  source: /note=“Description of Artificial Sequence Synthetic peptide”
NIGSKSDDDQVWDSGSDHVV

SEQ ID NO: 817 - protein, 20 aa, Artificial Sequence
  source: /note=“Description of Artificial Sequence Synthetic peptide”
NIGSKSDDSQVWDSSSDHVV

SEQ ID NO: 818 - protein, 20 aa, Artificial Sequence
  source: /note=“Description of Artificial Sequence Synthetic peptide”
NIGSKSDDSQVWDSSSDHVV

SEQ ID NO: 819 - protein, 22 aa, Artificial Sequence
  source: /note=“Description of Artificial Sequence Synthetic peptide”
SSNIGNNYENNGTWDSSLSAGV

SEQ ID NO: 820 - protein, 22 aa, Artificial Sequence
  source: /note=“Description of Artificial Sequence Synthetic peptide”
SSNIGAGYDGNSQSYDSSLSWV

SEQ ID NO: 821 - protein, 22 aa, Artificial Sequence
  source: /note=“Description of Artificial Sequence Synthetic peptide”
SSDVGGYNFGVSSSYRIRDSLV

SEQ ID NO: 822 - protein, 20 aa, Artificial Sequence
  source: /note=“Description of Artificial Sequence Synthetic peptide”
NIGSKSDDSQVWDSSSDHVV

SEQ ID NO: 823 - protein, 21 aa, Artificial Sequence
  source: /note=“Description of Artificial Sequence Synthetic peptide”
GGGIADNYDDDQSYDSAVPVV

SEQ ID NO: 824 - protein, 22 aa, Artificial Sequence
  source: /note=“Description of Artificial Sequence Synthetic peptide”
NIGSKSDDSQVWDSDNDNSEVI

SEQ ID NO: 825 - protein, 20 aa, Artificial Sequence
  source: /note=“Description of Artificial Sequence Synthetic peptide”
NIGSKNDDNQVWDSSSEHVV

SEQ ID NO: 826 - protein, 20 aa, Artificial Sequence
  source: /note=“Description of Artificial Sequence Synthetic peptide”
NIGSNSDDSQVWDSSSDHVV

SEQ ID NO: 827 - protein, 21 aa, Artificial Sequence
  source: /note=“Description of Artificial Sequence Synthetic peptide”
ILGHYHGKDNNSRDRSGTQVL

SEQ ID NO: 828 - protein, 23 aa, Artificial Sequence
  source: /note=“Description of Artificial Sequence Synthetic peptide”
SSDVGGYNYEVSSSYTSSSTLVV

SEQ ID NO: 829 - protein, 19 aa, Artificial Sequence
  source: /note=“Description of Artificial Sequence Synthetic peptide”
QSVSTNGASQQYNTWPPVR

SEQ ID NO: 830 - protein, 23 aa, Artificial Sequence
  source: /note=“Description of Artificial Sequence Synthetic peptide”
SSDVGGYNYDVSSSYTSSSTLVV

SEQ ID NO: 831 - protein, 23 aa, Artificial Sequence
  source: /note=“Description of Artificial Sequence Synthetic peptide”
SSDVGGYNYDVSSSYTSSSTLVV

SEQ ID NO: 832 - protein, 20 aa, Artificial Sequence
  source: /note=“Description of Artificial Sequence Synthetic peptide”
KIGSKIHDSQVWDVNTDHVV

SEQ ID NO: 833 - protein, 23 aa, Artificial Sequence
  source: /note=“Description of Artificial Sequence Synthetic peptide”
SSDVGGYNYEVTSSYTSSSTFVV

SEQ ID NO: 834 - protein, 20 aa, Artificial Sequence
  source: /note=“Description of Artificial Sequence Synthetic peptide”
SGSIVSNYEDNQSYDSGNVV

SEQ ID NO: 835 - protein, 19 aa, Artificial Sequence
  source: /note=“Description of Artificial Sequence Synthetic peptide”
QSVSSSYGASQQYGSSPLT

SEQ ID NO: 836 - protein, 20 aa, Artificial Sequence
  source: /note=“Description of Artificial Sequence Synthetic peptide”
SGSIATNYEDNQSYDSSTGV

SEQ ID NO: 837 - protein, 23 aa, Artificial Sequence
  source: /note=“Description of Artificial Sequence Synthetic peptide”
SSDVGGYNYDVSSSYTSSSTLVV

SEQ ID NO: 838 - protein, 18 aa, Artificial Sequence
  source: /note=“Description of Artificial Sequence Synthetic peptide”
NIESKSDDSQVWDSGHQV

SEQ ID NO: 839 - protein, 22 aa, Artificial Sequence
  source: /note=“Description of Artificial Sequence Synthetic peptide”
SSYIATNSSDSAAWDDSLNAYV

SEQ ID NO: 840 - protein, 23 aa, Artificial Sequence
  source: /note=“Description of Artificial Sequence Synthetic peptide”
SSDIGGYNYEVSSSYTSSSTLVV

SEQ ID NO: 841 - protein, 23 aa, Artificial Sequence
  source: /note=“Description of Artificial Sequence Synthetic peptide”
SSDVGGYNYDVSSSYTSSSTLVV

SEQ ID NO: 842 - protein, 24 aa, Artificial Sequence
  source: /note=“Description of Artificial Sequence Synthetic peptide”
SSNIGAGYDGNNATWDDSLNAPYV

SEQ ID NO: 843 - protein, 18 aa, Artificial Sequence
  source: /note=“Description of Artificial Sequence Synthetic peptide”
KLGNKYQDDQAWDSTYVV

SEQ ID NO: 844 - protein, 18 aa, Artificial Sequence
  source: /note=“Description of Artificial Sequence Synthetic peptide”
KLGDKYQDTQAWDSTTLV

SEQ ID NO: 845 - protein, 20 aa, Artificial Sequence
  source: /note=“Description of Artificial Sequence Synthetic peptide”
GGSIASNYKDNQSYGSGNVV

SEQ ID NO: 846 - protein, 22 aa, Artificial Sequence
  source: /note=“Description of Artificial Sequence Synthetic peptide”
SSNIASNTSNNSAWDDSLHTYV

SEQ ID NO: 847 - protein, 22 aa, Artificial Sequence
  source: /note=“Description of Artificial Sequence Synthetic peptide”
SSDVGGYNYEVSSSYAGSDTVV

SEQ ID NO: 848 - protein, 22 aa, Artificial Sequence
  source: /note=“Description of Artificial Sequence Synthetic peptide”
SSNIGNNYDNDGTWDNSLSAVV

SEQ ID NO: 849 - protein, 20 aa, Artificial Sequence
  source: /note=“Description of Artificial Sequence Synthetic peptide”
NIGSKSDDSQVWDSSSDHVV

SEQ ID NO: 850 - protein, 23 aa, Artificial Sequence
  source: /note=“Description of Artificial Sequence Synthetic peptide”
SSDVGGYNYDVSSSYTSSSTLVV

SEQ ID NO: 851 - protein, 22 aa, Artificial Sequence
  source: /note=“Description of Artificial Sequence Synthetic peptide”
SSNIGNNYENNGTWDSSLSAVV

SEQ ID NO: 852 - protein, 23 aa, Artificial Sequence
  source: /note=“Description of Artificial Sequence Synthetic peptide”
SSDVGGYDYEVSSSYTSSSTLVV

SEQ ID NO: 853 - protein, 19 aa, Artificial Sequence
  source: /note=“Description of Artificial Sequence Synthetic peptide”
NIGSKSADSQVWDSSFDVA

SEQ ID NO: 854 - protein, 20 aa, Artificial Sequence
  source: /note=“Description of Artificial Sequence Synthetic peptide”
NIGDKRYDTQVWDTDTNHAV

SEQ ID NO: 855 - protein, 22 aa, Artificial Sequence
  source: /note=“Description of Artificial Sequence Synthetic peptide”
SSDVGAYNYDVSSSYTTSSTLV

SEQ ID NO: 856 - protein, 18 aa, Artificial Sequence
  source: /note=“Description of Artificial Sequence Synthetic peptide”
KLGDKYQDSQTWDSSTVV

SEQ ID NO: 857 - protein, 18 aa, Artificial Sequence
  source: /note=“Description of Artificial Sequence Synthetic peptide”
KLGDKYQDIQAWDRSSYV

SEQ ID NO: 858 - protein, 23 aa, Artificial Sequence
  source: /note=“Description of Artificial Sequence Synthetic peptide”
SSDVGGYNYEVSSSYSGSNNLVV

SEQ ID NO: 859 - protein, 23 aa, Artificial Sequence
  source: /note=“Description of Artificial Sequence Synthetic peptide”
SSDVGGYNYDVNSSYTSSNTLVV

SEQ ID NO: 860 - protein, 25 aa, Artificial Sequence
  source: /note=“Description of Artificial Sequence Synthetic peptide”
SSNIGAGYDGNSQSYDSSLSGSGYV

SEQ ID NO: 861 - protein, 23 aa, Artificial Sequence
  source: /note=“Description of Artificial Sequence Synthetic peptide”
SSDVGGYNYEVSSSYTSSSTLVV

SEQ ID NO: 862 - protein, 23 aa, Artificial Sequence
  source: /note=“Description of Artificial Sequence Synthetic peptide”
SSDVGGYNYDVSSSYTSSSTLVV

SEQ ID NO: 863 - protein, 21 aa, Artificial Sequence
  source: /note=“Description of Artificial Sequence Synthetic peptide”
NIGSKSDDSQVWDSGNIHPVV

SEQ ID NO: 864 - protein, 18 aa, Artificial Sequence
  source: /note=“Description of Artificial Sequence Synthetic peptide”
GNNYENNGTWDSSLNVGV

SEQ ID NO: 865 - protein, 18 aa, Artificial Sequence
  source: /note=“Description of Artificial Sequence Synthetic peptide”
KLGNKYQDNQAWDSSTAV

SEQ ID NO: 866 - protein, 21 aa, Artificial Sequence
  source: /note=“Description of Artificial Sequence Synthetic peptide”
SSDVGGYNYDVSSSYAGSSVV

SEQ ID NO: 867 - protein, 23 aa, Artificial Sequence
  source: /note=“Description of Artificial Sequence Synthetic peptide”
SSDVGGYNYEVSSSYTSSSTLVV

SEQ ID NO: 868 - protein, 24 aa, Artificial Sequence
  source: /note=“Description of Artificial Sequence Synthetic peptide”
GSNIGAGYDGNIAAWDDSLNGLYV

SEQ ID NO: 869 - protein, 23 aa, Artificial Sequence
  source: /note=“Description of Artificial Sequence Synthetic peptide”
SSDVGGYNYDVSSSYTSSSTFVV

SEQ ID NO: 870 - protein, 22 aa, Artificial Sequence
  source: /note=“Description of Artificial Sequence Synthetic peptide”
SSNIGINTRNNAAWDDSLSGWV

SEQ ID NO: 871 - protein, 21 aa, Artificial Sequence
  source: /note=“Description of Artificial Sequence Synthetic peptide”
GSDIGDYKYDVTSPHTPSRVI

SEQ ID NO: 872 - protein, 23 aa, Artificial Sequence
  source: /note=“Description of Artificial Sequence Synthetic peptide”
SSNIGAGYDGNSAAWDDGPSGYV

SEQ ID NO: 873 - protein, 18 aa, Artificial Sequence
  source: /note=“Description of Artificial Sequence Synthetic peptide”
KLGDKYRDNQAWDSSTVV

SEQ ID NO: 874 - protein, 19 aa, Artificial Sequence
  source: /note=“Description of Artificial Sequence Synthetic peptide”
QSIDTSAASQQSYSTPQYT

SEQ ID NO: 875 - protein, 18 aa, Artificial Sequence
  source: /note=“Description of Artificial Sequence Synthetic peptide”
QSISSWKASQQYNTYFPT

SEQ ID NO: 876 - protein, 8 aa, Artificial Sequence
  source: /note=“Description of Artificial Sequence Synthetic peptide”
TSNIGANH

SEQ ID NO: 877 - protein, 9 aa, Artificial Sequence
  source: /note=“Description of Artificial Sequence Synthetic peptide”
SSDIGGYKY

SEQ ID NO: 878 - protein, 6 aa, Artificial Sequence
  source: /note=“Description of Artificial Sequence Synthetic peptide”
QSISSF

SEQ ID NO: 879 - protein, 6 aa, Artificial Sequence
  source: /note=“Description of Artificial Sequence Synthetic peptide”
QSVSSN

SEQ ID NO: 880 - protein, 6 aa, Artificial Sequence
  source: /note=“Description of Artificial Sequence Synthetic peptide”
QSVSSN

SEQ ID NO: 881 - protein, 6 aa, Artificial Sequence
  source: /note=“Description of Artificial Sequence Synthetic peptide”
NIGSKS

SEQ ID NO: 882 - protein, 6 aa, Artificial Sequence
  source: /note=“Description of Artificial Sequence Synthetic peptide”
NIGSKS

SEQ ID NO: 883 - protein, 9 aa, Artificial Sequence
  source: /note=“Description of Artificial Sequence Synthetic peptide”
SSNIGAGYD

SEQ ID NO: 884 - protein, 8 aa, Artificial Sequence
  source: /note=“Description of Artificial Sequence Synthetic peptide”
SGSITDDY

SEQ ID NO: 885 - protein, 6 aa, Artificial Sequence
  source: /note=“Description of Artificial Sequence Synthetic peptide”
QSVSSN

SEQ ID NO: 886 - protein, 6 aa, Artificial Sequence
  source: /note=“Description of Artificial Sequence Synthetic peptide”
NIGSKS

SEQ ID NO: 887 - protein, 7 aa, Artificial Sequence
  source: /note=“Description of Artificial Sequence Synthetic peptide”
QSVSSSY

SEQ ID NO: 888 - protein, 7 aa, Artificial Sequence
  source: /note=“Description of Artificial Sequence Synthetic peptide”
QSVTSNY

SEQ ID NO: 889 - protein, 9 aa, Artificial Sequence
  source: /note=“Description of Artificial Sequence Synthetic peptide”
TGAVTSGFY

SEQ ID NO: 890 - protein, 6 aa, Artificial Sequence
  source: /note=“Description of Artificial Sequence Synthetic peptide”
NIGSKS

SEQ ID NO: 891 - protein, 6 aa, Artificial Sequence
  source: /note=“Description of Artificial Sequence Synthetic peptide”
QTISSY

SEQ ID NO: 892 - protein, 6 aa, Artificial Sequence
  source: /note=“Description of Artificial Sequence Synthetic peptide”
NIGSKS

SEQ ID NO: 893 - protein, 6 aa, Artificial Sequence
  source: /note=“Description of Artificial Sequence Synthetic peptide”
NIGSKS

SEQ ID NO: 894 - protein, 7 aa, Artificial Sequence
  source: /note=“Description of Artificial Sequence Synthetic peptide”
QRVRSSY

SEQ ID NO: 895 - protein, 6 aa, Artificial Sequence
  source: /note=“Description of Artificial Sequence Synthetic peptide”
QTVSNN

SEQ ID NO: 896 - protein, 6 aa, Artificial Sequence
  source: /note=“Description of Artificial Sequence Synthetic peptide”
NIGSKS

SEQ ID NO: 897 - protein, 6 aa, Artificial Sequence
  source: /note=“Description of Artificial Sequence Synthetic peptide”
DIESKS

SEQ ID NO: 898 - protein, 7 aa, Artificial Sequence
  source: /note=“Description of Artificial Sequence Synthetic peptide”
QGVRASS

SEQ ID NO: 899 - protein, 6 aa, Artificial Sequence
  source: /note=“Description of Artificial Sequence Synthetic peptide”
QSISSY

SEQ ID NO: 900 - protein, 7 aa, Artificial Sequence
  source: /note=“Description of Artificial Sequence Synthetic peptide”
QSVSSSY

SEQ ID NO: 901 - protein, 6 aa, Artificial Sequence
  source: /note=“Description of Artificial Sequence Synthetic peptide”
NIGSKS

SEQ ID NO: 902 - protein, 6 aa, Artificial Sequence
  source: /note=“Description of Artificial Sequence Synthetic peptide”
NIGSKS

SEQ ID NO: 903 - protein, 8 aa, Artificial Sequence
  source: /note=“Description of Artificial Sequence Synthetic peptide”
SSNIGNNY

SEQ ID NO: 904 - protein, 6 aa, Artificial Sequence
  source: /note=“Description of Artificial Sequence Synthetic peptide”
NIGAKS

SEQ ID NO: 905 - protein, 11 aa, Artificial Sequence
  source: /note=“Description of Artificial Sequence Synthetic peptide”
QSLVYSDGNTY

SEQ ID NO: 906 - protein, 6 aa, Artificial Sequence
  source: /note=“Description of Artificial Sequence Synthetic peptide”
SLRSYY

SEQ ID NO: 907 - protein, 6 aa, Artificial Sequence
  source: /note=“Description of Artificial Sequence Synthetic peptide”
NIGSKS

SEQ ID NO: 908 - protein, 6 aa, Artificial Sequence
  source: /note=“Description of Artificial Sequence Synthetic peptide”
NIGSYS

SEQ ID NO: 909 - protein, 6 aa, Artificial Sequence
  source: /note=“Description of Artificial Sequence Synthetic peptide”
NIGSKS

SEQ ID NO: 910 - protein, 6 aa, Artificial Sequence
  source: /note=“Description of Artificial Sequence Synthetic peptide”
NLGGRY

SEQ ID NO: 911 - protein, 6 aa, Artificial Sequence
  source: /note=“Description of Artificial Sequence Synthetic peptide”
NIGSKS

SEQ ID NO: 912 - protein, 6 aa, Artificial Sequence
  source: /note=“Description of Artificial Sequence Synthetic peptide”
SLRSYY

SEQ ID NO: 913 - protein, 6 aa, Artificial Sequence
  source: /note=“Description of Artificial Sequence Synthetic peptide”
KLGDKY

SEQ ID NO: 914 - protein, 6 aa, Artificial Sequence
  source: /note=“Description of Artificial Sequence Synthetic peptide”
QSINSN

SEQ ID NO: 915 - protein, 6 aa, Artificial Sequence
  source: /note=“Description of Artificial Sequence Synthetic peptide”
QRISKY

SEQ ID NO: 916 - protein, 9 aa, Artificial Sequence
  source: /note=“Description of Artificial Sequence Synthetic peptide”
SSNIGAGYR

SEQ ID NO: 917 - protein, 6 aa, Artificial Sequence
  source: /note=“Description of Artificial Sequence Synthetic peptide”
QSVSSN

SEQ ID NO: 918 - protein, 6 aa, Artificial Sequence
  source: /note=“Description of Artificial Sequence Synthetic peptide”
NIGSKS

SEQ ID NO: 919 - protein, 6 aa, Artificial Sequence
  source: /note=“Description of Artificial Sequence Synthetic peptide”
NIGSKS

SEQ ID NO: 920 - protein, 6 aa, Artificial Sequence
  source: /note=“Description of Artificial Sequence Synthetic peptide”
QSVSSN

SEQ ID NO: 921 - protein, 6 aa, Artificial Sequence
  source: /note=“Description of Artificial Sequence Synthetic peptide”
NIGSKS

SEQ ID NO: 922 - protein, 6 aa, Artificial Sequence
  source: /note=“Description of Artificial Sequence Synthetic peptide”
NIGSKS

SEQ ID NO: 923 - protein, 6 aa, Artificial Sequence
  source: /note=“Description of Artificial Sequence Synthetic peptide”
NIGTKS

SEQ ID NO: 924 - protein, 6 aa, Artificial Sequence
  source: /note=“Description of Artificial Sequence Synthetic peptide”
NIGSKS

SEQ ID NO: 925 - protein, 6 aa, Artificial Sequence
  source: /note=“Description of Artificial Sequence Synthetic peptide”
NIGSKG

SEQ ID NO: 926 - protein, 8 aa, Artificial Sequence
  source: /note=“Description of Artificial Sequence Synthetic peptide”
SSNIGNNG

SEQ ID NO: 927 - protein, 6 aa, Artificial Sequence
  source: /note=“Description of Artificial Sequence Synthetic peptide”
NIGSKS

SEQ ID NO: 928 - protein, 8 aa, Artificial Sequence
  source: /note=“Description of Artificial Sequence Synthetic peptide”
GGSLASNY

SEQ ID NO: 929 - protein, 6 aa, Artificial Sequence
  source: /note=“Description of Artificial Sequence Synthetic peptide”
NLGGYS

SEQ ID NO: 930 - protein, 8 aa, Artificial Sequence
  source: /note=“Description of Artificial Sequence Synthetic peptide”
SGSIASNY

SEQ ID NO: 931 - protein, 6 aa, Artificial Sequence
  source: /note=“Description of Artificial Sequence Synthetic peptide”
NIGSKN

SEQ ID NO: 932 - protein, 9 aa, Artificial Sequence
  source: /note=“Description of Artificial Sequence Synthetic peptide”
SSDVGGYNY

SEQ ID NO: 933 - protein, 6 aa, Artificial Sequence
  source: /note=“Description of Artificial Sequence Synthetic peptide”
NIGNKN

SEQ ID NO: 934 - protein, 8 aa, Artificial Sequence
  source: /note=“Description of Artificial Sequence Synthetic peptide”
SGSIASNY

SEQ ID NO: 935 - protein, 9 aa, Artificial Sequence
  source: /note=“Description of Artificial Sequence Synthetic peptide”
SSNIGAGYD

SEQ ID NO: 936 - protein, 6 aa, Artificial Sequence
  source: /note=“Description of Artificial Sequence Synthetic peptide”
NIGNKN

SEQ ID NO: 937 - protein, 6 aa, Artificial Sequence
  source: /note=“Description of Artificial Sequence Synthetic peptide”
QGISSW

SEQ ID NO: 938 - protein, 8 aa, Artificial Sequence
  source: /note=“Description of Artificial Sequence Synthetic peptide”
SGSIASNY

SEQ ID NO: 939 - protein, 6 aa, Artificial Sequence
  source: /note=“Description of Artificial Sequence Synthetic peptide”
QGVNSD

SEQ ID NO: 940 - protein, 6 aa, Artificial Sequence
  source: /note=“Description of Artificial Sequence Synthetic peptide”
KLGDKY

SEQ ID NO: 941 - protein, 6 aa, Artificial Sequence
  source: /note=“Description of Artificial Sequence Synthetic peptide”
NIGSKS

SEQ ID NO: 942 - protein, 6 aa, Artificial Sequence
  source: /note=“Description of Artificial Sequence Synthetic peptide”
NIGSKS

SEQ ID NO: 943 - protein, 6 aa, Artificial Sequence
  source: /note=“Description of Artificial Sequence Synthetic peptide”
SLRDYY

SEQ ID NO: 944 - protein, 6 aa, Artificial Sequence
  source: /note=“Description of Artificial Sequence Synthetic peptide”
NIGRKS

SEQ ID NO: 945 - protein, 6 aa, Artificial Sequence
  source: /note=“Description of Artificial Sequence Synthetic peptide”
NIGSKS

SEQ ID NO: 946 - protein, 6 aa, Artificial Sequence
  source: /note=“Description of Artificial Sequence Synthetic peptide”
SLRSYY

SEQ ID NO: 947 - protein, 6 aa, Artificial Sequence
  source: /note=“Description of Artificial Sequence Synthetic peptide”
QNILTN

SEQ ID NO: 948 - protein, 6 aa, Artificial Sequence
  source: /note=“Description of Artificial Sequence Synthetic peptide”
KLGNKY

SEQ ID NO: 949 - protein, 6 aa, Artificial Sequence
  source: /note=“Description of Artificial Sequence Synthetic peptide”
QSISSY

SEQ ID NO: 950 - protein, 6 aa, Artificial Sequence
  source: /note=“Description of Artificial Sequence Synthetic peptide”
NIGSKS

SEQ ID NO: 951 - protein, 6 aa, Artificial Sequence
  source: /note=“Description of Artificial Sequence Synthetic peptide”
NIGSKS

SEQ ID NO: 952 - protein, 6 aa, Artificial Sequence
  source: /note=“Description of Artificial Sequence Synthetic peptide”
NVGTTS

SEQ ID NO: 953 - protein, 6 aa, Artificial Sequence
  source: /note=“Description of Artificial Sequence Synthetic peptide”
KIGSYS

SEQ ID NO: 954 - protein, 6 aa, Artificial Sequence
  source: /note=“Description of Artificial Sequence Synthetic peptide”
NIGSKS

SEQ ID NO: 955 - protein, 6 aa, Artificial Sequence
  source: /note=“Description of Artificial Sequence Synthetic peptide”
NIGSKS

SEQ ID NO: 956 - protein, 6 aa, Artificial Sequence
  source: /note=“Description of Artificial Sequence Synthetic peptide”
NIGSKS

SEQ ID NO: 957 - protein, 6 aa, Artificial Sequence
  source: /note=“Description of Artificial Sequence Synthetic peptide”
NIGSQS

SEQ ID NO: 958 - protein, 6 aa, Artificial Sequence
  source: /note=“Description of Artificial Sequence Synthetic peptide”
NIGRES

SEQ ID NO: 959 - protein, 6 aa, Artificial Sequence
  source: /note=“Description of Artificial Sequence Synthetic peptide”
NIGSKS

SEQ ID NO: 960 - protein, 6 aa, Artificial Sequence
  source: /note=“Description of Artificial Sequence Synthetic peptide”
NIGSKS

SEQ ID NO: 961 - protein, 6 aa, Artificial Sequence
  source: /note=“Description of Artificial Sequence Synthetic peptide”
NIGSKG

SEQ ID NO: 962 - protein, 8 aa, Artificial Sequence
  source: /note=“Description of Artificial Sequence Synthetic peptide”
GGSIASNY

SEQ ID NO: 963 - protein, 8 aa, Artificial Sequence
  source: /note=“Description of Artificial Sequence Synthetic peptide”
SGSIASNY

SEQ ID NO: 964 - protein, 6 aa, Artificial Sequence
  source: /note=“Description of Artificial Sequence Synthetic peptide”
NIGSKS

SEQ ID NO: 965 - protein, 6 aa, Artificial Sequence
  source: /note=“Description of Artificial Sequence Synthetic peptide”
KLGDKY

SEQ ID NO: 966 - protein, 6 aa, Artificial Sequence
  source: /note=“Description of Artificial Sequence Synthetic peptide”
NIGSKS

SEQ ID NO: 967 - protein, 9 aa, Artificial Sequence
  source: /note=“Description of Artificial Sequence Synthetic peptide”
SSDVGGYNY

SEQ ID NO: 968 - protein, 6 aa, Artificial Sequence
  source: /note=“Description of Artificial Sequence Synthetic peptide”
KLGDKY

SEQ ID NO: 969 - protein, 6 aa, Artificial Sequence
  source: /note=“Description of Artificial Sequence Synthetic peptide”
NIGSKS

SEQ ID NO: 970 - protein, 6 aa, Artificial Sequence
  source: /note=“Description of Artificial Sequence Synthetic peptide”
NIGSKS

SEQ ID NO: 971 - protein, 6 aa, Artificial Sequence
  source: /note=“Description of Artificial Sequence Synthetic peptide”
NIGSKS

SEQ ID NO: 972 - protein, 6 aa, Artificial Sequence
  source: /note=“Description of Artificial Sequence Synthetic peptide”
NIGSKS

SEQ ID NO: 973 - protein, 8 aa, Artificial Sequence
  source: /note=“Description of Artificial Sequence Synthetic peptide”
SSNIGNNY

SEQ ID NO: 974 - protein, 9 aa, Artificial Sequence
  source: /note=“Description of Artificial Sequence Synthetic peptide”
SSNIGAGYD

SEQ ID NO: 975 - protein, 9 aa, Artificial Sequence
  source: /note=“Description of Artificial Sequence Synthetic peptide”
SSDVGGYNF

SEQ ID NO: 976 - protein, 6 aa, Artificial Sequence
  source: /note=“Description of Artificial Sequence Synthetic peptide”
NIGSKS

SEQ ID NO: 977 - protein, 8 aa, Artificial Sequence
  source: /note=“Description of Artificial Sequence Synthetic peptide”
GGGIADNY

SEQ ID NO: 978 - protein, 6 aa, Artificial Sequence
  source: /note=“Description of Artificial Sequence Synthetic peptide”
NIGSKS

SEQ ID NO: 979 - protein, 6 aa, Artificial Sequence
  source: /note=“Description of Artificial Sequence Synthetic peptide”
NIGSKN

SEQ ID NO: 980 - protein, 6 aa, Artificial Sequence
  source: /note=“Description of Artificial Sequence Synthetic peptide”
NIGSNS

SEQ ID NO: 981 - protein, 6 aa, Artificial Sequence
  source: /note=“Description of Artificial Sequence Synthetic peptide”
ILGHYH

SEQ ID NO: 982 - protein, 9 aa, Artificial Sequence
  source: /note=“Description of Artificial Sequence Synthetic peptide”
SSDVGGYNY

SEQ ID NO: 983 - protein, 6 aa, Artificial Sequence
  source: /note=“Description of Artificial Sequence Synthetic peptide”
QSVSTN

SEQ ID NO: 984 - protein, 9 aa, Artificial Sequence
  source: /note=“Description of Artificial Sequence Synthetic peptide”
SSDVGGYNY

SEQ ID NO: 985 - protein, 9 aa, Artificial Sequence
  source: /note=“Description of Artificial Sequence Synthetic peptide”
SSDVGGYNY

SEQ ID NO: 986 - protein, 6 aa, Artificial Sequence
  source: /note=“Description of Artificial Sequence Synthetic peptide”
KIGSKI

SEQ ID NO: 987 - protein, 9 aa, Artificial Sequence
  source: /note=“Description of Artificial Sequence Synthetic peptide”
SSDVGGYNY

SEQ ID NO: 988 - protein, 8 aa, Artificial Sequence
  source: /note=“Description of Artificial Sequence Synthetic peptide”
SGSIVSNY

SEQ ID NO: 989 - protein, 7 aa, Artificial Sequence
  source: /note=“Description of Artificial Sequence Synthetic peptide”
QSVSSSY

SEQ ID NO: 990 - protein, 8 aa, Artificial Sequence
  source: /note=“Description of Artificial Sequence Synthetic peptide”
SGSIATNY

SEQ ID NO: 991 - protein, 9 aa, Artificial Sequence
  source: /note=“Description of Artificial Sequence Synthetic peptide”
SSDVGGYNY

SEQ ID NO: 992 - protein, 6 aa, Artificial Sequence
  source: /note=“Description of Artificial Sequence Synthetic peptide”
NIESKS

SEQ ID NO: 993 - protein, 8 aa, Artificial Sequence
  source: /note=“Description of Artificial Sequence Synthetic peptide”
SSYIATNS

SEQ ID NO: 994 - protein, 9 aa, Artificial Sequence
  source: /note=“Description of Artificial Sequence Synthetic peptide”
SSDIGGYNY

SEQ ID NO: 995 - protein, 9 aa, Artificial Sequence
  source: /note=“Description of Artificial Sequence Synthetic peptide”
SSDVGGYNY

SEQ ID NO: 996 - protein, 9 aa, Artificial Sequence
  source: /note=“Description of Artificial Sequence Synthetic peptide”
SSNIGAGYD

SEQ ID NO: 997 - protein, 6 aa, Artificial Sequence
  source: /note=“Description of Artificial Sequence Synthetic peptide”
KLGNKY

SEQ ID NO: 998 - protein, 6 aa, Artificial Sequence
  source: /note=“Description of Artificial Sequence Synthetic peptide”
KLGDKY

SEQ ID NO: 999 - protein, 8 aa, Artificial Sequence
  source: /note=“Description of Artificial Sequence Synthetic peptide”
GGSIASNY

SEQ ID NO: 1000 - protein, 8 aa, Artificial Sequence
  source: /note=“Description of Artificial Sequence Synthetic peptide”
SSNIASNT

SEQ ID NO: 1001 - protein, 9 aa, Artificial Sequence
  source: /note=“Description of Artificial Sequence Synthetic peptide”
SSDVGGYNY

SEQ ID NO: 1002 - protein, 8 aa, Artificial Sequence
  source: /note=“Description of Artificial Sequence Synthetic peptide”
SSNIGNNY

SEQ ID NO: 1003 - protein, 6 aa, Artificial Sequence
  source: /note=“Description of Artificial Sequence Synthetic peptide”
NIGSKS

SEQ ID NO: 1004 - protein, 9 aa, Artificial Sequence
  source: /note=“Description of Artificial Sequence Synthetic peptide”
SSDVGGYNY

SEQ ID NO: 1005 - protein, 8 aa, Artificial Sequence
  source: /note=“Description of Artificial Sequence Synthetic peptide”
SSNIGNNY

SEQ ID NO: 1006 - protein, 9 aa, Artificial Sequence
  source: /note=“Description of Artificial Sequence Synthetic peptide”
SSDVGGYDY

SEQ ID NO: 1007 - protein, 6 aa, Artificial Sequence
  source: /note=“Description of Artificial Sequence Synthetic peptide”
NIGSKS

SEQ ID NO: 1008 - protein, 6 aa, Artificial Sequence
  source: /note=“Description of Artificial Sequence Synthetic peptide”
NIGDKR

SEQ ID NO: 1009 - protein, 9 aa, Artificial Sequence
  source: /note=“Description of Artificial Sequence Synthetic peptide”
SSDVGAYNY

SEQ ID NO: 1010 - protein, 6 aa, Artificial Sequence
  source: /note=“Description of Artificial Sequence Synthetic peptide”
KLGDKY

SEQ ID NO: 1011 - protein, 6 aa, Artificial Sequence
  source: /note=“Description of Artificial Sequence Synthetic peptide”
KLGDKY

SEQ ID NO: 1012 - protein, 9 aa, Artificial Sequence
  source: /note=“Description of Artificial Sequence Synthetic peptide”
SSDVGGYNY

SEQ ID NO: 1013 - protein, 9 aa, Artificial Sequence
  source: /note=“Description of Artificial Sequence Synthetic peptide”
SSDVGGYNY

SEQ ID NO: 1014 - protein, 9 aa, Artificial Sequence
  source: /note=“Description of Artificial Sequence Synthetic peptide”
SSNIGAGYD

SEQ ID NO: 1015 - protein, 9 aa, Artificial Sequence
  source: /note=“Description of Artificial Sequence Synthetic peptide”
SSDVGGYNY

SEQ ID NO: 1016 - protein, 9 aa, Artificial Sequence
  source: /note=“Description of Artificial Sequence Synthetic peptide”
SSDVGGYNY

SEQ ID NO: 1017 - protein, 6 aa, Artificial Sequence
  source: /note=“Description of Artificial Sequence Synthetic peptide”
NIGSKS

SEQ ID NO: 1018 - protein, 4 aa, Artificial Sequence
  source: /note=“Description of Artificial Sequence Synthetic peptide”
GNNY

SEQ ID NO: 1019 - protein, 6 aa, Artificial Sequence
  source: /note=“Description of Artificial Sequence Synthetic peptide”
KLGNKY

SEQ ID NO: 1020 - protein, 9 aa, Artificial Sequence
  source: /note=“Description of Artificial Sequence Synthetic peptide”
SSDVGGYNY

SEQ ID NO: 1021 - protein, 9 aa, Artificial Sequence
  source: /note=“Description of Artificial Sequence Synthetic peptide”
SSDVGGYNY

SEQ ID NO: 1022 - protein, 9 aa, Artificial Sequence
  source: /note=“Description of Artificial Sequence Synthetic peptide”
GSNIGAGYD

SEQ ID NO: 1023 - protein, 9 aa, Artificial Sequence
  source: /note=“Description of Artificial Sequence Synthetic peptide”
SSDVGGYNY

SEQ ID NO: 1024 - protein, 8 aa, Artificial Sequence
  source: /note=“Description of Artificial Sequence Synthetic peptide”
SSNIGINT

SEQ ID NO: 1025 - protein, 9 aa, Artificial Sequence
  source: /note=“Description of Artificial Sequence Synthetic peptide”
GSDIGDYKY

SEQ ID NO: 1026 - protein, 9 aa, Artificial Sequence
  source: /note=“Description of Artificial Sequence Synthetic peptide”
SSNIGAGYD

SEQ ID NO: 1027 - protein, 6 aa, Artificial Sequence
  source: /note=“Description of Artificial Sequence Synthetic peptide”
KLGDKY

SEQ ID NO: 1028 - protein, 6 aa, Artificial Sequence
  source: /note=“Description of Artificial Sequence Synthetic peptide”
QSIDTS

SEQ ID NO: 1029 - protein, 6 aa, Artificial Sequence
  source: /note=“Description of Artificial Sequence Synthetic peptide”
QSISSW

SEQ ID NO: 1030 - protein, 3 aa, Artificial Sequence
  source: /note=“Description of Artificial Sequence Synthetic peptide”
TKN

SEQ ID NO: 1031 - protein, 3 aa, Artificial Sequence
  source: /note=“Description of Artificial Sequence Synthetic peptide”
DVT

SEQ ID NO: 1032 - protein, 3 aa, Artificial Sequence
  source: /note=“Description of Artificial Sequence Synthetic peptide”
AAS

SEQ ID NO: 1033 - protein, 3 aa, Artificial Sequence
  source: /note=“Description of Artificial Sequence Synthetic peptide”
GAS

SEQ ID NO: 1034 - protein, 3 aa, Artificial Sequence
  source: /note=“Description of Artificial Sequence Synthetic peptide”
GAS

SEQ ID NO: 1035 - protein, 3 aa, Artificial Sequence
  source: /note=“Description of Artificial Sequence Synthetic peptide”
DDS

SEQ ID NO: 1036 - protein, 3 aa, Artificial Sequence
  source: /note=“Description of Artificial Sequence Synthetic peptide”
DDS

SEQ ID NO: 1037 - protein, 3 aa, Artificial Sequence
  source: /note=“Description of Artificial Sequence Synthetic peptide”
SSN

SEQ ID NO: 1038 - protein, 3 aa, Artificial Sequence
  source: /note=“Description of Artificial Sequence Synthetic peptide”
EDH

SEQ ID NO: 1039 - protein, 3 aa, Artificial Sequence
  source: /note=“Description of Artificial Sequence Synthetic peptide”
GAS

SEQ ID NO: 1040 - protein, 3 aa, Artificial Sequence
  source: /note=“Description of Artificial Sequence Synthetic peptide”
DDS

SEQ ID NO: 1041 - protein, 3 aa, Artificial Sequence
  source: /note=“Description of Artificial Sequence Synthetic peptide”
GAS

SEQ ID NO: 1042 - protein, 3 aa, Artificial Sequence
  source: /note=“Description of Artificial Sequence Synthetic peptide”
GAS

SEQ ID NO: 1043 - protein, 3 aa, Artificial Sequence
  source: /note=“Description of Artificial Sequence Synthetic peptide”
SAT

SEQ ID NO: 1044 - protein, 3 aa, Artificial Sequence
  source: /note=“Description of Artificial Sequence Synthetic peptide”
DDS

SEQ ID NO: 1045 - protein, 3 aa, Artificial Sequence
  source: /note=“Description of Artificial Sequence Synthetic peptide”
GAS

SEQ ID NO: 1046 - protein, 3 aa, Artificial Sequence
  source: /note=“Description of Artificial Sequence Synthetic peptide”
DDS

SEQ ID NO: 1047 - protein, 3 aa, Artificial Sequence
  source: /note=“Description of Artificial Sequence Synthetic peptide”
DDS

SEQ ID NO: 1048 - protein, 3 aa, Artificial Sequence
  source: /note=“Description of Artificial Sequence Synthetic peptide”
GAS

SEQ ID NO: 1049 - protein, 3 aa, Artificial Sequence
  source: /note=“Description of Artificial Sequence Synthetic peptide”
DAS

SEQ ID NO: 1050 - protein, 3 aa, Artificial Sequence
  source: /note=“Description of Artificial Sequence Synthetic peptide”
DDS

SEQ ID NO: 1051 - protein, 3 aa, Artificial Sequence
  source: /note=“Description of Artificial Sequence Synthetic peptide”
DDS

SEQ ID NO: 1052 - protein, 3 aa, Artificial Sequence
  source: /note=“Description of Artificial Sequence Synthetic peptide”
AAS

SEQ ID NO: 1053 - protein, 3 aa, Artificial Sequence
  source: /note=“Description of Artificial Sequence Synthetic peptide”
AAS

SEQ ID NO: 1054 - protein, 3 aa, Artificial Sequence
  source: /note=“Description of Artificial Sequence Synthetic peptide”
GAS

SEQ ID NO: 1055 - protein, 3 aa, Artificial Sequence
  source: /note=“Description of Artificial Sequence Synthetic peptide”
DDS

SEQ ID NO: 1056 - protein, 3 aa, Artificial Sequence
  source: /note=“Description of Artificial Sequence Synthetic peptide”
DDS

SEQ ID NO: 1057 - protein, 3 aa, Artificial Sequence
  source: /note=“Description of Artificial Sequence Synthetic peptide”
DNN

SEQ ID NO: 1058 - protein, 3 aa, Artificial Sequence
  source: /note=“Description of Artificial Sequence Synthetic peptide”
DDS

SEQ ID NO: 1059 - protein, 3 aa, Artificial Sequence
  source: /note=“Description of Artificial Sequence Synthetic peptide”
KVS

SEQ ID NO: 1060 - protein, 3 aa, Artificial Sequence
  source: /note=“Description of Artificial Sequence Synthetic peptide”
GKN

SEQ ID NO: 1061 - protein, 3 aa, Artificial Sequence
  source: /note=“Description of Artificial Sequence Synthetic peptide”
DDS

SEQ ID NO: 1062 - protein, 3 aa, Artificial Sequence
  source: /note=“Description of Artificial Sequence Synthetic peptide”
DDS

SEQ ID NO: 1063 - protein, 3 aa, Artificial Sequence
  source: /note=“Description of Artificial Sequence Synthetic peptide”
DDS

SEQ ID NO: 1064 - protein, 3 aa, Artificial Sequence
  source: /note=“Description of Artificial Sequence Synthetic peptide”
QDL

SEQ ID NO: 1065 - protein, 3 aa, Artificial Sequence
  source: /note=“Description of Artificial Sequence Synthetic peptide”
DDS

SEQ ID NO: 1066 - protein, 3 aa, Artificial Sequence
  source: /note=“Description of Artificial Sequence Synthetic peptide”
GKN

SEQ ID NO: 1067 - protein, 3 aa, Artificial Sequence
  source: /note=“Description of Artificial Sequence Synthetic peptide”
QDT

SEQ ID NO: 1068 - protein, 3 aa, Artificial Sequence
  source: /note=“Description of Artificial Sequence Synthetic peptide”
GAS

SEQ ID NO: 1069 - protein, 3 aa, Artificial Sequence
  source: /note=“Description of Artificial Sequence Synthetic peptide”
GSS

SEQ ID NO: 1070 - protein, 3 aa, Artificial Sequence
  source: /note=“Description of Artificial Sequence Synthetic peptide”
GDN

SEQ ID NO: 1071 - protein, 3 aa, Artificial Sequence
  source: /note=“Description of Artificial Sequence Synthetic peptide”
GAS

SEQ ID NO: 1072 - protein, 3 aa, Artificial Sequence
  source: /note=“Description of Artificial Sequence Synthetic peptide”
DDS

SEQ ID NO: 1073 - protein, 3 aa, Artificial Sequence
  source: /note=“Description of Artificial Sequence Synthetic peptide”
DDS

SEQ ID NO: 1074 - protein, 3 aa, Artificial Sequence
  source: /note=“Description of Artificial Sequence Synthetic peptide”
GAS

SEQ ID NO: 1075 - protein, 3 aa, Artificial Sequence
  source: /note=“Description of Artificial Sequence Synthetic peptide”
DDS

SEQ ID NO: 1076 - protein, 3 aa, Artificial Sequence
  source: /note=“Description of Artificial Sequence Synthetic peptide”
DDS

SEQ ID NO: 1077 - protein, 3 aa, Artificial Sequence
  source: /note=“Description of Artificial Sequence Synthetic peptide”
DDS

SEQ ID NO: 1078 - protein, 3 aa, Artificial Sequence
  source: /note=“Description of Artificial Sequence Synthetic peptide”
DDS

SEQ ID NO: 1079 - protein, 3 aa, Artificial Sequence
  source: /note=“Description of Artificial Sequence Synthetic peptide”
DDR

SEQ ID NO: 1080 - protein, 3 aa, Artificial Sequence
  source: /note=“Description of Artificial Sequence Synthetic peptide”
YDD

SEQ ID NO: 1081 - protein, 3 aa, Artificial Sequence
  source: /note=“Description of Artificial Sequence Synthetic peptide”
DDS

SEQ ID NO: 1082 - protein, 3 aa, Artificial Sequence
  source: /note=“Description of Artificial Sequence Synthetic peptide”
EDK

SEQ ID NO: 1083 - protein, 3 aa, Artificial Sequence
  source: /note=“Description of Artificial Sequence Synthetic peptide”
DDS

SEQ ID NO: 1084 - protein, 3 aa, Artificial Sequence
  source: /note=“Description of Artificial Sequence Synthetic peptide”
EDN

SEQ ID NO: 1085 - protein, 3 aa, Artificial Sequence
  source: /note=“Description of Artificial Sequence Synthetic peptide”
DDT

SEQ ID NO: 1086 - protein, 3 aa, Artificial Sequence
  source: /note=“Description of Artificial Sequence Synthetic peptide”
EVS

SEQ ID NO: 1087 - protein, 3 aa, Artificial Sequence
  source: /note=“Description of Artificial Sequence Synthetic peptide”
DDK

SEQ ID NO: 1088 - protein, 3 aa, Artificial Sequence
  source: /note=“Description of Artificial Sequence Synthetic peptide”
EHN

SEQ ID NO: 1089 - protein, 3 aa, Artificial Sequence
  source: /note=“Description of Artificial Sequence Synthetic peptide”
GNS

SEQ ID NO: 1090 - protein, 3 aa, Artificial Sequence
  source: /note=“Description of Artificial Sequence Synthetic peptide”
DDS

SEQ ID NO: 1091 - protein, 3 aa, Artificial Sequence
  source: /note=“Description of Artificial Sequence Synthetic peptide”
GAS

SEQ ID NO: 1092 - protein, 3 aa, Artificial Sequence
  source: /note=“Description of Artificial Sequence Synthetic peptide”
EDN

SEQ ID NO: 1093 - protein, 3 aa, Artificial Sequence
  source: /note=“Description of Artificial Sequence Synthetic peptide”
GAS

SEQ ID NO: 1094 - protein, 3 aa, Artificial Sequence
  source: /note=“Description of Artificial Sequence Synthetic peptide”
EDT

SEQ ID NO: 1095 - protein, 3 aa, Artificial Sequence
  source: /note=“Description of Artificial Sequence Synthetic peptide”
DDS

SEQ ID NO: 1096 - protein, 3 aa, Artificial Sequence
  source: /note=“Description of Artificial Sequence Synthetic peptide”
DDS

SEQ ID NO: 1097 - protein, 3 aa, Artificial Sequence
  source: /note=“Description of Artificial Sequence Synthetic peptide”
GKN

SEQ ID NO: 1098 - protein, 3 aa, Artificial Sequence
  source: /note=“Description of Artificial Sequence Synthetic peptide”
DDT

SEQ ID NO: 1099 - protein, 3 aa, Artificial Sequence
  source: /note=“Description of Artificial Sequence Synthetic peptide”
DDS

SEQ ID NO: 1100 - protein, 3 aa, Artificial Sequence
  source: /note=“Description of Artificial Sequence Synthetic peptide”
GKN

SEQ ID NO: 1101 - protein, 3 aa, Artificial Sequence
  source: /note=“Description of Artificial Sequence Synthetic peptide”
AAS

SEQ ID NO: 1102 - protein, 3 aa, Artificial Sequence
  source: /note=“Description of Artificial Sequence Synthetic peptide”
ENN

SEQ ID NO: 1103 - protein, 3 aa, Artificial Sequence
  source: /note=“Description of Artificial Sequence Synthetic peptide”
AAS

SEQ ID NO: 1104 - protein, 3 aa, Artificial Sequence
  source: /note=“Description of Artificial Sequence Synthetic peptide”
DDS

SEQ ID NO: 1105 - protein, 3 aa, Artificial Sequence
  source: /note=“Description of Artificial Sequence Synthetic peptide”
DDS

SEQ ID NO: 1106 - protein, 3 aa, Artificial Sequence
  source: /note=“Description of Artificial Sequence Synthetic peptide”
DDT

SEQ ID NO: 1107 - protein, 3 aa, Artificial Sequence
  source: /note=“Description of Artificial Sequence Synthetic peptide”
DDS

SEQ ID NO: 1108 - protein, 3 aa, Artificial Sequence
  source: /note=“Description of Artificial Sequence Synthetic peptide”
DDS

SEQ ID NO: 1109 - protein, 3 aa, Artificial Sequence
  source: /note=“Description of Artificial Sequence Synthetic peptide”
DDS

SEQ ID NO: 1110 - protein, 3 aa, Artificial Sequence
  source: /note=“Description of Artificial Sequence Synthetic peptide”
DDS

SEQ ID NO: 1111 - protein, 3 aa, Artificial Sequence
  source: /note=“Description of Artificial Sequence Synthetic peptide”
DDS

SEQ ID NO: 1112 - protein, 3 aa, Artificial Sequence
  source: /note=“Description of Artificial Sequence Synthetic peptide”
DDS

SEQ ID NO: 1113 - protein, 3 aa, Artificial Sequence
  source: /note=“Description of Artificial Sequence Synthetic peptide”
DDS

SEQ ID NO: 1114 - protein, 3 aa, Artificial Sequence
  source: /note=“Description of Artificial Sequence Synthetic peptide”
DDS

SEQ ID NO: 1115 - protein, 3 aa, Artificial Sequence
  source: /note=“Description of Artificial Sequence Synthetic peptide”
DDS

SEQ ID NO: 1116 - protein, 3 aa, Artificial Sequence
  source: /note=“Description of Artificial Sequence Synthetic peptide”
KDN

SEQ ID NO: 1117 - protein, 3 aa, Artificial Sequence
  source: /note=“Description of Artificial Sequence Synthetic peptide”
EHN

SEQ ID NO: 1118 - protein, 3 aa, Artificial Sequence
  source: /note=“Description of Artificial Sequence Synthetic peptide”
DDS

SEQ ID NO: 1119 - protein, 3 aa, Artificial Sequence
  source: /note=“Description of Artificial Sequence Synthetic peptide”
HDT

SEQ ID NO: 1120 - protein, 3 aa, Artificial Sequence
  source: /note=“Description of Artificial Sequence Synthetic peptide”
YDS

SEQ ID NO: 1121 - protein, 3 aa, Artificial Sequence
  source: /note=“Description of Artificial Sequence Synthetic peptide”
EVS

SEQ ID NO: 1122 - protein, 3 aa, Artificial Sequence
  source: /note=“Description of Artificial Sequence Synthetic peptide”
QNN

SEQ ID NO: 1123 - protein, 3 aa, Artificial Sequence
  source: /note=“Description of Artificial Sequence Synthetic peptide”
DDS

SEQ ID NO: 1124 - protein, 3 aa, Artificial Sequence
  source: /note=“Description of Artificial Sequence Synthetic peptide”
DDD

SEQ ID NO: 1125 - protein, 3 aa, Artificial Sequence
  source: /note=“Description of Artificial Sequence Synthetic peptide”
DDS

SEQ ID NO: 1126 - protein, 3 aa, Artificial Sequence
  source: /note=“Description of Artificial Sequence Synthetic peptide”
DDS

SEQ ID NO: 1127 - protein, 3 aa, Artificial Sequence
  source: /note=“Description of Artificial Sequence Synthetic peptide”
ENN

SEQ ID NO: 1128 - protein, 3 aa, Artificial Sequence
  source: /note=“Description of Artificial Sequence Synthetic peptide”
GNS

SEQ ID NO: 1129 - protein, 3 aa, Artificial Sequence
  source: /note=“Description of Artificial Sequence Synthetic peptide”
GVS

SEQ ID NO: 1130 - protein, 3 aa, Artificial Sequence
  source: /note=“Description of Artificial Sequence Synthetic peptide”
DDS

SEQ ID NO: 1131 - protein, 3 aa, Artificial Sequence
  source: /note=“Description of Artificial Sequence Synthetic peptide”
DDD

SEQ ID NO: 1132 - protein, 3 aa, Artificial Sequence
  source: /note=“Description of Artificial Sequence Synthetic peptide”
DDS

SEQ ID NO: 1133 - protein, 3 aa, Artificial Sequence
  source: /note=“Description of Artificial Sequence Synthetic peptide”
DDN

SEQ ID NO: 1134 - protein, 3 aa, Artificial Sequence
  source: /note=“Description of Artificial Sequence Synthetic peptide”
DDS

SEQ ID NO: 1135 - protein, 4 aa, Artificial Sequence
  source: /note=“Description of Artificial Sequence Synthetic peptide”
GKDN

SEQ ID NO: 1136 - protein, 3 aa, Artificial Sequence
  source: /note=“Description of Artificial Sequence Synthetic peptide”
EVS

SEQ ID NO: 1137 - protein, 3 aa, Artificial Sequence
  source: /note=“Description of Artificial Sequence Synthetic peptide”
GAS

SEQ ID NO: 1138 - protein, 3 aa, Artificial Sequence
  source: /note=“Description of Artificial Sequence Synthetic peptide”
DVS

SEQ ID NO: 1139 - protein, 3 aa, Artificial Sequence
  source: /note=“Description of Artificial Sequence Synthetic peptide”
DVS

SEQ ID NO: 1140 - protein, 3 aa, Artificial Sequence
  source: /note=“Description of Artificial Sequence Synthetic peptide”
HDS

SEQ ID NO: 1141 - protein, 3 aa, Artificial Sequence
  source: /note=“Description of Artificial Sequence Synthetic peptide”
EVT

SEQ ID NO: 1142 - protein, 3 aa, Artificial Sequence
  source: /note=“Description of Artificial Sequence Synthetic peptide”
EDN

SEQ ID NO: 1143 - protein, 3 aa, Artificial Sequence
  source: /note=“Description of Artificial Sequence Synthetic peptide”
GAS

SEQ ID NO: 1144 - protein, 3 aa, Artificial Sequence
  source: /note=“Description of Artificial Sequence Synthetic peptide”
EDN

SEQ ID NO: 1145 - protein, 3 aa, Artificial Sequence
  source: /note=“Description of Artificial Sequence Synthetic peptide”
DVS

SEQ ID NO: 1146 - protein, 3 aa, Artificial Sequence
  source: /note=“Description of Artificial Sequence Synthetic peptide”
DDS

SEQ ID NO: 1147 - protein, 3 aa, Artificial Sequence
  source: /note=“Description of Artificial Sequence Synthetic peptide”
SDS

SEQ ID NO: 1148 - protein, 3 aa, Artificial Sequence
  source: /note=“Description of Artificial Sequence Synthetic peptide”
EVS

SEQ ID NO: 1149 - protein, 3 aa, Artificial Sequence
  source: /note=“Description of Artificial Sequence Synthetic peptide”
DVS

SEQ ID NO: 1150 - protein, 3 aa, Artificial Sequence
  source: /note=“Description of Artificial Sequence Synthetic peptide”
GNN

SEQ ID NO: 1151 - protein, 3 aa, Artificial Sequence
  source: /note=“Description of Artificial Sequence Synthetic peptide”
QDD

SEQ ID NO: 1152 - protein, 3 aa, Artificial Sequence
  source: /note=“Description of Artificial Sequence Synthetic peptide”
QDT

SEQ ID NO: 1153 - protein, 3 aa, Artificial Sequence
  source: /note=“Description of Artificial Sequence Synthetic peptide”
KDN

SEQ ID NO: 1154 - protein, 3 aa, Artificial Sequence
  source: /note=“Description of Artificial Sequence Synthetic peptide”
SNN

SEQ ID NO: 1155 - protein, 3 aa, Artificial Sequence
  source: /note=“Description of Artificial Sequence Synthetic peptide”
EVS

SEQ ID NO: 1156 - protein, 3 aa, Artificial Sequence
  source: /note=“Description of Artificial Sequence Synthetic peptide”
DND

SEQ ID NO: 1157 - protein, 3 aa, Artificial Sequence
  source: /note=“Description of Artificial Sequence Synthetic peptide”
DDS

SEQ ID NO: 1158 - protein, 3 aa, Artificial Sequence
  source: /note=“Description of Artificial Sequence Synthetic peptide”
DVS

SEQ ID NO: 1159 - protein, 3 aa, Artificial Sequence
  source: /note=“Description of Artificial Sequence Synthetic peptide”
ENN

SEQ ID NO: 1160 - protein, 3 aa, Artificial Sequence
  source: /note=“Description of Artificial Sequence Synthetic peptide”
EVS

SEQ ID NO: 1161 - protein, 3 aa, Artificial Sequence
  source: /note=“Description of Artificial Sequence Synthetic peptide”
ADS

SEQ ID NO: 1162 - protein, 3 aa, Artificial Sequence
  source: /note=“Description of Artificial Sequence Synthetic peptide”
YDT

SEQ ID NO: 1163 - protein, 3 aa, Artificial Sequence
  source: /note=“Description of Artificial Sequence Synthetic peptide”
DVS

SEQ ID NO: 1164 - protein, 3 aa, Artificial Sequence
  source: /note=“Description of Artificial Sequence Synthetic peptide”
QDS

SEQ ID NO: 1165 - protein, 3 aa, Artificial Sequence
  source: /note=“Description of Artificial Sequence Synthetic peptide”
QDI

SEQ ID NO: 1166 - protein, 3 aa, Artificial Sequence
  source: /note=“Description of Artificial Sequence Synthetic peptide”
EVS

SEQ ID NO: 1167 - protein, 3 aa, Artificial Sequence
  source: /note=“Description of Artificial Sequence Synthetic peptide”
DVN

SEQ ID NO: 1168 - protein, 3 aa, Artificial Sequence
  source: /note=“Description of Artificial Sequence Synthetic peptide”
GNS

SEQ ID NO: 1169 - protein, 3 aa, Artificial Sequence
  source: /note=“Description of Artificial Sequence Synthetic peptide”
EVS

SEQ ID NO: 1170 - protein, 3 aa, Artificial Sequence
  source: /note=“Description of Artificial Sequence Synthetic peptide”
DVS

SEQ ID NO: 1171 - protein, 3 aa, Artificial Sequence
  source: /note=“Description of Artificial Sequence Synthetic peptide”
DDS

SEQ ID NO: 1172 - protein, 3 aa, Artificial Sequence
  source: /note=“Description of Artificial Sequence Synthetic peptide”
ENN

SEQ ID NO: 1173 - protein, 3 aa, Artificial Sequence
  source: /note=“Description of Artificial Sequence Synthetic peptide”
QDN

SEQ ID NO: 1174 - protein, 3 aa, Artificial Sequence
  source: /note=“Description of Artificial Sequence Synthetic peptide”
DVS

SEQ ID NO: 1175 - protein, 3 aa, Artificial Sequence
  source: /note=“Description of Artificial Sequence Synthetic peptide”
EVS

SEQ ID NO: 1176 - protein, 3 aa, Artificial Sequence
  source: /note=“Description of Artificial Sequence Synthetic peptide”
GNI

SEQ ID NO: 1177 - protein, 3 aa, Artificial Sequence
  source: /note=“Description of Artificial Sequence Synthetic peptide”
DVS

SEQ ID NO: 1178 - protein, 3 aa, Artificial Sequence
  source: /note=“Description of Artificial Sequence Synthetic peptide”
RNN

SEQ ID NO: 1179 - protein, 3 aa, Artificial Sequence
  source: /note=“Description of Artificial Sequence Synthetic peptide”
DVT

SEQ ID NO: 1180 - protein, 3 aa, Artificial Sequence
  source: /note=“Description of Artificial Sequence Synthetic peptide”
GNS

SEQ ID NO: 1181 - protein, 3 aa, Artificial Sequence
  source: /note=“Description of Artificial Sequence Synthetic peptide”
RDN

SEQ ID NO: 1182 - protein, 3 aa, Artificial Sequence
  source: /note=“Description of Artificial Sequence Synthetic peptide”
AAS

SEQ ID NO: 1183 - protein, 3 aa, Artificial Sequence
  source: /note=“Description of Artificial Sequence Synthetic peptide”
KAS

SEQ ID NO: 1184 - protein, 11 aa, Artificial Sequence
  source: /note=“Description of Artificial Sequence Synthetic peptide”
AAWDDSLRGWT

SEQ ID NO: 1185 - protein, 11 aa, Artificial Sequence
  source: /note=“Description of Artificial Sequence Synthetic peptide”
GSYSSSSSHYV

SEQ ID NO: 1186 - protein, 9 aa, Artificial Sequence
  source: /note=“Description of Artificial Sequence Synthetic peptide”
QQSYSTPWT

SEQ ID NO: 1187 - protein, 11 aa, Artificial Sequence
  source: /note=“Description of Artificial Sequence Synthetic peptide”
QHYNNWPPQIT

SEQ ID NO: 1188 - protein, 9 aa, Artificial Sequence
  source: /note=“Description of Artificial Sequence Synthetic peptide”
QQYGYSQIT

SEQ ID NO: 1189 - protein, 11 aa, Artificial Sequence
  source: /note=“Description of Artificial Sequence Synthetic peptide”
QVWDSSSDHVV

SEQ ID NO: 1190 - protein, 11 aa, Artificial Sequence
  source: /note=“Description of Artificial Sequence Synthetic peptide”
QVWDSSSDHVV

SEQ ID NO: 1191 - protein, 12 aa, Artificial Sequence
  source: /note=“Description of Artificial Sequence Synthetic peptide”
QSFDPSLSDSWV

SEQ ID NO: 1192 - protein, 9 aa, Artificial Sequence
  source: /note=“Description of Artificial Sequence Synthetic peptide”
QSYDAESWV

SEQ ID NO: 1193 - protein, 9 aa, Artificial Sequence
  source: /note=“Description of Artificial Sequence Synthetic peptide”
QQYGYSQIT

SEQ ID NO: 1194 - protein, 12 aa, Artificial Sequence
  source: /note=“Description of Artificial Sequence Synthetic peptide”
QVWDSSSDLLYV

SEQ ID NO: 1195 - protein, 9 aa, Artificial Sequence
  source: /note=“Description of Artificial Sequence Synthetic peptide”
QQYGRSPFT

SEQ ID NO: 1196 - protein, 8 aa, Artificial Sequence
  source: /note=“Description of Artificial Sequence Synthetic peptide”
QQYGSSPT

SEQ ID NO: 1197 - protein, 11 aa, Artificial Sequence
  source: /note=“Description of Artificial Sequence Synthetic peptide”
LLYYGGAQPWV

SEQ ID NO: 1198 - protein, 11 aa, Artificial Sequence
  source: /note=“Description of Artificial Sequence Synthetic peptide”
QLWDGASDLVI

SEQ ID NO: 1199 - protein, 9 aa, Artificial Sequence
  source: /note=“Description of Artificial Sequence Synthetic peptide”
QQSYSTPQT

SEQ ID NO: 1200 - protein, 11 aa, Artificial Sequence
  source: /note=“Description of Artificial Sequence Synthetic peptide”
QVWDSSSDHVV

SEQ ID NO: 1201 - protein, 11 aa, Artificial Sequence
  source: /note=“Description of Artificial Sequence Synthetic peptide”
QVWDSSSDHVV

SEQ ID NO: 1202 - protein, 11 aa, Artificial Sequence
  source: /note=“Description of Artificial Sequence Synthetic peptide”
QQYGSSPPRII

SEQ ID NO: 1203 - protein, 9 aa, Artificial Sequence
  source: /note=“Description of Artificial Sequence Synthetic peptide”
QQYGSSPLT

SEQ ID NO: 1204 - protein, 11 aa, Artificial Sequence
  source: /note=“Description of Artificial Sequence Synthetic peptide”
QVWDSSSDHVV

SEQ ID NO: 1205 - protein, 11 aa, Artificial Sequence
  source: /note=“Description of Artificial Sequence Synthetic peptide”
QVWDGIINQVV

SEQ ID NO: 1206 - protein, 8 aa, Artificial Sequence
  source: /note=“Description of Artificial Sequence Synthetic peptide”
QQYGRSPT

SEQ ID NO: 1207 - protein, 10 aa, Artificial Sequence
  source: /note=“Description of Artificial Sequence Synthetic peptide”
QQSYSTPPYT

SEQ ID NO: 1208 - protein, 10 aa, Artificial Sequence
  source: /note=“Description of Artificial Sequence Synthetic peptide”
QQYGSSPQYT

SEQ ID NO: 1209 - protein, 11 aa, Artificial Sequence
  source: /note=“Description of Artificial Sequence Synthetic peptide”
QVWGSSNDPVV

SEQ ID NO: 1210 - protein, 11 aa, Artificial Sequence
  source: /note=“Description of Artificial Sequence Synthetic peptide”
QVWDSSSDHVV

SEQ ID NO: 1211 - protein, 11 aa, Artificial Sequence
  source: /note=“Description of Artificial Sequence Synthetic peptide”
GTWDSSLSAVV

SEQ ID NO: 1212 - protein, 13 aa, Artificial Sequence
  source: /note=“Description of Artificial Sequence Synthetic peptide”
QVWDNTGDHPRVI

SEQ ID NO: 1213 - protein, 9 aa, Artificial Sequence
  source: /note=“Description of Artificial Sequence Synthetic peptide”
MQGKHWPPT

SEQ ID NO: 1214 - protein, 11 aa, Artificial Sequence
  source: /note=“Description of Artificial Sequence Synthetic peptide”
NSRDSSGNHWV

SEQ ID NO: 1215 - protein, 12 aa, Artificial Sequence
  source: /note=“Description of Artificial Sequence Synthetic peptide”
QVWDSSSDHSVV

SEQ ID NO: 1216 - protein, 11 aa, Artificial Sequence
  source: /note=“Description of Artificial Sequence Synthetic peptide”
QVWDSSSDHVI

SEQ ID NO: 1217 - protein, 11 aa, Artificial Sequence
  source: /note=“Description of Artificial Sequence Synthetic peptide”
QVWDSSSDHVV

SEQ ID NO: 1218 - protein, 9 aa, Artificial Sequence
  source: /note=“Description of Artificial Sequence Synthetic peptide”
QAWDTYTVV

SEQ ID NO: 1219 - protein, 11 aa, Artificial Sequence
  source: /note=“Description of Artificial Sequence Synthetic peptide”
QVWDSSSDHVV

SEQ ID NO: 1220 - protein, 11 aa, Artificial Sequence
  source: /note=“Description of Artificial Sequence Synthetic peptide”
NSRDSSGNHVV

SEQ ID NO: 1221 - protein, 10 aa, Artificial Sequence
  source: /note=“Description of Artificial Sequence Synthetic peptide”
QAWDSSTNYV

SEQ ID NO: 1222 - protein, 9 aa, Artificial Sequence
  source: /note=“Description of Artificial Sequence Synthetic peptide”
QQFEQWPLT

SEQ ID NO: 1223 - protein, 9 aa, Artificial Sequence
  source: /note=“Description of Artificial Sequence Synthetic peptide”
QQSDSVPIT

SEQ ID NO: 1224 - protein, 11 aa, Artificial Sequence
  source: /note=“Description of Artificial Sequence Synthetic peptide”
QSHDESLNSKV

SEQ ID NO: 1225 - protein, 9 aa, Artificial Sequence
  source: /note=“Description of Artificial Sequence Synthetic peptide”
QQYGSSPLT

SEQ ID NO: 1226 - protein, 11 aa, Artificial Sequence
  source: /note=“Description of Artificial Sequence Synthetic peptide”
QLWDGASDLVI

SEQ ID NO: 1227 - protein, 11 aa, Artificial Sequence
  source: /note=“Description of Artificial Sequence Synthetic peptide”
QVWDSSSDHVV

SEQ ID NO: 1228 - protein, 11 aa, Artificial Sequence
  source: /note=“Description of Artificial Sequence Synthetic peptide”
QQYNNWPPQYT

SEQ ID NO: 1229 - protein, 11 aa, Artificial Sequence
  source: /note=“Description of Artificial Sequence Synthetic peptide”
QVWDSSSDYVV

SEQ ID NO: 1230 - protein, 11 aa, Artificial Sequence
  source: /note=“Description of Artificial Sequence Synthetic peptide”
QVWDSLSDHVI

SEQ ID NO: 1231 - protein, 11 aa, Artificial Sequence
  source: /note=“Description of Artificial Sequence Synthetic peptide”
QVWDHSNDHVV

SEQ ID NO: 1232 - protein, 11 aa, Artificial Sequence
  source: /note=“Description of Artificial Sequence Synthetic peptide”
SAWDSSLTAVV

SEQ ID NO: 1233 - protein, 11 aa, Artificial Sequence
  source: /note=“Description of Artificial Sequence Synthetic peptide”
QVWDTNSQHVV

SEQ ID NO: 1234 - protein, 11 aa, Artificial Sequence
  source: /note=“Description of Artificial Sequence Synthetic peptide”
ATWDDRLKGYV

SEQ ID NO: 1235 - protein, 11 aa, Artificial Sequence
  source: /note=“Description of Artificial Sequence Synthetic peptide”
QVWDSSSDQGV

SEQ ID NO: 1236 - protein, 11 aa, Artificial Sequence
  source: /note=“Description of Artificial Sequence Synthetic peptide”
QSYDSANPLVV

SEQ ID NO: 1237 - protein, 11 aa, Artificial Sequence
  source: /note=“Description of Artificial Sequence Synthetic peptide”
QVWDSSSDLVV

SEQ ID NO: 1238 - protein, 10 aa, Artificial Sequence
  source: /note=“Description of Artificial Sequence Synthetic peptide”
QSYDTSNLVV

SEQ ID NO: 1239 - protein, 11 aa, Artificial Sequence
  source: /note=“Description of Artificial Sequence Synthetic peptide”
QVWDRNTGHVV

SEQ ID NO: 1240 - protein, 11 aa, Artificial Sequence
  source: /note=“Description of Artificial Sequence Synthetic peptide”
SSYTSSSTLVV

SEQ ID NO: 1241 - protein, 12 aa, Artificial Sequence
  source: /note=“Description of Artificial Sequence Synthetic peptide”
QVWDTSEYQNRV

SEQ ID NO: 1242 - protein, 11 aa, Artificial Sequence
  source: /note=“Description of Artificial Sequence Synthetic peptide”
QSYDNSNPHVV

SEQ ID NO: 1243 - protein, 12 aa, Artificial Sequence
  source: /note=“Description of Artificial Sequence Synthetic peptide”
QSYDSSLSGFYV

SEQ ID NO: 1244 - protein, 11 aa, Artificial Sequence
  source: /note=“Description of Artificial Sequence Synthetic peptide”
QVWDSSSDHVV

SEQ ID NO: 1245 - protein, 9 aa, Artificial Sequence
  source: /note=“Description of Artificial Sequence Synthetic peptide”
QQANSFPIT

SEQ ID NO: 1246 - protein, 10 aa, Artificial Sequence
  source: /note=“Description of Artificial Sequence Synthetic peptide”
QSYDSSNHVV

SEQ ID NO: 1247 - protein, 9 aa, Artificial Sequence
  source: /note=“Description of Artificial Sequence Synthetic peptide”
QQYNNWPWT

SEQ ID NO: 1248 - protein, 9 aa, Artificial Sequence
  source: /note=“Description of Artificial Sequence Synthetic peptide”
QAWDTSAVV

SEQ ID NO: 1249 - protein, 11 aa, Artificial Sequence
  source: /note=“Description of Artificial Sequence Synthetic peptide”
QLWDDSSDHVV

SEQ ID NO: 1250 - protein, 11 aa, Artificial Sequence
  source: /note=“Description of Artificial Sequence Synthetic peptide”
QVWDSSSDHVV

SEQ ID NO: 1251 - protein, 11 aa, Artificial Sequence
  source: /note=“Description of Artificial Sequence Synthetic peptide”
NSRDSSGNHVV

SEQ ID NO: 1252 - protein, 11 aa, Artificial Sequence
  source: /note=“Description of Artificial Sequence Synthetic peptide”
QLYDSDSDNVV

SEQ ID NO: 1253 - protein, 11 aa, Artificial Sequence
  source: /note=“Description of Artificial Sequence Synthetic peptide”
QVWDSSSDHPV

SEQ ID NO: 1254 - protein, 11 aa, Artificial Sequence
  source: /note=“Description of Artificial Sequence Synthetic peptide”
NSRDSSGNLGV

SEQ ID NO: 1255 - protein, 9 aa, Artificial Sequence
  source: /note=“Description of Artificial Sequence Synthetic peptide”
QQSYSIPWT

SEQ ID NO: 1256 - protein, 9 aa, Artificial Sequence
  source: /note=“Description of Artificial Sequence Synthetic peptide”
QAWDSSTAV

SEQ ID NO: 1257 - protein, 9 aa, Artificial Sequence
  source: /note=“Description of Artificial Sequence Synthetic peptide”
QQSYSTSWT

SEQ ID NO: 1258 - protein, 12 aa, Artificial Sequence
  source: /note=“Description of Artificial Sequence Synthetic peptide”
AAWDDSLNGQVV

SEQ ID NO: 1259 - protein, 11 aa, Artificial Sequence
  source: /note=“Description of Artificial Sequence Synthetic peptide”
QVWDSSSDHVV

SEQ ID NO: 1260 - protein, 11 aa, Artificial Sequence
  source: /note=“Description of Artificial Sequence Synthetic peptide”
QVWDSSSDHVI

SEQ ID NO: 1261 - protein, 11 aa, Artificial Sequence
  source: /note=“Description of Artificial Sequence Synthetic peptide”
QVWDTYGDQVV

SEQ ID NO: 1262 - protein, 11 aa, Artificial Sequence
  source: /note=“Description of Artificial Sequence Synthetic peptide”
QVWDSSSDHPV

SEQ ID NO: 1263 - protein, 11 aa, Artificial Sequence
  source: /note=“Description of Artificial Sequence Synthetic peptide”
QVWDSGSDFVV

SEQ ID NO: 1264 - protein, 11 aa, Artificial Sequence
  source: /note=“Description of Artificial Sequence Synthetic peptide”
QVWDSSSDHPV

SEQ ID NO: 1265 - protein, 11 aa, Artificial Sequence
  source: /note=“Description of Artificial Sequence Synthetic peptide”
QVWDGSNDHVV

SEQ ID NO: 1266 - protein, 11 aa, Artificial Sequence
  source: /note=“Description of Artificial Sequence Synthetic peptide”
QVWDSSIDHVV

SEQ ID NO: 1267 - protein, 11 aa, Artificial Sequence
  source: /note=“Description of Artificial Sequence Synthetic peptide”
QVWDSSSDHVV

SEQ ID NO: 1268 - protein, 11 aa, Artificial Sequence
  source: /note=“Description of Artificial Sequence Synthetic peptide”
QVWDSSSDHVV

SEQ ID NO: 1269 - protein, 11 aa, Artificial Sequence
  source: /note=“Description of Artificial Sequence Synthetic peptide”
QVWDNSSDSVV

SEQ ID NO: 1270 - protein, 9 aa, Artificial Sequence
  source: /note=“Description of Artificial Sequence Synthetic peptide”
QSYGSGNVV

SEQ ID NO: 1271 - protein, 11 aa, Artificial Sequence
  source: /note=“Description of Artificial Sequence Synthetic peptide”
QSFDRNNPKWV

SEQ ID NO: 1272 - protein, 12 aa, Artificial Sequence
  source: /note=“Description of Artificial Sequence Synthetic peptide”
QVWDSSSDHLVV

SEQ ID NO: 1273 - protein, 11 aa, Artificial Sequence
  source: /note=“Description of Artificial Sequence Synthetic peptide”
QVWDGTTDHFL

SEQ ID NO: 1274 - protein, 11 aa, Artificial Sequence
  source: /note=“Description of Artificial Sequence Synthetic peptide”
QVWDSVSDPVM

SEQ ID NO: 1275 - protein, 10 aa, Artificial Sequence
  source: /note=“Description of Artificial Sequence Synthetic peptide”
SSYAGSNNLV

SEQ ID NO: 1276 - protein, 9 aa, Artificial Sequence
  source: /note=“Description of Artificial Sequence Synthetic peptide”
QAWDSSAVV

SEQ ID NO: 1277 - protein, 13 aa, Artificial Sequence
  source: /note=“Description of Artificial Sequence Synthetic peptide”
QVWDSTSDHPEVV

SEQ ID NO: 1278 - protein, 11 aa, Artificial Sequence
  source: /note=“Description of Artificial Sequence Synthetic peptide”
QVWDSGSDHVV

SEQ ID NO: 1279 - protein, 11 aa, Artificial Sequence
  source: /note=“Description of Artificial Sequence Synthetic peptide”
QVWDSSSDHVV

SEQ ID NO: 1280 - protein, 11 aa, Artificial Sequence
  source: /note=“Description of Artificial Sequence Synthetic peptide”
QVWDSSSDHVV

SEQ ID NO: 1281 - protein, 11 aa, Artificial Sequence
  source: /note=“Description of Artificial Sequence Synthetic peptide”
GTWDSSLSAGV

SEQ ID NO: 1282 - protein, 10 aa, Artificial Sequence
  source: /note=“Description of Artificial Sequence Synthetic peptide”
QSYDSSLSWV

SEQ ID NO: 1283 - protein, 10 aa, Artificial Sequence
  source: /note=“Description of Artificial Sequence Synthetic peptide”
SSYRIRDSLV

SEQ ID NO: 1284 - protein, 11 aa, Artificial Sequence
  source: /note=“Description of Artificial Sequence Synthetic peptide”
QVWDSSSDHVV

SEQ ID NO: 1285 - protein, 10 aa, Artificial Sequence
  source: /note=“Description of Artificial Sequence Synthetic peptide”
QSYDSAVPVV

SEQ ID NO: 1286 - protein, 13 aa, Artificial Sequence
  source: /note=“Description of Artificial Sequence Synthetic peptide”
QVWDSDNDNSEVI

SEQ ID NO: 1287 - protein, 11 aa, Artificial Sequence
  source: /note=“Description of Artificial Sequence Synthetic peptide”
QVWDSSSEHVV

SEQ ID NO: 1288 - protein, 11 aa, Artificial Sequence
  source: /note=“Description of Artificial Sequence Synthetic peptide”
QVWDSSSDHVV

SEQ ID NO: 1289 - protein, 11 aa, Artificial Sequence
  source: /note=“Description of Artificial Sequence Synthetic peptide”
NSRDRSGTQVL

SEQ ID NO: 1290 - protein, 11 aa, Artificial Sequence
  source: /note=“Description of Artificial Sequence Synthetic peptide”
SSYTSSSTLVV

SEQ ID NO: 1291 - protein, 10 aa, Artificial Sequence
  source: /note=“Description of Artificial Sequence Synthetic peptide”
QQYNTWPPVR

SEQ ID NO: 1292 - protein, 11 aa, Artificial Sequence
  source: /note=“Description of Artificial Sequence Synthetic peptide”
SSYTSSSTLVV

SEQ ID NO: 1293 - protein, 11 aa, Artificial Sequence
  source: /note=“Description of Artificial Sequence Synthetic peptide”
SSYTSSSTLVV

SEQ ID NO: 1294 - protein, 11 aa, Artificial Sequence
  source: /note=“Description of Artificial Sequence Synthetic peptide”
QVWDVNTDHVV

SEQ ID NO: 1295 - protein, 11 aa, Artificial Sequence
  source: /note=“Description of Artificial Sequence Synthetic peptide”
SSYTSSSTFVV

SEQ ID NO: 1296 - protein, 9 aa, Artificial Sequence
  source: /note=“Description of Artificial Sequence Synthetic peptide”
QSYDSGNVV

SEQ ID NO: 1297 - protein, 9 aa, Artificial Sequence
  source: /note=“Description of Artificial Sequence Synthetic peptide”
QQYGSSPLT

SEQ ID NO: 1298 - protein, 9 aa, Artificial Sequence
  source: /note=“Description of Artificial Sequence Synthetic peptide”
QSYDSSTGV

SEQ ID NO: 1299 - protein, 11 aa, Artificial Sequence
  source: /note=“Description of Artificial Sequence Synthetic peptide”
SSYTSSSTLVV

SEQ ID NO: 1300 - protein, 9 aa, Artificial Sequence
  source: /note=“Description of Artificial Sequence Synthetic peptide”
QVWDSGHQV

SEQ ID NO: 1301 - protein, 11 aa, Artificial Sequence
  source: /note=“Description of Artificial Sequence Synthetic peptide”
AAWDDSLNAYV

SEQ ID NO: 1302 - protein, 11 aa, Artificial Sequence
  source: /note=“Description of Artificial Sequence Synthetic peptide”
SSYTSSSTLVV

SEQ ID NO: 1303 - protein, 11 aa, Artificial Sequence
  source: /note=“Description of Artificial Sequence Synthetic peptide”
SSYTSSSTLVV

SEQ ID NO: 1304 - protein, 12 aa, Artificial Sequence
  source: /note=“Description of Artificial Sequence Synthetic peptide”
ATWDDSLNAPYV

SEQ ID NO: 1305 - protein, 9 aa, Artificial Sequence
  source: /note=“Description of Artificial Sequence Synthetic peptide”
QAWDSTYVV

SEQ ID NO: 1306 - protein, 9 aa, Artificial Sequence
  source: /note=“Description of Artificial Sequence Synthetic peptide”
QAWDSTTLV

SEQ ID NO: 1307 - protein, 9 aa, Artificial Sequence
  source: /note=“Description of Artificial Sequence Synthetic peptide”
QSYGSGNVV

SEQ ID NO: 1308 - protein, 11 aa, Artificial Sequence
  source: /note=“Description of Artificial Sequence Synthetic peptide”
SAWDDSLHTYV

SEQ ID NO: 1309 - protein, 10 aa, Artificial Sequence
  source: /note=“Description of Artificial Sequence Synthetic peptide”
SSYAGSDTVV

SEQ ID NO: 1310 - protein, 11 aa, Artificial Sequence
  source: /note=“Description of Artificial Sequence Synthetic peptide”
GTWDNSLSAVV

SEQ ID NO: 1311 - protein, 11 aa, Artificial Sequence
  source: /note=“Description of Artificial Sequence Synthetic peptide”
QVWDSSSDHVV

SEQ ID NO: 1312 - protein, 11 aa, Artificial Sequence
  source: /note=“Description of Artificial Sequence Synthetic peptide”
SSYTSSSTLVV

SEQ ID NO: 1313 - protein, 11 aa, Artificial Sequence
  source: /note=“Description of Artificial Sequence Synthetic peptide”
GTWDSSLSAVV

SEQ ID NO: 1314 - protein, 11 aa, Artificial Sequence
  source: /note=“Description of Artificial Sequence Synthetic peptide”
SSYTSSSTLVV

SEQ ID NO: 1315 - protein, 10 aa, Artificial Sequence
  source: /note=“Description of Artificial Sequence Synthetic peptide”
QVWDSSFDVA

SEQ ID NO: 1316 - protein, 11 aa, Artificial Sequence
  source: /note=“Description of Artificial Sequence Synthetic peptide”
QVWDTDTNHAV

SEQ ID NO: 1317 - protein, 10 aa, Artificial Sequence
  source: /note=“Description of Artificial Sequence Synthetic peptide”
SSYTTSSTLV

SEQ ID NO: 1318 - protein, 9 aa, Artificial Sequence
  source: /note=“Description of Artificial Sequence Synthetic peptide”
QTWDSSTVV

SEQ ID NO: 1319 - protein, 9 aa, Artificial Sequence
  source: /note=“Description of Artificial Sequence Synthetic peptide”
QAWDRSSYV

SEQ ID NO: 1320 - protein, 11 aa, Artificial Sequence
  source: /note=“Description of Artificial Sequence Synthetic peptide”
SSYSGSNNLVV

SEQ ID NO: 1321 - protein, 11 aa, Artificial Sequence
  source: /note=“Description of Artificial Sequence Synthetic peptide”
SSYTSSNTLVV

SEQ ID NO: 1322 - protein, 13 aa, Artificial Sequence
  source: /note=“Description of Artificial Sequence Synthetic peptide”
QSYDSSLSGSGYV

SEQ ID NO: 1323 - protein, 11 aa, Artificial Sequence
  source: /note=“Description of Artificial Sequence Synthetic peptide”
SSYTSSSTLVV

SEQ ID NO: 1324 - protein, 11 aa, Artificial Sequence
  source: /note=“Description of Artificial Sequence Synthetic peptide”
SSYTSSSTLVV

SEQ ID NO: 1325 - protein, 12 aa, Artificial Sequence
  source: /note=“Description of Artificial Sequence Synthetic peptide”
QVWDSGNIHPVV

SEQ ID NO: 1326 - protein, 11 aa, Artificial Sequence
  source: /note=“Description of Artificial Sequence Synthetic peptide”
GTWDSSLNVGV

SEQ ID NO: 1327 - protein, 9 aa, Artificial Sequence
  source: /note=“Description of Artificial Sequence Synthetic peptide”
QAWDSSTAV

SEQ ID NO: 1328 - protein, 9 aa, Artificial Sequence
  source: /note=“Description of Artificial Sequence Synthetic peptide”
SSYAGSSVV

SEQ ID NO: 1329 - protein, 11 aa, Artificial Sequence
  source: /note=“Description of Artificial Sequence Synthetic peptide”
SSYTSSSTLVV

SEQ ID NO: 1330 - protein, 12 aa, Artificial Sequence
  source: /note=“Description of Artificial Sequence Synthetic peptide”
AAWDDSLNGLYV

SEQ ID NO: 1331 - protein, 11 aa, Artificial Sequence
  source: /note=“Description of Artificial Sequence Synthetic peptide”
SSYTSSSTFVV

SEQ ID NO: 1332 - protein, 11 aa, Artificial Sequence
  source: /note=“Description of Artificial Sequence Synthetic peptide”
AAWDDSLSGWV

SEQ ID NO: 1333 - protein, 9 aa, Artificial Sequence
  source: /note=“Description of Artificial Sequence Synthetic peptide”
SPHTPSRVI

SEQ ID NO: 1334 - protein, 11 aa, Artificial Sequence
  source: /note=“Description of Artificial Sequence Synthetic peptide”
AAWDDGPSGYV

SEQ ID NO: 1335 - protein, 9 aa, Artificial Sequence
  source: /note=“Description of Artificial Sequence Synthetic peptide”
QAWDSSTVV

SEQ ID NO: 1336 - protein, 10 aa, Artificial Sequence
  source: /note=“Description of Artificial Sequence Synthetic peptide”
QQSYSTPQYT

SEQ ID NO: 1337 - protein, 9 aa, Artificial Sequence
  source: /note=“Description of Artificial Sequence Synthetic peptide”
QQYNTYFPT

SEQ ID NO: 1338 - protein, 29 aa, Artificial Sequence
  source: /note=“Description of Artificial Sequence Synthetic peptide”
GDSISSGYWISYDGSNKARVGSGGWTPDY

SEQ ID NO: 1339 - protein, 31 aa, Artificial Sequence
  source: /note=“Description of Artificial Sequence Synthetic polypeptide”
GDSVSSNSAAINPNSGGTAREVATIPAHFDY

SEQ ID NO: 1340 - protein, 30 aa, Artificial Sequence
  source: /note=“Description of Artificial Sequence Synthetic polypeptide”
GDSVSSNSAAISAYNGNTARDYDILTGLDY

SEQ ID NO: 1341 - protein, 32 aa, Artificial Sequence
  source: /note=“Description of Artificial Sequence Synthetic polypeptide”
GDSVSSNSAAISGSGGRTAKDWAGYINGWYGN

SEQ ID NO: 1342 - protein, 32 aa, Artificial Sequence
  source: /note=“Description of Artificial Sequence Synthetic polypeptide”
GDSVSSNSAAISGSGGSTAKDWAGYVNGWYGN

SEQ ID NO: 1343 - protein, 33 aa, Artificial Sequence
  source: /note=“Description of Artificial Sequence Synthetic polypeptide”
GDSVSSNSAAISGSGGSTAKDWGTSLLYGYFDY

SEQ ID NO: 1344 - protein, 30 aa, Artificial Sequence
  source: /note=“Description of Artificial Sequence Synthetic polypeptide”
GDSVSSNSAAISYDGSNKARVGSGGWTPDY

SEQ ID NO: 1345 - protein, 29 aa, Artificial Sequence
  source: /note=“Description of Artificial Sequence Synthetic peptide”
GDSVSSNSAAIYSGGSTARDFEGSGALDV

SEQ ID NO: 1346 - protein, 34 aa, Artificial Sequence
  source: /note=“Description of Artificial Sequence Synthetic polypeptide”
GDSVSSNSAATYYSSKWYNARGGSSEFYYYGMDV

SEQ ID NO: 1347 - protein, 32 aa, Artificial Sequence
  source: /note=“Description of Artificial Sequence Synthetic polypeptide”
GDSVSSDSASISGSGGITAKDWAGYTNGWYGS

SEQ ID NO: 1348 - protein, 32 aa, Artificial Sequence
  source: /note=“Description of Artificial Sequence Synthetic polypeptide”
GGSISGSNYYISGSGGITAKDWAGYTNGWYGS

SEQ ID NO: 1349 - protein, 31 aa, Artificial Sequence
  source: /note=“Description of Artificial Sequence Synthetic polypeptide”
GGSISSSNWISGSGGSTAKDRSRRAPYYFDY

SEQ ID NO: 1350 - protein, 29 aa, Artificial Sequence
  source: /note=“Description of Artificial Sequence Synthetic peptide”
GGSISSSNWISGSGGSTAKVYRGYDAFDI

SEQ ID NO: 1351 - protein, 28 aa, Artificial Sequence
  source: /note=“Description of Artificial Sequence Synthetic peptide”
GGSISSSNWIYPGDSDTARHAGDGQIDY

SEQ ID NO: 1352 - protein, 33 aa, Artificial Sequence
  source: /note=“Description of Artificial Sequence Synthetic polypeptide”
GGSISSSNWTYYRSKWYNAREGSGLYYYYGMDV

SEQ ID NO: 1353 - protein, 31 aa, Artificial Sequence
  source: /note=“Description of Artificial Sequence Synthetic polypeptide”
GGSVSSNSAAISGSGGSTARGGSGWYHYFDY

SEQ ID NO: 1354 - protein, 30 aa, Artificial Sequence
  source: /note=“Description of Artificial Sequence Synthetic polypeptide”
GGTFSSYAISGTGGRTAKDWAGYINGWYGS

SEQ ID NO: 1355 - protein, 28 aa, Artificial Sequence
  source: /note=“Description of Artificial Sequence Synthetic peptide”
GGTFSSYAISYDGSNKARVGSGGWTPDY

SEQ ID NO: 1356 - protein, 27 aa, Artificial Sequence
  source: /note=“Description of Artificial Sequence Synthetic peptide”
GGTFSSYAIWYDGSNKARLGSGWSLDY

SEQ ID NO: 1357 - protein, 30 aa, Artificial Sequence
  source: /note=“Description of Artificial Sequence Synthetic polypeptide”
GFTFNTYAISGSGDRTAKDWAGYINGWFGN

SEQ ID NO: 1358 - protein, 30 aa, Artificial Sequence
  source: /note=“Description of Artificial Sequence Synthetic polypeptide”
GFTFNTYAISGSGDITAKDWAGYVNGWYGN

SEQ ID NO: 1359 - protein, 28 aa, Artificial Sequence
  source: /note=“Description of Artificial Sequence Synthetic peptide”
GFTFNTYAISYDGSNKARVGSGGWTPDY

SEQ ID NO: 1360 - protein, 36 aa, Artificial Sequence
  source: /note=“Description of Artificial Sequence Synthetic polypeptide”
GFTFDDYAINAGNGNTARGGYCSSTSCYPDYNWFDP

SEQ ID NO: 1361 - protein, 30 aa, Artificial Sequence
  source: /note=“Description of Artificial Sequence Synthetic polypeptide”
GFTFDDYAISGSGDRTAKDWAGYINGWYAN

SEQ ID NO: 1362 - protein, 29 aa, Artificial Sequence
  source: /note=“Description of Artificial Sequence Synthetic peptide”
GFTFDDYAIYSGGSTARDRRGGNWYEFDY

SEQ ID NO: 1363 - protein, 28 aa, Artificial Sequence
  source: /note=“Description of Artificial Sequence Synthetic peptide”
GFTFDDYAIYSGGSTAREGLAMAGYFDY

SEQ ID NO: 1364 - protein, 29 aa, Artificial Sequence
  source: /note=“Description of Artificial Sequence Synthetic peptide”
GFTFGNHGIKHDGSEQARVAVGANLAFDI

SEQ ID NO: 1365 - protein, 30 aa, Artificial Sequence
  source: /note=“Description of Artificial Sequence Synthetic polypeptide”
GFTFSRYGISGSGDRTAKDWAGYINGWYGN

SEQ ID NO: 1366 - protein, 28 aa, Artificial Sequence
  source: /note=“Description of Artificial Sequence Synthetic peptide”
GFTFSNAWIIPIFGTAARGMAQSPAFDY

SEQ ID NO: 1367 - protein, 30 aa, Artificial Sequence
  source: /note=“Description of Artificial Sequence Synthetic polypeptide”
GFTFSNAWISGSGGRTAKDWAGYINGWYGN

SEQ ID NO: 1368 - protein, 26 aa, Artificial Sequence
  source: /note=“Description of Artificial Sequence Synthetic peptide”
GFTFSNAWTYYNSKWYNARETGGFDY

SEQ ID NO: 1369 - protein, 30 aa, Artificial Sequence
  source: /note=“Description of Artificial Sequence Synthetic polypeptide”
GFTFSNYAINTDGGNTARDPVRGDGYNFDY

SEQ ID NO: 1370 - protein, 30 aa, Artificial Sequence
  source: /note=“Description of Artificial Sequence Synthetic polypeptide”
GFTFSNYAISGSGDITAKDWAGYVNGWYGN

SEQ ID NO: 1371 - protein, 33 aa, Artificial Sequence
  source: /note=“Description of Artificial Sequence Synthetic polypeptide”
GFTFSNYAISGSGGSTAKATGYSSGWYGAYFDY

SEQ ID NO: 1372 - protein, 24 aa, Artificial Sequence
  source: /note=“Description of Artificial Sequence Synthetic peptide”
GFTFSNYAIYHSGSTARDRGSMDV

SEQ ID NO: 1373 - protein, 31 aa, Artificial Sequence
  source: /note=“Description of Artificial Sequence Synthetic polypeptide”
GFTFSNYAIYPGDSDTARLGRTSHQSWDLGY

SEQ ID NO: 1374 - protein, 27 aa, Artificial Sequence
  source: /note=“Description of Artificial Sequence Synthetic peptide”
GFTFSNYAIYPGDSDTASGASPYYFDY

SEQ ID NO: 1375 - protein, 28 aa, Artificial Sequence
  source: /note=“Description of Artificial Sequence Synthetic peptide”
GFTFSNYAIYSGGSTARESNTANTHFDY

SEQ ID NO: 1376 - protein, 32 aa, Artificial Sequence
  source: /note=“Description of Artificial Sequence Synthetic polypeptide”
GFTFSNYATYYRSKWYNARGGVGATWYYGMDV

SEQ ID NO: 1377 - protein, 29 aa, Artificial Sequence
  source: /note=“Description of Artificial Sequence Synthetic peptide”
GFTFSNYGISYDGSNKAKQQWLGTWYFDL

SEQ ID NO: 1378 - protein, 31 aa, Artificial Sequence
  source: /note=“Description of Artificial Sequence Synthetic polypeptide”
GFTFSNYGISYDGSNKAKGLLVASIYDAFDI

SEQ ID NO: 1379 - protein, 27 aa, Artificial Sequence
  source: /note=“Description of Artificial Sequence Synthetic peptide”
GFTFSDYAISWNSGSIAKDIAAGGLDS

SEQ ID NO: 1380 - protein, 30 aa, Artificial Sequence
  source: /note=“Description of Artificial Sequence Synthetic polypeptide”
GFTFSDYYVSGSGTSTAKDWAGYINGWYGN

SEQ ID NO: 1381 - protein, 29 aa, Artificial Sequence
  source: /note=“Description of Artificial Sequence Synthetic peptide”
GFTFSSYAINPNSGDTAREQWLGPAHFDY

SEQ ID NO: 1382 - protein, 31 aa, Artificial Sequence
  source: /note=“Description of Artificial Sequence Synthetic polypeptide”
GFTFSSYAINPNSGGTARERNRAGEFSAFDI

SEQ ID NO: 1383 - protein, 25 aa, Artificial Sequence
  source: /note=“Description of Artificial Sequence Synthetic peptide”
GFTFSSYAIEPGNGDTARGASGLDF

SEQ ID NO: 1384 - protein, 30 aa, Artificial Sequence
  source: /note=“Description of Artificial Sequence Synthetic polypeptide”
GFTFSSYAIKQDGSEKARDLHCGSSCGPEA

SEQ ID NO: 1385 - protein, 33 aa, Artificial Sequence
  source: /note=“Description of Artificial Sequence Synthetic polypeptide”
GFTFSSYAISAYNGNTARDPVYSSSWGGYAFDI

SEQ ID NO: 1386 - protein, 31 aa, Artificial Sequence
  source: /note=“Description of Artificial Sequence Synthetic polypeptide”
GFTFSSYAISAYNGNTARDTFGGGSYYGHGY

SEQ ID NO: 1387 - protein, 29 aa, Artificial Sequence
  source: /note=“Description of Artificial Sequence Synthetic peptide”
GFTFSSYAISNDGVNNARENSNAWKVMDV

SEQ ID NO: 1388 - protein, 30 aa, Artificial Sequence
  source: /note=“Description of Artificial Sequence Synthetic polypeptide”
GFTFSSYAISGSGDRTAKDWAGYINGWYGN

SEQ ID NO: 1389 - protein, 30 aa, Artificial Sequence
  source: /note=“Description of Artificial Sequence Synthetic polypeptide”
GFTFSSYAISGSGGRTAKDWAGYINGWYGN

SEQ ID NO: 1390 - protein, 30 aa, Artificial Sequence
  source: /note=“Description of Artificial Sequence Synthetic polypeptide”
GFTFSSYAISGSGGRTAKDWAGYIDGWYGN

SEQ ID NO: 1391 - protein, 30 aa, Artificial Sequence
  source: /note=“Description of Artificial Sequence Synthetic polypeptide”
GFTFSSYAISGSGGRTAKDWGAYSSGWYGD

SEQ ID NO: 1392 - protein, 30 aa, Artificial Sequence
  source: /note=“Description of Artificial Sequence Synthetic polypeptide”
GFTFSSYAISGSGGNIAKDWAGYSNGWYGS

SEQ ID NO: 1393 - protein, 30 aa, Artificial Sequence
  source: /note=“Description of Artificial Sequence Synthetic polypeptide”
GFTFSSYAISGSGGITAKDWAGYSNGWFGS

SEQ ID NO: 1394 - protein, 28 aa, Artificial Sequence
  source: /note=“Description of Artificial Sequence Synthetic peptide”
GFTFSSYAISYDGGNKARVGSGGWTPDY

SEQ ID NO: 1395 - protein, 30 aa, Artificial Sequence
  source: /note=“Description of Artificial Sequence Synthetic polypeptide”
GFTFSSYAISYDGSNQAVGVGFITDGYFQH

SEQ ID NO: 1396 - protein, 28 aa, Artificial Sequence
  source: /note=“Description of Artificial Sequence Synthetic peptide”
GFTFSSYAISYDGSNKARVGSGGWTPDY

SEQ ID NO: 1397 - protein, 28 aa, Artificial Sequence
  source: /note=“Description of Artificial Sequence Synthetic peptide”
GFTFSSYAISYDGSNKARVGSGGWTPDY

SEQ ID NO: 1398 - protein, 29 aa, Artificial Sequence
  source: /note=“Description of Artificial Sequence Synthetic peptide”
GFTFSSYAISYDGSNKAKQQWLGTWYFDL

SEQ ID NO: 1399 - protein, 34 aa, Artificial Sequence
  source: /note=“Description of Artificial Sequence Synthetic polypeptide”
GFTFSSYAISYDGSNKAKEWGGGDSPTDMGLFDY

SEQ ID NO: 1400 - protein, 28 aa, Artificial Sequence
  source: /note=“Description of Artificial Sequence Synthetic peptide”
GFTFSSYAISYDGSNKTRVGSGGWTPDY

SEQ ID NO: 1401 - protein, 29 aa, Artificial Sequence
  source: /note=“Description of Artificial Sequence Synthetic peptide”
GFTFSSYAIWYDGNNKARDNSGSYNWFNP

SEQ ID NO: 1402 - protein, 28 aa, Artificial Sequence
  source: /note=“Description of Artificial Sequence Synthetic peptide”
GFTFSSYAIYPGDSDTARSHGGSNWFDP

SEQ ID NO: 1403 - protein, 27 aa, Artificial Sequence
  source: /note=“Description of Artificial Sequence Synthetic peptide”
GFTFSSYAIYPGDSDTATSLGDDAFDI

SEQ ID NO: 1404 - protein, 29 aa, Artificial Sequence
  source: /note=“Description of Artificial Sequence Synthetic peptide”
GFTFSSYAIYPGDSETARLGHSGSWYFDL

SEQ ID NO: 1405 - protein, 27 aa, Artificial Sequence
  source: /note=“Description of Artificial Sequence Synthetic peptide”
GFTFSSYAIYSGGSTARDLSYSDAFDI

SEQ ID NO: 1406 - protein, 27 aa, Artificial Sequence
  source: /note=“Description of Artificial Sequence Synthetic peptide”
GFTFSSYAIYSGGSTARDMTTVDAFDI

SEQ ID NO: 1407 - protein, 26 aa, Artificial Sequence
  source: /note=“Description of Artificial Sequence Synthetic peptide”
GFTFSSYAIYSGGSTARDTASGGMDV

SEQ ID NO: 1408 - protein, 27 aa, Artificial Sequence
  source: /note=“Description of Artificial Sequence Synthetic peptide”
GFTFSSYAFYSGGSTAREPYPGGPFDI

SEQ ID NO: 1409 - protein, 26 aa, Artificial Sequence
  source: /note=“Description of Artificial Sequence Synthetic peptide”
GFTFSSYGISASGGSTANLYGDYNAY

SEQ ID NO: 1410 - protein, 30 aa, Artificial Sequence
  source: /note=“Description of Artificial Sequence Synthetic polypeptide”
GFTFSSYGISGSGDRTAKDWAGYINGWYGN

SEQ ID NO: 1411 - protein, 30 aa, Artificial Sequence
  source: /note=“Description of Artificial Sequence Synthetic polypeptide”
GFTFSSYGISGSGGRTAKDWAGYINGWYGN

SEQ ID NO: 1412 - protein, 30 aa, Artificial Sequence
  source: /note=“Description of Artificial Sequence Synthetic polypeptide”
GFTFSSYGISGSGGITAKDWAGYTNGWYGS

SEQ ID NO: 1413 - protein, 23 aa, Artificial Sequence
  source: /note=“Description of Artificial Sequence Synthetic peptide”
GFTFSSYGISGSGGSTAKDLVLG

SEQ ID NO: 1414 - protein, 31 aa, Artificial Sequence
  source: /note=“Description of Artificial Sequence Synthetic polypeptide”
GFTFSSYGISWNSGSIAKDWDSSGYWPLFDY

SEQ ID NO: 1415 - protein, 28 aa, Artificial Sequence
  source: /note=“Description of Artificial Sequence Synthetic peptide”
GFTFSSYGISYDGSNKARVGSGGWTPDY

SEQ ID NO: 1416 - protein, 28 aa, Artificial Sequence
  source: /note=“Description of Artificial Sequence Synthetic peptide”
GFTFSSYGISYDGSNKARVGSGGWTPDY

SEQ ID NO: 1417 - protein, 28 aa, Artificial Sequence
  source: /note=“Description of Artificial Sequence Synthetic peptide”
GFTFSSYGIWYDGSNKAREVVGSYYLDY

SEQ ID NO: 1418 - protein, 32 aa, Artificial Sequence
  source: /note=“Description of Artificial Sequence Synthetic polypeptide”
GFTFSSYPINPNSGGTARGGDCSSTSCYDPDY

SEQ ID NO: 1419 - protein, 30 aa, Artificial Sequence
  source: /note=“Description of Artificial Sequence Synthetic polypeptide”
GFTFSSYPIKQDGSEKARIGRFGRKYGMDV

SEQ ID NO: 1420 - protein, 27 aa, Artificial Sequence
  source: /note=“Description of Artificial Sequence Synthetic peptide”
GFTFSSYPISAYNGNTARGLGDSSSSY

SEQ ID NO: 1421 - protein, 30 aa, Artificial Sequence
  source: /note=“Description of Artificial Sequence Synthetic polypeptide”
GFTFSSYPISGSGDITAKDWAGYVNGWYGN

SEQ ID NO: 1422 - protein, 30 aa, Artificial Sequence; identical to SEQ ID NO: 1421
  source: /note=“Description of Artificial Sequence Synthetic polypeptide”

SEQ ID NO: 1423 - protein, 30 aa, Artificial Sequence
  source: /note=“Description of Artificial Sequence Synthetic polypeptide”
GFTFSSYPISGSGGRTAKDWAGYINGWYGN

SEQ ID NO: 1424 - protein, 30 aa, Artificial Sequence
  source: /note=“Description of Artificial Sequence Synthetic polypeptide”
GFTFSSYPISGSGGRTAKDWGAYSSGWYGD

SEQ ID NO: 1425 - protein, 30 aa, Artificial Sequence
  source: /note=“Description of Artificial Sequence Synthetic polypeptide”
GFTFSSYPISGSGGITAKDWAGYTNGWYGS

SEQ ID NO: 1426 - protein, 30 aa, Artificial Sequence
  source: /note=“Description of Artificial Sequence Synthetic polypeptide”
GFTFSSYPISGTGGRTAKDWAGYINGWYGS

SEQ ID NO: 1427 - protein, 31 aa, Artificial Sequence
  source: /note=“Description of Artificial Sequence Synthetic polypeptide”
GFTFSSYPISYDATNNAKERFTGGYYTYFDY

SEQ ID NO: 1428 - protein, 25 aa, Artificial Sequence
  source: /note=“Description of Artificial Sequence Synthetic peptide”
GFTFSSYPIYHSGSTARAGGLHLDY

SEQ ID NO: 1429 - protein, 27 aa, Artificial Sequence
  source: /note=“Description of Artificial Sequence Synthetic peptide”
GFTFSSYPIYPGDSDTARGNGDGGFDY

SEQ ID NO: 1430 - protein, 30 aa, Artificial Sequence
  source: /note=“Description of Artificial Sequence Synthetic polypeptide”
GFTFSSYSISGSGGRTAKDWAGYINGWYGN

SEQ ID NO: 1431 - protein, 30 aa, Artificial Sequence
  source: /note=“Description of Artificial Sequence Synthetic polypeptide”
GFTFSSYWISGSGDITAKDWAGYVNGWYGN

SEQ ID NO: 1432 - protein, 30 aa, Artificial Sequence
  source: /note=“Description of Artificial Sequence Synthetic polypeptide”
GFTFSSYWISYDGSNKARDRGVEGAYGMDV

SEQ ID NO: 1433 - protein, 31 aa, Artificial Sequence
  source: /note=“Description of Artificial Sequence Synthetic polypeptide”
GFTFSSYWISYDGSNKAKGLLVASIYDAFDI

SEQ ID NO: 1434 - protein, 24 aa, Artificial Sequence
  source: /note=“Description of Artificial Sequence Synthetic peptide”
GFTFSSYWIYHSGSTARGSNIFDI

SEQ ID NO: 1435 - protein, 27 aa, Artificial Sequence
  source: /note=“Description of Artificial Sequence Synthetic peptide”
GFTFSTYAIKSKNDGGTTTTAPSLMDV

SEQ ID NO: 1436 - protein, 30 aa, Artificial Sequence
  source: /note=“Description of Artificial Sequence Synthetic polypeptide”
GFTFSTYAISAYNGNTARDLTFGSGPTRDY

SEQ ID NO: 1437 - protein, 30 aa, Artificial Sequence
  source: /note=“Description of Artificial Sequence Synthetic polypeptide”
GFTFSTYAISGSGDITAKDWAGYTNGWYGS

SEQ ID NO: 1438 - protein, 30 aa, Artificial Sequence
  source: /note=“Description of Artificial Sequence Synthetic polypeptide”
GFTFSTYAISGSGDITAKDWAGYVNGWYGN

SEQ ID NO: 1439 - protein, 30 aa, Artificial Sequence
  source: /note=“Description of Artificial Sequence Synthetic polypeptide”
GFTFSTYAISGSGGRTAKDWGAYSSGWYGD

SEQ ID NO: 1440 - protein, 30 aa, Artificial Sequence
  source: /note=“Description of Artificial Sequence Synthetic polypeptide”
GFTFSTYAISGSGGSTAKDWAGYINGWYGN

SEQ ID NO: 1441 - protein, 31 aa, Artificial Sequence
  source: /note=“Description of Artificial Sequence Synthetic polypeptide”
GFTFSTYAISGSGGSTAKDWTNQWLDAYFDY

SEQ ID NO: 1442 - protein, 37 aa, Artificial Sequence
  source: /note=“Description of Artificial Sequence Synthetic polypeptide”
GFTFSTYAISGSGGSTAKETILYDILTGYYNEGAFDI

SEQ ID NO: 1443 - protein, 32 aa, Artificial Sequence
  source: /note=“Description of Artificial Sequence Synthetic polypeptide”
GFTFSTYAISYDGSNKAKDWGRFGELLEGSPY

SEQ ID NO: 1444 - protein, 35 aa, Artificial Sequence
  source: /note=“Description of Artificial Sequence Synthetic polypeptide”
GFTFSTYATYYRSKWYNAREFQDSSSWYEGRAFDI

SEQ ID NO: 1445 - protein, 32 aa, Artificial Sequence
  source: /note=“Description of Artificial Sequence Synthetic polypeptide”
GFTVSSNYINPNSGGTARDWGRGVGDSGFVDY

SEQ ID NO: 1446 - protein, 27 aa, Artificial Sequence
  source: /note=“Description of Artificial Sequence Synthetic peptide”
GFTVSSNYINPKSGGAARDFVGASLDY

SEQ ID NO: 1447 - protein, 30 aa, Artificial Sequence
  source: /note=“Description of Artificial Sequence Synthetic polypeptide”
GFTVSSNYISGSGDRTAKDWAGYINGWYGN

SEQ ID NO: 1448 - protein, 31 aa, Artificial Sequence
  source: /note=“Description of Artificial Sequence Synthetic polypeptide”
GFTVSSNYISSSGSTIARGYLGAWNPDFYDY

SEQ ID NO: 1449 - protein, 28 aa, Artificial Sequence
  source: /note=“Description of Artificial Sequence Synthetic peptide”
GFTVSSNYISYDGSNKARVGSGGWTPDY

SEQ ID NO: 1450 - protein, 29 aa, Artificial Sequence
  source: /note=“Description of Artificial Sequence Synthetic peptide”
GFTVSSNYITGSGGTAKDWAGYINGWFGS

SEQ ID NO: 1451 - protein, 27 aa, Artificial Sequence
  source: /note=“Description of Artificial Sequence Synthetic peptide”
GFTVSSNYIYPGDSDTARLGDGSNFDY

SEQ ID NO: 1452 - protein, 33 aa, Artificial Sequence
  source: /note=“Description of Artificial Sequence Synthetic polypeptide”
GFTVSSNYTYYRSKWYNAREKIAVAGYYYGMDV

SEQ ID NO: 1453 - protein, 30 aa, Artificial Sequence
  source: /note=“Description of Artificial Sequence Synthetic polypeptide”
GFTVSSNYTYYNRKWINARDGGWSGSALDV

SEQ ID NO: 1454 - protein, 27 aa, Artificial Sequence
  source: /note=“Description of Artificial Sequence Synthetic peptide”
GYRFTSYWIYSGGSTARDLHSAAGFDY

SEQ ID NO: 1455 - protein, 27 aa, Artificial Sequence
  source: /note=“Description of Artificial Sequence Synthetic peptide”
GYSFTRYWIKSKNDGGTTTTAPSLMDV

SEQ ID NO: 1456 - protein, 30 aa, Artificial Sequence
  source: /note=“Description of Artificial Sequence Synthetic polypeptide”
GYSFTSYWISGSGDRTAKDWAGYINGWYGN

SEQ ID NO: 1457 - protein, 30 aa, Artificial Sequence; identical to SEQ ID NO: 1456
  source: /note=“Description of Artificial Sequence Synthetic polypeptide”

SEQ ID NO: 1458 - protein, 30 aa, Artificial Sequence
  source: /note=“Description of Artificial Sequence Synthetic polypeptide”
GYSFTSYWISYDGSNKAKGSSPYYYYGMDV

SEQ ID NO: 1459 - protein, 26 aa, Artificial Sequence
  source: /note=“Description of Artificial Sequence Synthetic peptide”
GYSFTSYWIYHSGSTARDGGSGWYDY

SEQ ID NO: 1460 - protein, 26 aa, Artificial Sequence
  source: /note=“Description of Artificial Sequence Synthetic peptide”
GYSFTSYWIYSGGSTARDTASGGMDV

SEQ ID NO: 1461 - protein, 32 aa, Artificial Sequence
  source: /note=“Description of Artificial Sequence Synthetic polypeptide”
GYSFTSYWTYYRSKWYNARGVTVPYYYYGMDV

SEQ ID NO: 1462 - protein, 29 aa, Artificial Sequence
  source: /note=“Description of Artificial Sequence Synthetic peptide”
GYSFTSYWTYYRSKWYNARSSGSYGYFQH

SEQ ID NO: 1463 - protein, 32 aa, Artificial Sequence
  source: /note=“Description of Artificial Sequence Synthetic polypeptide”
GYTFTRNATYYRSKWYNAREGTDIYYYYGMDV

SEQ ID NO: 1464 - protein, 28 aa, Artificial Sequence
  source: /note=“Description of Artificial Sequence Synthetic peptide”
GYTFTGYYIDYSGSTARDGWIRKEAFDP

SEQ ID NO: 1465 - protein, 27 aa, Artificial Sequence
  source: /note=“Description of Artificial Sequence Synthetic peptide”
GYTFTGYYIKSKNDGGTTTTAPSLMDV

SEQ ID NO: 1466 - protein, 31 aa, Artificial Sequence
  source: /note=“Description of Artificial Sequence Synthetic polypeptide”
GYTFTGYYISAYNGNTARDPGGYYYYYGMDV

SEQ ID NO: 1467 - protein, 28 aa, Artificial Sequence
  source: /note=“Description of Artificial Sequence Synthetic peptide”
GYTFTGYYISYDGSNKARVGSGGWTPDY

SEQ ID NO: 1468 - protein, 31 aa, Artificial Sequence
  source: /note=“Description of Artificial Sequence Synthetic polypeptide”
GYTFTGYYISYDGSNKAKLGGSYSIYYGMDV

SEQ ID NO: 1469 - protein, 27 aa, Artificial Sequence
  source: /note=“Description of Artificial Sequence Synthetic peptide”
GYTFTGYYIYPGDSETARDGGNYQFDY

SEQ ID NO: 1470 - protein, 32 aa, Artificial Sequence
  source: /note=“Description of Artificial Sequence Synthetic polypeptide”
GYTFTSYAIIPIFGTAARTGRSGSYYSDAFDI

SEQ ID NO: 1471 - protein, 31 aa, Artificial Sequence
  source: /note=“Description of Artificial Sequence Synthetic polypeptide”
GYTFTSYGINPSGGSTAREDHDYSNQGGFDY

SEQ ID NO: 1472 - protein, 33 aa, Artificial Sequence
  source: /note=“Description of Artificial Sequence Synthetic polypeptide”
GYTFTSYGIIPIFGTAAARAPGGSSYYYYGMDV

SEQ ID NO: 1473 - protein, 31 aa, Artificial Sequence
  source: /note=“Description of Artificial Sequence Synthetic polypeptide”
GYTFTSYGISAYNGNTARDPGYDFWSGYSDV

SEQ ID NO: 1474 - protein, 30 aa, Artificial Sequence
  source: /note=“Description of Artificial Sequence Synthetic polypeptide”
GYTFTSYGISGSGGRTAKDWAGYINGWYGN

SEQ ID NO: 1475 - protein, 35 aa, Artificial Sequence
  source: /note=“Description of Artificial Sequence Synthetic polypeptide”
GYTFTSYGISWNSGSIAKDMWGSLSIVGATRAFDY

SEQ ID NO: 1476 - protein, 29 aa, Artificial Sequence
  source: /note=“Description of Artificial Sequence Synthetic peptide”
GYTFTSYGITGSGGTAKDWAGYINGWFGS

SEQ ID NO: 1477 - protein, 35 aa, Artificial Sequence
  source: /note=“Description of Artificial Sequence Synthetic polypeptide”
GYTFTSYGIYHSGSTARGPLLIAAAGTDYYYGMDV

SEQ ID NO: 1478 - protein, 30 aa, Artificial Sequence
  source: /note=“Description of Artificial Sequence Synthetic polypeptide”
GYTFTSYYISGSGGSTASSYGGNPLDAFDI

SEQ ID NO: 1479 - protein, 35 aa, Artificial Sequence
  source: /note=“Description of Artificial Sequence Synthetic polypeptide”
GDSVSSNSAATYYRSKWYNAREKIAVAGYYYGMDV

SEQ ID NO: 1480 - protein, 37 aa, Artificial Sequence
  source: /note=“Description of Artificial Sequence Synthetic polypeptide”
GDSVSSNSAATYYRSKWYNAREFQDSSSWYEGRAFDI

SEQ ID NO: 1481 - protein, 34 aa, Artificial Sequence
  source: /note=“Description of Artificial Sequence Synthetic polypeptide”
GDSVSSNSAATYYRSKWYNARGGVGATWYYGMDV

SEQ ID NO: 1482 - protein, 27 aa, Artificial Sequence
  source: /note=“Description of Artificial Sequence Synthetic peptide”
GFTFDDYAISWNSGSIAKDIAAGGLDS

SEQ ID NO: 1483 - protein, 27 aa, Artificial Sequence
  source: /note=“Description of Artificial Sequence Synthetic peptide”
GFTFSNAWIKSKNDGGTTTTAPSLMDV

SEQ ID NO: 1484 - protein, 27 aa, Artificial Sequence
  source: /note=“Description of Artificial Sequence Synthetic peptide”
GFTFSNAWIKSKNDGGTTTTAPSLMDV

SEQ ID NO: 1485 - protein, 30 aa, Artificial Sequence
  source: /note=“Description of Artificial Sequence Synthetic polypeptide”
GFTFSSYAISYDGSNKARDRGVEGAYGMDV

SEQ ID NO: 1486 - protein, 33 aa, Artificial Sequence
  source: /note=“Description of Artificial Sequence Synthetic polypeptide”
GFTFSSYGISGSGGSTAKATGYSSGWYGAYFDY

SEQ ID NO: 1487 - protein, 30 aa, Artificial Sequence
  source: /note=“Description of Artificial Sequence Synthetic polypeptide”
GFTFSSYGISYDGSNKAKGSSPYYYYGMDV

SEQ ID NO: 1488 - protein, 29 aa, Artificial Sequence
  source: /note=“Description of Artificial Sequence Synthetic peptide”
GFTFSSYGIWYDGNNKARDNSGSYNWFNP

SEQ ID NO: 1489 - protein, 28 aa, Artificial Sequence
  source: /note=“Description of Artificial Sequence Synthetic peptide”
GFTFSSYGIWYDGSNKAREVVGSYYLDY

SEQ ID NO: 1490 - protein, 28 aa, Artificial Sequence
  source: /note=“Description of Artificial Sequence Synthetic peptide”
GFTFSSYPISYDGGNKARVGSGGWTPDY

SEQ ID NO: 1491 - protein, 28 aa, Artificial Sequence
  source: /note=“Description of Artificial Sequence Synthetic peptide”
GFTFSSYPISYDGSNKARVGSGGWTPDY

SEQ ID NO: 1492 - protein, 28 aa, Artificial Sequence
  source: /note=“Description of Artificial Sequence Synthetic peptide”
GFTFSSYPISYDGSNKARVGSGGWTPDY

SEQ ID NO: 1493 - protein, 28 aa, Artificial Sequence
  source: /note=“Description of Artificial Sequence Synthetic peptide”
GFTFSSYPISYDGSNKARVGSGGWTPDY

SEQ ID NO: 1494 - protein, 28 aa, Artificial Sequence
  source: /note=“Description of Artificial Sequence Synthetic peptide”
GFTFSSYPISYDGSNKARVGSGGWTPDY

SEQ ID NO: 1495 - protein, 28 aa, Artificial Sequence
  source: /note=“Description of Artificial Sequence Synthetic peptide”
GFTFSSYPISYDGSNKTRVGSGGWTPDY

SEQ ID NO: 1496 - protein, 27 aa, Artificial Sequence
  source: /note=“Description of Artificial Sequence Synthetic peptide”
GFTFSSYSIWYDGSNKARLGSGWSLDY

SEQ ID NO: 1497 - protein, 30 aa, Artificial Sequence
  source: /note=“Description of Artificial Sequence Synthetic polypeptide”
GFTFSSYWIKQDGSEKARDLHCGSSCGPEA

SEQ ID NO: 1498 - protein, 27 aa, Artificial Sequence
  source: /note=“Description of Artificial Sequence Synthetic peptide”
GFTVSSNYIYSGGSTARDLHSAAGFDY

SEQ ID NO: 1499 - protein, 27 aa, Artificial Sequence
  source: /note=“Description of Artificial Sequence Synthetic peptide”
GFTVSSNYIYSGGSTARDLSYSDAFDI

SEQ ID NO: 1500 - protein, 27 aa, Artificial Sequence
  source: /note=“Description of Artificial Sequence Synthetic peptide”
GFTVSSNYIYSGGSTARDFEGSGALDV

SEQ ID NO: 1501 - protein, 26 aa, Artificial Sequence
  source: /note=“Description of Artificial Sequence Synthetic peptide”
GFTVSSNYIYSGGSTARDTASGGMDV

SEQ ID NO: 1502 - protein, 26 aa, Artificial Sequence
  source: /note=“Description of Artificial Sequence Synthetic peptide”
GFTVSSNYIYSGGSTARDTASGGMDV

SEQ ID NO: 1503 - protein, 27 aa, Artificial Sequence
  source: /note=“Description of Artificial Sequence Synthetic peptide”
GYSFTSYWIYPGDSDTASGASPYYFDY

SEQ ID NO: 1504 - protein, 32 aa, Artificial Sequence
  source: /note=“Description of Artificial Sequence Synthetic polypeptide”
GYTFTGYYINPNSGGTARGGDCSSTSCYDPDY

SEQ ID NO: 1505 - protein, 33 aa, Artificial Sequence
  source: /note=“Description of Artificial Sequence Synthetic polypeptide”
GYTFTSYGISAYNGNTARDPVYSSSWGGYAFDI

SEQ ID NO: 1506 - protein, 27 aa, Artificial Sequence
  source: /note=“Description of Artificial Sequence Synthetic peptide”
GYTFTSYGISAYNGNTARGLGDSSSSY

SEQ ID NO: 1507 - protein, 31 aa, Artificial Sequence
  source: /note=“Description of Artificial Sequence Synthetic polypeptide”
GYTFTSYYINPSGGSTAREDHDYSNQGGFDY